SEQUENCE LISTING (62 sequences)

SEQ ID NO: 1 - DNA, 3014 bp, Homo sapiens
ggggccgcgtcgacgcggaaaagaggccgtggggggcctcccagcgctggcagacaccgtgaggctggcagccgccggcacgcacacctagtccgcagtcccgaggaacatgtccgcagccagggcgcggagcagagtcccgggcaggagaaccaagggagggcgtgtgctgtggcggcggcggcagcggcagcggagccgctagtcccctccctcctgggggagcagctgccgccgctgccgccgccgccaccaccatcagcgcgcggggcccggccagagcgagccgggcgagcggcgcgctagggggagggcgggggcggggaggggggtgggcgaagggggcgggagggcgtggggggagggtctcgctctcccgactaccagagcccgagggagaccctggcggcggcggcggcgcctgacactcggcgcctcctgccgtgctccggggcggcatgtccgaggctggcggggccgggccgggcggctgcggggcaggagccggggcaggggccgggcccggggcgctgcccccgcagcctgcggcgcttccgcccgcgcccccgcagggctccccctgcgccgctgccgccgggggctcgggcgcctgcggtccggcgacggcagtggctgcagcgggcacggccgaaggaccgggaggcggtggctcggcccgaatcgccgtgaagaaagcgcaactacgctccgctccgcgggccaagaaactggagaaactcggagtgtactccgcctgcaaggccgaggagtcttgtaaatgtaatggctggaaaaaccctaacccctcacccactccccccagagccgacctgcagcaaataattgtcagtctaacagaatcctgtcggagttgtagccatgccctagctgctcatgtttcccacctggagaatgtgtcagaggaagaaatgaacagactcctgggaatagtattggatgtggaatatctctttacctgtgtccacaaggaagaagatgcagataccaaacaagtttatttctatctatttaagctcttgagaaagtctattttacaaagaggaaaacctgtggttgaaggctctttggaaaagaaacccccatttgaaaaacctagcattgaacagggtgtgaataactttgtgcagtacaaatttagtcacctgccagcaaaagaaaggcaaacaatagttgagttggcaaaaatgttcctaaaccgcatcaactattggcatctggaggcaccatctcaacgaagactgcgatctcccaatgatgatatttctggatacaaagagaactacacaaggtggctgtgttactgcaacgtgccacagttctgcgacagtctacctcggtacgaaaccacacaggtgtttgggagaacattgcttcgctcggtcttcactgttatgaggcgacaactcctggaacaagcaagacaggaaaaagataaactgcctcttgaaaaacgaactctaatcctcactcatttcccaaaatttctgtccatgctagaagaagaagtatatagtcaaaactctcccatctgggatcaggattttctctcagcctcttccagaaccagccagctaggcatccaaacagttatcaatccacctcctgtggctgggacaatttcatacaattcaacctcatcttcccttgagcagccaaacgcagggagcagcagtcctgcctgcaaagcctcttctggacttgaggcaaacccaggagaaaagaggaaaatgactgattctcatgttctggaggaggccaagaaaccccgagttatgggggatattccgatggaattaatcaacgaggttatgtctaccatcacggaccctgcagcaatgcttggaccagagaccaattttctgtcagcacactcggccagggatgaggcggcaaggttggaagagcgcaggggtgtaattgaatttcacgtggttggcaattccctcaaccagaaaccaaacaagaagatcctgatgtggctggttggcctacagaacgttttctcccaccagctgccccgaatgccaaaagaatacatcacacggctcgtctttgacccgaaacacaaaacccttgctttaattaaagatggccgtgttattggtggtatctgtttccgtatgttcccatctcaaggattcacagagattgtcttctgtgctgtaacctcaaatgagcaagtcaagggctatggaacacacctgatgaatcatttgaaagaatatcacataaagcatgacatcctgaacttcctcacatatgcagatgaatatgcaattggatactttaagaaacagggtttctccaaagaaattaaaatacctaaaaccaaatatgttggctatatcaaggattatgaaggagccactttaatgggatgtgagctaaatccacggatcccgtacacagaattttctgtcatcattaaaaagcagaaggagataattaaaaaactgattgaaagaaaacaggcacaaattcgaaaagtttaccctggactttcatgttttaaagatggagttcgacagattcctatagaaagcattcctggaattagagagacaggctggaaaccgagtggaaaagagaaaagtaaagagcccagagaccctgaccagctttacagcacgctcaagagcatcctccagcaggtgaagagccatcaaagcgcttggcccttcatggaacctgtgaagagaacagaagctccaggatattatgaagttataaggttccccatggatctgaaaaccatgagtgaacgcctcaagaataggtactacgtgtctaagaaattattcatggcagacttacagcgagtctttaccaattgcaaagagtacaacgccgctgagagtgaatactacaaatgtgccaatatcctggagaaattcttcttcagtaaaattaaggaagctggattaattgacaagtgattttttttccccctctgcttcttagaaactcaccaagcagtgtgcctaaagcaaggt

SEQ ID NO: 2 - protein, 832 aa, Homo sapiens
MSEAGGAGPGGCGAGAGAGAGPGALPPQPAALPPAPPQGSPCAAAAGGSGACGPATAVAAAGTAEGPGGGGSARIAVKKAQLRSAPRAKKLEKLGVYSACKAEESCKCNGWKNPNPSPTPPRADLQQIIVSLTESCRSCSHALAAHVSHLENVSEEEMNRLLGIVLDVEYLFTCVHKEEDADTKQVYFYLFKLLRKSILQRGKPVVEGSLEKKPPFEKPSIEQGVNNFVQYKFSHLPAKERQTIVELAKMFLNRINYWHLEAPSQRRLRSPNDDISGYKENYTRWLCYCNVPQFCDSLPRYETTQVFGRTLLRSVFTVMRRQLLEQARQEKDKLPLEKRTLILTHFPKFLSMLEEEVYSQNSPIWDQDFLSASSRTSQLGIQTVINPPPVAGTISYNSTSSSLEQPNAGSSSPACKASSGLEANPGEKRKMTDSHVLEEAKKPRVMGDIPMELINEVMSTITDPAAMLGPETNFLSAHSARDEAARLEERRGVIEFHVVGNSLNQKPNKKILMWLVGLQNVFSHQLPRMPKEYITRLVFDPKHKTLALIKDGRVIGGICFRMFPSQGFTEIVFCAVTSNEQVKGYGTHLMNHLKEYHIKHDILNFLTYADEYAIGYFKKQGFSKEIKIPKTKYVGYIKDYEGATLMGCELNPRIPYTEFSVIIKKQKEIIKKLIERKQAQIRKVYPGLSCFKDGVRQIPIESIPGIRETGWKPSGKEKSKEPRDPDQLYSTLKSILQQVKSHQSAWPFMEPVKRTEAPGYYEVIRFPMDLKTMSERLKNRYYVSKKLFMADLQRVFTNCKEYNAAESEYYKCANILEKFFFSKIKEAGLIDK

SEQ ID NO: 3 - protein, 26 aa, Artificial Sequence
  synthetic bromodomain peptide
FXXXPXXXXXXXXXXYXXXXXXPXXD

SEQ ID NO: 4 - protein, 12 aa, Artificial Sequence
  synthetic bromodomain peptide
ISYGRXKRRQRR

SEQ ID NO: 5 - protein, 14 aa, Artificial Sequence
  synthetic bromodomain peptide
ARKSTGGXAPRKQL

SEQ ID NO: 6 - protein, 14 aa, Artificial Sequence
  synthetic bromodomain peptide
QSTSRHKXLMFKTE

SEQ ID NO: 7 - protein, 110 aa, Homo sapiens
SKEPRDPDQLYSTLKSILQQVKSHQSAWPFMEPVKRTEAPGYYEVIRSPMDLKTMSERLKNRYYVSKKLFMADLQRVFTNCKEYNAPESEYYKCANILEKFFFSKIKEAG

SEQ ID NO: 8 - protein, 110 aa, Homo sapiens
GKELKDPDQLYTTLKNLLAQIKSHPSAWPFMEPVKKSEAPDYYEVIRFPIDLKTMTERLRSRYYVTRKLFVADLQRVIANCREYNPPDSEYCRCASALEKFFYFKLKEGG

SEQ ID NO: 9 - protein, 109 aa, Tetrahymena thermophila
LKKSKERSFNLQCANVIENMKRHKQSWPFLDPVNKDDVPDYYDVITDPIDIKAIEKKLQNNQYVDKDQFIKDVKRIFTNAKIYNQPDTIYYKAAKELEDFVEPYLTKLK

SEQ ID NO: 10 - protein, 109 aa, Saccharomyces cerevisiae
AQRPKRGPHDAAIQNILTELQNHAAAWPFLQPVNKEEVPDYYDFIKEPMDLSTMEIKLESNKYQKMEDFIYDARLVFNNCRMYNGENTSYYKYANRLEKFFNNKVKEIP

SEQ ID NO: 11 - protein, 112 aa, Homo sapiens
KKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVM

SEQ ID NO: 12 - protein, 112 aa, Homo sapiens
KKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVM

SEQ ID NO: 13 - protein, 112 aa, Mus musculus
KKIFKPEELRQALMPTLEALYRQDPESLPFRQPVDPQLLGIPDYFDIVKNPMDLSTIKRKLDTGQYQEPWQYVDDVRLMFNNAWLYNRKTSRVYKFCSKLAEVFEQEIDPVM

SEQ ID NO: 14 - protein, 111 aa, Caenorhabditis elegans
DTVFSQEDLIKFLLPVWEKLDKSEDAAPFRVPVDAKLLNIPDYHEIIKRPMDLETVHKKLYAGQYQNAGQFCDDIWLMLDNAWLYNRKNSKVYKYGLKLSEMFVSEMDPVM

SEQ ID NO: 15 - protein, 110 aa, Homo sapiens
RRRTDPMVTLSSILESIINDMRDLPNTYPFHTPVNAKVVKDYYKIITRPMDLQTLRENVRKRLYPSREEFREHLELIVKNSATYNGPKHSLTQISQSMLDLCDEKLKEKE

SEQ ID NO: 16 - protein, 110 aa, Mesocricetus auratus; identical to SEQ ID NO: 15

SEQ ID NO: 17 - protein, 111 aa, Homo sapiens
LLDDDDQVAFSFILDNIVTQKMMAVPDSWPFHHPVNKKFVPDYYKVIVNPMDLETIRKNISKHKYQSRESFLDDVNLILANSVKYNGPESQYTKTAQEIVNVCYQTLTEYD

SEQ ID NO: 18 - protein, 111 aa, Mesocricetus auratus
LLDDDDQVAFSFILDNIVTQKMMAVPDSWPFHHPVNKKFVPDYYKVIVSPMDLETIRKNISKHKYQSRESFLDDVNLILANSVKYNGSESQYTKTAQEIVNVCYQTLTEYD

SEQ ID NO: 19 - protein, 111 aa, Homo sapiens
KPGRVTNQLQYLHKVVMKALWKHQFAWPFRQPVDAVKLGLPDYHKIIKQPMDMGTIKRRLENNYYWAASECMQDFNTMFTNCYIYNKPTDDIVLMAQTLEKIFLQKVASMP

SEQ ID NO: 20 - protein, 111 aa, Homo sapiens
KPGRKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQMP

SEQ ID NO: 21 - protein, 111 aa, Drosophila melanogaster
RPGRNTNQLQYLIKTVMKVIWKHHFSWPFQQPVDAKKLNLPDYHKIIKQPMDMGTIKKRLENNYYWSAKETIQDFNTMFNNCYVYNKPGEDVVVMAQTLEKVFLQKIESMP

SEQ ID NO: 22 - protein, 109 aa, Saccharomyces cerevisiae
NPIPKHQQKHALLAIKAVKRLKDARPFLQPVDPVKLDIPFYFNYIKRPMDLSTIERKLNVGAYEVPEQITEDFNLMVNNSIKFNGPNAGISQMARNIQASFEKHMLNMP

SEQ ID NO: 23 - protein, 113 aa, Homo sapiens
KKGKLSEQLKHCNGILKELLSKKHAAYAWPFYKPVDASALGLHDYHDIIKHPMDLSTVKRKMENRDYRDAQEFAADVRLMFSNCYKYNPPDHDVVAMARKLQDVFEFRYAKMP

SEQ ID NO: 24 - protein, 113 aa, Homo sapiens
KKGKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMP

SEQ ID NO: 25 - protein, 113 aa, Drosophila melanogaster
NKEKLSDALKSCNEILKELFSKKHSGYAWPFYKPVDAEMLGLHDYHDIIKKPMDLGTVKRKMDNREYKSAPEFAADVRLIFTNCYKYNPPDHDVVAMGRKLQDVFEMRYANIP

SEQ ID NO: 26 - protein, 113 aa, Saccharomyces cerevisiae
KSKRLQQAMKFCQSVLKELMAKKHASYNYPFLEPVDPVSMNLPTYFDYVKEPMDLGTIAKKLNDWQYQTMEDFEREVRLVFKNCYTFNPDGTIVNMMGHRLEEVFNSKWADRP

SEQ ID NO: 27 - protein, 108 aa, Homo sapiens
MEMQLTPFLILLRKTLEQLQEKDTGNIFSEPVPLSEVPDYLDHIKKPMDFFTMKQNLEAYRYLNFDDFEEDFNLIVSNCLKYNAKDTIFYRAAVRLREQGGAVVRQAR

SEQ ID NO: 28 - protein, 113 aa, Homo sapiens
SEDQEAIQAQKIWKKAIMLVWRAAANHRYANVFLQPVTDDIAPGYHSIVQRPMDLSTIKKNIENGLIRSTAEFQRDIMLMFQNAVMYNSSDHDVYHMAVEMQRDVLEQIQQFL

SEQ ID NO: 29 - protein, 106 aa, Gallus gallus
NLPTVDPIAVCHELYNTIRDYKDEQGRLLCELFIRAPKRRNQPDYYEVVSQPIDLMKIQQKLKMEEYDDVNVLTADFQLLFNNAKAYYKPDSPEYKAACKLWELYL

SEQ ID NO: 30 - protein, 112 aa, Gallus gallus
SSPGYLKEILEQLLEAVAVATNPSGRLISELFQKLPSKVQYPDYYAIIKEPIDLKTIAQRIQNGTYKSIHAMAKDIDLLAKNAKTYNEPGSQVFKDANAIKKIFNMKKAEIE

SEQ ID NO: 31 - protein, 112 aa, Gallus gallus
TSFMDTSNPLYQLYDTVRSCRNNQGQLISEPFFQLPSKKKYPDYYQQIKTPISLQQIRAKLKNHEYETLDQLEADLNLMFENAKRYNVPNSAIYKRVLKMQQVMQAKKKELA

SEQ ID NO: 32 - protein, 113 aa, Gallus gallus
SKKNMRKQRMKILYNAVLEARESGTQRRLCDLFMVKPSKKDYPDYYKIILEPMDLKMIEHNIRNDKYVGEEAMIDDMKLMFRNARHYNEEGSQVYNDAHMLEKILKEKRKELG

SEQ ID NO: 33 - protein, 115 aa, Gallus gallus
KKSKYMTPMQQKLNEVYEAVKNYTDKRGRRLSAIFLRLPSRSELPDYYITIKKPVDMEKIRSHMMANKYQDIDSMVEDFVMMFNNACTYNEPESLIYKDALVLHKVLLETRREIE

SEQ ID NO: 34 - protein, 112 aa, Schizosaccharomyces pombe
HNAPFDKTKFDEVLEALVGLKDNEGNPFDDIFEELPSKRYFPDYYQIIQKPICYKMMRNKAKTGKYLSMGDFYDDIRLMVSNAQTYNMPGSLVYECSVLIANTANSLESKDG

SEQ ID NO: 35 - protein, 113 aa, Unknown
  source of sequence could not be determined
GTNEIDVPKVIQNILDALHEEKDEQGRFLIDIFIDLPSKRLYPDYYEIIKSPMTIKMLEKRFKKGEYTTLESFVKDLNQMFINAKTYNAPGSFVYEDAEKLSQLSSSLISSFS

SEQ ID NO: 36 - protein, 113 aa, Homo sapiens; identical to SEQ ID NO: 35

SEQ ID NO: 37 - protein, 114 aa, Homo sapiens
SPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIE

SEQ ID NO: 38 - protein, 113 aa, Gallus gallus
SPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIA

SEQ ID NO: 39 - protein, 114 aa, Gallus gallus
SPNPPNLTKKMKKIVDAVIKYKDSSSGRQLSEVFIQLPSRKELPEYYELIRKPVDFKKIKERIRNHKYRSLNDLEKDVMLLCQNAQTFNLEVSLIYEDSIVLQSVFTSVRQKIE

SEQ ID NO: 40 - protein, 105 aa, Homo sapiens
AKLSPANQRKCERVLLALFCHEPCRPLHQLATDSTFSLDQPGGTLDLTLIRARLQEKLSPPYSSPQEFAQDVGRMFKQFNKLTEDKADVQSIIGLQRFFETRMNE

SEQ ID NO: 41 - protein, 105 aa, Mus musculus
AKLSPANQRKCERVLLALFCHEPCRPLHQLATDSTFSMEQPGGTLDLTLIRARLQEKLSPPYSSPQEFAQDVGRMFKQFNKLTEDKADVQSIIGLQRFFETRMND

SEQ ID NO: 42 - protein, 108 aa, Mus sp.
TKLTPIDKRKCERLLLFLYCHEMSLAFQDPVPLTVPDYYKIIKNPMDLSTIKKRLQEDYCMYTKPEDFVADFRLIFQNCAEFNEPDSEVANAGIKLESYFEELLKNLY

SEQ ID NO: 43 - protein, 27 aa, Artificial Sequence
  synthetic bromodomain peptide
XXFXXXPXXXXXXXXXXYXXXXXXPXD

SEQ ID NO: 44 - protein, 20 aa, Artificial Sequence
  synthetic bromodomain peptide
WPFMEPVKRTEAPGYYEVIR

SEQ ID NO: 45 - protein, 101 aa, Homo Sapiens
MEPVDPRLEPWKHPGSQPKTASNNCYCKRCCLHCQVCFTKKGLGISYGRKKRRQRRRAPQDSKTHQVSLSKQPASQPRGDPTGPKESKKKVERETETDPED

SEQ ID NO: 46 - protein, 9 aa, Artificial Sequence
  Synthetic HIV-1 Tat peptide
YGRKXXXRQ

SEQ ID NO: 47 - protein, 10 aa, Artificial Sequence
  synthetic HIV-1 Tat peptide
SYGRKKRRQR

SEQ ID NO: 48 - protein, 21 aa, Artificial Sequence
  synthetic bromodomain peptide
FXXXXVXXXXEXXXXYXXXVX

SEQ ID NO: 49 - protein, 62 aa, Artificial Sequence
  synthetic bromodomain peptide
FMEPVKRTEAPGYYEVIRFPMDLKTMSERLKNRYYVSKKLFMADLQRVFTNCKEYNAAESEY

SEQ ID NO: 50 - protein, 11 aa, Artificial Sequence
  synthetic HIV-1 Tat peptide
SYGRXKRRQRC

SEQ ID NO: 51 - protein, 11 aa, Artificial Sequence
  synthetic HIV-1 Tat peptide
SAGRXKRRQRC

SEQ ID NO: 52 - protein, 11 aa, Artificial Sequence
  synthetic HIV-1 Tat peptide
SYGAXKRRQRC

SEQ ID NO: 53 - protein, 11 aa, Artificial Sequence
  synthetic HIV-1 Tat peptide
SYGRXARRQRC

SEQ ID NO: 54 - protein, 11 aa, Artificial Sequence
  synthetic HIV-1 Tat peptide
SYGRXKARQRC

SEQ ID NO: 55 - protein, 11 aa, Artificial Sequence
  synthetic HIV-1 Tat peptide
SYGRXKRAQRC

SEQ ID NO: 56 - protein, 11 aa, Artificial Sequence
  synthetic HIV-1 Tat peptide
SYGRXKRRARC

SEQ ID NO: 57 - protein, 11 aa, Artificial Sequence
  synthetic HIV-1 Tat peptide
SYGRKXRRQRC

SEQ ID NO: 58 - protein, 11 aa, Artificial Sequence
  synthetic HIV-1 Tat peptide
TNCYCKXCCFH

SEQ ID NO: 59 - protein, 20 aa, Artificial Sequence
  synthetic histone H4 AcK16 peptide
SGRGKGGKGLGKGGAXRHRK

SEQ ID NO: 60 - protein, 11 aa, Artificial Sequence
  synthetic HIV-1 Tat peptide
SYGRKKRRQRC

SEQ ID NO: 61 - protein, 12 aa, Artificial Sequence
  synthetic H4 histone peptide
SGRGKGGXGLGK

SEQ ID NO: 62 - protein, 12 aa, Artificial Sequence
  synthetic H3 histone peptide
RKSTGGXAPRKQ

(19) United States
(12) Patent Application Publication
(10) Pub. No.: US 2012/0028912 A1
(43) Pub. Date: Feb. 2, 2012

(54) Methods of modulating bromodomains

(75) Inventors: Ming-Ming Zhou, Greenwich, CT (US); Aneel K. Aggarwal, Edgewater, NJ (US); Melanie Ott, San Francisco, CA (US); Eric Verdin, San Francisco, CA (US)

(73) Assignees: J.David Gladstone Institute; Mount Sinai School of Medicine

(21) Appl. No.: 12/590,069

(22) Filed: Nov. 2, 2009

Related U.S. Application Data
(63) Continuation-in-part of application No. 12/154,158, filed on May 19, 2008.
Application No. 12/154,158 is a continuation-in-part of application No. 10/209,201, filed on Jul. 31, 2002, now abandoned.
Application No. 10/209,201 is a division of application No. 09/784,553, filed on Feb. 16, 2001, now Pat. No. 7,589,167.
Application No. 09/784,553 is a continuation-in-part of application No. 09/510,314, filed on Feb. 22, 2000, now Pat. No. 7,919,276.

(51) Int. Cl. A61K 38/10; A61K 31/135; A61P 31/18; A61K 31/16; A61K 31/352; A61K 38/08; A61K 31/195

(52) U.S. Cl. 514/21.5; 514/21.7; 514/21.6; 514/649; 514/564; 514/651; 514/630; 514/457

(57) ABSTRACT

The present invention features compounds useful for and methods for preventing or inhibiting the binding of bromodomains to acetyl-lysine residues of proteins and methods for treating HIV infection and HIV related disease.

### CROSS-REFERENCE TO RELATED APPLICATIONS

The present application is a continuation-in-part of U.S. Ser. No. 10/209,201, filed Jul. 31, 2002 which is a division of U.S. Ser. No. 09/784,553, filed Feb. 16, 2001 which is a continuation in part of U.S. Ser. No. 09/510,314, filed Feb. 22, 2000, the disclosures of which are hereby incorporated by reference in their entireties. Applicants claim the benefits of these applications under 35 U.S.C. §120.

### FIELD OF THE INVENTION

The present invention provides the three-dimensional structure of a histone acetyltransferase bromodomain. The interaction between bromodomains and their binding partners play a crucial role in various cellular functions, including in the regulation/modulation of DNA transcription. Therefore, the present invention provides methods for modulating the interaction of bromodomains and their binding partners by such agents as, for example, small molecules.

### BACKGROUND OF THE INVENTION

In recent years great strides have been made in the elucidation of the steps involved in intercellular and intracellular signaling. Indeed, the individual steps of the cascade of events involved in a number of cellular signal transduction processes have been determined. For example, intercellular signal transduction generally begins with an intercellular ligand binding the extracellular portion of a receptor of the plasma membrane. The bound receptor then either directly or indirectly initiates the activation of one or more cellular factors. An activated cellular factor may act as transcription factor by entering the nucleus to interact with its corresponding genomic response element, or alternatively, it may interact with other cellular factors depending on the complexity of the process. In either case, one or more transcription factors ultimately bind to one or more specific genomic response elements. This binding plays a crucial role in the up and/or down regulation of the transcription of the specific genes that are under the control of these genomic response elements. However, the process of re-organizing the chromatin of eukaryotic cells, which is a prerequisite for the binding of the transcription factor to the genomic response elements, has remained a mystery.

Chromatin contains several highly conserved histone proteins including: H3, H4, H2A, H2B, and H1. These histone proteins package eukaryotic DNA into repeating nucleosomal units that are folded into higher-order chromatin fibers (Luger and Richmond, Curr. Opin. Genet. Dev. 8:140-146 (1998)). A portion of the histone that comprises roughly a quarter of the protein protrudes from the chromatin surface, and is thereby sensitive to proteolytic enzymes (van Holde, in Chromatin (Rich, A., ed., Springer, N.Y.) pages 111-148 (1988); Hect et al., Cell 80:583-592 (1995)). This portion of the histone is known as the “histone tail”. Histone tails tend to be free for protein-protein interaction, and are also the portion of the histone most prone to post-translational modification. Such post-translational modification includes acetylation, phosphorylation, methylation, ubiquitination, and ADP-ribosylation (van Holde, in Chromatin (Rich, A,. ed., Springer, N.Y.) pages 111-148 (1988)).

Of all classes of proteins, histones are amongst the most susceptible to post-translational modification. Perhaps the best studied post-translational modification of histones is the acetylation of specific lysine residues (Grunstin, M., Nature, 389:349-352 (1997)). Indeed, acetylation of histone lysine residues has been suggested to play a pivotal role in chromatin remodeling and gene activation. Consistently, distinct classes of enzymes, namely histone acetyltransferases (HATs) and histone deacetylases (HDACs), acetylate or de-acetylate specific histone lysine residues (Struhl, Genes Dev. 12:599-606 (1998)).

Nearly all known nuclear HATs contain an approximately 110 amino acid sequence known as the bromodomain (Jeanmougin et al., Trends in Biochemical Sciences, 22:151-153 (1997)), a protein motif that was initially discovered in Drosophila brahma protein. Bromodomains are found in a large number of chromatin-associated proteins and have now been identified in approximately 42 human proteins, often adjacent to other protein motifs (Jeanmougin et al., Trends in Biochemical Sciences, 22:151-153 (1997); Tamkun et al., Cell, 68:561-572 (1992): Hanes et al., Nucleic Acids Research, 20:2603 (1992); Sanchez, R. & Zhou, M.-M., Current Opinion in Drug Discovery & Development, 12(5):659-665 (2009)). Proteins that contain a bromodomain often contain a second bromodomain. However, despite the wide occurrence of bromodomains and their likely role in chromatin regulation, their three-dimensional structure and binding partners heretofore have remained unknown.

The bromodomain, present in chromatin associated proteins and histone lysine acetyltransferases,^{6a} is an acetyl-lysine binding domain.^{6b} Bromodomain/AcK binding plays an important role in control of chromatin remodeling and gene transcription.^{6c} BRDs adopt the highly conserved structural fold of a left-handed four-helix bundle (αZ, αA, αB and αC), as first shown in the PCAF BRD (Zeng, et al., FEBS Letters (2002) 513, 124-8) (FIG. 1A). The ZA and BC loops at one end of the bundle form a hydrophobic pocket for AcK binding. The structure of the PCAF BRD bound to a Tat-AcK50 peptide shows that AcK50 interacts with protein residues V752, Y802 and Y809, Y47(AcK−3) with V763, and R53(AcK+3) and Q54(AcK+4) with E756, conferring a specific intermolecular association. The structures of CBP BRD/p53-AcK382 and GCN5p BRD/H4-AcK16 complexes show that the residues in BRDs important for AcK recognition are largely conserved, whereas sequence variations in the ZA and BC loops enable discrimination of different binding targets (Mujtaba, et al., Mol Cell. (2004) 13, 251-63). Notably, as compared to the other parts of the protein, the ZA and BC loops contain significant sequence variations with amino acid deletion or insertion, supporting the notion that different sets of residues in the ZA and/or BC loops dictate BRD ligand specificity by interacting with residues flanking the acetyl-lysine in a target protein.

Therefore, there is a need to identify binding partners for a bromodomain. In addition, there is a need to identify agonists or antagonists to the bromodomain-binding partner complex. Since a preferred method of drug-screening relies on structure based drug design, there is also a need to determine the three-dimensional structure of a bromodomain. In this case, once the three dimensional structure of bromodomain is determined, potential agonists and/or potential antagonists can be designed with the aid of computer modeling (Bugg et al., Scientific American, December: 92-98 (1993); West et al., TIPS, 16:67-74 (1995); Dunbrack et al., Folding & Design, 2:27-42 (1997). However, heretofore the three-dimensional structure of the bromodomain has remained unknown. Therefore, there is a need for obtaining a form of the bromodomain that is amenable for NMR analysis and/or X-ray crystallographic analysis. Furthermore, there is a need for the determination of the three-dimensional structure of the bromodomain. Finally, there is a need for procedures for related structural based drug design predicated on such structural data.

The replication cycle of the human immunodeficiency virus (HIV) presents several viable targets for anti-HIV chemotherapy. The current anti-HIV drugs specifically target the viral reverse transcriptase, protease and integrase (Garg, et al., Chem. Rev. (1999) 99, 3525-601) However, because of the development of viral drug resistance from mutations in the targeted proteins, continuous viral production by chronically infected cells contributes to HIV-mediated immune dysfunction (Ho, et al., Nature. Med. (2000) 6, 757-61; Wei et al., Nature (1995) 373, 117-22) and there is still no cure for AIDS. A rapid growing AIDS epidemic calls for new therapeutic strategies targeting different steps in the viral life cycle. Therapeutic intervention at the stage of HIV gene expression can complement the existing therapy to interfere with virus production. Transcription of the integrated HIV provirus is regulated by the concerted action between cellular transcription factors and a unique viral trans-activator Tat. Tat binds to a viral RNA TAR and recruits cyclin T1 and cyclin-dependent kinase 9 that hyper-phosphorylates and enhances elongation efficiency of the RNA polymerase II (Keen et al., J. EMBO. J. (1997) 16, 5260-72; Karn, J. Mol. Biol. (1999) 293, 235-54; Jones, Genes. Dev. (1997) 11, 2593-99; Kao, et al., Nature (1987) 330, 489-93).

Tat transactivation requires acetylation of its lysine 50 and recruitment of histone lysine acetyltransferase transcriptional coactivators for remodeling nucleosome that contains the integrated proviral DNA (Ott et al., Curr. Biol. (1999) 9, 1489-92). Our recent study shows that Tat coactivator recruitment requires its acetylated lysine 50 (AcK50) binding to the bromodomain (BRD) of the coactivator PCAF (Mujtaba, et al., Mol. Cell. (2002) 9, 575-86), and microinjection of anti-PCAF BRD antibody blocks Tat transactivation (Dorr et al., EMBO. J. (2002) 21, 2715-33). These data suggest that Tat/PCAF recruitment via a BRD-AcK binding is essential for HIV transcription, and this interaction serves as a new therapeutic target for intervening HIV replication.

Small molecule inhibitors that block Tat/PCAF binding by targeting the BRD of PCAF are needed. Targeting a host cell protein essential for viral reproduction, rather than a viral protein, may minimize the problem of drug resistance due to mutations of the viral counterpart as observed with protease inhibitors.

The citation of any reference herein should not be construed as an admission that such reference is available as “Prior Art” to the instant application.

### SUMMARY OF THE INVENTION

The present invention is based in part on the discovery that bromodomains bind to acetyl-lysine residues of proteins. The present invention provides the three-dimensional structure of a bromodomain as well as the three-dimensional structure of a bromodomain-acetyl-histamine complex. The structural information provided can be employed in methods of identifying drugs that can modulate the cellular processes that involve bromodomain-acetyl-lysine interactions. These interactions include chromatin remodeling, which is a required step in eukaryotic transcription. In a particular embodiment, the three-dimensional structural information is used in the identification and/design of an inhibitor of human leukemia. In another embodiment, the three-dimensional structural information is used in the identification and/design of an inhibitor of HIV-1 infection and/or AIDS.

The present invention provides an isolated nucleic acid that encodes a peptide of about 21 to 40 amino acids that contains all or a part of a ZA loop of a bromodomain. In a preferred embodiment the peptide is about 23 to 34 amino acids. The isolated nucleic acid may further contain a heterologous nucleotide sequence.

In a preferred embodiment the peptide contains the amino acid sequence of SEQ ID NO:3. In another embodiment the peptide contains the amino acid sequence of SEQ ID NO:43. In particular embodiments the ZA loop is obtained from the bromodomain having the amino acid sequence of SEQ ID NO:7, or SEQ ID NO:8, or SEQ ID NO:9, or SEQ ID NO:10, or SEQ ID NO:11, or SEQ ID NO: 12, or SEQ ID NO: 13, or SEQ ID NO: 14, or SEQ ID NO:15, or SEQ ID NO:16, or SEQ ID NO:17, or SEQ ID NO:18, or SEQ ID NO:19, or SEQ ID NO:20, or SEQ ID NO:21, or SEQ ID NO: 22, or SEQ ID NO:23, or SEQ ID NO:24, or SEQ ID NO:25, or SEQ ID NO:26, or SEQ ID NO:27, or SEQ ID NO:28, or SEQ ID NO:29, or SEQ ID NO:30, or SEQ ID NO: or SEQ ID NO:31, or SEQ ID NO:32, or SEQ ID NO: 33, or SEQ ID NO:34, or SEQ ID NO:35, or SEQ ID NO:36, or SEQ ID NO:37, or SEQ ID NO:38, or SEQ ID NO: or SEQ ID) NO:39, or SEQ ID NO:40, or SEQ ID NO:41, or SEQ ID NO:42.

The present invention further provides a recombinant DNA molecule that is an isolated nucleic acid of the present invention, as described above, with or without a heterologous nucleotide sequence. Such a recombinant DNA molecule can be operatively linked to an expression control sequence and can be part of an expression vector. The present invention further provides a cell that comprises such an expression vector. The cell can be either a eukaryotic or a prokaryotic cell. The present invention further provides a method of expressing the peptides of the present invention or fragments thereof in this cell. One such method comprises culturing the cell in an appropriate cell culture medium under conditions that provide for expression of the peptide by the cell.

The present invention further provides a peptide of about 21 to 40 amino acids that contains all or a part of a ZA loop of a bromodomain. In a preferred embodiment the peptide is about 23 to 34 amino acids. The present invention also provides fusion proteins or peptides having these peptides.

In a preferred embodiment the peptide contains the amino acid sequence of SEQ ID NO:3. In another embodiment the peptide contains the amino acid sequence of SEQ ID NO:43. In yet another preferred embodiment the peptide comprises the amino acid sequence of SEQ ID NO:48.

In particular embodiments the ZA loop is obtained from the bromodomain having the amino acid sequence of SEQ ID NO:7, or SEQ ID NO:8, or SEQ ID NO:9, or SEQ ID NO: 10, or SEQ ID NO: 11, or SEQ ID NO: 12, or SEQ ID NO: 13, or SEQ ID NO: 14, or SEQ ID NO:15, or SEQ ID NO:16, or SEQ ID NO:17, or SEQ ID NO:18, or SEQ ID NO:19, or SEQ ID NO:20, or SEQ ID NO:21, or SEQ ID NO: 22, or SEQ ID NO:23, or SEQ ID NO:24, or SEQ ID NO:25, or SEQ ID NO:26, or SEQ ID NO:27, or SEQ ID NO:28, or SEQ ID NO:29, or SEQ ID NO:30, or SEQ ID NO: or SEQ ID NO:31, or SEQ ID NO:32, or SEQ ID NO: 33, or SEQ ID NO:34, or SEQ ID NO:35, or SEQ ID NO:36, or SEQ ID NO:37, or SEQ ID NO:38, or SEQ ID NO: or SEQ ID NO:39, or SEQ ID NO:40, or SEQ ID NO:41, or SEQ ID NO:42.

In another aspect, the present invention provides antibodies raised against the peptides/proteins of the present invention, or raised against an antigenic fragment of these proteins/fragments. In a particular embodiment an antibody is raised against a fragment of the ZA loop of a bromodomain. In another embodiment an antibody is raised against a fragment of a protein or peptide that comprises an acetyl-lysine, so that the protein or peptide can bind to a bromodomain. Such fragments can be conjugated to a carrier protein or be part of a fusion protein. In one embodiment the antibody is a polyclonal antibody. In another embodiment, the antibody is a monoclonal antibody. A hybridoma that makes the monoclonal antibody is also part of the present invention. In a particular embodiment the antibody is a chimeric antibody. Antibodies that can specifically recognize acetyl-lysine residues involved bromodomain binding are also part of the present invention.

In another aspect the present invention provides a method for identifying a compound that modulates the affinity of a bromodomain for a ligand (and/or protein) that comprises an acetylated lysine or an analog of an acetylated lysine. In one embodiment the method features contacting the bromodomain and the ligand in the presence of a compound under conditions such that the bromodomain and the ligand bind in the absence of the compound. The affinity of the bromodomain for the ligand may then be determined or measured. A compound is identified as a compound that modulates the affinity of a bromodomain for a ligand when there is a change in the affinity of the bromodomain for the ligand in the presence of the compound. When the affinity of the bromodomain for the ligand increases in the presence of the compound, the compound is identified as a promoting agent for the bromodomain-ligand complex. When the affinity of the bromodomain for the ligand decreases in the presence of the compound, the compound is identified as an inhibitor of the bromodomain-ligand complex. In a preferred embodiment, the compound to be tested is pre-selected by performing rational drug design with the set of atomic coordinates obtained from one or more of Tables 1-6 of U.S. Ser. No. 09/784,553, filed Feb. 16, 2001, now U.S. Pat. No. 7,589,167, herein incorporated by reference. More preferably the selecting is performed in conjunction with computer modeling. In a particular embodiment, the compound is selected by performing rational drug design with the set of atomic coordinates obtained from a set of atomic coordinates defining the three-dimensional structure of a bromodomain consisting of the amino acid sequence of SEQ ID NO:7 alone or with acetyl-histamine.

In another aspect, the present invention provides a method of identifying a compound that modulates the stability of a bromodomain-ligand binding complex. Preferably the ligand comprises either an acetyl-lysine or an analog of acetyl-lysine. One such embodiment comprises contacting the bromodomain-ligand binding complex in the presence of the compound under conditions in which the bromodomain-ligand binding complex forms in the absence of the compound. The stability of the bromodomain-ligand binding complex is then determined (e.g., measured). A compound is identified as a compound that modulates the stability of the bromodomain-ligand binding complex when there is a change in the stability of the bromodomain-ligand binding complex in the presence of that compound. When the stability of the bromodomain-ligand binding complex increases in the presence of the compound, the compound is identified as a stabilizing agent. When the stability of the bromodomain-ligand binding complex decreases in the presence of the compound, the compound is identified as an inhibitor. In a preferred embodiment, the compound to be tested is pre-selected by performing rational drug design with the set of atomic coordinates obtained from one or more of Tables 1-6 of U.S. Ser. No. 09/784,553, filed Feb. 16, 2001, now U.S. Pat. No. 7,589,167, herein incorporated by reference. More preferably the selecting is performed in conjunction with computer modeling. In a particular embodiment, the compound is selected by performing rational drug design with the set of atomic coordinates obtained from a set of atomic coordinates defining the three-dimensional structure of a bromodomain consisting of the amino acid sequence of SEQ ID NO:7 alone or with acetyl-histamine.

As anyone having skill in the art of drug development readily understands, the potential drugs selected by the above methods can be refined by retesting in appropriate drug assays, including those disclosed herein. Chemical analogs of such potential drugs can be obtained (either through chemical synthesis or drug libraries) and be analogously tested. Therefore, methods comprising successive iterations of the steps of the individual drug assays, as exemplified herein, using either repetitive or different binding studies, or transcription activation studies or other such studies are envisioned in the present invention. In addition, potential drugs may be identified first by rapid throughput drug screening, as described below, prior to performing computer modeling on a potential drug using the three-dimensional structure of the bromodomain.

The present invention further provides potential, selected, and putative compounds (drugs) identified by the methods of the present invention, as well as the final drugs themselves identified using the methods of the present invention.

The present invention further provides methods for identifying potential binding partners for a protein (e.g., a histone) comprising an acetyl-lysine. One such embodiment comprises contacting the protein with a polypeptide comprising a bromodomain. In a preferred embodiment the bromodomain comprises the amino acid sequence of SEQ ID NO:3. In particular embodiments the bromodomain has the amino acid sequence of SEQ ID NO:7, or SEQ ID NO:8, or SEQ ID NO:9, or SEQ ID NO:10, or SEQ ID NO: 11, or SEQ ID NO: 12, or SEQ ID NO: 13, or SEQ ID NO: 14, or SEQ ID NO:15, or SEQ ID NO:16, or SEQ ID NO: 17, or SEQ ID NO:18, or SEQ ID NO:19, or SEQ ID NO:20, or SEQ ID NO:21, or SEQ ID NO: 22, or SEQ ID NO:23, or SEQ ID NO:24, or SEQ ID NO:25, or SEQ ID NO:26, or SEQ ID NO:27, or SEQ ID NO:28, or SEQ ID NO:29, or SEQ ID NO:30, or SEQ ID NO: or SEQ ID NO:31, or SEQ ID NO:32, or SEQ ID NO: 33, or SEQ ID NO:34, or SEQ ID NO:35, or SEQ ID NO:36, or SEQ ID NO:37, or SEQ ID NO:38, or SEQ ID NO: or SEQ ID NO:39, or SEQ ID NO:40, or SEQ ID NO:41, or SEQ ID NO:42.

The present invention further provides methods for identifying a protein having a bromodomain. One such embodiment comprises contacting a cellular extract with a peptide comprising an acetyl-lysine and/or an acetyl-lysine analog.

The present invention further provides agents that can inhibit the binding of a bromodomain with a protein comprising an acetyl-lysine (AcK). In one embodiment the agent is ISYGR-AcK-KRRQRR (SEQ ID NO:4). In another embodiment the agent is ARKSTGG-AcK-APRKQL (SEQ ID NO:5). In still another embodiment the agent is QSTSRHK-AcK-LMFKTE (SEQ ID NO:6). In yet another embodiment the agent is an analog of acetyl-lysine (see FIGS. 12 and 13). One particular analog of acetyl-lysine is acetyl-histamine. In still another embodiment the agent is an antibody that recognizes an acetyl-lysine of a protein binding partner of a bromodomain. In a preferred embodiment the agent is an antibody raised against a ZA loop of a bromodomain. These agents can be used as pharmaceuticals in compositions that contain a pharmaceutically acceptable carrier for example, or in the various drug assays of the present invention, serving as controls to demonstrate specificity.

The present invention further provides compounds such as small molecules that can inhibit the binding of a bromodomain with a protein comprising an acetyl-lysine. In a related aspect, the present invention provides methods for preventing or inhibiting the binding of bromodomains to acetyl-lysine residues of proteins comprising the step of administering a therapeutically effective amount of a pharmaceutical composition comprising a compound such as a small molecule.

Some useful small molecules of the invention that can inhibit the binding of a bromodomain with a protein comprising an acetyl-lysine may be represented by the following general formula (I) wherein:

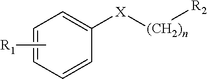

R¹ is selected from the group consisting of hydrogen, lower alkyl, aryl, aralkyl; substituted aralkyl, heteroaryl; substituted heteroaryl, phenyl, SO₂, NH₂, NO₂, SO₂, CH₃, CH₂CH₃, OCH₃, OCOCH₃, CH₂COCH₃, OH, CN and halogen;
R² is selected from the group consisting of hydrogen; lower alkyl, aryl, phenyl, aralkyl; substituted aralkyl, heteroaryl; substituted heteroaryl, SO₂, NH₂, NH₃⁺NO₂, SO₂, CH₃, CH₂CH₃, OCH₃, OCOCH₃, CH₂COCH₃, OH, halogen, carboxy, and alkoxy;
X is selected from the group consisting of lower alkyl, SO₂, NH, NO₂, O, carboxy, and alkoxy; and
n is an integer from 0 to 10;
   and their pharmaceutically acceptable salts of acids or bases;

Other useful small molecules of the invention that can inhibit the binding of a bromodomain with a protein comprising an acetyl-lysine may be represented by general formula (II) wherein:

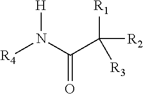

R¹ R² and R³ are independently selected from the group consisting of hydrogen; lower alkyl, aryl, phenyl, aralkyl; substituted aralkyl, heteroaryl; substituted heteroaryl, SO₂, NH₂, NH₃⁺NO₂, SO₂, CH₃, CH₂CH₃, OCH₃, OCOCH₃, CH₂COCH₃, OH, SH, halogen, carboxy, and alkoxy;
R⁴ is selected from the group consisting of lower alkyl, aryl, SO₂, NH, NO₂, CH₃, CH₂CH₃, OCH₃, OCOCH₃, CH₂COCH₃, OH, carboxy, and alkoxy;
   and their pharmaceutically acceptable salts of acids or bases;

The general formula (H) includes every stereoisomer, epimer and diastereoisomer, as a mixture or in isolated form. In especially preferred embodiments, R¹ R² and R³ are independently selected from the group consisting of hydrogen, lower alkyl, NH₃⁺, OH, SH, and halogen. Also in especially preferred embodiments, R⁴ is selected from the group consisting of lower alkyl and aryl.

Other useful small molecules of the invention that can inhibit the binding of a bromodomain with a protein comprising an acetyl-lysine may be represented by general formula (III) wherein

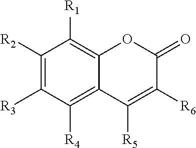

R¹ R², R³, R⁴ R⁵, and R⁶ are independently selected from the group consisting of hydrogen; lower alkyl, aryl, phenyl, aralkyl; substituted aralkyl, heteroaryl, substituted heteroaryl, SO₂, NH₂, NH₃⁺, NO₂, SO₂, CH₃, CH₂CH₃, OCH₃, OCOCH₃, CH₂COCH₃, OCH₂CH₃, OCH(CH₃)₂, OCH₂COOH, OCHCH₃COOH, OCH₂COCH₃, OCH₂CONH₂, OCOCH(CH₃)₂, OCH₂CH₂OH, OCH₂CH₂CH₃, O(CH₂)₃CH₃, OCHCH₃COOCH₃, OCH₂CON(CH₃)₂, NH(CH₂)₃N(CH₃)₂, NH(CH₂)₂N(CH₃)₂, NH(CH₂)₂OH, NH(CH₂)₃CH₃, NHCH₃, SH, halogen, carboxy, and alkoxy. In especially preferred embodiments,
R¹ and R⁴ are selected from the group consisting of hydrogen and OH;
R² is selected from the group consisting of hydrogen, OH, and CH₃, CH₂CH₃, OCH₃, OCOCH₃, CH₂COCH₃, OCH₂CH₃, OCH(CH₃)₂, OCH₂COOH, OCHCH₃COOH, OCH₂COCH₃, OCH₂CONH₂, OCOCH(CH₃)₂, OCH₂CH₂OH, OCH₂CH₂CH₃, O(CH₂)₃CH₃, OCHCH₃COOCH₃, OCH₂CON(CH₃)₂;
R³ is selected from the group consisting of hydrogen, OCH₂CH₃, and NHCOCH₃;
R⁵ is selected from the group consisting of hydrogen, lower alkyl, aryl, phenyl, aralkyl, NH(CH₂)₃N(CH₃)₂, NH(CH₂)₂N(CH₃)₂ , NH(CH₂)₂OH, NH(CH₂)₃; and
R⁶ is selected from the group consisting of hydrogen, and NH₂.

The general formula (III) includes every stereoisomer, epimer and diastereoisomer, as a mixture or in isolated form.

Other useful small molecules of the invention that can inhibit the binding of a bromodomain with a protein comprising an acetyl-lysine may be one of the following:

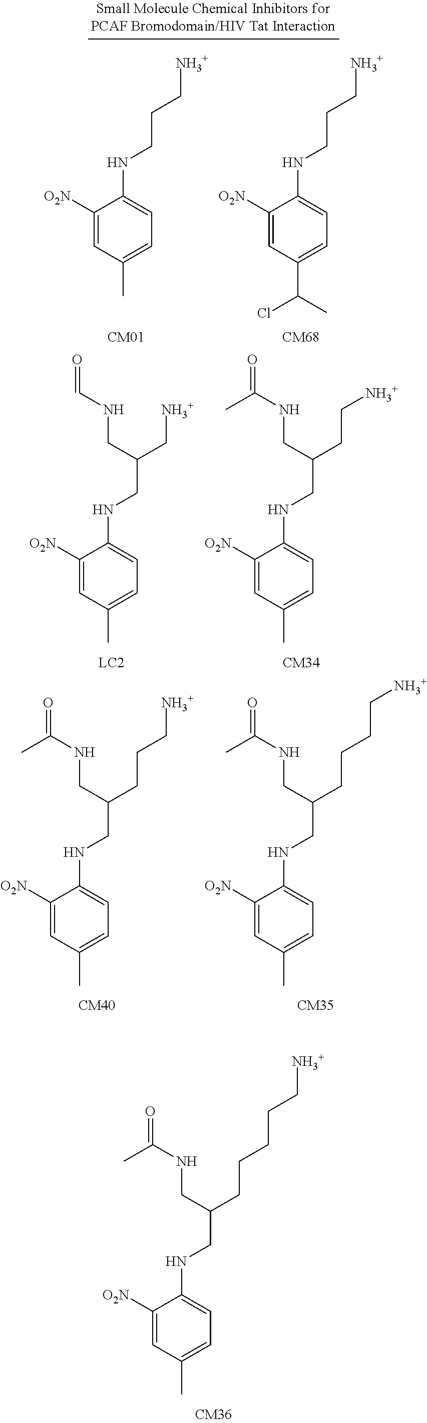

The present invention further provides an apparatus that comprises a representation of a bromodomain or a bromodomain-ligand complex (e.g., the Tat-P/CAF complex). One such apparatus is a computer that comprises the representation of the bromodomain or a bromodomain-ligand complex in computer memory. In one embodiment, the computer comprises a machine-readable data storage medium which contains data storage material that is encoded with machine-readable data which comprises the atomic coordinates from a bromodomain or a bromodomain-ligand complex. Preferably the computer comprises a machine-readable data storage medium which contains data storage material that is encoded with machine-readable data which comprises a portion or all of the structural coordinates contained in Tables 1-6 and 10-14 of U.S. Ser. No. 09/784,553, filed Feb. 16, 2001, now U.S. Pat. No. 7,589,167, herein incorporated by reference. In one embodiment, the computer comprises a machine-readable data storage medium which contains data storage material that is encoded with machine-readable data which comprises the structural coordinates for the Tat-P/CAF complex. More preferably the computer further comprises a working memory for storing instructions for processing the machine-readable data, a central processing unit coupled to both the working memory and to the machine-readable data storage medium for processing the machine readable data into a three-dimensional representation of the Tat-P/CAF complex, for example. In a preferred embodiment, the computer also comprises a display that is coupled to the central-processing unit for displaying the three-dimensional representation.

In addition, the present invention provides methods of identifying compounds that modulate the affinity of P/CAF for Tat that is acetylated at the lysine residue at position 50 of SEQ ID NO:45. In one such embodiment the method comprises contacting the bromodomain of P/CAF or a fragment thereof with a binding partner in the presence of the compound under conditions in which the bromodomain of P/CAF and the binding partner bind in the absence of the compound. The affinity of the bromodomain of P/CAF and the binding partner is then determined (e.g., measured). When there is a change in the affinity of the bromodomain of P/CAF for the binding partner in the presence of the compound, the compound is identified as a modulator. In one embodiment of this type the binding partner is Tat that is acetylated at the lysine residue at position 50 of SEQ ID NO:45. In a preferred embodiment the binding partner is a fragment of Tat comprising an acetyl-lysine at position 50. In still another embodiment the binding partner is an analog of the fragment of Tat comprising an acetyl-lysine at position 50. When the affinity of the bromodomain of P/CAF for the binding partner increases in the presence of the compound, the compound is identified as a Tat-P/CAF complex promoting agent, whereas when the affinity of the bromodomain of P/CAF for the binding partner decreases in the presence of the compound, the compound is identified as an inhibitor of the Tat-P/CAF complex.

In a preferred embodiment the compound is selected by performing rational drug design with the set of atomic coordinates obtained from one or more of Tables 1-5 and 10-14 of U.S. Ser. No. 09/784,553, filed Feb. 16, 2001, now U.S. Pat. No. 7,589,167, herein incorporated by reference. More preferably the selection is performed in conjunction with computer modeling. Compounds selected by these methods are also part of the present invention. Preferably the compound is a small organic molecule. More preferably the compound is an analog of acetyl-lysine. Even more preferably, the compound is not included in FIG. 13.

The present invention also provides methods of identifying a compound that modulates the stability of the binding complex formed between P/CAF and Tat that is acetylated at the lysine residue at position 50 of SEQ ID NO:45. In one such embodiment the method comprises contacting the bromodomain of P/CAF or a fragment thereof with a binding partner in the presence of the compound under conditions in which the bromodomain of P/CAF and the binding partner bind in the absence of the compound. The stability of the bromodomain of P/CAF and the binding partner is then determined (e.g., measured). When there is a change in the stability of the binding complex between the bromodomain of P/CAF and the binding partner in the presence of the compound, the compound is identified as a modulator. In one embodiment of this type the binding partner is Tat that is acetylated at the lysine residue at position 50 of SEQ ID NO:45. In a preferred embodiment the binding partner is a fragment of Tat comprising an acetyl-lysine at position 50. In still another embodiment the binding partner is an analog of the fragment of Tat comprising an acetyl-lysine at position 50. When the stability of the bromodomain of P/CAF for the binding partner increases in the presence of the compound, the compound is identified as a stabilizing agent, whereas when the stability of the bromodomain of P/CAF for the binding partner decreases in the presence of the compound, the compound is identified as an inhibitor of the Tat-P/CAF complex. In a preferred embodiment the compound is selected by performing rational drug design with the set of atomic coordinates obtained from one or more of Tables 1-5 and 10-14 of U.S. Ser. No. 09/784,553, filed Feb. 16, 2001, now U.S. Pat. No. 7,589,167, herein incorporated by reference. More preferably the selection is performed in conjunction with computer modeling. Compounds identified by these methods are also part of the present invention. Preferably the compound is an analog of acetyl-lysine. More preferably the compound is a small organic molecule not included in FIG. 13.

The present invention also provides agents that can modulate the binding of P/CAF and Tat. In a preferred embodiment the agent is a small organic molecule. Preferably the agent inhibits and/or destabilizes the binding of P/CAF with Tat. The agent may be, for instance, an analog of acetyl-lysine or any of the small molecules described herein.

Another aspect of the present invention provides methods of preventing, retarding the progression and treating HIV infection in an individual. One embodiment features administering to the individual a compound that modulates the Tat-P/CAF complex. In some embodiments the compound administered may be an acetyl-lysine analog or a small molecule. The compound may in some instance either de-stabilize or inhibit the Tat-P/CAF complex.

In another aspect, the present invention features methods for treating viral infection by administering a therapeutically effective amount of a pharmaceutical composition comprising a compound that inhibits interaction of Tat and P/CAF. In some preferred embodiments, the viral infection is HIV infection.

Accordingly, it is a principal object of the present invention to provide the three-dimensional coordinates of a bromodomain. It is a further object of the present invention to provide the three-dimensional coordinates of a bromodomain complexed with acetyl-histamine. It is a further object of the present invention to provide the three-dimensional coordinates of the Tat-P/CAF complex. It is a further object of the present invention to provide an assay for identifying proteins that contain bromodomains that bind proteins that comprise acetyl-lysine. It is a further object of the present invention to provide methods of identifying drugs that can modulate the bromodomain-acetyl-lysine binding complex. It is a further object of the present invention to provide methods of identifying drugs that can inhibit the binding of a bromodomain to a protein containing acetyl-lysine. It is a further object of the present invention to provide methods of identifying drugs that can modulate the Tat-P/CAF binding complex. It is a further object of the present invention to provide methods of identifying drugs that can inhibit the binding/formation of the Tat-P/CAF binding complex. It is a further object of the present invention to provide methods that incorporate the use of rational design for identifying such drugs. It is a further object of the present invention to provide a method of identifying drugs that can treat leukemia. It is a further object of the present invention to provide a method of identifying drugs that can treat, retard the progression, prevent and/or cure AIDS.

These and other aspects of the present invention will be better appreciated by reference to the following drawings and detailed description.

### BRIEF DESCRIPTION OF THE FIGURES

FIG. 1 provides the structure-based sequence alignment of a selected number of bromodomains. The sequences were aligned based on the NMR-derived structure of the P/CAF bromodomain, and the predicated four a-helices are shown in green boxes. Bromodomains are grouped on the basis of the sequence and/or functional similarities as described by Jeanmougin et al., (Trends in Biochemical Sciences, 22:151-153 (1997)). Residue numbers of the P/CAF bromodomain are indicated above its sequence. Three absolutely conserved residues, corresponding to Pro751, Pro767, and Asn803 in the P/CAF bromodomain, are shown in red. Highly conserved residues are colored in blue. The residues of the P/CAF bromodomain that interact with acetyl-histamine, as determined by intermolecular NOEs, are indicated by asterisks. The ZA loop, which is critical for acetyl-lysine binding, for each of the indicated bromodomains is also identified. The underlined residues were changed individually by site-directed mutagenesis to Ala. Genbank accession numbers for the proteins are as indicated in Table 8 6 of U.S. Ser. No. 09/784,553, filed Feb. 16, 2001, now U.S. Pat. No. 7,589,167, herein incorporated by reference, in the Example below, along with the SEQ ID NOs. for the bromodomain sequences.

FIGS. 2A-2H depict the structure of the P/CAF bromodomain. FIGS. 2A-2B shows the stereoview of the Cₐₗₚₕₐ. trace of 30 superimposed NMR-derived structures of the bromodomain (residues 722-830). The N-terminal four residues (SKEP) which are structurally disordered are omitted for clarity. For the final 30 structures, the root-mean-square deviations (RMSDs) of the backbone and all heavy atoms are 0.63±0.11 angstrom (□) and 1.15±0.12 angstrom for residues 723-830, respectively. The RMSDs of the backbone and all heavy atoms for the four α-helices (residues 727-743, 770-776, 785-802, and 807-827), are 0.34±0.04 angstrom and 0.87±0.06 angstrom, respectively. FIGS. 2C-2D show the stereoview of the bromodomain structures from the bottom of the protein, which is rotated approximately 90° from the orientation in FIGS. 2A-2B. FIG. 2E shows the Ribbons (Carson, Appl. Crystallogr. 24:958-961 (1991)) depiction of the averaged minimized NMR structure of the P/CAF bromodomain. The orientation of FIG. 2E is as shown in FIGS. 2A-2B. FIGS. 2F-2G are schematic representations of the overall topology of the up-and-down four-helix bundle folds with the opposite handedness. The left-handed fold is seen in bromodomain, cytochrome b₅, and T4 lysozyme (left, FIG. 2F), whereas the right-handed four-helix bundles are observed in proteins such as hemerythrin and cytochrome b₅₆₂ (right, FIG. 2G) (Richardson, Adv. Protein Chem., 34:167-339 (1989); Presnell, et al., Proc. Natl. Acad. Sci. USA 86:6592-6596 (1989)). FIG. 2H is a molecular surface representation of the electrostatic potential (blue=positive; red=negative) of the bromodomain calculated in GRASP (Nicholls et al., Biophys. J. 64:166-170 (1993)). The hydrophobic and aromatic residues (Tyr809, Tyr802, Tyr760, Ala757, and Val752) located between the ZA and BC loops are indicated.

FIGS. 3A-3C show the binding of the P/CAF bromodomain to AcK. FIG. 3A shows the superimposed region of the 2D¹⁵N-HSQC spectra of the bromodomain (approximately 0.5 mM) in its free form (red) and complexed to the AcK-containing H4 peptide (molar ratio 1:6) (black). FIG. 3B is the Ribbon and dotted-surface diagram of the bromodomain depicting the location of the lysine-acetylated H4 peptide binding site. The color coding reflects the chemical shift changes (.DELTA . . . delta.) of the backbone amide ¹H and ¹⁵N resonances upon binding to the AcK peptide as observed in the ¹⁵N-HSQC spectra. The normalized weighted average of the chemical shift changes was calculated by Δₐᵥ/Δₘₐₓ=(Δ²_{NH+}ΔΔ²_{N/25)/2})^{1/2/}Δₘₐₓ, where Δₘₐₓ is the maximum weighted chemical shift difference observed for Tyr809 (0.16 ppm). The backbone atoms are color-coded in red, yellow, or green for residues that have αₐᵥ/Δₘₐₓ of >0.6 (Tyr809, Glu808, Asn803, and Ala757), 0.2-0.6 (Ala813, Tyr802, Tyr760, and Val752), or <0.2 (Cys812, Ser807, Cys799, Phe796, and Phe748), respectively. The non-perturbed residues are shown in blue. FIG. 3C shows the chemical structures of acetyl-lysine, acetyl-histamine, and acetyl-histidine.

FIG. 4 depicts the acetyl-lysine binding pocket. This is the Ribbons (Carson, Appl. Crystallogr. 24:958-961 (1991)) depiction of a portion of the P/CAF bromodomain complexed with the acetyl-histamine. The ligand is color-coded by atom type.

FIGS. 5A-5B show the binding of various bromodomains from P/CAF, CBP and TIF1b to the N-terminal biotinylated and lysine-acetylated Tat peptide that was immobilized on streptavidin agarose.

FIGS. 6A-6D show the lysine-acetylated HIV-1 Tat protein interactions with bromodomains using 2D 1H-15N-HSQC spectra of the P/CAF or CBP bromodomain in the presence (red) or absence (black) of the lysine-acetylated peptides. Binding of the P/CAF bromodomain to the Tat AcK 50 peptide SYGR-AcK-KRRQRC (SEQ ID NO:50) is shown in FIG. 6A, to the Tat AcK 28 peptide TNCYCK-AcK-CCFH (SEQ IUD NO:58) is shown in FIG. 6B, and to histone H4 AcK16 peptide SGRGKGGKGLGKGGA-AcK-RHRK (SEQ ID NO:59) is shown in FIG. 6C. FIG. 6D shows the binding of the CBP of the bromodomain to the Tat AcK50 peptide. AcK is an acetyl-lysine residue

FIG. 7 is a bar graph of the measurement of superinduction of Tat transactivation activity by P/CAF. Tat-KK is the wild type Tat protein, and Tat-RR is the double mutant Tat carrying lysine to arginine mutations at K50 and K51 positions.

FIGS. 8A-8B show a western blot assay to detect P/CAF interaction with the Tat protein. Note that the protein-protein interaction was only observed with the wild type Tat but not with the Tat K50R/K51R mutant protein. The FLAG was joined to the Tat peptide, whereas the HA-tag was joined to P/CAF.

FIG. 9 depicts the structure of the P/CAF bromodomain in the complex with the lysine-acetylated Tat peptide (SYGR-AcK-KRRQRC, SEQ ID NO:50, where AcK is acetyl-lysine residue). The side chains of the amino acid residues on both the protein (green) and peptide (dark orange) that showed intermolecular NOEs in the NMR spectra are displayed.

FIGS. 10A-10B show the results of the mutational analyses of the P/CAF bromodomain binding to the HIV-1 Tat. FIG. 10A shows the effects of the point mutation of the individual residues of the bromodomain to alanine on the protein binding to the lysine-acetylated Tat peptide. FIG. 10B is an assessment of the peptide residue mutation on its binding to the P/CAF bromodomain.

FIG. 11 depicts a schematic of a computer comprising a central processing unit (“CPU”), a working memory, a mass storage memory, a display terminal, and a keyboard that are interconnected by a conventional bidirectional system bus. The computer can be used to display and manipulate the structural data of the present invention.

FIG. 12 depicts the chemical structure common of some acetyl-lysine analogs of the present invention. R₁, R₂, and R₃ can be H, CH₃, a halogen (e.g., F, Cl, Br, I etc.), OH, SH, or NH₃⁺. R4 can be an alkyl (including a peptide/protein attached thereto such as a peptide comprising an acetyl-lysine in which the “N” of the structure depicted is the epsilon nitrogen (i.e., N) of a lysyl residue), or an aryl group. See also FIG. 13 for examples.

FIG. 13 depicts examples of acetyl-lysine analogs.

FIG. 14 demonstrates the structural Basis of IBC Binding by BrDs. PCAF BrD binding of (A), (B) MIB; (C), (D) selective NP1. CBP BrD only binds to (E) MIB, but not (F) NP1, as shown by 15N-BrD HSQC with (red) or without a ligand (black).

FIG. 15 demonstrates lead optimization by linked fragment. Structures of PCAF BRD bound to (A) NP1 and EAA; (B) linked ligand LC2; (C) Lead optimization by chemical tethering.

FIG. 16 demonstrates specific Interaction of HIV Tat/PCAF in Viral Transcription. (A) Specific Tat-K50ac binding to PCAF BrD, but not other BrDs; (B) HIV-LTR activation mediated by Tat-K50ac/PCAF BrD interaction.

FIG. 17 demonstrates dose dependent inhibition of Tat mediated HIV transcription by PCAF BRD ligands, as measured using a HIV-LTR luciferase reporter assay.

FIG. 18 demonstrates the effect of PCAF BRD inhibitors on Tat transactivation. HeLa cells were transfected with LTR-luciferase and 20 ng Tat-expression vector. PCAF BRD inhibitors were added after transfection, and cells were harvested after 8 hours.

FIG. 19 demonstrates the effect of PCAF BRD inhibitors on viral infection. Jurkat T cells were infected with an LTR-Tat-IRES-GFP virus. PCAF BRD inhibitors were added after overnight infection. The percentage of infection was monitored 48 hours later by FACS analysis.

### DETAILED DESCRIPTION OF THE INVENTION

The present invention identifies a general binding partner (ligand) for the protein motif known as the bromodomain. Indeed, by combining structural and site-directed mutagenesis studies the present invention demonstrates that bromodomains can interact specifically with acetyl-lysine (AcK), making them the first protein modules known to exhibit such interactions. Like other modular domains, such as Src homology-2 (SH2) and phosphotyrosine binding (PTB) domains, which specifically interact with phosphotyrosine-containing proteins, the bromodomain/acetyl-lysine recognition provides a means to regulate protein-protein interactions via protein lysine acetylation. The nature of the acetyl-lysine recognition by the bromodomain is similar to that of histone acetyltransferase interaction with acetyl-CoA. The present invention therefore couples for the first time, the functionality of the bromodomain with the HAT activity of coactivators in the regulation of gene transcription.

The present invention further provides both a nuclear magnetic resonance (NMR) structure of the bromodomain from the HAT coactivator P/CAF (p300/CBP-associated factor) as well as the structure for the P/CAF bromodomain in complex with acetyl-histamine. The structure reveals an unusual left-handed up-and-down four-helix bundle.

The results disclosed herein explain prior deletion experiments which showed that the bromodomain is indispensable for the function of GCN5 in yeast. Bromodomain-AcK binding also appears to be important for the assembly and activity of multiprotein complexes in transcriptional activation. The results reported herein therefore form the foundation for identifying specific biological ligands and for defining the molecular mechanisms by which the extensive family of bromodomains participate in chromatin remodeling and transcriptional activation

As disclosed herein, the binding partner for the bromodomain is a peptide or protein comprising an acetyl-lysine (AcK). Interestingly, whereas a free acetyl-lysine does not appear to bind the bromodomain, an analog of the acetyl-lysine, acetyl-histamine, does. This is most likely due to the additional charge present in the free amino acid. Consistently, free acetyl-histidine also does not to bind the bromodomain.

In addition, as disclosed herein, the gene transactivation of HIV-1 Tat protein requires lysine-acetylation at amino acid residue 50 of Tat (see SEQ ID NO:45) by the transcription co-activator p300/CBP and the subsequent formation of a binding complex between the Tat having the acetylated lysine with P/CAF. The binding complex between P/CAF and Tat is mediated via the bromodomain of P/CAF and the acetylated lysine of Tat. Indeed, this binding is required for the gene transactivation activity of Tat and thus, for HV-1 expression and replication.

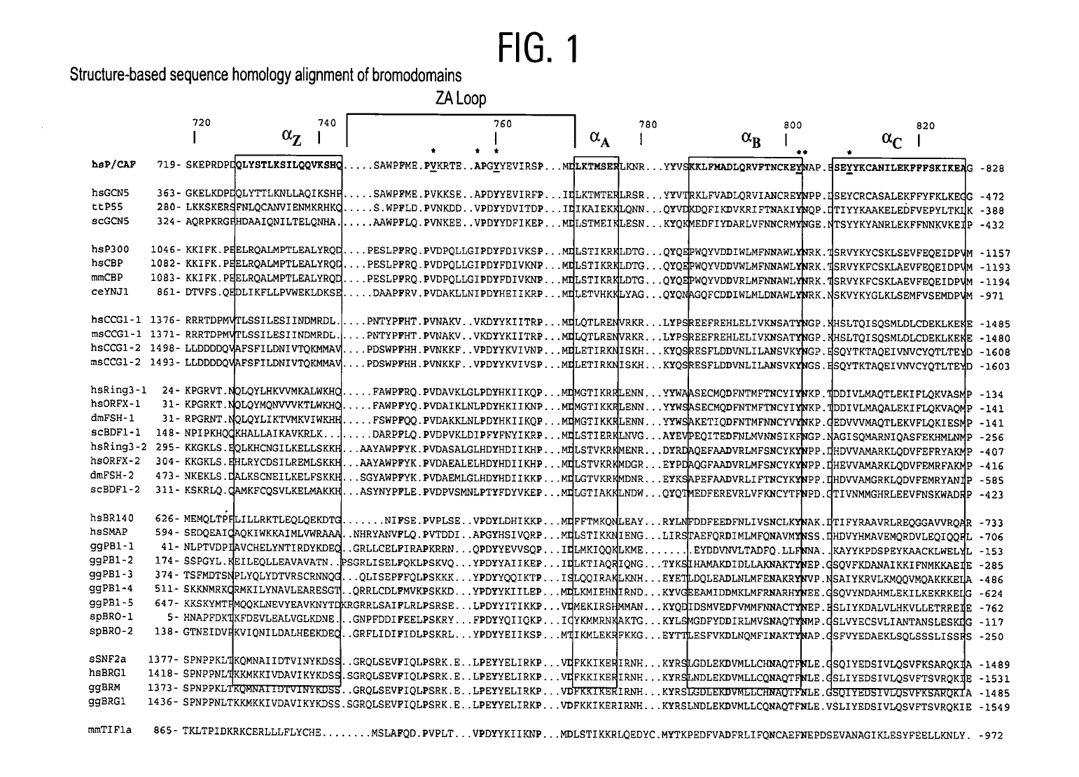

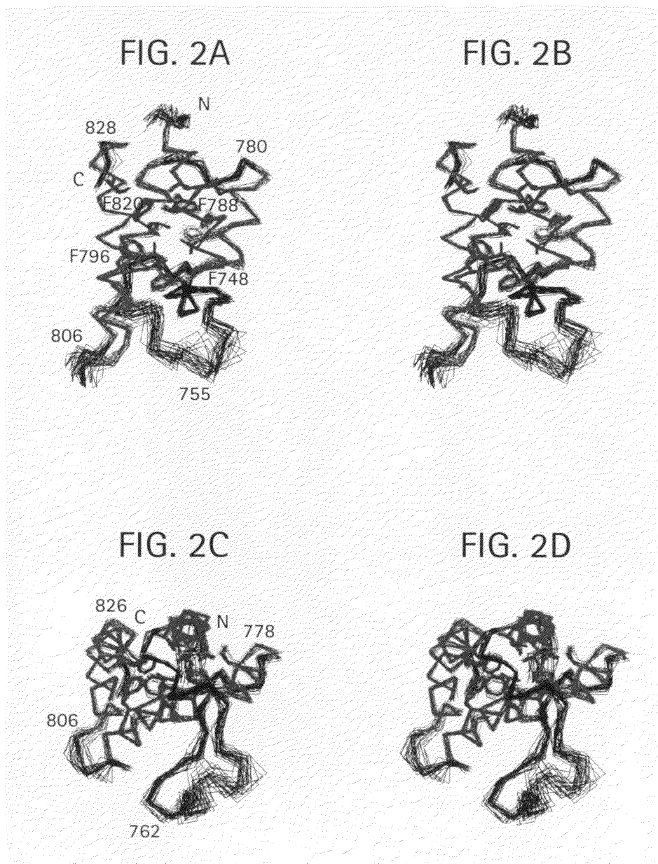

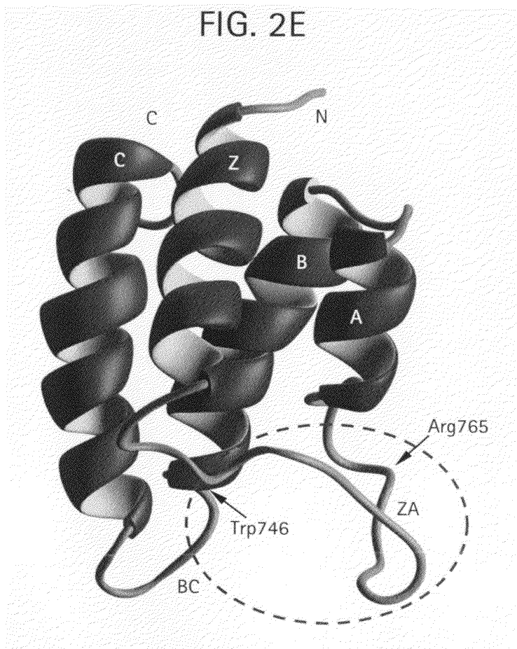

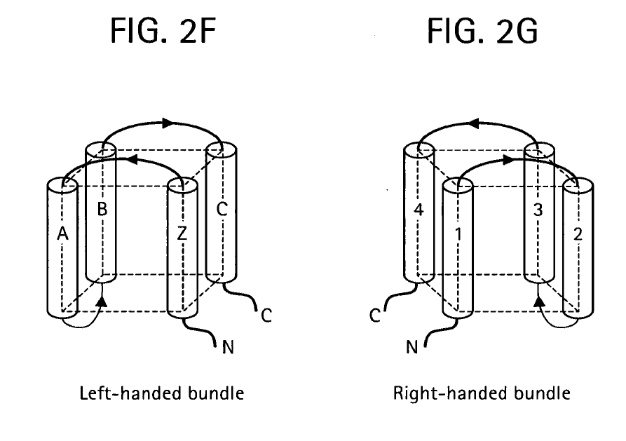

The present invention further provides a key region of the bromodomain for the interaction with its acetyl-lysine binding partner, the ZA loop. The amino acid sequence of the ZA loop is defined in FIG. 1 for a number of bromodomains and is depicted in FIG. 2A for P/CAF. In a particular embodiment, the ZA loop has between about 21 and 40 amino acid residues comprising the amino acid sequence:

(SEQ ID NO: 3)
F X₂₋₃ P X₅₋₈ J_{P/K/H X} Y J_{Y/F/H} X₅ P J_{M/I/V} D

more preferably the ZA loop has about 23 to 34 amino acid residues and comprises the amino acid sequence:

(SEQ ID NO: 43)
X_{2F} X₂₋₃ P X₅₋₈ J_{P/K/H} X Y J_{Y/F/H} X₅ P J_{M/I/V} D

In a specific embodiment, the ZA loop has between about 20 and 64 amino acid residues comprising the amino acid sequence:

(SEQ ID NO: 48)
F X₂₋₄ V X₂₋₄ E X₂₋₄ Y X₁₋₃ VJ_{I/L/M/V}

(1) The single letter amino acid code is used in this description, i.e., “F” for phenylalanine; “P” for proline; “Y” for tyrosine; and “D” for aspartic acid.

(2) “X” indicates any amino acid (an undesignated amino acid); and X, X₂, X₂₋₃, X₅, and X₅₋₈ indicates one undesignated amino acid, two consecutive undesignated amino acids, two or three consecutive undesignated amino acids, five consecutive undesignated amino acids, and five to eight consecutive undesignated amino acids respectively.

(3) “J” indicates that identity of the amino acid is restricted to a particular group, again the one letter code is used

(i) J_{P/K/H} is either proline, lysine or histidine.

(ii) J_{Y/F/H} is either tyrosine, phenylalanine or histidine.

(iii) J_{M/I/V} is either methionine, isoleucine, or valine.

(iv) J_{I/L/M/V}is either isoleucine, leucine, methionine, or valine

Since this region of the bromodomain is important in binding its acetyl-lysine binding partner, antibodies specifically raised against this region are also included in the present invention. In a particular embodiment, the antibody is a humanized chimeric antibody that can be used in therapeutic treatment. Thus monoclonal, chimeric, and polyclonal antibodies raised against bromodomains, preferably against amino acid residues in the ZA loop region are part of the present invention. In a specific embodiment the antibody is raised against a peptide, fusion peptide or conjugated peptide consisting of amino acid residues 746 to 765 of SEQ ID NO:2, i.e., WPFMEPVKRTEAPGYYEVIR (SEQ ID NO:44). In another embodiment the antibody is raised against a peptide, fusion peptide or conjugated peptide consisting of amino acid residues 748 to 809 of SEQ ID NO:2 (which is SEQ ID NO:49).

Such antibodies can be used in the treatment of leukemia or AIDs for example. Alternatively, these antibodies can be used in drug discovery assays.

Analogously, the present invention provides peptides derived from the HIV-1 Tat protein. In one such embodiment the peptide comprises 7 to 21 amino acid residues comprising the amino acid sequence

| | |
|---|---|
| YGRKX₁-3RQ | (SEQ ID NO: 46) |

In a specific embodiment the peptide fragment of Tat has ten amino acid residues and the amino acid sequence:

| | |
|---|---|
| SYGRKKRRQR | (SEQ ID NO: 47) |

Preferably the lysine corresponding to lysine 50 of Tat (see SEQ ID NO:45) is acetylated. These peptide fragments can be used in the drug assays of the present invention and/or as antigens for antibodies that specifically interfere with the interaction (e.g., binding) of Tat with P/CAF interaction.

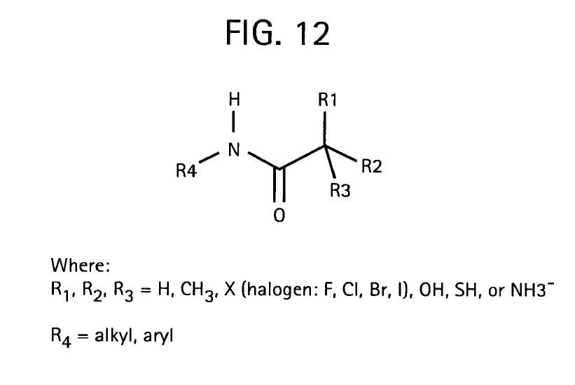

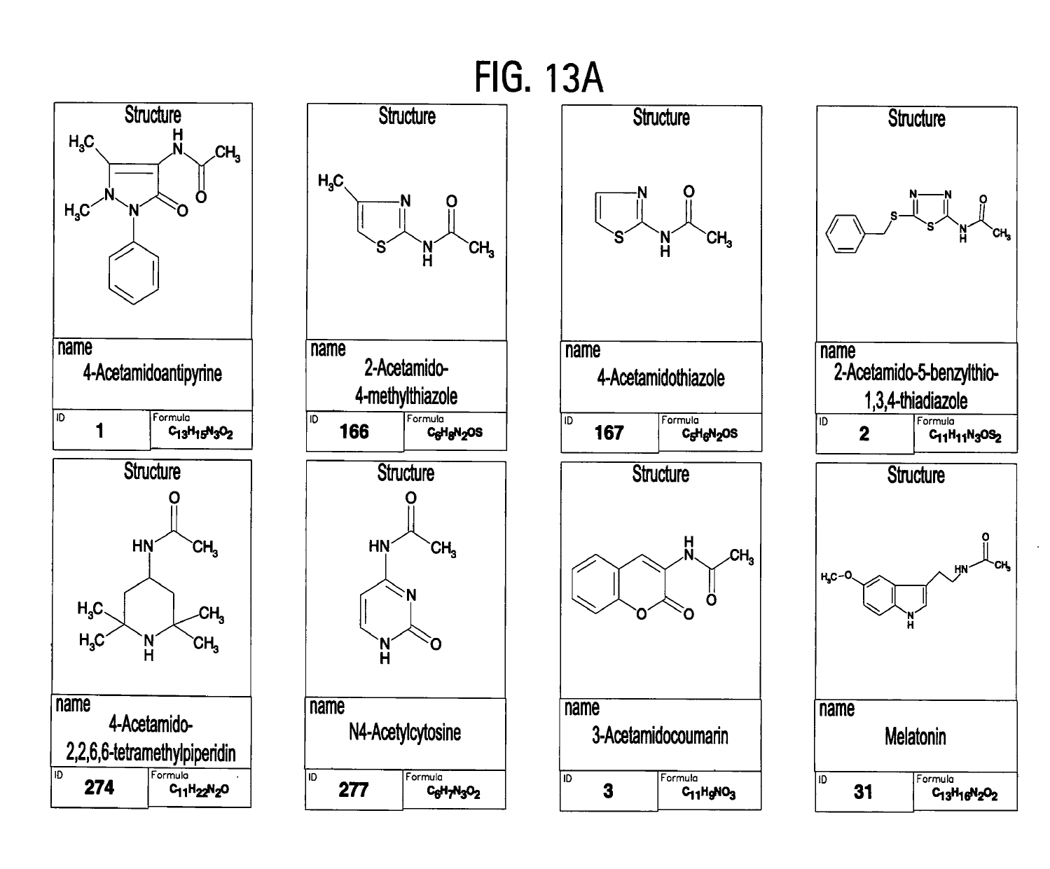

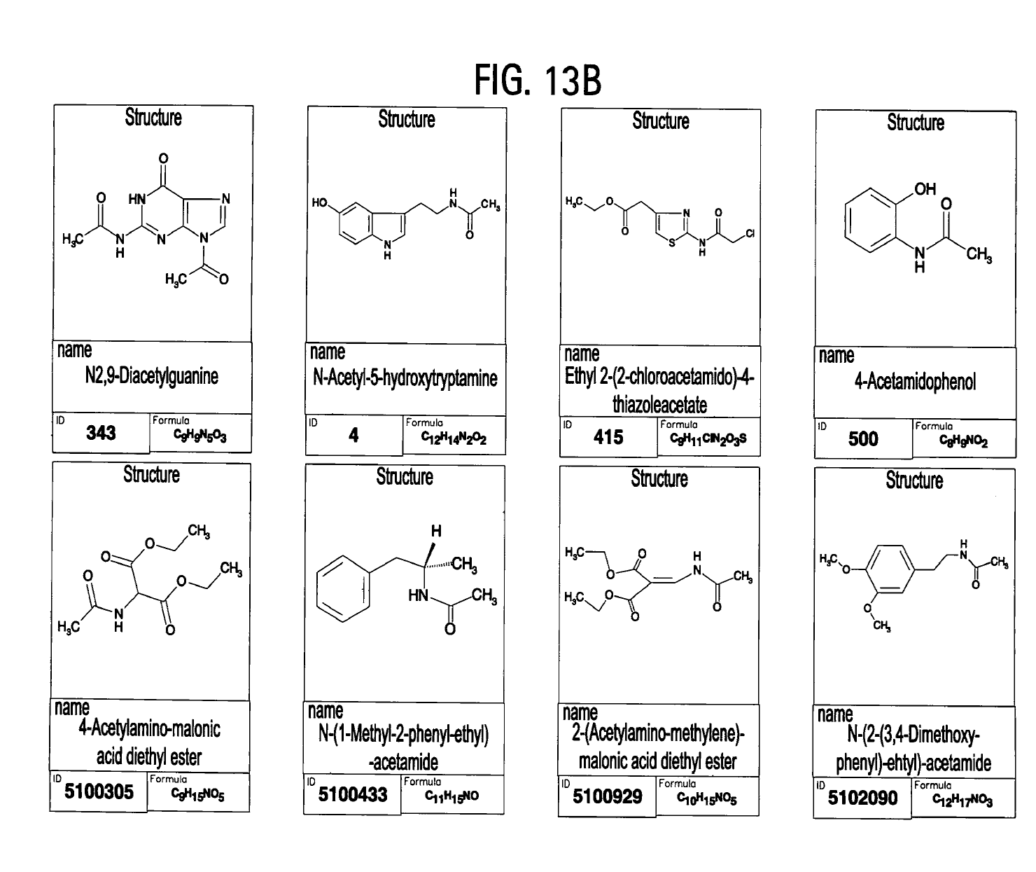

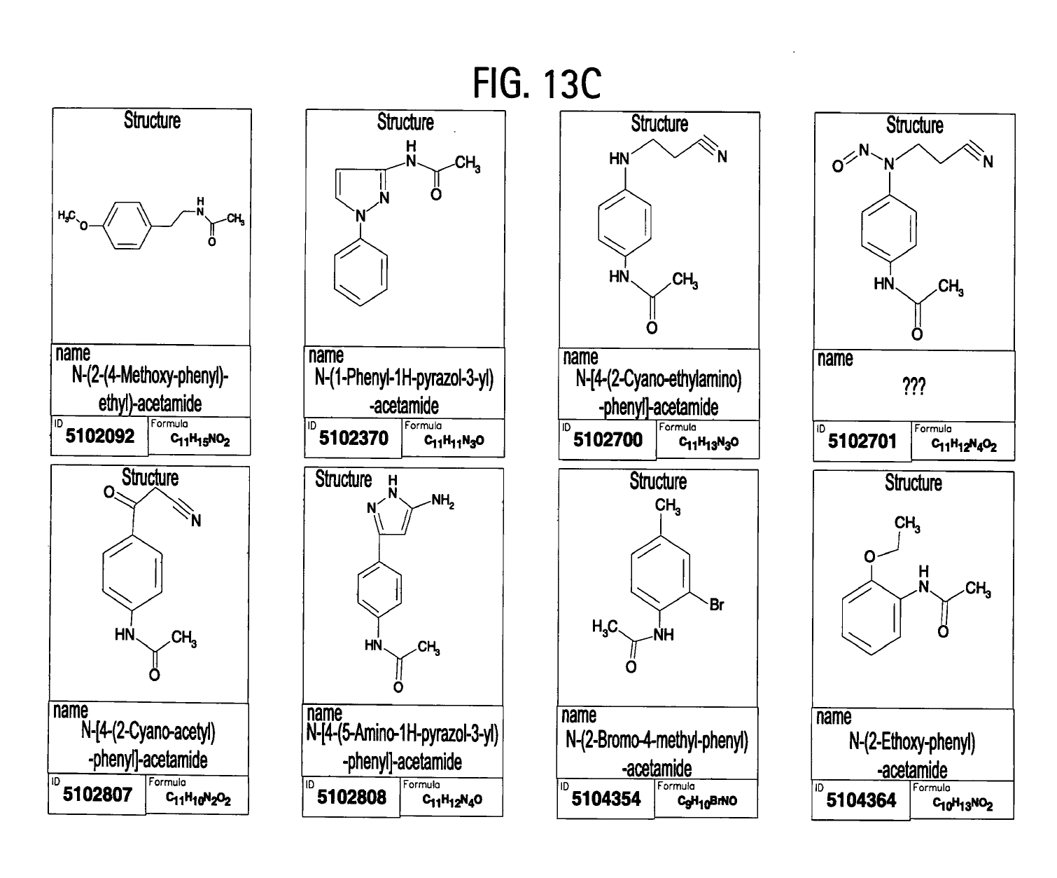

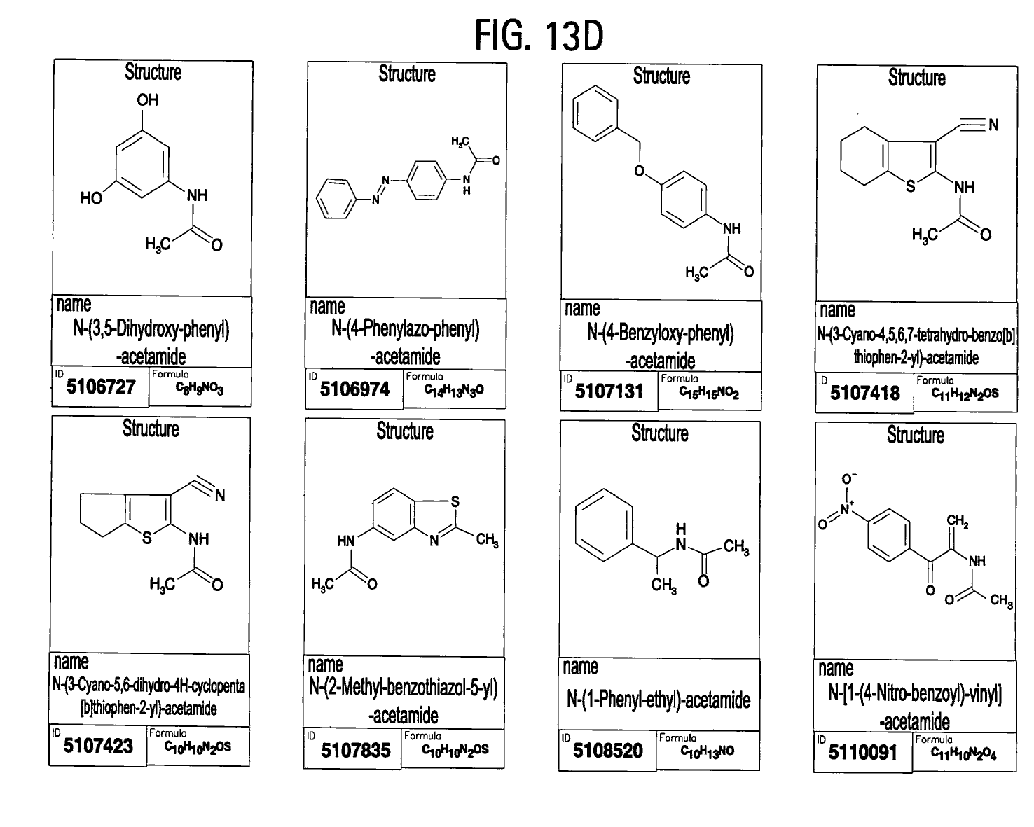

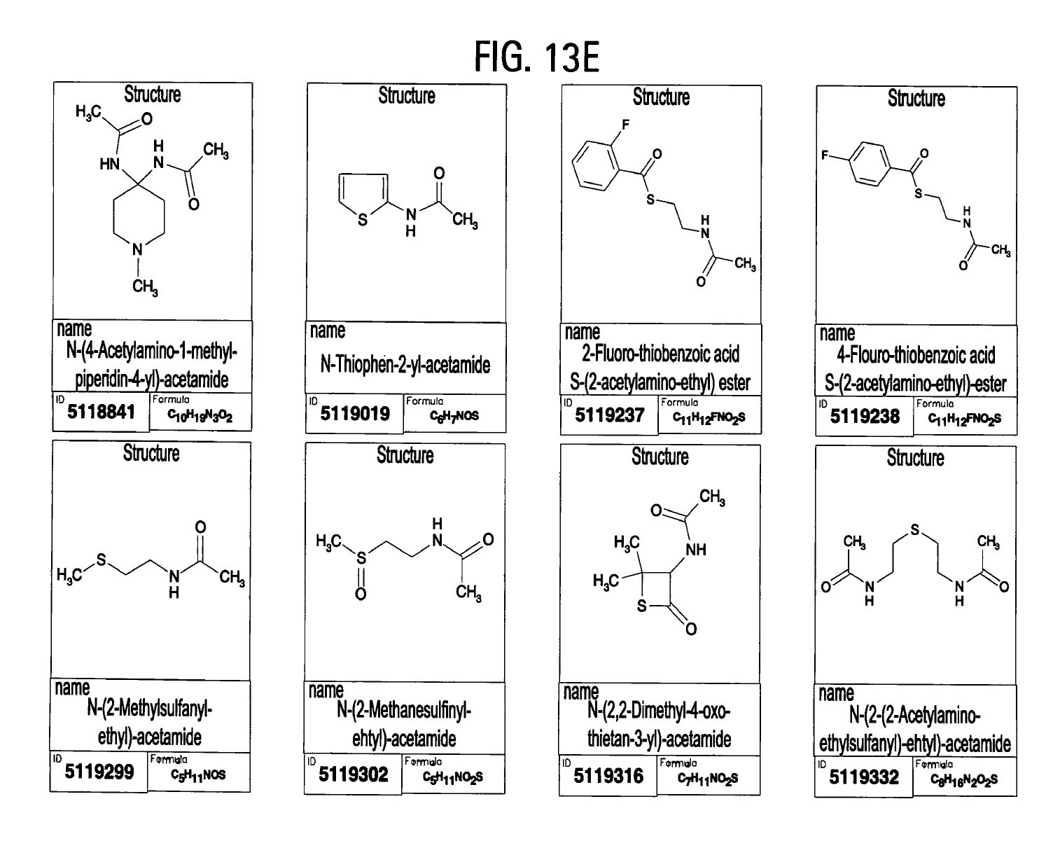

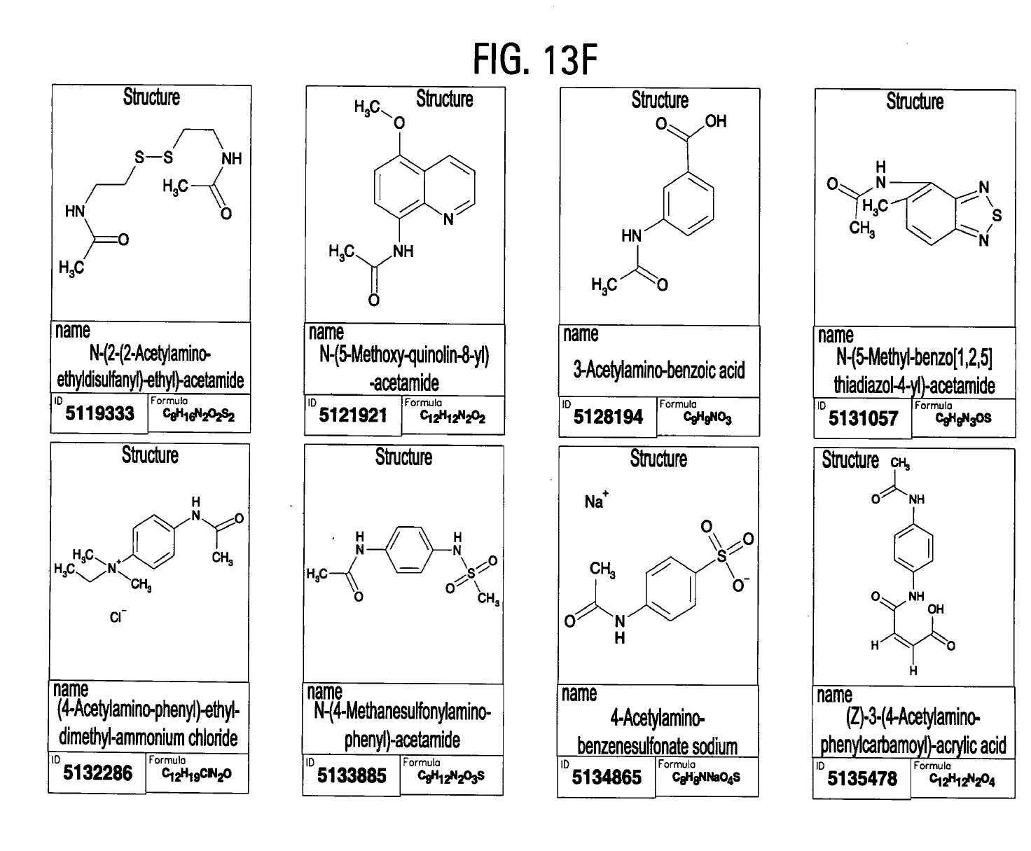

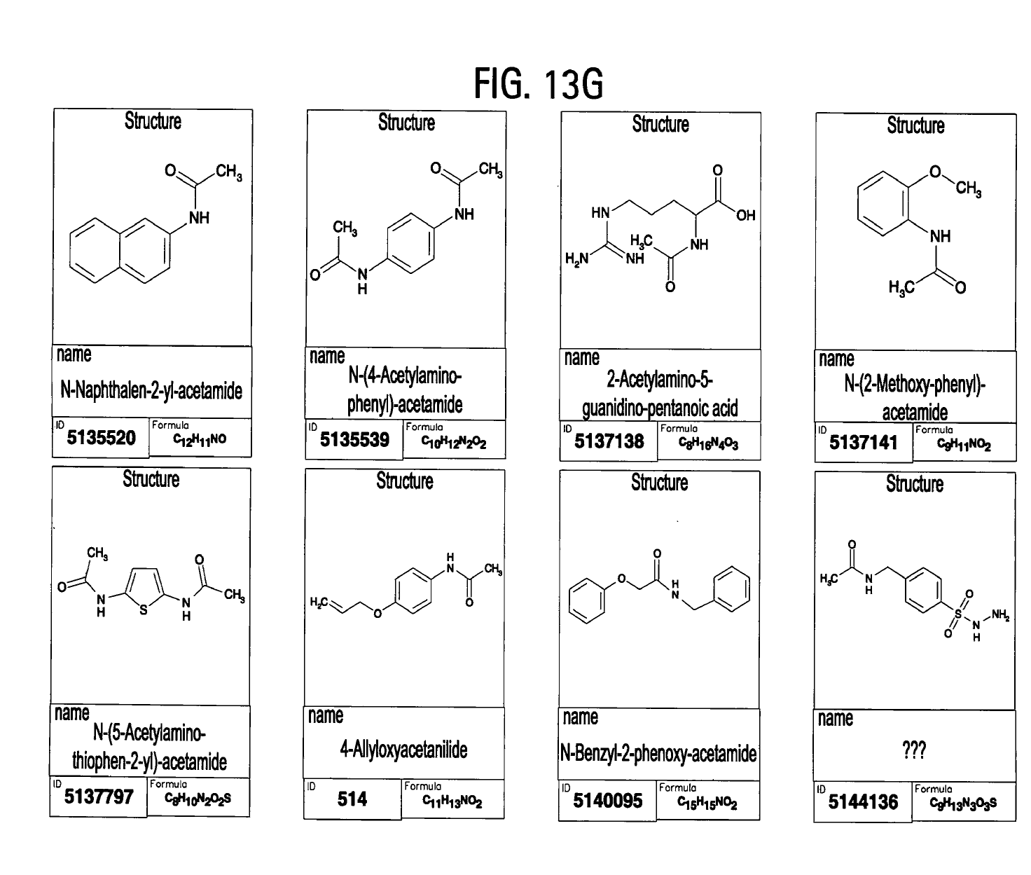

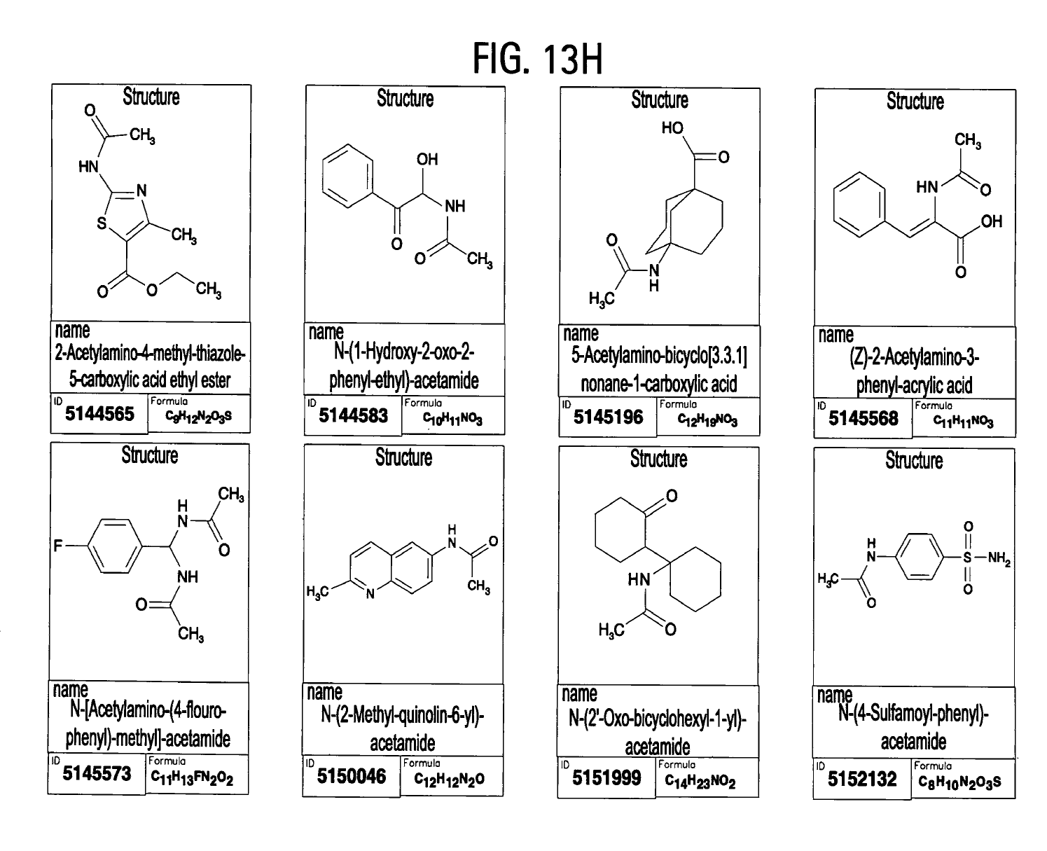

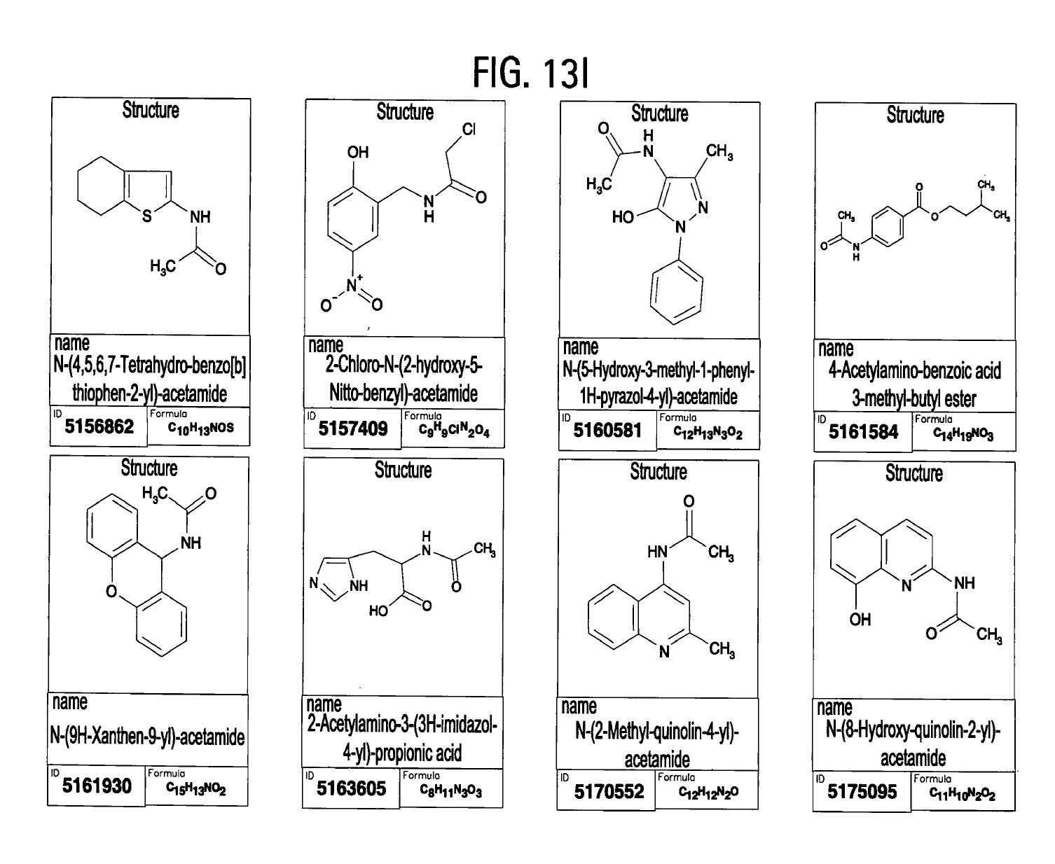

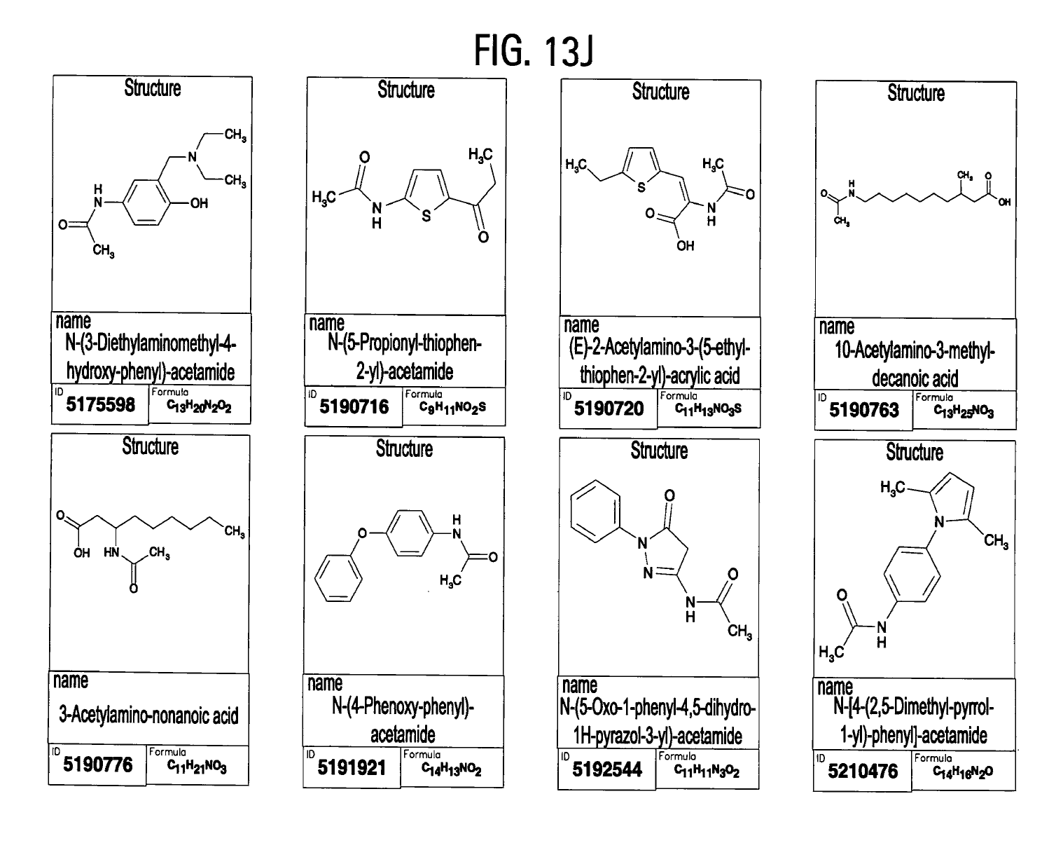

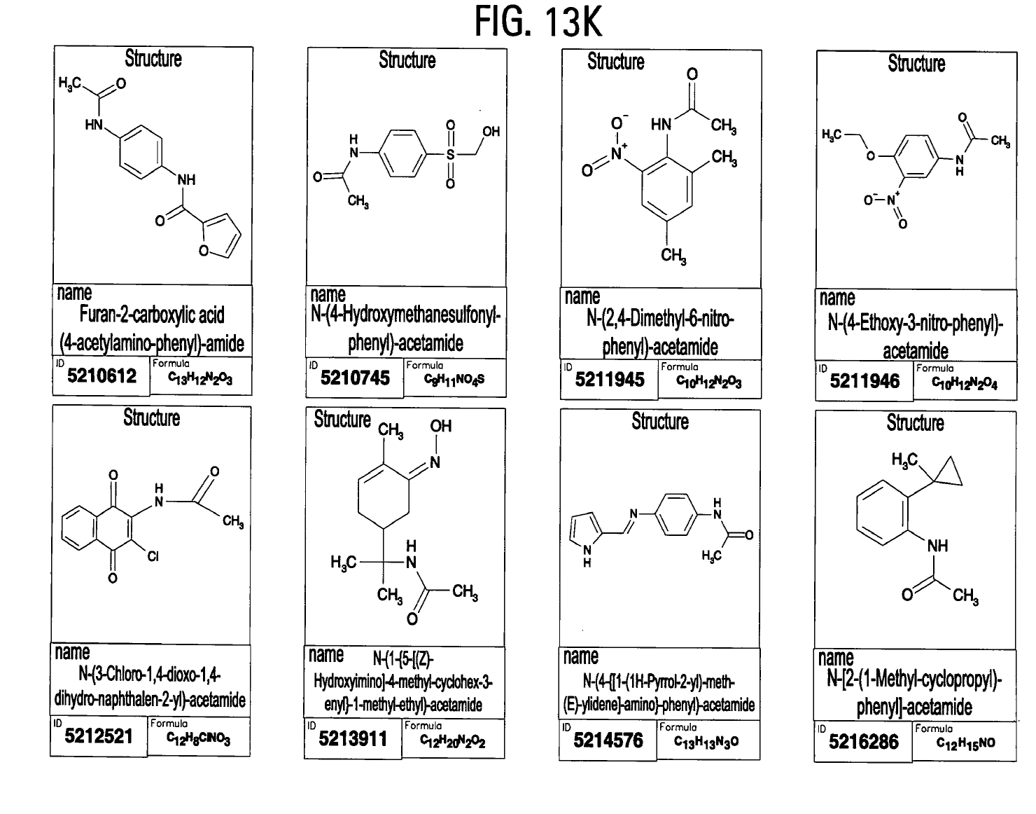

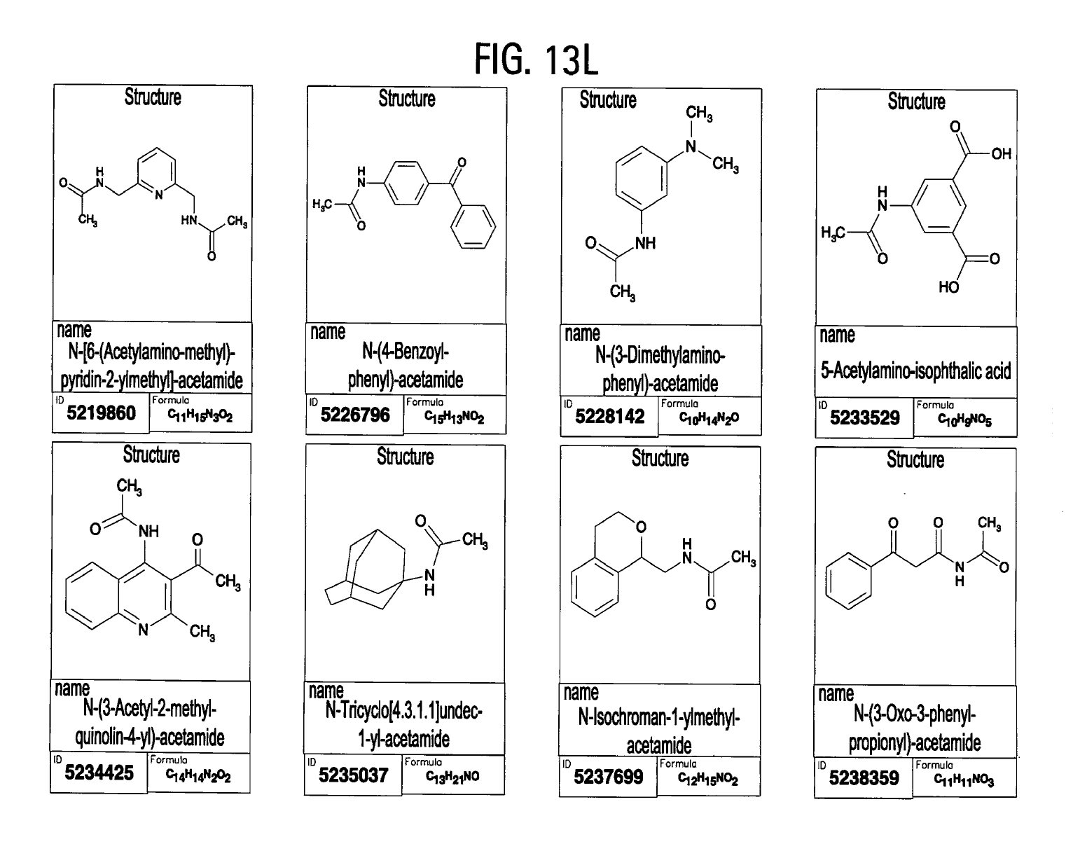

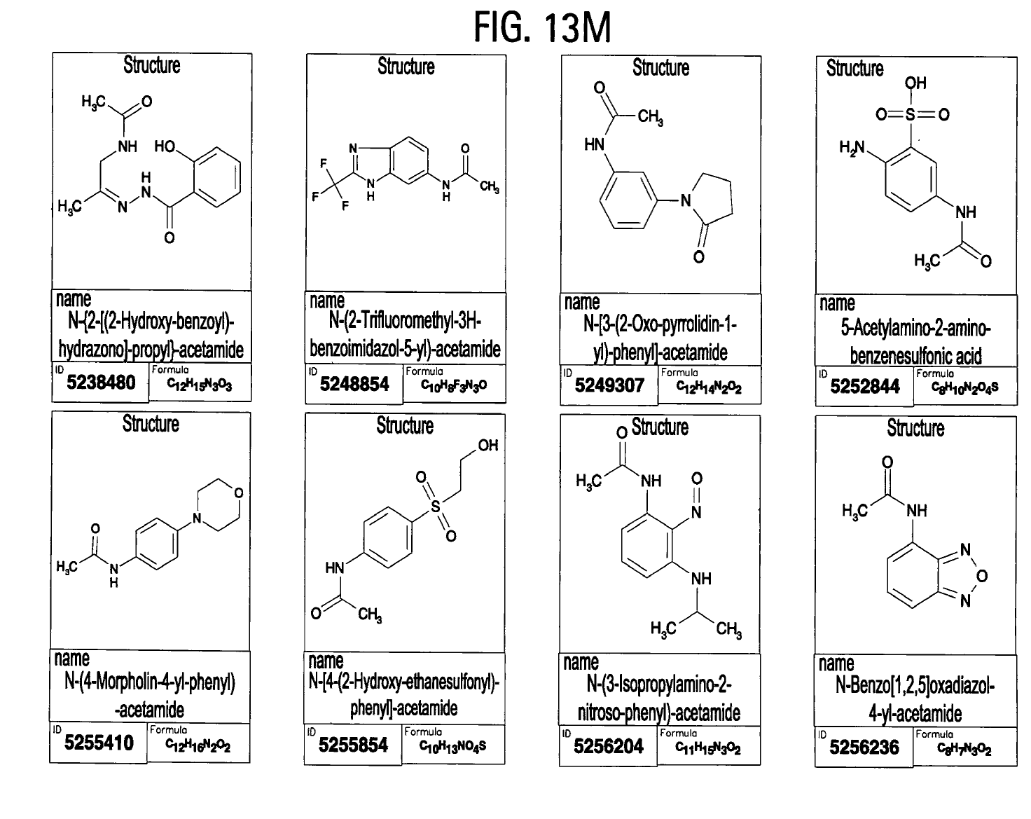

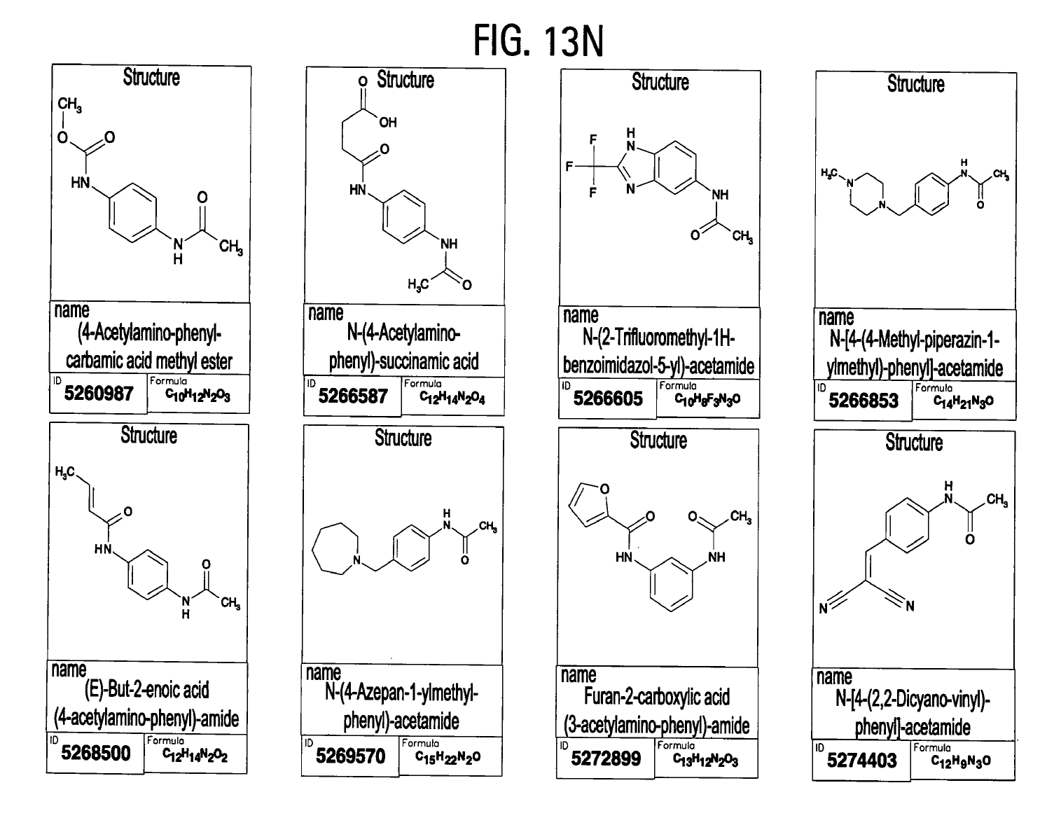

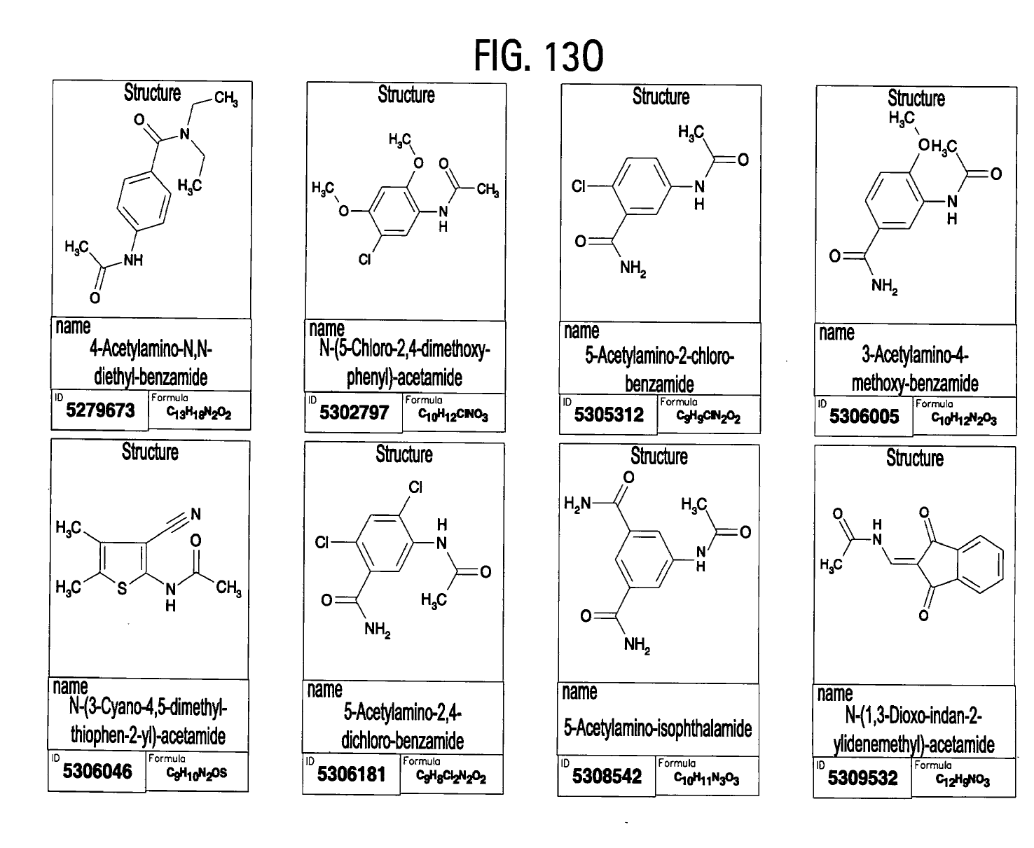

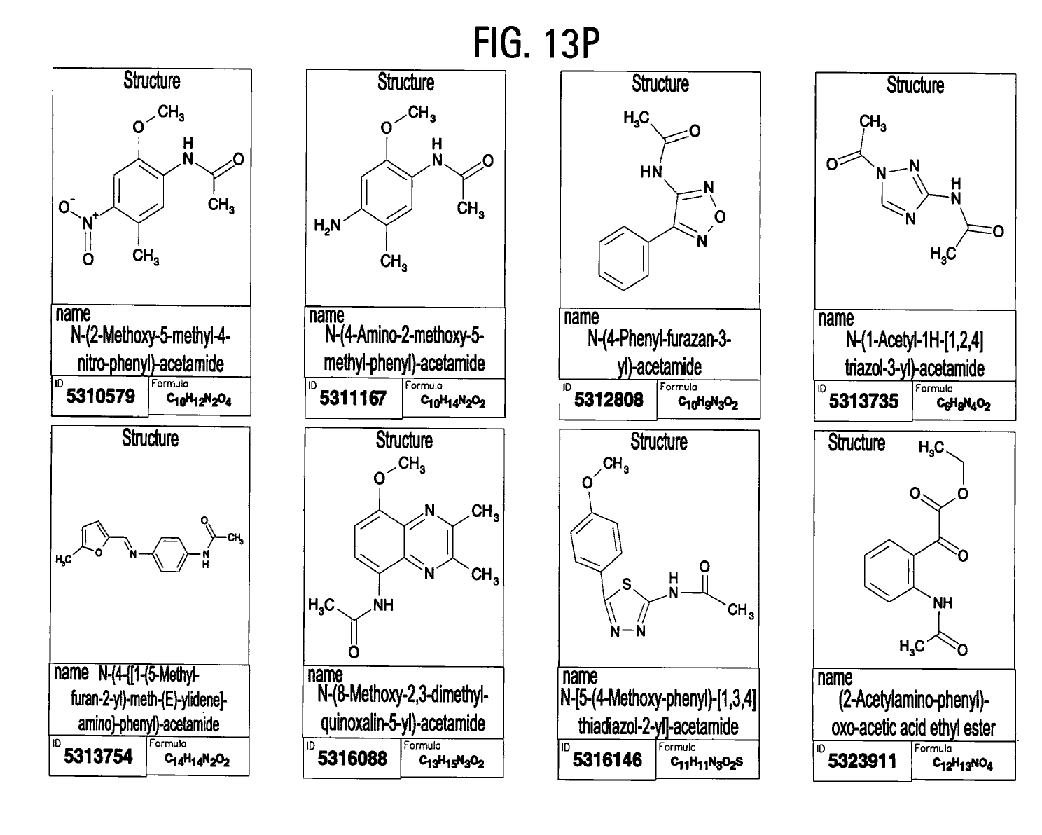

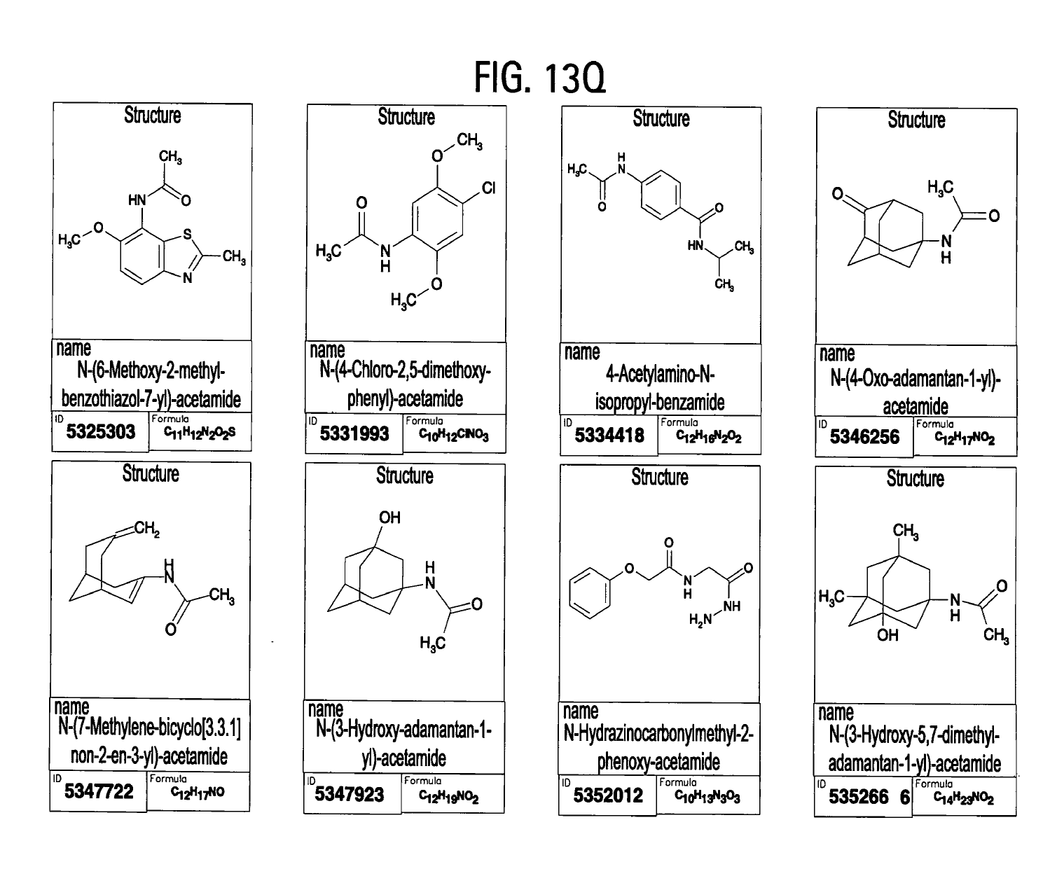

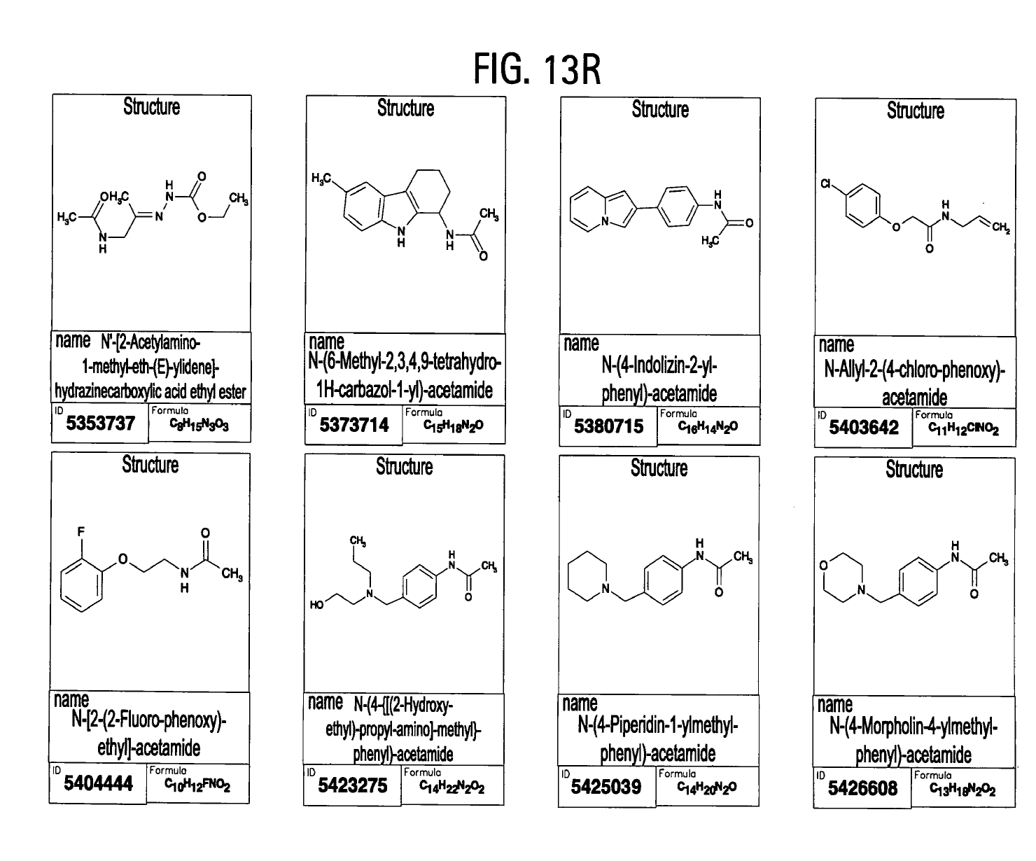

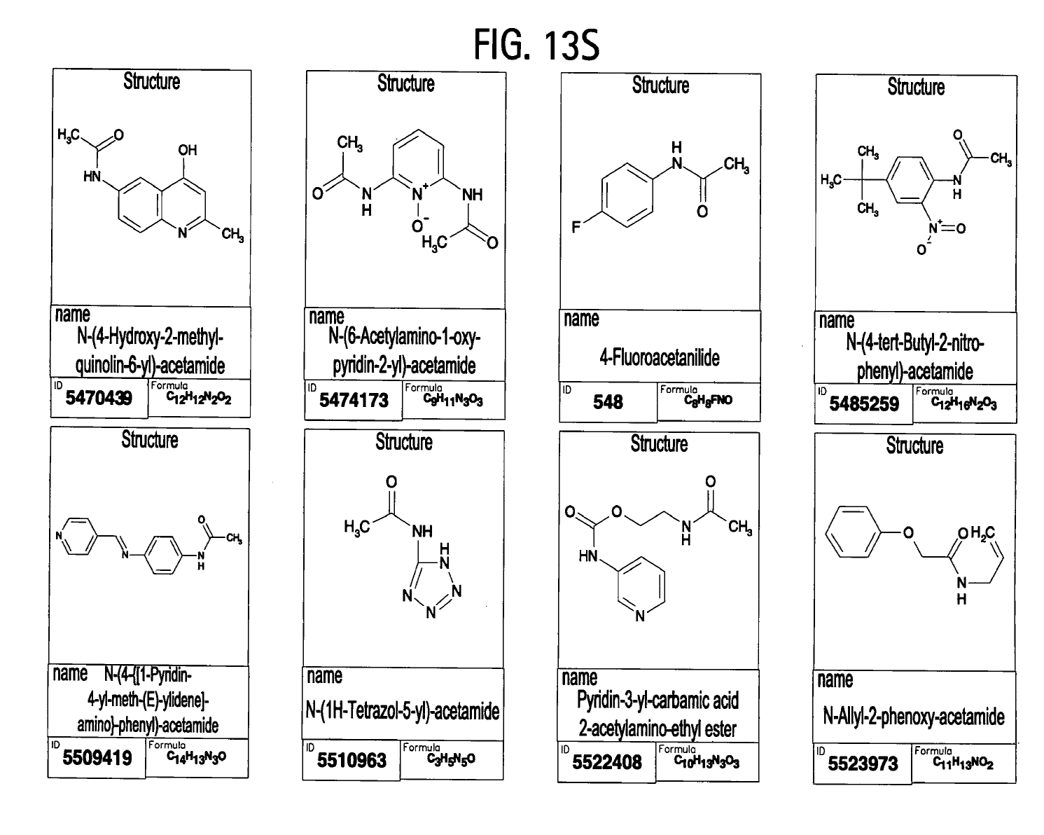

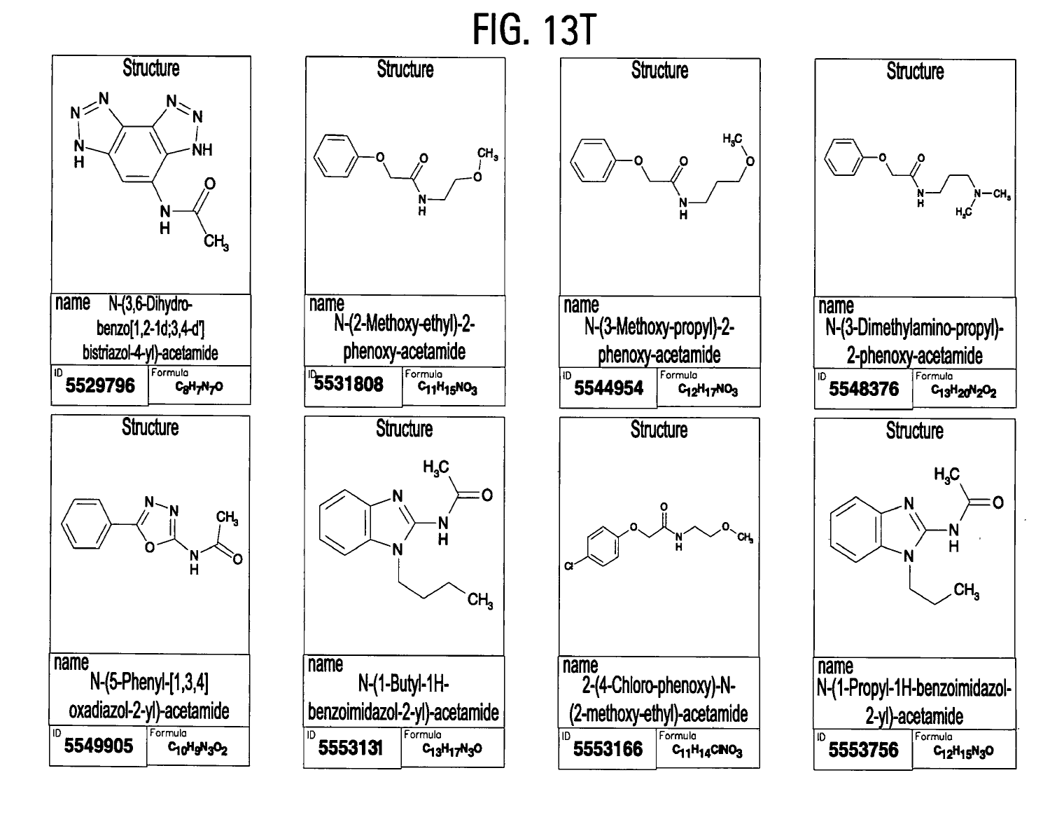

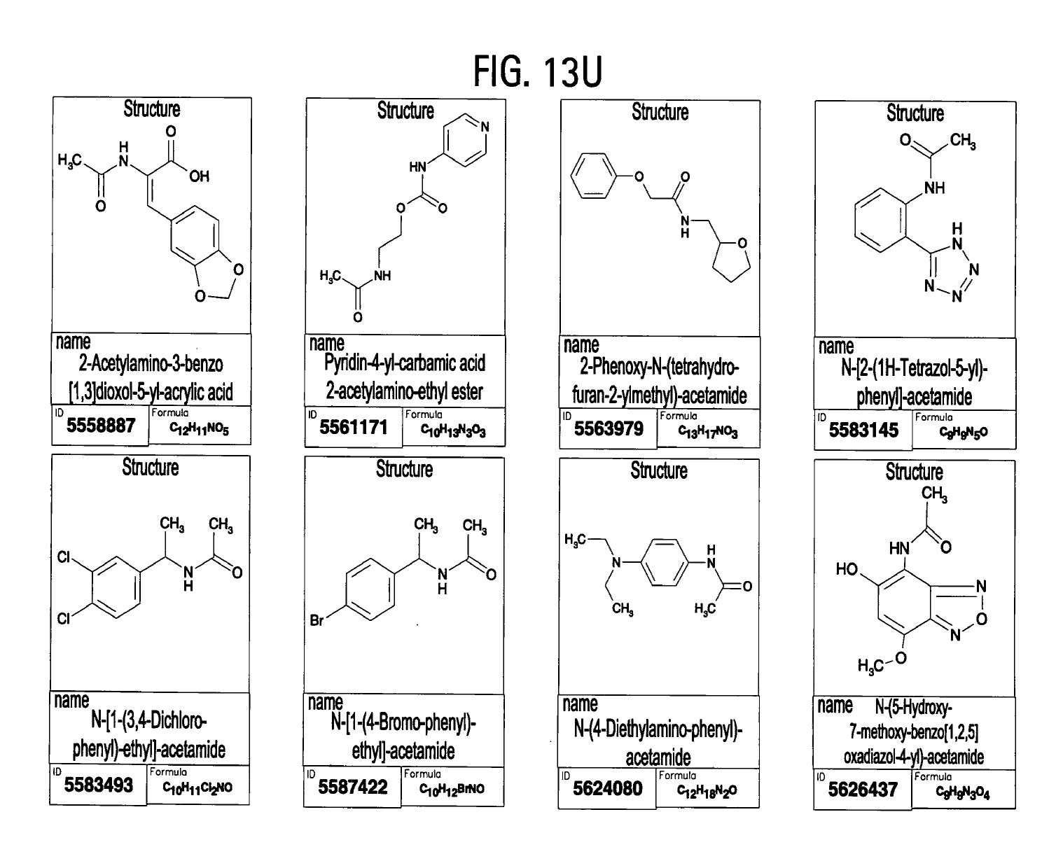

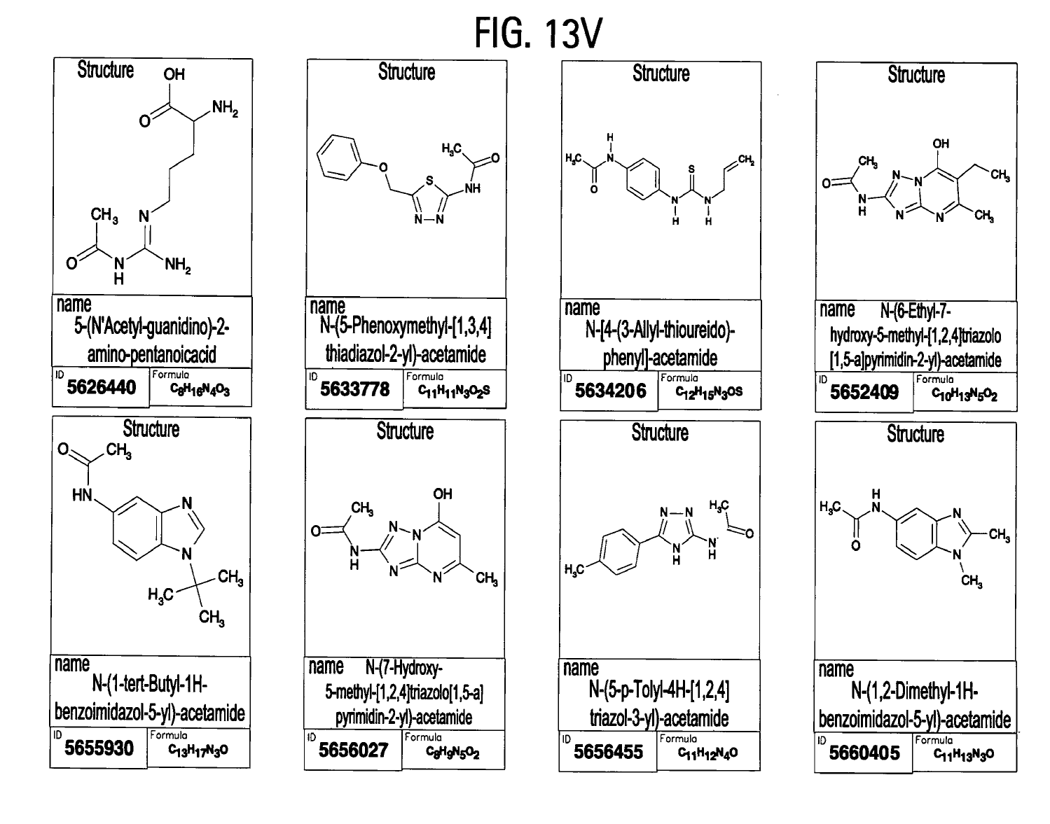

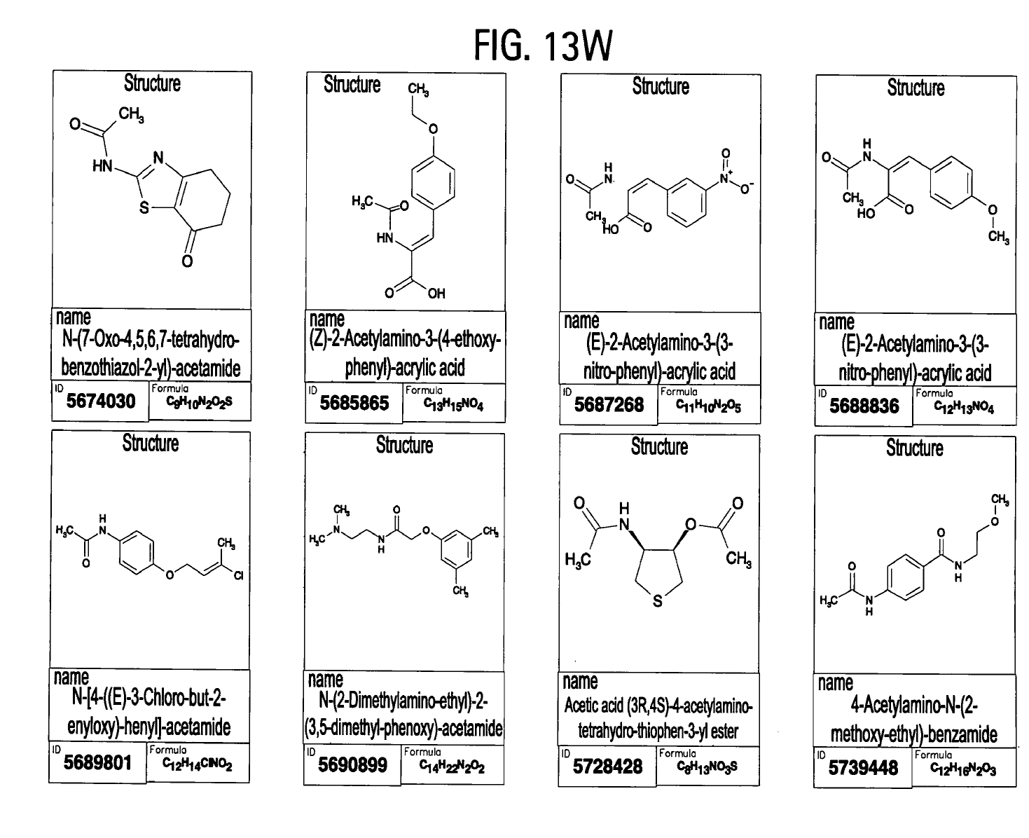

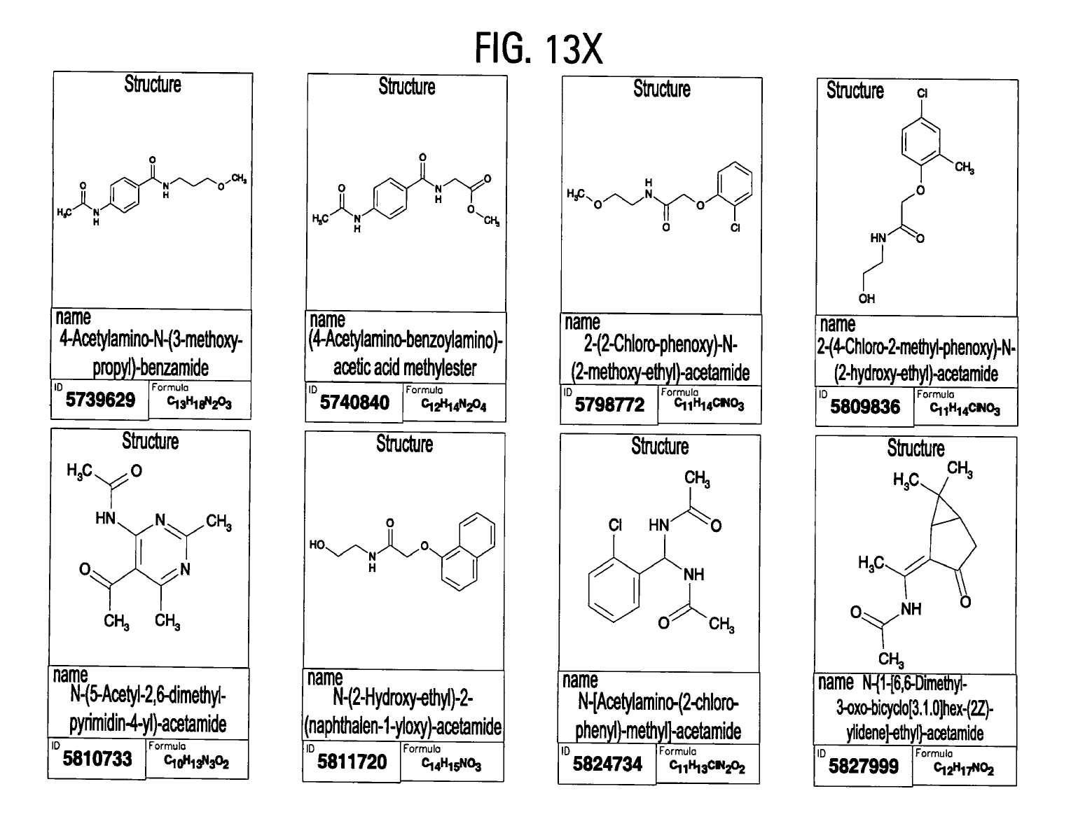

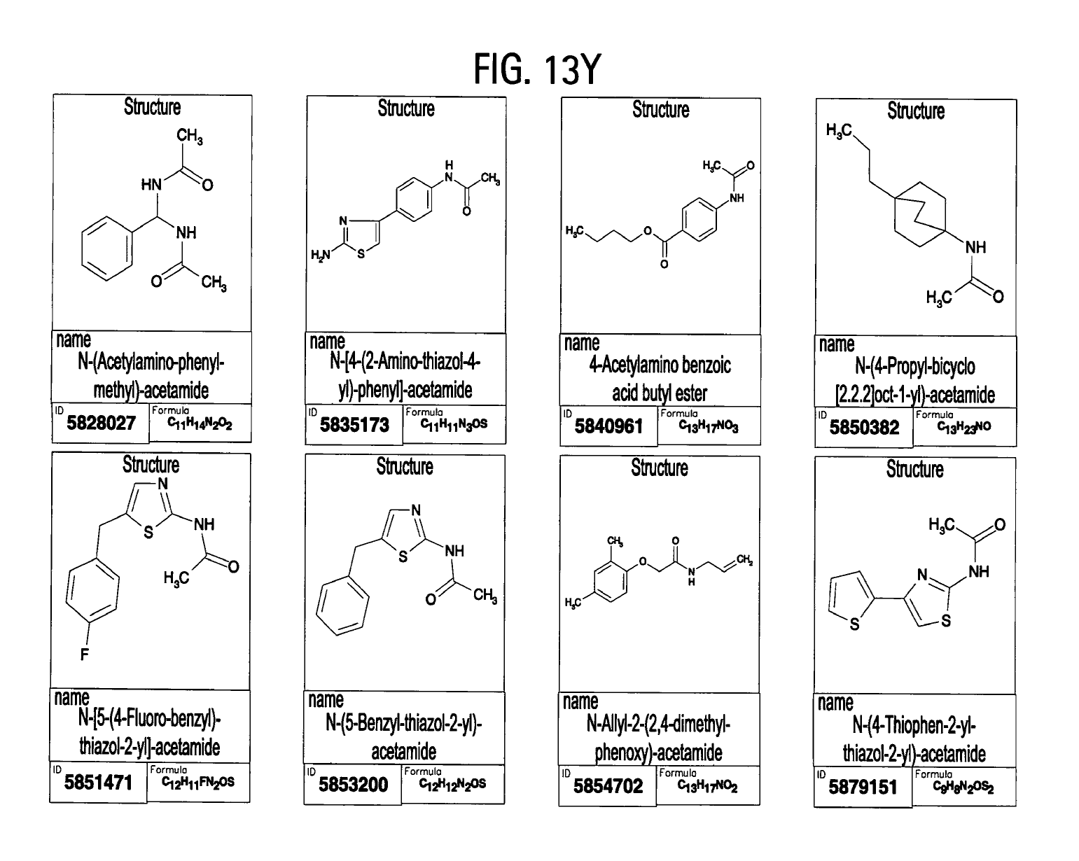

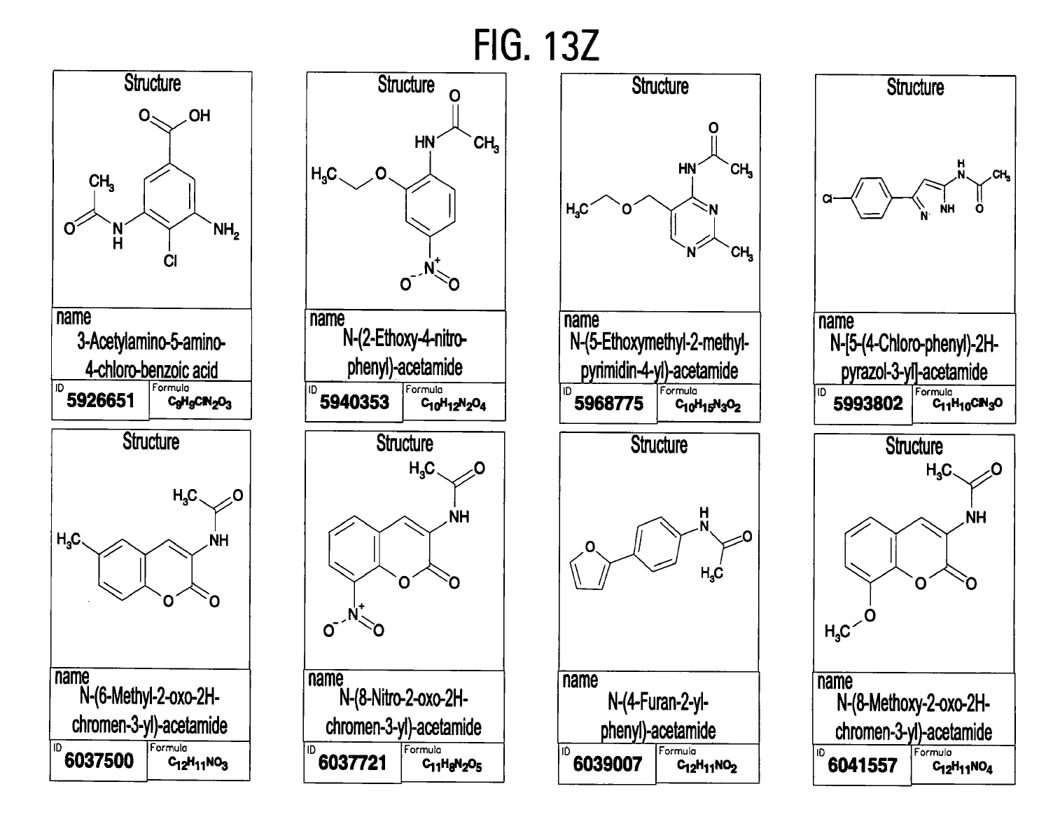

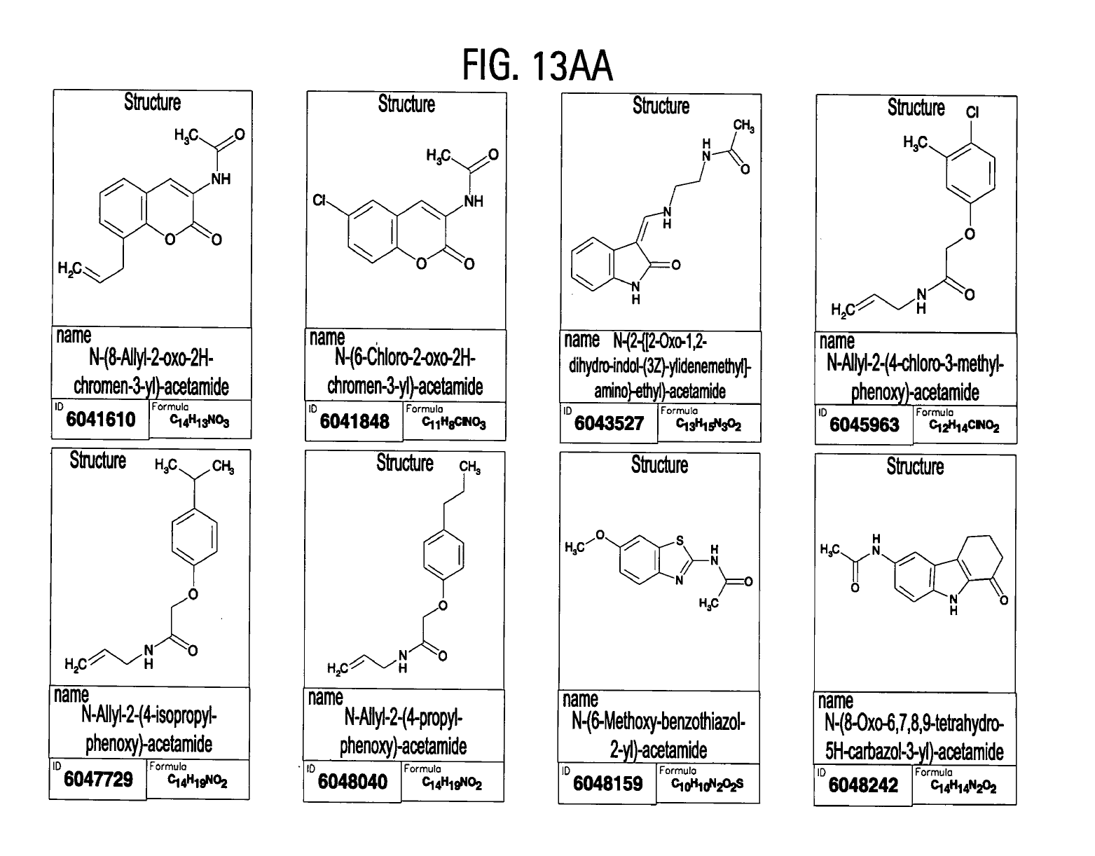

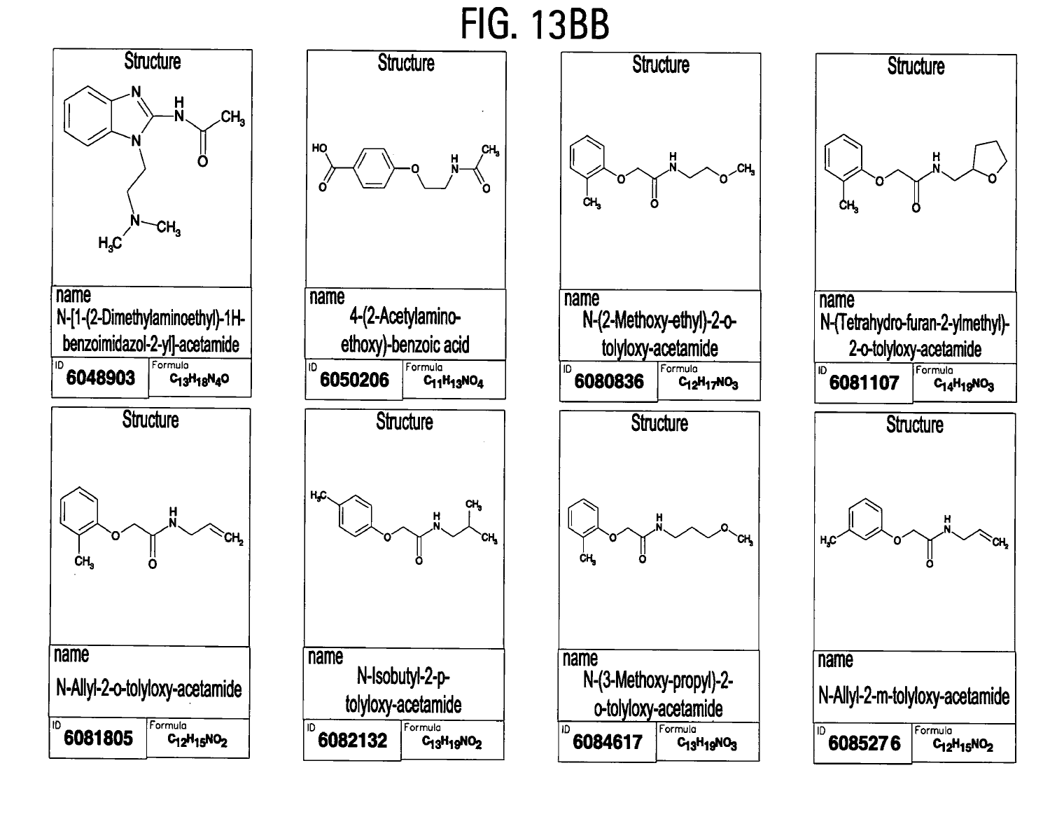

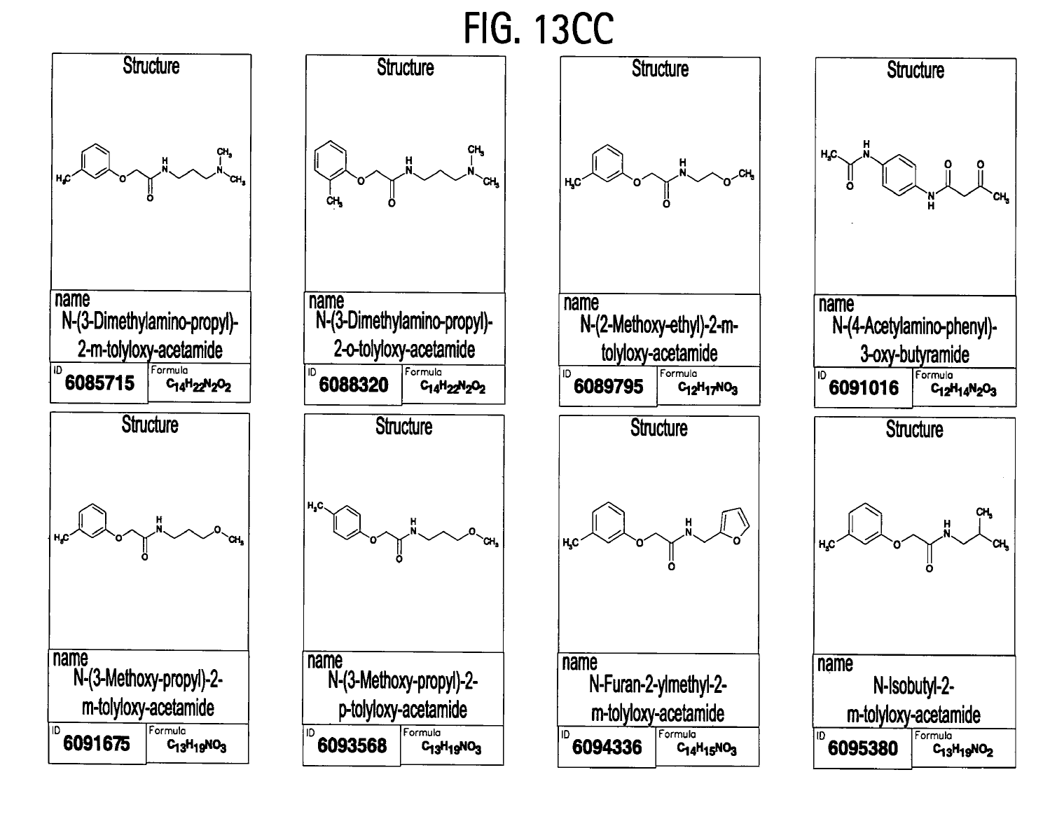

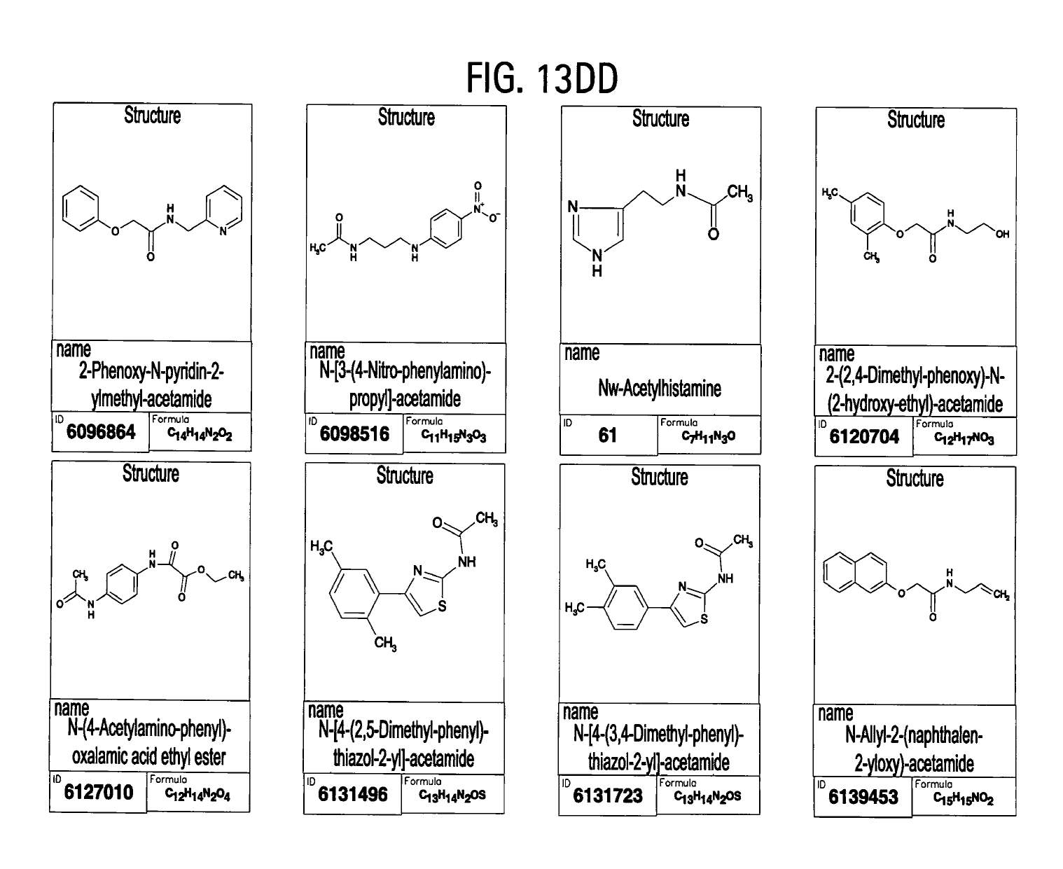

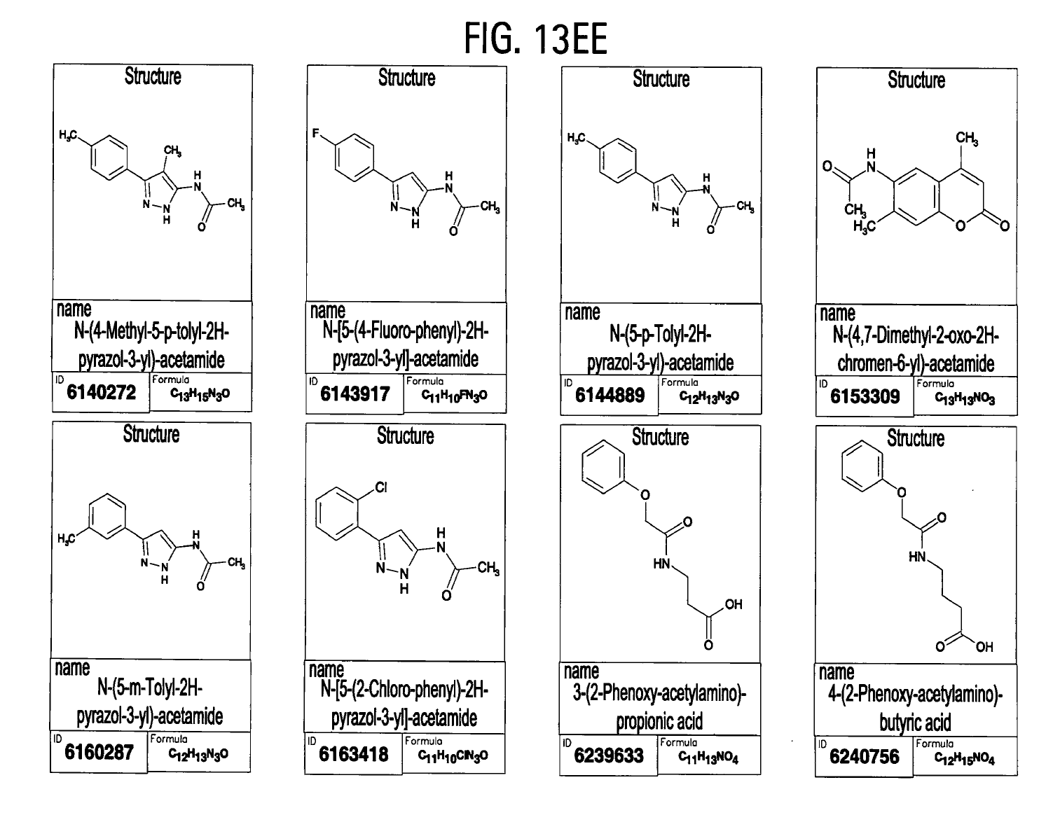

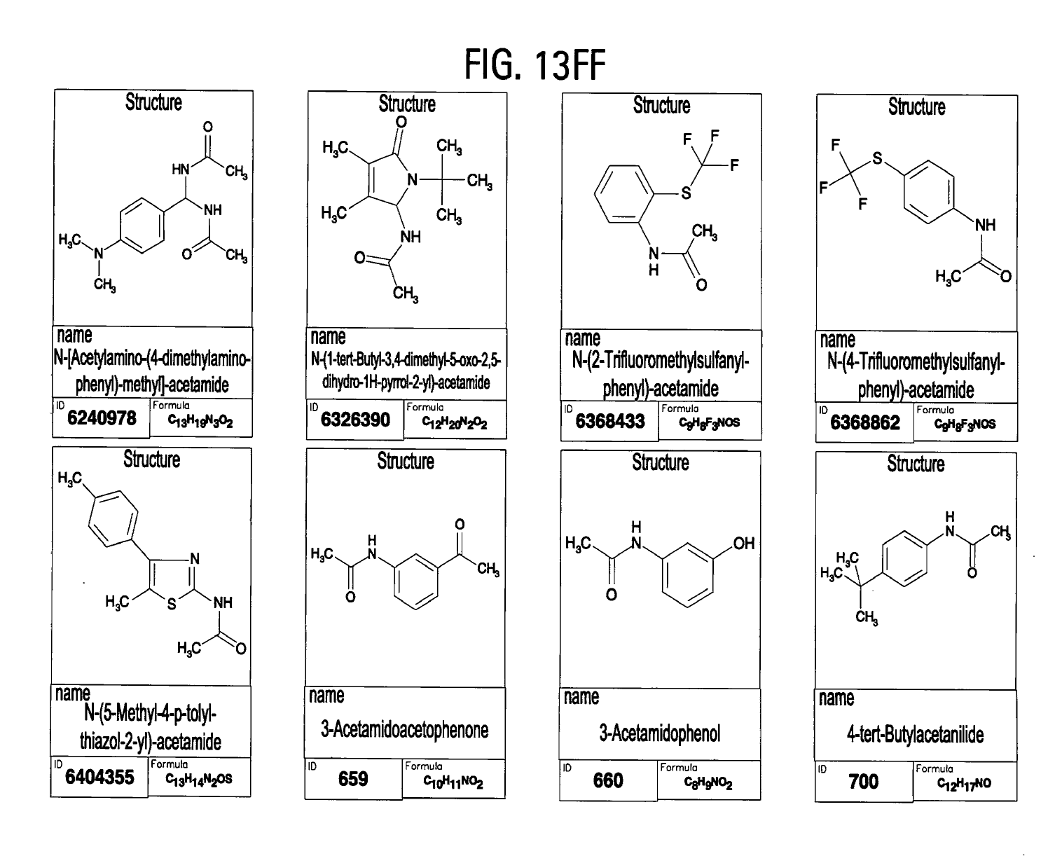

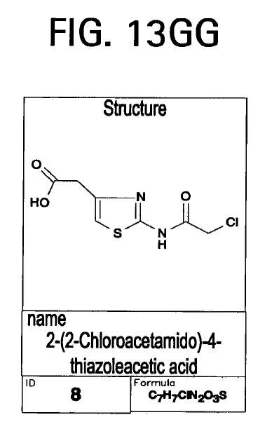

The present invention provides the first detailed structural information regarding a bromodomain and a bromodomain complexed with its acetylated binding partner. The present invention therefore provides the three-dimensional structure of the bromodomain and a bromodomain acetylated binding partner complex. Since the interaction of the bromodomain with a histone for example, can play a significant role in chromatin remodeling/regulation, the structural information provided herein can be employed in methods of identifying drugs that can modulate basic cell processes by modulating the transcription. In a particular embodiment, the three-dimensional structural information is used in the design of a small organic molecule for the treatment of cancer or as disclosed below, HV-1 infection and/or AIDs. In addition, the present invention provides a critical structural feature for a class of inhibitors (acetyl-lysine analogs) of the interaction between bromodomains and their protein binding partners which contain an acetylated-lysine (e.g., Tat with P/CAF), see FIG. 12, as well as a compilation of compounds that share this critical feature, see FIG. 13.

Indeed, the bromodomain and lysine-acetylated protein interaction can now be implicated to play a causal role in the development of a number of diseases including cancers such as leukemia. For example, chromatin remodeling plays a central role in the etiology of viral infection and cancer (Archer, et al., Curr. Opin. Genet. Biol. 9:171-174 (1999); Jacobson, et al., Curr. Opin. Genet. Biol. 9:175-184 (1999)). Both altered histone acetylation/deacetylation and aberrant forms of chromatin-remodeling complexes are associated with human diseases. Furthermore, chromosomal translocation of various cellular genes with those encoding HATs and subunits of chromatin remodeling complexes have been implicated in leukomogenesis. The MOZ (monocytic leukemia zinc finger) and MLL/ALL-1 genes are frequently fused to the gene encoding the co-activator HAT CBP (Sobulo et al., Proc. Natl. Acad. Sci. USA 94:8732-8737(1997)). The resulting fusion protein MLL-CBP contains the tandem bromodomain-PHD finger-HAT domain of CBP. It also has been shown that both the bromodomain and HAT domain of CBP are required for leukomogenesis, because deletion of either the bromodomain or the HAT domain results in loss of the MLL-CBP fusion protein's ability for cell transform. These results indicate that the CBP bromodomain, and more particularly, the ZA loop of the CBP bromodomain, is an excellent target for developing drugs that interfere with the bromodomain acetyl-lysine interaction that can be used in the treatment of human acute leukemia. In addition, an antibody (e.g., a humanized antibody) raised specifically against a peptide from the ZA loop of the CBP bromodomain could also be effective for treating these conditions.

In addition, it is now known that the human immunodeficiency virus type 1 (HIV-1) trans-activator protein, Tat, is tightly regulated by lysine acetylation (Kiernan et al., EMBO Journal 18:6106-6118 (1999)). HIV-1 Tat transcriptional activity is absolutely required for productive HIV viral replication (Jeang, et al., Curr. Top. Microbiol. Immunol., 188:123-144(1994)). Therefore, the interaction of the acetyl-lysine of Tat with one or more bromodomain-containing proteins associated with chromatin remodeling could mediate gene transcription. More particularly, it is disclosed herein that acetylated lysine50 of Tat specifically binds to the bromodomain of P/CAF. Therefore, this particular bromodomain/lysine-acetylated Tat interaction serves as a drug target for blocking HIV replication in cells. As indicated above, an antibody raised specifically against a peptide from the ZA loop of the P/CALF bromodomain could also be effective for treating and/or preventing HIV infections including those that lead to AIDs.

In addition, based on the new structural information disclosed herein, the key amino acid residues for the binding of a given bromodomain and its binding partner can be identified and further elucidated using basic mutagenesis and standard isothermal titration calorimetry, for example. Indeed, both the critical amino acids for the bromodomain and the binding partner (i.e., apart from the acetyl-lysine) can be readily determined and are also part of the present invention.

Therefore, the results obtained from the structural and functional studies disclosed herein provide the foundation for both high throughput drug screening and structure-based rational drug design. The agents identified by this procedure are useful for ameliorating conditions involving chromatin remodeling/regulation, and/or in the treatment of cancer and/or AIDS, as indicated above.

Structure based rational drug design is the most efficient method of drug development. However, heretofore, no information has been disclosed regarding the structure of the bromodomain or more importantly, its interaction with the acetyl-lysine of its binding partner. Obtaining detailed structural information requires an extensive NMR or X-ray crystallographic analysis. By determining and then exploiting the detailed structural information of the bromodomain and of the bromodomain/acetyl-histamine (exemplified by NMR analysis below) the present invention provides novel methods for developing new drugs through structure based rational drug design.

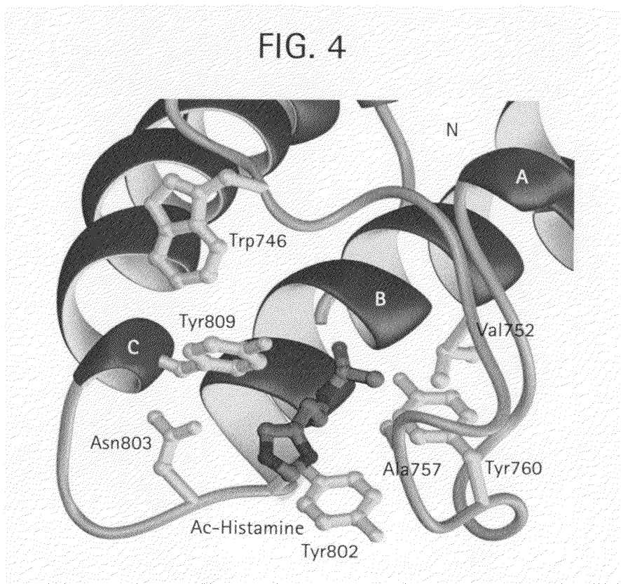

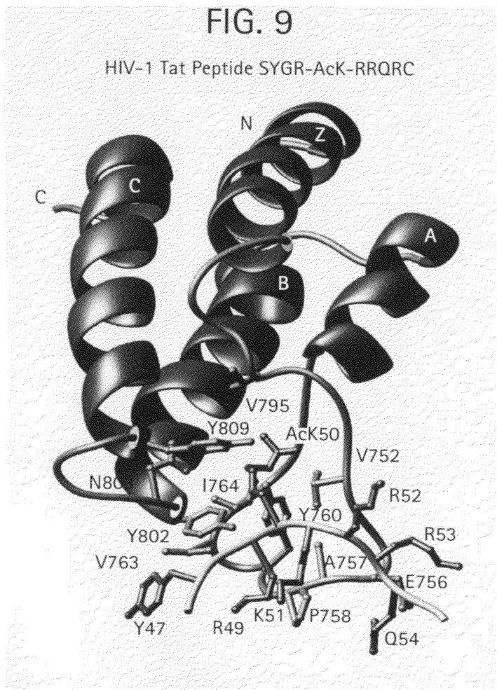

Thus the present invention provides representative sets of the atomic structure coordinates of the free form of the P/CAF bromodomain (Table 5 of U.S. Ser. No. 09/784,553, filed Feb. 16, 2001, now U.S. Pat. No. 7,589,167, the disclosure of which is hereby incorporated by reference), of the P/CAF bromodomain-acetyl-histamine complex (Table 6 of U.S. Ser. No. 09/784,553, filed Feb. 16, 2001, now U.S. Pat. No. 7,589,167, the disclosure of which is hereby incorporated by reference) and of the Tat-P/CAF complex (Table 10 of U.S. Ser. No. 09/784,553, filed Feb. 16, 2001, now U.S. Pat. No. 7,589,167, the disclosure of which is hereby incorporated by reference) which were all obtained by NMR analysis. A Ribbon diagram of the three-dimensional structure of the P/CAF bromodomain is depicted in FIG. 2E, whereas the P/CAF bromodomain acetyl-lysine binding pocket is depicted in FIG. 4 and the Tat-P/CAF complex is depicted in FIG. 9. The present invention also provides the NOE-derived distance restraints, and NMR chemical shift assignments of the P/CAF bromodomain. and the Tat-P/CAF complex. The NMR chemical shift assignments of the P/CAF bromodomain are included in the chemical shift table (Table 1 of U.S. Ser. No. 09/784,553, filed Feb. 16, 2001, now U.S. Pat. No. 7,589,167, the disclosure of which is hereby incorporated by reference) for the ^{1H}-^{15N} HSQC spectrum of P/CAF bromodomain. The unambiguous NOE-derived Inter-proton Distance Restraints (Table 2 of U.S. Ser. No. 09/784,553, filed Feb. 16, 2001, now U.S. Pat. No. 7,589,167, the disclosure of which is hereby incorporated by reference), the ambiguous NOE-derived Inter-proton Distance Restraints (Table 3 of U.S. Ser. No. 09/784,553, filed Feb. 16, 2001, now U.S. Pat. No. 7,589,167, the disclosure of which is hereby incorporated by reference) and the ¹H bonding restraints (Table 4 of U.S. Ser. No. 09/784,553, filed Feb. 16, 2001, now U.S. Pat. No. 7,589,167, the disclosure of which is hereby incorporated by reference) are also disclosed herein. The NMR chemical shift assignments of the Tat-P/CAF complex are included in the chemical shift table (Table 11 of U.S. Ser. No. 09/784,553, filed Feb. 16, 2001, now U.S. Pat. No. 7,589,167, the disclosure of which is hereby incorporated by reference) for the ¹H-¹⁵N HSQC spectrum of P/CAF bromodomain. The unambiguous NOE-derived Inter-proton Distance Restraints (Table 13 of U.S. Ser. No. 09/784,553, filed Feb. 16, 2001, now U.S. Pat. No. 7,589,167, the disclosure of which is hereby incorporated by reference), the ambiguous NOE-derived Inter-proton Distance Restraints (Table 14 of U.S. Ser. No. 09/784,553, filed Feb. 16, 2001, now U.S. Pat. No. 7,589,167, the disclosure of which is hereby incorporated by reference) and the ¹H bonding restraints (Table 12 of U.S. Ser. No. 09/784,553, filed Feb. 16, 2001, now U.S. Pat. No. 7,589,167, the disclosure of which is hereby incorporated by reference) are also disclosed herein. The sample atomic coordinate data provided enable the skilled artisan to practice the invention.

In addition, Tables 1-6 and/or 10-14 of U.S. Ser. No. 09/784,553, filed Feb. 16, 2001, now U.S. Pat. No. 7,589,167, the disclosure of which is hereby incorporated by reference, are also capable of being placed into a computer readable form which is also part of the present invention. Furthermore, methods of using these coordinates and chemical shifts and related information (including in computer readable forms) either individually or together in drug assays are also provided. More particularly, such atomic coordinates can be used to identify potential ligands or drugs which will modulate the binding of a bromodomain with its binding partner.

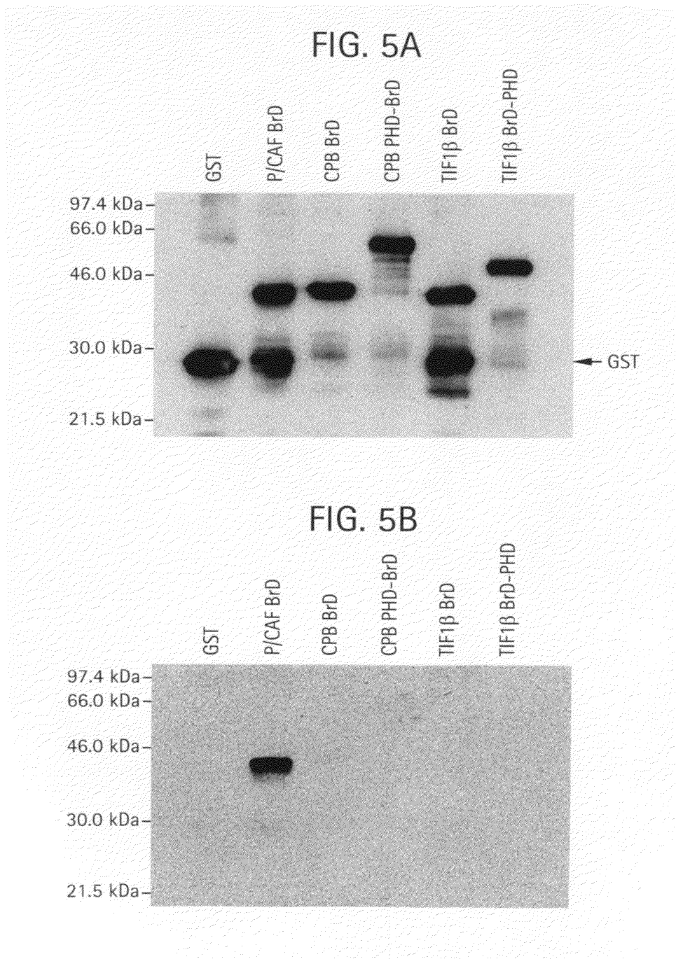

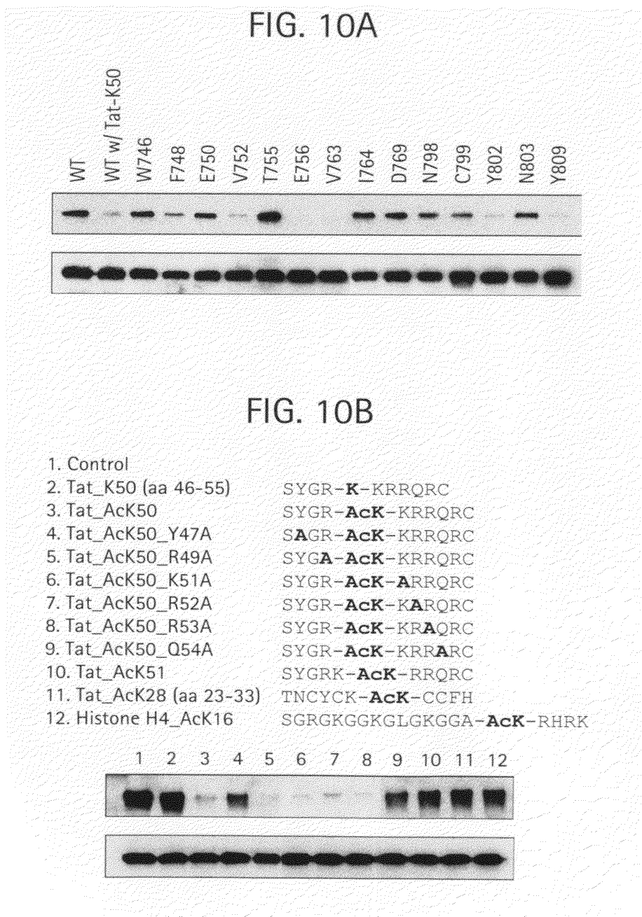

In a particular aspect of the present invention, the lysine-acetylated Tat is shown herein to specifically bind to the bromodomain of the p300/CBP-associated factor (P/CAF) in vitro and in vivo. Structural and mutational analyses provides the identification of key amino acid residues on both the bromodomain and Tat that are important for the binding complex. The identification of these important amino acid residues further demonstrates the biological importance of this interaction for Tat transactivation activity. Together, the findings disclosed herein indicate a novel mechanism by which the lysine-acetylated Tat recruits P/CAF via a bromodomain interaction, leading to chromatin remodeling-mediated transcriptional activation of HIV-1. Furthermore, the extreme specificity of the Tat-P/CAF binding (see e.g., FIGS. 5A-5B and 10A-10B) indicates that compounds that interfere with this binding complex are not likely to interfere to otherwise related bromodomain-ligand interactions.

Therefore, the three-dimensional structural information provided by the present invention allows the identification and/or design of specific compounds that can act as modulators of crucial processes. In the case of the Tat-P/CAF interaction, such compounds can be used as drugs to inhibit HIV-1 expression in a cell and/or subsequent infection of other cells. Therefore, the inhibitors identified and/or designed by the methods disclosed can be used to prevent, treat, retard the progression, and potentially cure HIV-1 infections and AIDS.

Therefore, if appearing herein, the following terms shall have the definitions set out below.

As used herein a “bromodomain-acetyl-lysine binding complex” is a binding complex between a bromodomain or fragment thereof and either a peptide/polypeptide comprising an acetyl-lysine (or an analog of acetyl-lysine), or a free analog of acetyl-lysine, such as acetyl-histamine disclosed in the Example below. Preferably, the peptide comprises at least six amino acids in addition to the acetyl-lysine. A fragment of a bromodomain preferably comprises a ZA loop as defined below. The dissociation constant of a bromodomain-acetyl-lysine binding complex is dependent on whether the lysine residue or analog thereof is acetylated or not, such that the affinity for the bromodomain and the peptide comprising the lysine residue (for example) significantly decreases when that lysine residue is not acetylated. One example of a bromodomain-acetyl-lysine binding complex is that formed between P/CAF with Tat (the “Tat-P/CAF complex”) as exemplified below.

As used herein the term “acetyl-lysine analog” is used interchangeably with the term “analog of acetyl-lysine” and is a compound that contains the acetyl-amine-like structure as depicted in FIG. 12. Examples of acetyl-lysine analogs are included in FIG. 13.

As used herein a “ZA loop” of a bromodomain is a key portion of a bromodomain that is involved in the binding of the bromodomain to the acetyl-lysine. The structure of the actual ZA loop of the bromodomain of P/CAF is depicted in FIG. 2A. As used herein, however, a ZA loop has between about 20 and 40 amino acids and preferably comprises the amino acid sequence of SEQ ID NO:3 and/or SEQ ID NO:48. More preferably the ZA loop comprises between about 23 to 34 amino acids. In a specific embodiment the ZA loop has the amino acid sequence SEQ ID NO:43. The amino acid sequence of the ZA loop for a representative number of individual bromodomains is shown in FIG. 1.

A “polypeptide” or “peptide” comprising a fragment of a bromodomain, such as the ZA loop, or a peptide or polypeptide comprising an acetyl-lysine, as used herein can be the “fragment” alone, or a larger chimeric or fusion peptide/protein which contains the “fragment”.

As used herein the terms “fusion protein” and “fusion peptide” are used interchangeably and encompass “chimeric proteins and/or chimeric peptides” and fusion “intein proteins/peptides”. A fusion protein comprises at least a portion of a protein or peptide of the present invention, e.g., a bromodomain, joined via a peptide bond to at least a portion of another protein or peptide including e.g., a second bromodomain in a chimeric fusion protein. In a particular embodiment the portion of the bromodomain is antigenic. Fusion proteins can comprise a marker protein or peptide, or a protein or peptide that aids in the isolation and/or purification of the protein, for example.

As used herein, and unless otherwise specified, the terms “agent”, “potential drug”, “compound”, “test compound” or “potential compound” are used interchangeably, and refer to chemicals which potentially have a use as an inhibitor or activator/stabilizer of bromodomain-acetyl-lysine binding. Therefore, such “agents”, “potential drugs”, “compounds” and “potential compounds” may be used, as described herein, in drug assays and drug screens and the like.

As used herein a “small organic molecule” is an organic compound, including a peptide (or organic compound complexed with an inorganic compound (e.g., metal)) that has a molecular weight of less than 3 Kilodaltons. Such small organic molecules can be included as agents, etc. as defined above.

As used herein the term “binds to” is meant to include all such specific interactions that result in two or more molecules showing a preference for one another relative to some third molecule. This includes processes such as covalent, ionic, hydrophobic and hydrogen bonding but does not include non-specific associations such as solvent preferences.

As used herein the term “about” signifies that a value is within twenty percent of the indicated value i.e., a peptide containing “about” 20 amino acid residues can contain between 16 and 24 amino acid residues.

General Techniques for Constructing Nucleic Acids That Encode the Bromodomains and Fragments Thereof (Including, ZA Loops), and the Bromodomain Binding Partners of the Present Invention.

In accordance with the present invention there may be employed conventional molecular biology, microbiology, and recombinant DNA techniques within the skill of the art. Such techniques are explained fully in the literature. See, e.g., Sambrook, et al. Molecular Cloning: A Laboratory Manual, Third Edition (2001) Vols. I-E, Cold Spring Harbor Laboratory Press, Cold Spring Harbor, N.Y. (herein “Sambrook and Russell, 2001”), Sambrook, et al., Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRE Press, (1986)); Perbal, A Practical Guide To Molecular Cloning (1984); Ausubel et al. (eds.), Current Protocols in Molecular Biology, John Wiley & Sons, Inc. (1994).

As used herein, the term “gene” refers to an assembly of nucleotides that encode a polypeptide, and includes cDNA and genomic DNA nucleic acids.

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogues thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989 supra, Sambrook and Russell, 2001). The conditions of temperature and ionic strength determine the “stringency” of the hybridization. For preliminary screening for homologous nucleic acids, low stringency hybridization conditions, corresponding to a Tₘ of 55°, can be used, e.g., 5.times.SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5.times.or 6.times.SCC. High stringency hybridization conditions correspond to the highest Tₘ, e.g., 50% formamide, 5× or 6×SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., 1989 supra, 9.50-10.51, Sambrook and Russell, 2001). For hybridization with shorter nucleic acids, i.e., oligonucleotides, the position of mismatches becomes more important, and the length of the oligonucleotide determines its specificity (see Sambrook et al., 1989 supra, 11.7-11.8, Sambrook and Russell, 2001). Preferably a minimum length for a hybridizable nucleic acid is at least about 12 nucleotides; preferably at least about 18 nucleotides; and more preferably the length is at least about 27 nucleotides; and most preferably 36 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences and synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

As used herein, the term “homologous” refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell, 50:667 (1987)). Such proteins have sequence homology as reflected by their high degree of sequence similarity.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and not a common evolutionary origin.

Two DNA sequences are “substantially homologous” when at least about 60% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art (See, e.g., Sambrook et al., 1989 supra; DNA Cloning, Vols. I & II, supra; Nucleic Acid Hybridization, supra., and Sambrook and Russell, 2001)

As used herein an amino acid sequence is 100% “homologous” to a second amino acid sequence if the two amino acid sequences are identical, and/or differ only by neutral or conservative substitutions as defined below. Accordingly, an amino acid sequence is 50% “homologous” to a second amino acid sequence if 50% of the two amino acid sequences are identical, and/or differ only by neutral or conservative substitutions.

As used herein, DNA and protein sequence percent identity can be determined using MacVector 6.0.1, Oxford Molecular Group PLC (1996) and the Clustal W algorithm with the alignment default parameters, and default parameters for identity. These commercially available programs can also be used to determine sequence similarity using the same or analogous default parameters.

The term “corresponding to” is used herein to refer similar or homologous sequences, whether the exact position is identical or different from the molecule to which the similarity or homology is measured. Thus, the term “corresponding to” refers to the sequence similarity, and not the numbering of the amino acid residues or nucleotide bases.

As used herein a “heterologous nucleotide sequence” is a nucleotide sequence that is added to a nucleotide sequence of the present invention by recombinant methods to form a nucleic acid which is not naturally formed in nature. Such nucleic acids can encode fusion proteins or peptides, including chimeric proteins and peptides. Thus the heterologous nucleotide sequence can encode peptides and/or proteins which contain regulatory and/or structural properties. In another such embodiment the heterologous nucleotide can encode a protein or peptide that functions as a means of detecting the protein or peptide encoded by the nucleotide sequence of the present invention after the recombinant nucleic acid is expressed. In still another such embodiment the heterologous nucleotide can function as a means of detecting a nucleotide sequence of the present invention. A heterologous nucleotide sequence can comprise non-coding sequences including restriction sites, regulatory sites, promoters and the like.

The present invention also relates to cloning vectors containing nucleic acids encoding analogs and derivatives of the bromodomains of the present invention and polypeptides/peptides that can bind a bromodomain when a lysine of the polypeptide/peptide is acetylated, including modified fragments, that have the same or homologous functional activity as the individual fragments, and homologs thereof. The production and use of derivatives and analogs related to the fragments are within the scope of the present invention.

Due to the degeneracy of nucleotide coding sequences, other DNA sequences which encode substantially the same amino acid sequence as a nucleic acid encoding a protein comprising bromodomain or bromodomain binding partner (i.e., when post-transcriptionally acetylated) of the present invention for example, may be used in the practice of the present invention. These include but are not limited to allelic genes, homologous genes from other species, which are altered by the substitution of different codons that encode the same amino acid residue within the sequence, thus producing a silent change. Likewise, the peptides and polypeptides of the present invention include, but are not limited to, those containing, as a primary amino acid sequence, analogous portions of their respective amino acid sequences including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, and lysine. The negatively charged (acidic) amino acids include aspartic acid and glutamic acid.

Particularly preferred conserved amino acid exchanges are:

(a) Lys for Arg or vice versa such that a positive charge may be maintained;

(b) Glu for Asp or vice versa such that a negative charge may be maintained;

(c) Ser for Thr or vice versa such that a free —OH can be maintained;

(d) Gln for Asn or vice versa such that a free NH₂ can be maintained;

(e) Ile for Leu or for Val or vice versa as roughly equivalent hydrophobic amino acids; and

(f) Phe for Tyr or vice versa as roughly equivalent aromatic amino acids.

A conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein. Specific amino acid residues for the P/CAF bromodomain have been identified that are important for binding, indicating a potential lower stringency for the substitution of the remaining amino acids residues.

All of the peptides/fragments of the present invention can be modified by being placed in a fusion or chimeric peptide or protein, or labeled e.g., to have an N-terminal FLAG-tag, or H6 tag. In a particular embodiment the P/CAF bromodomain fragment can be modified to contain a marker protein such as green fluorescent protein as described in U.S. Pat. No. 5,625,048 filed Apr. 29, 1997 and WO 97/26333, published Jul. 24, 1997 each of which are hereby incorporated by reference herein in their entireties.

The nucleic acids encoding peptides and protein fragments of the present invention and analogs thereof can be produced by various methods known in the art. The manipulations which result in their production can occur at the gene or protein level (Sambrook et al., 1989, supra; Sambrook and Russell, 2001, supra). The nucleotide sequence can be cleaved at appropriate sites with restriction endonuclease(s), followed by further enzymatic modification if desired, isolated, and ligated in vitro. In addition a nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson et al., J. Biol. Chem., 253:6551 (1978); Zoller, et al, DNA, 3:479-488 (1984); Oliphant et al., Gene, 44:177 (1986); Hutchinson et al., Proc. Natl. Acad. Sci. U.S.A., 83:710 (1986)), use of TABφ linkers (Pharmacia), etc. PCR techniques are preferred for site directed mutagenesis (see Higuchi, “Using PCR to Engineer DNA”, in PCR Technology: Principles and Applications for DNA Amplification, H. Erlich, ed., Stockton Press, Chapter 6, pp. 61-70 (1989)).

The identified and isolated nucleic acids can then be inserted into an appropriate cloning vector. A large number of vector-host systems known in the art may be used.

### Protein Expression and Purification

A bacterial protein expression system can be used to make various stable isotopically labeled (¹³C, ¹⁵N, and ²H) protein samples that are useful for a three-dimensional NMR structural determination of a protein complex. For example a pET14b (Novagen) bacterial expression vector can be constructed which expresses the recombinant P/CAF bromodomain as an amino-terminal His-tagged fusion protein.

Protein expression and purification can be conducted using standard procedures for His-tagged proteins (Zhou et al., J. Biol. Chem. 270:31119-31123 (1995)). To optimize the level of protein expression, various bacterial growth and expression conditions can be screened, which include different E. Coli cell lines, and growth and protein induction temperatures. Generally, it is preferred to obtain the maximum amount of soluble protein while still inducing protein expression with a relatively low IPTG concentration e.g., .about.0.2 mM (final concentration) at 16° C. As exemplified below, the bromodomain of P/CAF (residues 719-832 of SEQ ID NO:2 which is SEQ ID NO:7) was subcloned into the pET14b expression vector (Novagen) and expressed in Escherichia coli BL21(DE3) cells. Uniformly ¹⁵N- and ¹⁵N/¹³C-labeled proteins were prepared by growing bacteria in a minimal medium containing ¹⁵NH₄Cl with or without ¹³C₆-glucose. A uniformly ¹⁵N/¹³C-labeled and fractionally deuterated protein sample was prepared by growing the cells in 75% ²H₂O. The bromodomain was purified by affinity chromatography on a nickel-IDA column (Invitrogen) followed by the removal of poly-His tag by thrombin cleavage. The final purification of the protein was achieved by size-exclusion chromatography. The acetyl-lysine-containing peptides were prepared on a MilliGen 9050 peptide synthesizer (Perkin Elmer) using Fmoc/HBTU chemistry. Acetyl-lysine was incorporated using the reagent Fmoc-Ac-Lys with HBTU/DIPEA activation. NMR samples contained approximately 1 mM protein in 100 mM phosphate buffer of pH 6.5 and 5 mM perdeuterated DTT and 0.5 mM EDTA in H₂O/²H₂O (9/1) or ²H₂O.

One major advantage of using the heteronuclear multidimensional approach, as exemplied herein, is that the NMR resonance assignments of a protein are obtained in a sequence-specific manner which assures accuracy and greatly facilitates data analysis and structure determination (Clore, et al., Meth. Enzymol. 239:249-363 (1994)). In addition, the signal overlapping problems in the protein spectra are minimized by the use of multidimensional NMR spectra, which separates the proton signals according to the chemical shifts of their attached hetero-nuclei (such as ¹⁵N and ¹³C). This NMR approach has been proven very powerful for structural analysis of large proteins (Clore, et al., Meth. Enzymol. 239:249-363 (1994)). To facilitate sequence-specific resonance assignments for the structural study, a uniformly ¹³C, ¹⁵N-labeled and fractionally (75%) deuterated protein sample of the bromodomain can be prepared by growing bacterial cells in 75% ²H₂O as exemplified below. Such protein samples can be used for triple-resonance NMR experiments. A triple-labeled protein sample is useful for high-resolution NMR structural studies. Because of the favorable ¹H, ¹³C, and ¹⁵N relaxation rates caused by the partial deuteration of the protein, constant-time triple-resonance NMR spectra can be acquired with higher digital resolution and sensitivity (Sattler, et al., Structure 4:1245-1249 (1996)). In addition, various stable-isotopically labeled (¹⁵N and ¹³C/¹⁵N) proteins can also be prepared using this procedure.

### Synthetic Polypeptides

The term “polypeptide” is used in its broadest sense to refer to a compound of two or more subunit amino acids, amino acid analogs, or peptidomimetics. The subunits are linked by peptide bonds. The terms “polypeptide”, “protein”, and “peptide” are used interchangeably herein, though preferably as used herein a “peptide” refers to a compound of at least two but less than fifty subunit amino acids, and a polypeptide or protein refers to compound of fifty or more amino acids. The polypeptides of the present invention may be chemically synthesized or as detailed above, genetically engineered or isolated from natural sources.

In addition, potential drugs or agents that may be tested in the drug screening assays of the present invention may also be chemically synthesized. When the peptide is to be modified, e.g., acetylated, the modification can be at any time during the peptide synthesis, including using an acetyl-lysine as a starting material or acetylating a lysine residue of a peptide after the peptide has been synthesized. In the Example below, the acetyl-lysine-containing peptides were prepared on a MilliGen 9050 peptide synthesizer (Perkin Elmer) using Fmoc/HITU chemistry. Acetyl-lysine was incorporated using the reagent Fmoc-Ac-Lys with BBTUIDIPEA activation.

Thus, synthetic polypeptides, prepared using the well known techniques of solid phase, liquid phase, or peptide condensation techniques, or any combination thereof, can include natural and unnatural amino acids. Amino acids used for peptide synthesis may be standard Boc (N^{alpha}.-amino protected N^{alpha}.-t-butyloxycarbonyl)amino acid resin with the standard deprotecting, neutralization, coupling and wash protocols of the original solid phase procedure of Merrifield (J. Am. Chem. Soc., 85:2149-2154 (1963)), or the base-labile N^{alpha}.-amino protected 9-fluorenylmethoxycarbonyl (Fmoc) amino acids first described by Carpino and Han (J. Org. Chem., 37:3403-3409 (1972)). Both Fmoc and Boc N_{alpha.-amino} protected amino acids can be obtained from Fluka, Bachem, Advanced Chemtech, Sigma, Cambridge Research Biochemical, Bachem, or Peninsula Labs or other chemical companies familiar to those who practice this art. In addition, the method of the invention can be used with other Nₐₗₚₕₐ-protecting groups that are familiar to those skilled in this art. Solid phase peptide synthesis may be accomplished by techniques familiar to those in the art and provided, for example, in Stewart and Young (Solid Phase Synthesis, Second Edition, Pierce Chemical Co., Rockford, Ill. (1984)) and Fields and Noble (Int. J. Pept. Protein Res., 35:161-214 (1990)), or using automated synthesizers, such as sold by ABS. Thus, polypeptides of the invention may comprise D-amino acids, a combination of D- and L-amino acids, and various “designer” amino acids (e.g., β-methyl amino acids, Ca-methyl amino acids, and Nα-methyl amino acids, etc.) to convey special properties. Alternative synthetic amino acids that can be used include ornithine for lysine, fluorophenylalanine for phenylalanine, and norleucine for leucine or isoleucine. Other synthetic amino acids include 2-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, sarcosine, N-methylisoleucine, 6-N-methyllysine, and N-methylvaline. Additionally, by assigning specific amino acids at specific coupling steps, α-helices, β turns, β sheets, γ-turns, and cyclic peptides can be generated.

In a further embodiment, subunits of peptides that confer useful chemical and structural properties will be chosen. For example, peptides comprising D-amino acids will be resistant to L-amino acid-specific proteases in vivo. In addition, the present invention envisions preparing peptides that have more well defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a molecule would be resistant to peptide bond hydrolysis, e.g., protease activity. Such peptides would provide ligands with unique function and activity, such as extended half-lives in vivo due to resistance to metabolic breakdown, or protease activity. Furthermore, it is well known that in certain systems constrained peptides show enhanced functional activity (Hruby, Life Sciences, 31:189-199 (1982); Hruby et al., Biochem J., 268:249-262 (1990)); the present invention provides a method to produce a constrained peptide that incorporates random sequences at all other positions.

Constrained and cyclic peptides. A constrained, cyclic or rigidized peptide may be prepared synthetically, provided that in at least two positions in the sequence of the peptide an amino acid or amino acid analog is inserted that provides a chemical functional group capable of crosslinking to constrain, cyclise or rigidize the peptide after treatment to form the crosslink. Cyclization will be favored when a turn-inducing amino acid is incorporated. Examples of amino acids capable of crosslinking a peptide are cysteine to form disulfides, aspartic acid to form a lactone or a lactam, and a chelator such as γ-carboxyl-glutamic acid (Gla) (Bachem) to chelate a transition metal and form a cross-link. Protected γ-carboxyl glutamic acid may be prepared by modifying the synthesis described by Zee-Cheng and Olson (Biophys. Biochem. Res. Commun., 94:1128-1132 (1980)). A peptide in which the peptide sequence comprises at least two amino acids capable of crosslinking may be treated, e.g., by oxidation of cysteine residues to form a disulfide or addition of a metal ion to form a chelate, so as to crosslink the peptide and form a constrained, cyclic or rigidized peptide.

The present invention provides strategies to systematically prepare cross-links. For example, if four cysteine residues are incorporated in the peptide sequence, different protecting groups may be used (Hiskey, in The Peptides: Analysis, Synthesis, Biology, Vol. 3, Gross, et al., eds., Academic Press: New York, pp. 137-167 (1981); Ponsanti et al., Tetrahedron, 46:8255-8266 (1990)). The first pair of cysteines may be deprotected and oxidized, then the second set may be deprotected and oxidized. In this way a defined set of disulfide cross-links may be formed. Alternatively, a pair of cysteines and a pair of chelating amino acid analogs may be incorporated so that the cross-links are of a different chemical nature.

Non-classical amino acids that induce conformational constraints. The following non-classical amino acids may be incorporated in the peptide in order to introduce particular conformational motifs: 1,2,3,4-tetrahydroisoquinoline-3-carboxylate (Kazmierski et al., J. Am. Chem. Soc., 113:2275-2283 (1991)); (2S,3S)-methyl-phenylalanine, (2S,3R)-methyl-phenylalanine, (2R,3S)-methyl-phenylalanine and (2R,3R)-methyl-phenylalanine (Kazinierski, et al., Tetrahedron Lett. (1991)); 2-aminotetrahydronaphthalene-2-carboxylic acid (Landis, Ph.D. Thesis, University of Arizona (1989)); hydroxy-1,2,3,4-tetrahydroisoquino-line-3-carboxylate (Miyake et al., J. Takeda Res. Labs., 43:53-76 (1989)); β-carboline (D and L) (Kazmierski, Ph.D. Thesis, University of Arizona (1988)); HIC (histidine isoquinoline carboxylic acid) (Zechel et al., Int. J. Pep. Protein Res., 43 (1991)); and HIC (histidine cyclic urea) (Dharanipragada).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al., J. Org. Chem., 50:5834-5838 (1985)); β-sheet inducing analogs (Kemp et al., Tetrahedron Lett., 29:5081-5082 (1988); β-turn inducing analogs (Kemp et al., Tetrahedron Lett., 29:5057-5060 (1988)); .varies.-helix inducing analogs (Kemp et al., Tetrahedron Lett., 29:4935-4938 (1988)); γ-turn inducing analogs (Kemp et al., J. Org. Chem., 54:109:115 (1989)); and analogs provided by the following references: Nagai, et al., Tetrahedron Len., 26:647-650 (1985); DiMaio et al., J. Chem. Soc. Perkin Trans., p. 1687 (1989); also a Gly-Ala turn analog (Kahn et al., Tetrahedron Lett., 30:2317 (1989)); amide bond isostere (Jones et al., Tetrahedron Lett., 29:3853-3856 (1988)); tretrazol (Zabrocki et al., J. Am. Chem. Soc., 110:5875-5880 (1988)); DTC (Samanen et al., Int. J. Protein Pep. Res., 35:501:509 (1990)); and analogs taught in Olson et al., J. Am. Chem. Sci., 112:323-333 (1990) and Garvey et al., J. Org. Chem., 56:436 (1990). Conformationally restricted mimetics of beta turns and beta bulges, and peptides containing them, are described in U.S. Pat. No. 5,440,013, issued Aug. 8, 1995 to Kahn.

### Structure-Based Mutation Analysis

Protein structural analysis using NMR spectroscopy has several unique advantages. In addition to high-resolution three-dimensional structural information, the chemical shift assignments for the protein obtained in the structural study further provides a map of the entire protein at the atomic level, which can be used for structure-based biochemical analysis of protein-protein interactions. For example, the information generated from the NMR structural analysis can also serve to identify specific amino acid residues in the peptide-binding site for complementary mutagenesis studies. Specific focus can be placed on those residues that display long-range NOEs (particularly the side-chain NOEs in the ¹³C-NOESY data) between the bromoomain and a peptide comprising an acetyl-lysine.

To ensure mutant proteins are valid for functional analysis, it can be determined as to whether a mutation results in any significant perturbation of the overall conformation of the bromodomain, particularly the effects of mutation on the acetyl-lysine binding sites. NMR spectroscopy is a powerful method for examining the effects of such a mutation on the conformation of the protein. One can readily obtain information about the global conformation of a mutant protein from the proton (¹H) ID spectrum, by examining the chemical shift dispersion and peak line-width of NMR signals of amide, aromatic and aliphatic protons. Moreover, 2D ¹H-¹⁵N HSQC spectra reveal details of the effects of a mutation on both local and global conformation of the protein, since every single ¹H/¹⁵N signal (both the chemical shift and line-shape) in the NMR spectrum is a “reporter” for a particular amino acid residue. Thus, to assess how mutations affect protein stability and the overall protein conformation, the ¹⁵N HSQC spectra of mutated proteins can be compared to that of the wild-type protein bromodomain.

Chemical-shift perturbations due to ligand binding have proven to be a reliable and sensitive probe for the ligand binding site of the protein. This is because the chemical-shift changes of the backbone amide groups are likely to reflect any changes in protein conformation and/or hydrogen bonding due to the peptide/ligand binding. To examine the effects of a mutation on the ligand binding (in this case the ligand is a peptide comprising an acetyl-lysine), peptide titration experiments can be conducted by following the changes of ¹H/¹⁵N signals of the mutant proteins as a function of the peptide concentration. These experiments indicate whether the acetyl-lysine binding site remains the same or changes in the mutants relative to the wild type protein. The effects of the mutation on the peptide binding affinity can also be examined by NMR spectroscopy. If the mutated proteins result in the reduction of the binding affinity, a change of the exchange phenomenon between the free and the ligand-bound signals should be observed in NMR spectrum. If the reduction in binding affinity causes the peptide binding to change from a slow exchange rate to a fast exchange rate, on the NMR time scale, then the peptide binding affinity can be determined from the NMR titration experiment. From these mutation analyses key amino acid residues that are important for binding a peptide comprising the acetyl-lysine can be identified. Such analysis has been exemplified below.

### Protein Structure Determination by NMR Spectroscopy

The NMR results from the present invention are summarized by the atomic structure coordinates of the free form of the P/CAF bromodomain (Table 5 of U.S. Ser. No. 09/784,553, filed Feb. 16, 2001, now U.S. Pat. No. 7,589,167, the disclosure of which is hereby incorporated by reference), of the P/CAF bromodomain-acetyl-histamine complex (Table 6 of U.S. Ser. No. 09/784,553, filed Feb. 16, 2001, now U.S. Pat. No. 7,589,167, the disclosure of which is hereby incorporated by reference), and the Tat-P/CAF complex (Table 10 of U.S. Ser. No. 09/784,553, filed Feb. 16, 2001, now U.S. Pat. No. 7,589,167, the disclosure of which is hereby incorporated by reference). The NMR chemical shift assignments of the P/CAF bromodomain are included in the chemical shift table (Table 1 of U.S. Ser. No. 09/784,553, filed Feb. 16, 2001, now U.S. Pat. No. 7,589,167, the disclosure of which is hereby incorporated by reference) for the ¹H-¹⁵N HSQC spectrum of P/CAF bromodomain. The unambiguous NOE-derived Inter-proton Distance Restraints for the P/CAF bromodomain are in Table 2 of U.S. Ser. No. 09/784,553, filed Feb. 16, 2001, now U.S. Pat. No. 7,589,167, the disclosure of which is hereby incorporated by reference, the ambiguous NOE-derived Inter-proton Distance Restraints are in Table 3, and the ¹H bonding restraints are disclosed in Table 4 of U.S. Ser. No. 09/784,553, filed Feb. 16, 2001, now U.S. Pat. No. 7,589,167, the disclosure of which is hereby incorporated by reference. The NMR chemical shift assignments of the Tat-P/CAF complex are included in the chemical shift table (Table 11 of U.S. Ser. No. 09/784,553, filed Feb. 16, 2001, now U.S. Pat. No. 7,589,167, the disclosure of which is hereby incorporated by reference) for the ¹H-¹⁵N HSQC spectrum of Tat-P/CAF complex The unambiguous NOE-derived Inter-proton Distance Restraints for the Tat-P/CAF complex are in Table 13, the ambiguous NOE-derived Inter-proton Distance Restraints are in Table 14 of U.S. Ser. No. 09/784,553, filed Feb. 16, 2001, now U.S. Pat. No. 7,589,167, the disclosure of which is hereby incorporated by reference, and the ¹H bonding restraints are disclosed in Table 12 of U.S. Ser. No. 09/784,553, filed Feb. 16, 2001, now U.S. Pat. No. 7,589,167, the disclosure of which is hereby incorporated by reference.

Backbone and Side-chain Assignments: Sequence-specific backbone assignment can be achieved by using a suite of deuterium-decoupled triple-resonance 3D NMR experiments which include UNCA, HN(CO)CA, HN(CA)CB, HN(COCA)CB, HNCO, and HN(CA)CO experiments (Yamazaki, et al., J. Am. Chem. Soc. 116:11655-11666 (1994)). The water flip-back scheme is used in these NMR pulse programs to minimize amide signal attenuation from water exchange. Sequential side-chain assignments are typically accomplished from a series of 3D NMR experiments with alternative approaches to confirm the assignments. These experiments include 3D ¹⁵N TOCSY-HSQC, HCCH-TOCSY, (H)C(CO)NH-TOCSY, and H(C)(CO)NH-TOCSY (see Clore, et al., Enzymol. 239:249-363 (1994); Sattler et al., Prog. in Nuclear Magnetic Resonance Spec. 4:93-158 (1999)).

Stereospecific Methyl Groups: Stereospecific assignments of methyl groups of Valine and Leucine residues can be obtained from an analysis of carbon signal multiplet splitting using a fractionally ¹³C-labeled protein sample, which can be readily prepared using M9 minimal medium containing 10% ¹³C-/90% ¹²C-glucose mixture (see Neri, et al., Biochemistry 28:7510-7516 (1989)).

Dihedral Angle Restraints: Backbone dihedral angle (.PHI.) constraints can be generated from the.^{3J}_{HNH}α coupling constants measured in a HNHA-J experiment (see Vuister, et al., J. Am. Chem. Soc. 115:7772-7777 (1993)). Side-chain dihedral angles (_{chi}.1) can be obtained from short mixing time ^{15N}-edited 3D TOCSY-HSQC (see Clore, et al., J. Biomol. NMR 1:13-22 (1991)) and 3D HNH experiments (see Matson et al., J. Biomol. NMR 3:239-244 (1993)), which can also provide stereospecific assignments of β methylene protons.

Hydrogen Bonds Restraints: Amide protons that are involved in hydrogen bonds can be identified from an analysis of amide exchange rates measured from a series of 2D¹H/¹⁵N HSQC spectra recorded after adding ²H₂^{o} to the protein sample.

NOE Distance Restraints: Distance restraints are obtained from analysis of ¹⁵N, and ¹³C-edited 3D NOESY data, which can be collected with different mixing times to 30 minimize spin diffusion problems. The nuclear Overhauser effect (NOE)-derived restraints are categorized as strong (1.8-3 .ANG.), medium (1.8-4 A) or weak (1.8-5. ANG.) based on the observed NOE intensities. A recently developed procedure for the iterative automated NOE analysis by using ARIA (see Nilges et al., Prog. NMR Spectroscopy 32:107-139 (1998)) can be employed which integrates with X-PLOR (Brunger, X-PLOR Version 3.1: A system for X-Ray crystallography and NMR, Yale University Press, New Haven, Conn., (1993)) for structural calculations. To ensure the success of ARIA/X-PLOR-assisted NOE analysis and structure calculations, the ARIA assigned NOE peaks can be manually confirmed.

Intermolecular NOE Distance Restrains: For the structural determination of a protein/peptide complex, intermolecular NOE distance restraints can be obtained from a ¹³C-edited (F,) and ¹⁵N, and ¹³C-filtered (F₃) 3D NOESY data set collected for a sample containing isotope-labeled protein and non-labeled peptide.

Structure Calculations and Refinements: Structures of the protein can be generated using a distance geometry/simulated annealing protocol with the X-PLOR program (see Nilges, et al., FEBS Lett. 229:317-324 (1988); Kuszewski, et al., J. Biolmol. NMR 2:33-56 (1992); Brtinger, A. T. X-PLOR Version 3.1: A system for X-Ray crystallography and NMR (Yale University Press, New Haven, Conn., 1993)). The structure calculations can employ inter-proton distance restraints obtained from ¹⁵N- and ¹³C-resolved NOESY spectra. The initial low-resolution structures can be used to facilitate NOE assignments, and help identify hydrogen bonding partners for slowly exchanging amide protons. The experimental restraints of dihedral angles and hydrogen bonds can be included in the distance restraints for structure refinements.

### Protein-Structure Based Design of Agonists and Antagonists of the Bromodomain-Acetyl-Lysine Binding Complex

Once the three-dimensional structure of the bromodomain and the bromodomain-acetyl-lysine binding complex are determined, a potential drug or agent (antagonist or agonist) can be examined through the use of computer modeling using a docking program such as GRAM, DOCK, or AUTODOCK (Dunbrack et al., 1997, supra). This procedure can include computer fitting of potential agents to the bromodomain, for example, to ascertain how well the shape and the chemical structure of the potential ligand will complement or interfere with the interaction between the bromodomain and the acetyl-lysine (Bugg et al., Scientific American, December: 92-98 (1993); West et al., TIPS, 16:67-74 (1995)). Computer programs can also be employed to estimate the attraction, repulsion, and steric hindrance of the agent to the dimer-dimer binding site, for example. Generally the tighter the fit (e.g., the lower the steric hindrance, and/or the greater the attractive force) the more potent the potential drug will be since these properties are consistent with a tighter binding constant. Furthermore, the more specificity in the design of a potential drug the more likely that the drug will not interfere with related proteins. This will minimize potential side-effects due to unwanted interactions with other proteins.

Initially a potential drug could be obtained by screening a random peptide library produced by recombinant bacteriophage for example, (Scott, et al., Science, 249:386-390 (1990); Cwirla et al., Proc. Natl. Acad. Sci., 87:6378-6382 (1990); Devlin et al., Science, 249:404-406 (1990)) or a chemical library. In particular, based on the NMR structural analysis provided herein, compounds that comprise an “acetyl-amine-like” structure as depicted in FIG. 12 are particularly good candidates. Examples of such “acetyl-lysine analogs” are included in FIG. 13.

An agent selected in this manner could be then be systematically modified (if necessary) by computer modeling programs until one or more promising potential drugs are identified. Such analysis has been shown to be effective in the development of HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)).

Such computer modeling allows the selection of a finite number of rational chemical modifications, as opposed to the countless number of essentially random chemical modifications that could be made, any one of which might lead to a useful drug. Each chemical modification requires additional chemical steps, which while being reasonable for the synthesis of a finite number of compounds, quickly becomes overwhelming if all possible modifications needed to be synthesized. Thus, through the use of the three-dimensional structural analysis disclosed herein and computer modeling, a large number of these compounds can be rapidly screened on the computer monitor screen, and a few likely candidates can be determined without the laborious synthesis of untold numbers of compounds.

Once a potential drug (agonist or antagonist) is identified it can be either selected from a library of chemicals as are commercially available from most large chemical companies including Merck, GlaxoWelcome, Bristol Meyers Squib, Monsanto/Searle, Eli Lilly, Novartis and Pharmacia UpJohn, or alternatively the potential drug may be synthesized de novo. As mentioned above, the de novo synthesis of one or even a relatively small group of specific compounds is reasonable in the art of drug design.

The potential drug can then be tested in any standard binding assay (including in high throughput binding assays) for its ability to bind to the ZA loop of a bromodomain. Alternatively the potential drug can be tested for its ability to modulate the binding of a bromodomain to acetylated histamine, for example. When a suitable potential drug is identified, a second NMR structural analysis can optionally be performed on the binding complex formed between the bromodomain-acetyl-lysine binding complex, or the bromodomain alone and the potential drug. Computer programs that can be used to aid in solving such three-dimensional structures include QUANTA, CHARMM, INSIGHT, SYBYL, MACROMODE, and ICM, MOLMOL, RASMOL, AND GRASP (Kraulis, Appl Crystallogr. 24:946-950 (1991)). Most if not all of these programs and others as well can be also obtained from the WorldWideWeb through the internet.

Using the approach described herein and equipped with the structural analysis disclosed herein, the three-dimensional structures of other bromodomain-acetyl-lysine binding complexes can more readily be obtained and analyzed. Such analysis will, in turn, allow corresponding drug screening methodology to be performed using the three-dimensional structures of such related complexes.

For all of the drug screening assays described herein further refinements to the structure of the drug will generally be necessary and can be made by the successive iterations of any and/or all of the steps provided by the particular drug screening assay, including further structural analysis by NMR, for example.

Phage libraries for Drug Screening: Phage libraries have been constructed which when infected into host E. coli produce random peptide sequences of approximately 10 to 15 amino acids (Parmnley, et al., Gene 73:305-318 (1988), Scott, et al., Science 249:386-249 (1990)). Specifically, the phage library can be mixed in low dilutions with permissive E. coli in low melting point LB agar which is then poured on top of LB agar plates. After incubating the plates at 37° C. for a period of time, small clear plaques in a lawn of E. coli will form which represents active phage growth and lysis of the E. coli. A representative of these phages can be absorbed to nylon filters by placing dry filters onto the agar plates. The filters can be marked for orientation, removed, and placed in washing solutions to block any remaining absorbent sites. The filters can then be placed in a solution containing, for example, a radioactive bromodomain. After a specified incubation period, the filters can be thoroughly washed and developed for autoradiography. Plaques containing the phage that bind to the radioactive bromodomain can then be identified. These phages can be further cloned and then retested for their ability to bind to the bromodomain as before. Once the phage has been purified, the binding sequence contained within the phage can be determined by standard DNA sequencing techniques. Once the DNA sequence is known, synthetic peptides can be generated which are encoded by these sequences. These peptides can be tested, for example, for their ability to modulate the affinity of the bromodomain for its binding partner (e.g., Tat or a fragment of Tat containing the acetyl-lysine corresponding to position 50 of SEQ ID NO:45).

The effective peptide(s) can be synthesized in large quantities for use in in vivo models and eventually in humans to treat certain tumors. It should be emphasized that synthetic peptide production is relatively non-labor intensive, easily manufactured, quality controlled and thus, large quantities of the desired product can be produced quite cheaply. Similar combinations of mass produced synthetic peptides have been used with great success (Patarroyo, Vaccine, 10:175-178 (1990)).

### Drug Screening Assays

The drug screening assays of the present invention may use any of a number of means for determining the interaction between an agent/drug (e.g., an acetyl-lysine analog) and a peptide comprising an acetyl-lysine and/or a bromodomain. Thus, standard high throughput drug screening procedures can be employed using a library of low molecular weight compounds, for example that can be screened to identify a binding partner for the bromodoamin. Any such chemical library can be used including those discussed above.

In a particular assay, a bromodomain (e.g., from P/CAF) is placed on or coated onto a solid support. Methods for placing the peptides or proteins on the solid support are well known in the art and include such things as linking biotin to the protein and linking avidin to the solid support. An agent is allowed to equilibrate with the bromodomain to test for binding. Generally, the solid support is washed and agents that are retained are selected as potential drugs. Alternatively, a peptide comprising an acetyl-lysine is placed on or coated onto a solid support. In a particular embodiment of this type, the peptide comprises the amino acid sequence of SEQ ID NO:4. In a preferred embodiment, the peptide comprises the amino acid sequence of SEQ ID NO:46.

The agent may be labeled. For example, in one embodiment radiolabeled agents are used to measure the binding of the agent. In another embodiment the agents have fluorescent markers. In yet another embodiment, a Biocore chip (Pharmacia) coated with the bromodomain is used, for example and the change in surface conductivity can be measured.

In addition, since a number of proteins have been identified that contain bromodomains, and the binding partners of many of these proteins are known, the fact that the bromodomain specifically binds to an acetylated lysine as disclosed herein allows the identification and preparation of a number of potential modulators of the bromodomain-acetyl-lysine binding complex based on the amino acid sequences of the binding partners to the proteins. Such potential modulators include: ISYGR-AcK-KRRQRR (SEQ ID NO:4), ARKSTGG-AcK-APRKQL (SEQ ID NO:5) and QSTSRHK-AcK-LMFKTE (SEQ ID NO:6) which bind to the P/CAF bromodomain as shown in the Example, below. Such peptides also can be used, for example, as a starting point for the design of an inhibitor of the bromodomain-acetyl-lysine binding complex.

Alternatively, a drug can be specifically designed to bind to the ZA loop of a bromodomain for example, such as the P/CAF bromodomain, and be assayed through NMR based methodology (Shuker et al., Science 274:1531-1534 (1996) hereby incorporated by reference in its entirety.) In a particular embodiment, analogs of the binding partner of the bromodomain can be used in this analysis. One such peptide has the amino acid sequence of SEQ D NO:4. In another embodiment of this type, the peptide has the amino acid sequence of SEQ ID NO:5. In another such embodiment of this type, the peptide has the amino acid sequence of SEQ ID NO:6.

The assay begins with contacting a compound with a ¹⁵N-labeled bromodomain. Binding of the compound with the ZA loop of the bromodomain can be determined by monitoring the ¹⁵N- or ¹H-amide chemical shift changes in two dimensional ¹⁵N-heteronuclear single-quantum correlation (¹⁵N-HSQC) spectra upon the addition of the compound to the ¹⁵N-labeled bromodomain. Since these spectra can be rapidly obtained, it is feasible to screen a large number of compounds (Shuker et al., Science 274:1531-1534 (1996)). A compound is identified as a potential ligand if it binds to the ZA loop of the bromodomain. In a further embodiment, the potential ligand can then be used as a model structure, and analogs to the compound can be obtained (e.g., from the vast chemical libraries commercially available, or alternatively through de novo synthesis). The analogs are then screened for their ability to bind the ZA loop of the bromodomain thus to obtain a ligand. An analog of the potential ligand is chosen as a ligand when it binds to the ZA loop of the bromodomain with a higher binding affinity than the potential ligand. In a preferred embodiment of this type the analogs are screened by monitoring the ¹⁵N- or ¹H-amide chemical shift changes in two dimensional ¹⁵N-heteronuclear single-quantum correlation (¹⁵N-HSQC) spectra upon the addition of the analog to the ¹⁵N-labeled bromodomain as described above.

In another further embodiment, compounds are screened for binding to two nearby sites on the bromodomain. In this case, a compound that binds a first site of the bromodomain does not bind a second nearby site. Binding to the second site can be determined by monitoring changes in a different set of amide chemical shifts in either the original screen or a second screen conducted in the presence of a ligand (or potential ligand) for the first site. From an analysis of the chemical shift changes the approximate location of a potential ligand for the second site is identified. Optimization of the second ligand for binding to the site is then carried out by screening structurally related compounds (e.g., analogs as described above). When ligands for the first site and the second site are identified, their location and orientation in the ternary complex can be determined experimentally either by NMR spectroscopy or X-ray crystallography. On the basis of this structural information, a linked compound is synthesized in which the ligand for the first site and the ligand for the second site are linked. In a preferred embodiment of this type the two ligands are covalently linked. This linked compound is tested to determine if it has a higher binding affinity for the bromodomain than either of the two individual ligands. A linked compound is selected as a ligand when it has a higher binding affinity for the bromodomain than either of the two ligands. In a preferred embodiment the affinity of the linked compound with the bromodomain is determined monitoring the ¹⁵N- or ¹H-amide chemical shift changes in two dimensional .sup.15N-heteronuclear single-quantum correlation (¹⁵N-HSQC) spectra upon the addition of the linked compound to the ¹⁵N-labeled bromodomain as described above.

A larger linked compound can be constructed in an analogous manner, e.g., linking three ligands which bind to three nearby sites on the bromodomain to form a multilinked compound that has an even higher affinity for the bromodomain than the linked compound.

### Identification of New Bromodomains

By disclosing that protein bound acetyl-lysine is a binding partner for bromodomains, the present invention provides a method of identifying novel proteins that contain bromodomains. In short, a protein fragment or analog thereof comprising an acetyl-lysine or an acetyl-lysine analog can be used as bait to identify a binding partner that comprises a bromodomain. Any one of a number of procedures can be carried out to identify such a binding partner. One such assay comprises passing a cell extract over the bait peptide which is attached to a solid support. After washing the solid support to remove any non-specific binders, the bromodomain containing protein can be eluted from the solid support with an appropriate eluant. In a particular embodiment, the free bait peptide can be used in the elution. Other methodology includes the use of a yeast two-hybrid system, a GST pull down assay, ELISA, immunometric assays, and a modification of the CORT procedure of Schlessinger et al., (U.S. Pat. No. 5,858,686, Issued on Jan. 12, 1999 which is hereby incorporated by reference in its entirety) for use with the bromodomain-acetyl-lysine binding complex.

### Labels

Suitable labels include enzymes, fluorophores (e.g., fluorescein isothiocyanate (FITC), phycoerythrin (PE), Texas red (TR), rhodamine, free or chelated lanthamide series salts, especially Eu.³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the test and control marker gene.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. U.V. light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932 and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70:419-439 (1980) and in U.S. Pat. No. 4,857,453.

Suitable enzymes include, but are not limited to, alkaline phosphatase, β-galactosidase, green fluorescent protein and its derivatives, luciferase, and horseradish peroxidase.

Other labels for use in the invention include magnetic beads or magnetic resonance imaging labels.

### Three-Dimensional Representation of the Structure of the Bromodomains

In addition, the present invention provides a computer that contains a representation of a bromodomain (or a bromodomain-ligand complex, e.g., the Tat-P/CAF complex) in computer memory that can be used to screen for compounds that will or are likely to inhibit the bromodomain-ligand interaction. In a particular embodiment of the present invention the bromodomain-ligand complex is the Tat-P/CAF complex and the compound identified by the screen can used to prevent, retard the progression, treat and/or cure AIDS.

In a related embodiment, the computer can be used in the design of altered bromodomains that have either enhanced, or alternatively diminished binding activity activity. Preferably, the computer comprises portions of and/or all of the information contained in Tables 1-6 and 10-14 of U.S. Ser. No. 09/784,553, filed Feb. 16, 2001, now U.S. Pat. No. 7,589,167, the disclosure of which is hereby incorporated by reference. In a particular embodiment, the computer comprises: (i) a machine-readable data storage material encoded with machine-readable data, (ii) a working memory for storing instructions for processing the machine readable data, (iii) a central processing unit coupled to the working memory and the machine-readable data storage material for processing the machine-readable data into a three-dimensional representation, and (iv) a display coupled to the central processing unit for displaying the three-dimensional representation.

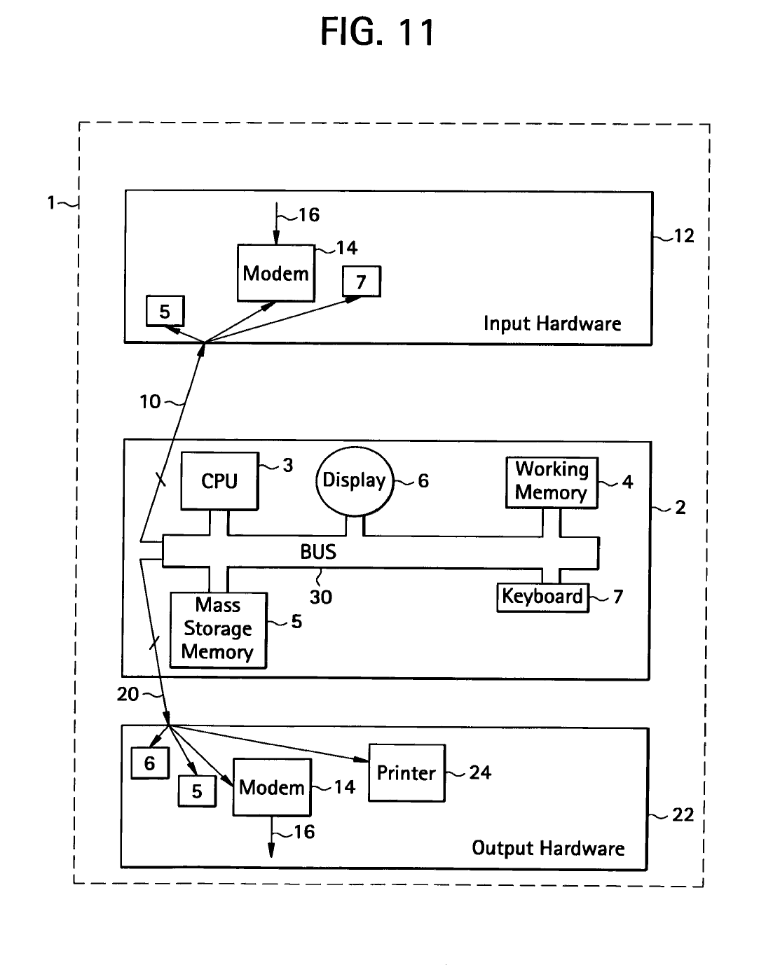

Thus the machine-readable data storage medium comprises a data storage material encoded with machine readable data which can comprise portions and/or all of the structural information contained in Tables 1-6 and 10-14 of U.S. Ser. No. 09/784,553, filed Feb. 16, 2001, now U.S. Pat. No. 7,589,167, the disclosure of which is hereby incorporated by reference. One embodiment for manipulating and displaying the structural data provided by the present invention is schematically depicted in FIG. 11. As depicted, the System 1, includes a computer 2 comprising a central processing unit (“CPU”) 3, a working memory 4 which may be random-access memory or “core” memory, mass storage memory 5 (e.g., one or more disk or CD-ROM drives), a display terminal 6 (e.g., a cathode-ray tube), one or more keyboards 7, one or more input lines 10, and one or more output lines 20, all of which are interconnected by a conventional bidirectional system bus 30.

Input hardware 12, coupled to the computer 2 by input lines 10, may be implemented in a variety of ways. Machine-readable data may be inputted via the use of one or more modems 14 connected by a telephone line or dedicated data line 16. Alternatively or additionally, the input hardware 12 may comprise CD-ROM or disk drives 5. In conjunction with the display terminal 6, the keyboard 7 may also be used as an input device. Output hardware 22, coupled to computer 2 by output lines 20, may similarly be implemented by conventional devices. Output hardware 22 may include a display terminal 6 for displaying the three dimensional data. Output hardware might also include a printer 24, so that a hard copy output may be produced, or a disk drive 5, to store system output for later use, see also U.S. Pat. No. 5,978,740, Issued Nov. 2, 1999, the disclosure of which is hereby incorporated by reference in its entirety.

In operation, the CPU 3 (i) coordinates the use of the various input and output devices 12 and 22; (ii) coordinates data accesses from mass storage 5 and accesses to and from working memory 4; and (iii) determines the sequence of data processing steps. Any of a number of programs may be used to process the machine-readable data of this invention.

Antibodies to Portions of the Bromodomain that Interact with Acetyl-Lysine

According to the present invention, the bromodomains, and more particularly the ZA loops of the bromodomains and fragments thereof can be produced by a recombinant source, or through chemical synthesis, or through the modification of these peptides and fragments; and derivatives or analogs thereof, including fusion proteins, may be used as an immunogen to generate antibodies that specifically interfere with the formation of the bromodomain-acetyl-lysine binding complex. Similarly, antibodies can be raised against peptides that comprise one or more acetyl-lysine residues which also interfere with the formation of the bromodomain-acetyl-lysine binding complex. Such antibodies include but are not limited to polyclonal, monoclonal, chimeric, single chain, Fab fragments, and a Fab expression library.

Various procedures known in the art may be used for the production of the polyclonal antibodies. For the production of antibody, various host animals can be immunized by injection with the peptide having the amino acid sequence of SEQ ID NO:3, for example, or a derivative (e.g., or fusion protein) thereof, including but not limited to rabbits, mice, rats, sheep, goats, etc. In one embodiment, the peptide can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the peptides or protein fragments of the present invention, or analog, or derivative thereof, any technique that provides for the production of antibody molecules by continuous cell lines in culture may be used. These include but are not limited to the hybridoma technique originally developed by Kohler, et al. (Nature, 256:495-497 (1975)), as well as the trioma technique, the human B-cell hybridoma technique (Kozbor et al., Immunology Today, 4:72 (1983); Cote et al., Proc. Natl. Acad. Sci. U.S.A., 80:2026-2030 (1983)), and the EBV-hybridoma technique to produce human monoclonal antibodies (Cole et al., in Monoclonal Antibodies and Cancer Therapy, Alan R. Liss, Inc., pp. 77-96 (1985)). In an additional embodiment of the invention, monoclonal antibodies can be produced in germ-free animals utilizing technology described in PCT/US90/02545. In fact, according to the invention, techniques developed for the production of “chimeric antibodies” (Morrison et al., J. Bacteriol., 159:870 (1984); Neuberger et al., Nature, 312:604-608 (1984); Takeda et al., Nature, 314:452-454 (1985)) by splicing the genes from a mouse antibody molecule specific for the peptide having the amino acid sequence of SEQ ID NO:3, for example, together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies (Huston, U.S. Pat. Nos. 5,476,786 and 5,132,405; U.S. Pat. No. 4,946,778) can be adapted to produce specific single chain antibodies. An additional embodiment of the invention utilizes the techniques described for the construction of Fab expression libraries (Huse et al., Science, 246:1275-1281(1989)) to allow rapid and easy identification of monoclonal Fab fragments with the desired specificity.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a ZA loop of a bromodomain, for example, one may assay generated hybridomas for a product which binds to a bromodomain fragment containing such an epitope and choose those which do not cross-react with bromodomain fragments that do not include that epitope.

In a specific embodiment, antibodies that interfere with the formation of the bromodomain-acetyl-lysine complex can be generated. Such antibodies can be tested using the assays described and could potentially be used in anti-cancer therapies.

### Small Molecules

Compounds of the present in invention may function by modulating the stability of the binding complex formed between P/CAF and Tat that is acetylated at the lysine residue at position 50 of SEQ ID NO:45. In one such embodiment the the present invention features contacting the bromodomain of P/CAF or a fragment thereof with a binding partner in the presence of the compound under conditions in which the bromodomain of P/CAF and the binding partner bind in the absence of the compound. The stability of the bromodomain of P/CAF and the binding partner is then determined (e.g., measured). When there is a change in the stability of the binding complex between the bromodomain of P/CAF and the binding partner in the presence of the compound, the compound is identified as a modulator. In one embodiment of this type the binding partner is Tat that is acetylated at the lysine residue at position 50 of SEQ ID NO:45. In a preferred embodiment the binding partner is a fragment of Tat comprising an acetyl-lysine at position 50. In still another embodiment the binding partner is an analog of the fragment of Tat comprising an acetyl-lysine at position 50. When the stability of the bromodomain of P/CAF for the binding partner increases in the presence of the compound, the compound is identified as a stabilizing agent, whereas when the stability of the bromodomain of P/CAF for the binding partner decreases in the presence of the compound, the compound is identified as an inhibitor of the Tat-P/CAF complex. In a preferred embodiment the compound is selected by performing rational drug design with the set of atomic coordinates obtained from one or more of Tables 1-5 and 10-14 of U.S. Ser. No. 09/784,553, filed Feb. 16, 2001, now U.S. Pat. No. 7,589,167, the disclosure of which is hereby incorporated by reference. More preferably the selection is performed in conjunction with computer modeling. Compounds identified by these methods are also part of the present invention. Preferably the compound is an analog of acetyl-lysine. More preferably the compound is a small organic molecule not included in FIG. 13.

Compounds of the present invention may function by modulating the binding of P/CAF and Tat. In a preferred embodiment the agent is a small organic molecule. Preferably the agent inhibits and/or destabilizes the binding of P/CAF with Tat. The agent may be an analog of acetyl-lysine.

Compounds of the present in invention are useful for modulating preventing, and/or retarding the progression and/or treating HIV infection in an individual. One such method employs administering to the individual compounds that modulate the Tat-P/CAF complex selected by performing rational drug design with the set of atomic coordinates obtained from one or more of Tables 1-5 and 10-14 of U.S. Ser. No. 09/784,553, filed Feb. 16, 2001, now U.S. Pat. No. 7,589,167, the disclosure of which is hereby incorporated by reference. In a preferred embodiment the compound administered is an acetyl-lysine analog. In a particular embodiment this compound is a small organic molecule contained in FIG. 13. Preferably the compound either de-stabilizes or inhibits the Tat-P/CAF complex.

As used herein the following terms are defined as follows:
the terms “lower alkyl and lower alkoxy (see below)” are understood as meaning straight or branched alkyl and alkoxy groups having from 1 to 8 carbon atoms;
the term “aryl” is understood as meaning an aromatic group selected from phenyl and naphthyl groups;
the term “heteroaryl” is understood as meaning a mono- or bicyclic aromatic group, each cycle, or ring, comprising five or six atoms and said cycle, or ring, or both cycles, or rings, including in its carbon skeleton from one to three heteroatoms selected from nitrogen, oxygen and sulphur;
the terms “lower aralkyl” and “lower heteroaralkyl” are understood as meaning, in view of the definitions above, phenyl(C₁-C₈)alkyl or naphthyl(C₁-C₈)alkyl and heteroar(C₁-C₈)alkyl respectively;
the term “substituted” concerning the terms aryl, aralkyl, phenyl, radical (five-membered, including Z), heteroaryl, heteroaralkyl, as defined above, signifies that the groups in question are substituted on the aromatic part with one or more identical or different groups selected from the groups: (C₁-C₈)alkyl, trifluoromethyl, (C₁-C₈)alkoxy, hydroxy, nitro, amino, (C₁-C₈)alkylamino, di(C₁-C₈)alkylamino, sulphoxyl, sulphonyl, sulphonamide, sulpho(C₁-C₈)alkyl, carboxyl, carbalkoxyl, carbamide (it being possible for said (C₁-C₈)alkyl groups to be linear or branched) or substituted with one or more halogen atoms;
the term aminoacyl, which concerns the glutathionyl, cysteinyl, N-acetylcysteinyl or even the penicillaminyl group in the definition of X, signifies any natural aminoacid such as alanine, and leucine, for example.

As used herein, and unless otherwise specified, the terms “agent”, “potential drug”, “compound”, “test compound” or “potential compound” are used interchangeably, and refer to chemicals which potentially have a use as an inhibitor or activator/stabilizer of bromodomain-acetyl-lysine binding. Therefore, such “agents”, “potential drugs”, “compounds” and “potential compounds” may be used, as described herein, in drug assays and drug screens and the like.

As used herein a “small organic molecule” is an organic compound, including a peptide or organic compound complexed with an inorganic compound (e.g., metal) that has a molecular weight of less than 3 Kilodaltons. Such small organic molecules can be included as agents, etc. as defined above.

As used herein the term “binds to” is meant to include all such specific interactions that result in two or more molecules showing a preference for one another relative to some third molecule. This includes processes such as covalent, ionic, hydrophobic and hydrogen bonding but does not include non-specific associations such as solvent preferences.

### Administration

According to the invention, the component or components of a therapeutic composition, e.g., an agent of the invention that interferes with the bromodomain-acetyl-lysine binding complex such as the peptide having the amino acid sequence of SEQ ID NOs:4, 5, 6, 46, or 47, or an acetyl-lysine analog as defined by FIG. 12 and exemplified in FIG. 13, and a pharmaceutically acceptable carrier, may be introduced parenterally, transmucosally, e.g., orally, nasally, or rectally, or transdermally. Preferably, administration is parenteral, e.g., via intravenous injection, and also including, but is not limited to, intra-arteriole, intramuscular, intradermal, subcutaneous, intraperitoneal, intraventricular, and intracranial administration.

In a preferred aspect, the agent of the present invention can cross cellular and nuclear membranes, which would allow for intravenous or oral administration. Strategies are available for such crossing, including but not limited to, increasing the hydrophobic nature of a molecule; introducing the molecule as a conjugate to a carrier, such as a ligand to a specific receptor, targeted to a receptor; and the like.

The present invention also provides for conjugating targeting molecules to such an agent. “Targeting molecule” as used herein shall mean a molecule which, when administered in vivo, localizes to desired location(s). In various embodiments, the targeting molecule can be a peptide or protein, antibody, lectin, carbohydrate, or steroid. In one embodiment, the targeting molecule is a peptide ligand of a receptor on the target cell. In a specific embodiment, the targeting molecule is an antibody. Preferably, the targeting molecule is a monoclonal antibody. In one embodiment, to facilitate crosslinking the antibody can be reduced to two heavy and light chain heterodimers, or the F(ab′)₂ fragment can be reduced, and crosslinked to the agent via the reduced sulfhydryl. Antibodies for use as targeting molecule are specific for a cell surface antigen.

In another embodiment, the therapeutic compound can be delivered in a vesicle, in particular a liposome (see Langer, Science, 249:1527-1533 (1990); Treat et al., in Liposomes in the Therapy of Infectious Disease and Cancer, Lopez-Berestein, et al. (eds.), Liss: New York, pp. 353-365 (1989); Lopez-Berestein, ibid., pp. 317-327; see generally ibid.).

In yet another embodiment, the therapeutic compound can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref Biomed. Eng., 14:201 (1987); Buchwald et al., Surgery, 88:507 (1980); Saudek et al., N. Engl. J. Med., 321:574 (1989)). In another embodiment, polymeric materials can be used (see Medical Applications of Controlled Release, Langer, et al (eds.), CRC Press: Boca Raton, Fla. (1974); Controlled Drug Bioavailability, Drug Product Design and Performance, Smolen, et al. (eds.), Wiley: New York (1984); Ranger, et al., J. Macromol. Sci. Rev. Macromol. Chem., 23:61 (1983); see also Levy et al., Science, 228:190 (1985); During et al., Ann. Neurol., 25:351 (1989); Howard et al., J. Neurosurg., 71:105 (1989)). In yet another embodiment, a controlled release system can be placed in proximity of the therapeutic target, i.e., the bone marrow, thus requiring only a fraction of the systemic dose (see, e.g., Goodson, in Medical Applications of Controlled Release, supra, vol. 2, pp. 115-138 (1984)). Other controlled release systems are discussed in the review by Langer (Science, 249:1527-1533 (1990)).

Pharmaceutical Compositions. In yet another aspect the present invention provides pharmaceutical compositions of the above. Such pharmaceutical compositions may be for administration for injection, or for oral, pulmonary, nasal or other forms of administration. In general, the invention provides pharmaceutical compositions comprising effective amounts of a low molecular weight component or components, or derivative products, of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form.

Oral Delivery. Contemplated for use herein are oral solid dosage forms, which are described generally in Remington's Pharmaceutical Sciences, 18th Ed.1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include an agent of the present invention (or chemically modified forms thereof) and inert ingredients which allow for protection against the stomach environment, and release of the biologically active material in the intestine.

Also specifically contemplated are oral dosage forms of the above derivatized component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. An example of such a moiety is polyethylene glycol.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the protein (or derivative) or by release of the biologically active material beyond the stomach environment, such as in the intestine.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Binders also may be used to hold the therapeutic agent together to form a hard tablet and include materials from natural products such as acacia, tragacanth, starch and gelatin.

An anti-frictional agent may be included in the formulation of the therapeutic to prevent sticking during the formulation process. Lubricants may be used as a layer between the therapeutic and the die wall. Glidants that might improve the flow properties of the drug during formulation and to aid rearrangement during compression also might be added. The glidants may include starch, talc, pyrogenic silica and hydrated silicoaluminate.

In addition, to aid dissolution of the therapeutic into the aqueous environment a surfactant might be added as a wetting agent. Additives which potentially enhance uptake of the protein (or derivative) are for instance the fatty acids oleic acid, linoleic acid and linolenic acid.

Nasal Delivery. Nasal delivery of an agent of the present invention (or derivative) is also contemplated. Nasal delivery allows the passage of a peptide, for example, to the blood stream directly after administering the therapeutic product to the nose, without the necessity for deposition of the product in the lung. Formulations for nasal delivery include those with dextran or cyclodextran.

Transdermal administration. Various and numerous methods are known in the art for transdermal administration of a drug, e.g., via a transdermal patch. Transdermal patches are described in for example, U.S. Pat. No. 5,407,713, issued Apr. 18, 1995 to Rolando et al.; U.S. Pat. No. 5,352,456, issued Oct. 4, 1004 to Fallon et al.; U.S. Pat. No. 5,332,213 issued Aug. 9, 1994 to D'Angelo et al.; U.S. Pat. No. 5,336,168, issued Aug. 9, 1994 to Sibalis; U.S. Pat. No. 5,290,561, issued Mar. 1, 1994 to Farhadieh et al.; U.S. Pat. No. 5,254,346, issued Oct. 19, 1993 to Tucker et al.; U.S. Pat. No. 5,164,189, issued Nov. 17, 1992 to Berger et al.; U.S. Pat. No. 5,163,899, issued Nov. 17, 1992 to Sibalis; U.S. Pat. Nos. 5,088,977 and 5,087,240, both issued Feb. 18, 1992 to Sibalis; U.S. Pat. No. 5,008,110, issued Apr. 16, 1991 to Benecke et al.; and U.S. Pat. No. 4,921,475, issued May 1, 1990 to Sibalis, the disclosure of each of which is incorporated herein by reference in its entirety.

It can be readily appreciated that a transdermal route of administration may be enhanced by use of a dermal penetration enhancer, e.g., such as enhancers described in U.S. Pat. No. 5,164,189 (supra), U.S. Pat. No. 5,008,110 (supra), and U.S. Pat. No. 4,879,119, issued Nov. 7, 1989 to Aruga et al., the disclosure of each of which is incorporated herein by reference in its entirety.

Pulmonary Delivery. Also contemplated herein is pulmonary delivery of the pharmaceutical compositions of the present invention. A pharmaceutical composition of the present invention is delivered to the lungs of a mammal while inhaling and traverses across the lung epithelial lining to the blood stream. Other reports of this include Adjei et al. (Pharmaceutical Research, 7:565-569 (1990); Adjei et al., International Journal of Pharmaceutics, 63:135-144 (1990) (leuprolide acetate); Braquet et al., Journal of Cardiovascular Pharmacology, 13(suppl. 5):143-146 (1989) (endothelin-1); Hubbard et al., Annals of Internal Medicine, Vol. 111, pp. 206-212 (1989) (al-antitrypsin); Smith et al., J. Clin. Invest., 84:1145-1146 (1989) (a-1-proteinase); Oswein et al., “Aerosolization of Proteins”, Proceedings of Symposium on Respiratory Drug Delivery II, Keystone, Colo., March, (1990) (recombinant human growth hormone); Debs et al., J. Immunol., 140:3482-3488 (1988) (interferon-γ and tumor necrosis factor alpha); Platz et al., U.S. Pat. No. 5,284,656 (granulocyte colony stimulating factor)). A method and composition for pulmonary delivery of drugs for systemic effect is described in U.S. Pat. No. 5,451,569, issued Sep. 19, 1995 to Wong et al.

A subject in whom administration of an agent of the present invention is an effective therapeutic regiment for cancer, for example, is preferably a human, but can be any animal. Thus, as can be readily appreciated by one of ordinary skill in the art, the methods and pharmaceutical compositions of the present invention are particularly suited to administration to any animal, e.g., for veterinary medical use, particularly for a mammal, and including, but by no means limited to, domestic animals, such as feline or canine subjects, farm animals, including bovine, equine, caprine, ovine, and porcine subjects, wild animals (whether in the wild or in a zoological garden), research animals, such as mice, rats, rabbits, goats, sheep, pigs, dogs, cats, avian species, such as chickens, turkeys, and songbirds.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### DETAILED DESCRIPTION OF THE PREFERRED EMBODIMENTS

### EXAMPLE 1

### Structure and Ligand of a Histone Acetyltransferase Bromodomain

Sample preparation: The bromodomain of P/CAF (residues 719-832 of SEQ ID NO:2) was subcloned into the pET14b expression vector (Novagen) and expressed in Escherichia coli BL21(DE3) cells. Uniformly ¹⁵N- and ¹⁵N/¹³C-labelled proteins were prepared by growing bacteria in a minimal medium containing ¹⁵NH₄Cl with or without ¹³C₆-glucose. A uniformly ¹⁵N/¹³C-labelled and fractionally deuterated protein sample was prepared by growing the cells in 75% ²H₂O. The bromodomain was purified by affinity chromatography on a nickel-IDA column (Invitrogen) followed by the removal of poly-His tag by thrombin cleavage. The final purification of the protein was achieved by size-exclusion chromatography. The acetyl-lysine-containing peptides were prepared on a MilliGen 9050 peptide synthesizer (Perkin Elmer) using Fmoc/HBTU chemistry. Acetyl-lysine was incorporated using the reagent Fmoc-Ac-Lys with HBTU/DIPEA activation. NMR samples contained approximately 1 mM protein in 100 mM phosphate buffer of pH 6.5 and 5 mM perdeuterated DTT and 0.5 mM EDTA in H₂O/²H₂O (9/1) or ²H₂O.

NMR spectroscopy: All NMR spectra were acquired at 30° C. on a Bruker DRX600 or DRX500 spectrometer. The backbone assignments of the ¹H, ¹³C, and ¹⁵N resonances were achieved using deuterium-decoupled triple-resonance experiments of HNCACB and HN(CO)CACB (Yamazaki et al., J. Am. Chem. Soc. 116:11655-11666 (1994)) recorded using the uniformly ¹⁵N/¹³C-labeled and fractionally deuterated protein. The side-chain atoms were assigned from 3D HCCH-TOCSY (Clore, et al., Meth. Enzymol. 239:249-363 (1994)) and (H)C(CO)NH-TOCSY (Logan et al., J. Biolmol. NMR 3:225-231 (1993)) data collected on the uniformly ¹⁵N/¹³C-labeled protein. Stereospecific assignments of methyl groups of the Val and Leu residues were obtained using a fractionally ¹³C-labeled sample (Neri et al., Biochemistry 28:7510-7516 (1989)). The NOE-derived distance restraints were obtained from ¹⁵N- or ¹³C-edited 3D NOESY spectra. φ-angle restraints were determined based on the ³J_{HN,H} coupling constants measured in a 3D HNHA spectrum (Clore, et al., Meth. Enzymol. 239:249-363 (1994)). Slowly exchanging amide protons were identified from a series of 2D ¹⁵N-HSQC spectra recorded after the H₂O buffer was changed to a ²H₂O buffer. The intermolecular NOEs used in defining the structure of the bromodomain/Ac-histamine complex were detected in ¹³C-edited (F₁), ¹³C/¹⁵N-filtered (F₃) 3D NOESY spectrum (Clore, et al., Meth. Enzymol. 239:249-363 (1994)). All NMR spectra were processed with the NMRPipe/NMRDraw programs and analyzed using NMRView (Johnson, et al., J. Biomol., NMR 4:603-614 (1994)).

Structure calculations: Structures of the bromodomain were calculated with a distance geometry/simulated annealing protocol using the X-PLOR program (Brunger, X-PLOR Version 3.1: A system for X-Ray crystallography and NMR, Yale University Press, New Haven, Conn., (1993)). A total of 1324 manually assigned NOE-derived distance restraints were obtained from the ¹⁵N- and ¹³C-edited NOE spectra. Further analysis of the NOE spectra was carried out by the iterative automated assignment procedure using ARIA (Nilges, et al., Prog. NMR Spectroscopy 32:107-139 (1998)), which integrates with X-PLOR for structure calculations. A total of 1519 unambiguous and 590 ambiguous distance restraints were identified from the NOE data by ARIA, many of which were checked and confirmed manually. The ARIA-assigned distance restraints were in agreement with the structures calculated using only the manually assigned NOE distance restraints, 28 hydrogen-bond distance restraints for 14 hydrogen bonds, and 54□-angle restraints. The final structure calculations employed a total of 3515 NMR experimental restraints obtained from the manual and the ARIA-assisted assignments, 2843 of which were unambiguously assigned NOE-derived distance restraints that comprise of 1077 intra-residue, 621 sequential, 550 medium-range, and 595 long-range NOEs. For the ensemble of the final 30 structures, no distance and torsional angle restraints were violated by more than 0.3 Å and 5□, respectively. The total, distance violation, and dihedral violation energies were 178.7±2.4 kcal mol⁻¹, 41.6±0.9 kcal mol⁻¹, and 0.50±0.06 kcal mol⁻¹, respectively. The Lennard-Jones potential which was not used during any refinement stage, was −526.2±16.8 kcal mol⁻¹ for the final structures. Ramachandran plot analysis of the final structures (residues 727-828) with Procheck-NMR (Laskowski et al., J. Biolmol. NMR 8:477-486 (1996)) showed that 71.0±0.6%, 23.8±0.6%, 3.5±0.2%, and 1.7±0.2% of the non-Gly and non-Pro residues were in the most favorable, additionally allowed, generously allowed, and disallowed regions, respectively. The corresponding values for the residues in the four -helices (residues 727-743, 770-776, 785-802, and 807-827) were 88.9±0.4%, 11.0±0.4%, 0.1±0.1%, and 0.0±0.0%, respectively. The structure of the bromodomain/acetyl-histamine complex was determined using the free form structure and additional 25 intermolecular and 5 intra-ligand NOE-derived distance restraints.

Site-directed mutagenesis: Mutant proteins were prepared using the QuickChange site-directed mutagenesis kit (Stratagene). The presence of appropriate mutations was confirmed by DNA sequencing.

Ligand titration: Ligand titration experiments were performed by recording a series of 2D ¹⁵N- and ¹³C-HSQC spectra on the uniformly ¹⁵N-, and ¹⁵N/¹³C-labelled bromodomain (˜0.3 mM), respectively, in the presence of different amounts of ligand concentration ranging from 0 to approximately 2.0 mM. The protein sample and the stock solutions of the ligands were all prepared in the same aqueous buffer containing 100 mM phosphate and 5 mM perdeuterated DTT at pH 6.5.

The full length nucleic acid sequence of the human p300/CBP-associated factor (P/CAF) was obtained from GenBank. Accession No: U57317.2 (SEQ ID NO:1). The full length protein sequence of the human p300/CBP-associated factor (P/CAF) was obtained from GenBank. Accession No: U57317.2, (SEQ ID NO:2).

The full length nucleic acid sequence of the human p300/CBP-associated factor (P/CAF) was obtained from GenBank. Accession No: U57317.2 (SEQ ID NO: 1):

| | |
|---|---|
| 11 | ggggccgcgt cgacgcggaa aagaggccgt ggggggcctc ccagcgctgg cagacaccgt |
| 61 | gaggctggca gccgccggca cgcacaccta gtccgcagtc ccgaggaaca tgtccgcagc |
| 121 | cagggcgcgg agcagagtcc cgggcaggag aaccaaggga gggcgtgtgc tgtggcggcg |
| 181 | gcggcagcgg cagcggagcc gctagtcccc tccctcctgg gggagcagct gccgccgctg |
| 241 | ccgccgccgc caccaccatc agcgcgcggg gcccggccag agcgagccgg gcgagcggcg |
| 301 | cgctaggggg agggcggggg cggggagggg ggtgggcgaa gggggcggga gggcgtgggg |
| 361 | ggagggtctc gctctcccga ctaccagagc ccgagggaga ccctggcggc ggcggcggcg |
| 421 | cctgacactc ggcgcctcct gccgtgctcc ggggcggcat gtccgaggct ggcggggccg |
| 481 | ggccgggcgg ctgcggggca ggagccgggg caggggccgg gcccggggcg ctgcccccgc |
| 541 | agcctgcggc gcttccgccc gcgcccccgc agggctcccc ctgcgccgct gccgccgggg |
| 601 | gctcgggcgc ctgcggtccg gcgacggcag tggctgcagc gggcacggcc gaaggaccgg |
| 661 | gaggcggtgg ctcggcccga atcgccgtga agaaagcgca actacgctcc gctccgcggg |
| 721 | ccaagaaact ggagaaactc ggagtgtact ccgcctgcaa ggccgaggag tcttgtaaat |
| 781 | gtaatggctg gaaaaaccct aacccctcac ccactccccc cagagccgac ctgcagcaaa |
| 841 | taattgtcag tctaacagaa tcctgtcgga gttgtagcca tgccctagct gctcatgttt |
| 901 | cccacctgga gaatgtgtca gaggaagaaa tgaacagact cctgggaata gtattggatg |
| 961 | tggaatatct ctttacctgt gtccacaagg aagaagatgc agataccaaa caagtttatt |
| 1021 | tctatctatt taagctcttg agaaagtcta ttttacaaag aggaaaacct gtggttgaag |
| 1081 | gctctttgga aaagaaaccc ccatttgaaa aacctagcat tgaacagggt gtgaataact |
| 1141 | ttgtgcagta caaatttagt cacctgccag caaaagaaag gcaaacaata gttgagttgg |
| 1201 | caaaaatgtt cctaaaccgc atcaactatt ggcatctgga ggcaccatct caacgaagac |
| 1261 | tgcgatctcc caatgatgat atttctggat acaaagagaa ctacacaagg tggctgtgtt |
| 1321 | actgcaacgt gccacagttc tgcgacagtc tacctcggta cgaaaccaca caggtgtttg |
| 1381 | ggagaacatt gcttcgctcg gtcttcactg ttatgaggcg acaactcctg gaacaagcaa |
| 1441 | gacaggaaaa agataaactg cctcttgaaa aacgaactct aatcctcact catttcccaa |
| 1501 | aatttctgtc catgctagaa gaagaagtat atagtcaaaa ctctcccatc tgggatcagg |
| 1561 | attttctctc agcctcttcc agaaccagcc agctaggcat ccaaacagtt atcaatccac |
| 1621 | ctcctgtggc tgggacaatt tcatacaatt caacctcatc ttcccttgag cagccaaacg |
| 1681 | cagggagcag cagtcctgcc tgcaaagcct cttctggact tgaggcaaac ccaggagaaa |
| 1741 | agaggaaaat gactgattct catgttctgg aggaggccaa gaaaccccga gttatggggg |
| 1801 | atattccgat ggaattaatc aacgaggtta tgtctaccat cacggaccct gcagcaatgc |
| 1861 | ttggaccaga gaccaatttt ctgtcagcac actcggccag ggatgaggcg gcaaggttgg |
| 1921 | aagagcgcag gggtgtaatt gaatttcacg tggttggcaa ttccctcaac cagaaaccaa |
| 1981 | acaagaagat cctgatgtgg ctggttggcc tacagaacgt tttctcccac cagctgcccc |
| 2041 | gaatgccaaa agaatacatc acacggctcg tctttgaccc gaaacacaaa acccttgctt |
| 2101 | taattaaaga tggccgtgtt attggtggta tctgtttccg tatgttccca tctcaaggat |
| 2161 | tcacagagat tgtcttctgt gctgtaacct caaatgagca agtcaagggc tatggaacac |
| 2221 | acctgatgaa tcatttgaaa gaatatcaca taaagcatga catcctgaac ttcctcacat |
| 2281 | atgcagatga atatgcaatt ggatacttta agaaacaggg tttctccaaa gaaattaaaa |
| 2341 | tacctaaaac caaatatgtt ggctatatca aggattatga aggagccact ttaatgggat |
| 2401 | gtgagctaaa tccacggatc ccgtacacag aattttctgt catcattaaa aagcagaagg |
| 2461 | agataattaa aaaactgatt gaaagaaaac aggcacaaat tcgaaaagtt taccctggac |
| 2521 | tttcatgttt taaagatgga gttcgacaga ttcctataga aagcattcct ggaattagag |
| 2581 | agacaggctg gaaaccgagt ggaaaagaga aaagtaaaga gaccagagac cctgaccagc |
| 2641 | tttacagcac gctcaagagc atcctccagc aggtgaagag ccatcaaagc gcttggccct |
| 2701 | tcatggaacc tgtgaagaga acagaagctc caggatatta tgaagttata aggttcccca |
| 2761 | tggatctgaa aaccatgagt gaacgcctca agaataggta ctacgtgtct aagaaattat |
| 2321 | tcatggcaga cttacagcga gtctttacca attgcaaaga gtacaacgcc gctgagagtg |
| 2881 | aatactacaa atgtgccaat atcctggaga aattcttctt cagtaaaatt aaggaagctg |
| 2941 | gattaattga caagtgattt tttttccccc tctgcttctt agaaactcac caagcagtgt |
| 3001 | gcctaaagca aggt |

The full length protein sequence of the human p300/CBP-associated factor (P/CAF) was obtained from GenBank. Accession No: U57317.2, (SEQ ID NO:2):

| | |
|---|---|
| 21 | MSEAGGAGPG GCGAGAGAGA GPGALPPQPA ALPPAPPQGS PCAAAAGGSC ACGPATAVAA |
| 61 | ACTAEGPGGG GSARTAVKKA QLRSAPRAKK LEKLGVYSAC KAEESCKCNG WKNPNPSPTP |
| 121 | PRADLQQIIV SLTESCRSCS HALAAHVSHL ENVSEEEMNR LLGTVLDVEY LFTCVHKEED |
| 181 | ADTKQVYFYL FKLLRKSILQ RGICPVVEGSL EKKPPFEKPS IEQGVNNFVQ YKFSHLPAKE |
| 241 | RQTIVELK(M FLNRINYWHL EAPSQRRLRS PNDDTSGYKE NYTRWLCYCN VPQFCDSLPR |
| 301 | YETTQVFGRT LLRSVFTV34R RQLLEQARQE KDKLPLEKRT LILTHFPKFL SMLEEEVYSQ |
| 361 | NSPTWDQDFL SASSRTSQLG IQTVINPPPV AGTISYNSTS SSLEQPNAGS SSPACKASSG |
| 421 | LEANPGEKRK MTDSHVLEEA KKPRVMGDIP MELINEVMST ITDPAAMLGP ETNFLSAHSA |
| 481 | RDEAARLEER RGVTEFHVVG NSLNQKPNKK ILMWLVGLQN VFSHQLPRMP KEYITRLVFD |
| 541 | PKHKTLALIK DGRVIGGICF RMFPSQGFTE IVFCAVTSNE QVKGYGTHLM NHLKEYHIKH |
| 601 | DILNFLTYAD EYAIGYFKKQ GFSKEIKIFK TKYVGYIKDY EGATLMGCEL NPRIPYTEFS |
| 661 | VIIKKQKEII KKLIERKQAQ IRKVYPGLSC FKDGVRQIPI ESIPGIRETG WKPSGKEKSK |
| 721 | EPRDPDQLYS TLKSILQQVK SHQSAWPFME PVKRTEAPGY YEVIRFPMDL KTMSERLKNR |
| 781 | YYVSKKLFMA DLQRVFTNCK EYNAAESEYY KCANILEKFF FSKIKEAGLI DK |

Results. The P/CAF bromodomain represents an extensive family of bromodomains (FIG. 1). A large number of long-range nuclear Overhauser enhancement (NOE)-derived distance restraints were identified in the NMR data of the P/CAF bromodomain, yielding a well-defined three-dimensional structure (FIGS. 2A -2D). Table 2 of U.S. Ser. No. 09/784,553, filed Feb. 16, 2001, now U.S. Pat. No. 7,589,167, the disclosure of which is hereby incorporated by reference shows the NMR chemical shift assignment of the P/CAF bromodomain. Table 2 of U.S. Ser. No. 09/784,553, filed Feb. 16, 2001, now U.S. Pat. No. 7,589,167, the disclosure of which is hereby incorporated by reference, shows the Unambiguous NOE-derived distance restraints. Table 4 shows the Ambiguous NOE-derived distance restraints. Table 5 of U.S. Ser. No. 09/784,553, filed Feb. 16, 2001, now U.S. Pat. No. 7,589,167, the disclosure of which is hereby incorporated by reference, shows the hydrogen bond restraints. The NMR structure coordinates of the P/CAF bromodomain in the free and complexed to acetyl-histamine are shown in Tables 5 and 6 of U.S. Ser. No. 09/784,553, filed Feb. 16, 2001, now U.S. Pat. No. 7,589,167, the disclosure of which is hereby incorporated by reference, respectively. The structure consists of a four-helix bundle (helices _{Z, A, B,} and _{C}) with a left-handed twist, and a long intervening loop between helices _{Z} and _{A} (termed the ZA loop, FIG. 2E). The four amphipathic-helices are packed tightly against one another in an antiparallel manner, with crossing angles for adjacent helices of ˜16-20°. The up-and-down four-helix bundle can adapt two topological folds with opposite handedness (FIGS. 2F-2G). The right-handed four-helix bundle fold occurs more commonly and is seen in proteins such as hemerythrin and cytochrome b₅₆₂. The left-handed fold of the bromodomain structure is less common, but also observed in proteins such as cytochrome b₅ and T4 lysozyme (Richardson, Adv. Protein Chem., 34:167-339 (1989); Presnell, et al., Proc. Natl. Acad. Sci. USA 86:6592-6596 (1989)). This topological difference arises from the orientation of the loop between the first two helices (FIG. 2F-2G). The right-handed four-helix bundle proteins have a relatively short hairpin-like connection between the first two helices, which makes the “preferred” turn to the right at the top of the first helix (Richardson, Adv. Protein Chem., 34:167-339 (1989); Presnell, et al., Proc. Natl. Acad. Sci. USA 86:6592-6596 (1989); Weber, et al., Nature 287:82-84 (1980)). In contrast, proteins with the left-handed fold usually have a long loop after the first helix and often contain additional secondary structural elements at the base of the helix bundle (Richardson, Adv. Protein Chem., 34:167-339 (1989); Presnell, et al., Proc. Natl. Acad. Sci. USA 86:6592-6596 (1989)). In the bromodomain structure, this long ZA loop has a defined conformation and is packed against the loop between helices _{B} and _{C} (termed the BC loop) to form a hydrophobic pocket. These tertiary interactions between the two loops appear to favor the left turn of the ZA loop, resulting in the left-handed four-helix bundle fold of the bromodomain. The hydrophobic pocket formed by loops ZA and BC is lined by residues Val752, Ala757, Tyr760, Val763, Tyr802 and Tyr809 (FIG. 2H), and appears to be a site for protein-protein interactions (see below). The pocket is located at one end of the four-helix bundle, opposite to the N- and C-termini of the protein. Interestingly, the ZA loop varies in length amongst different bromodomains, but almost always contains residues corresponding to Phe748, Pro751, Pro758, Tyr760, and Pro767 (FIG. 1). The conservation of these residues within the ZA loop as well as residues within the -helical regions implies a similar left-handed four-helix bundle structure for the large family of bromodomains.

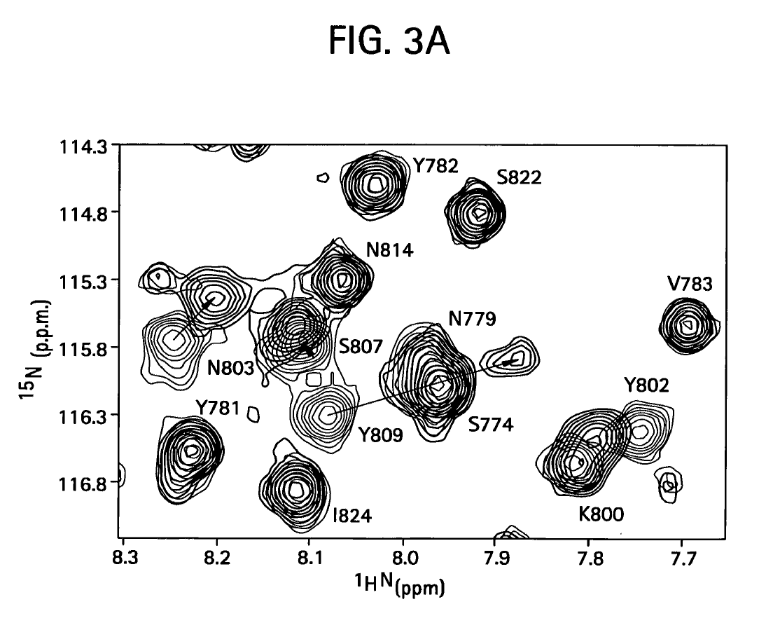

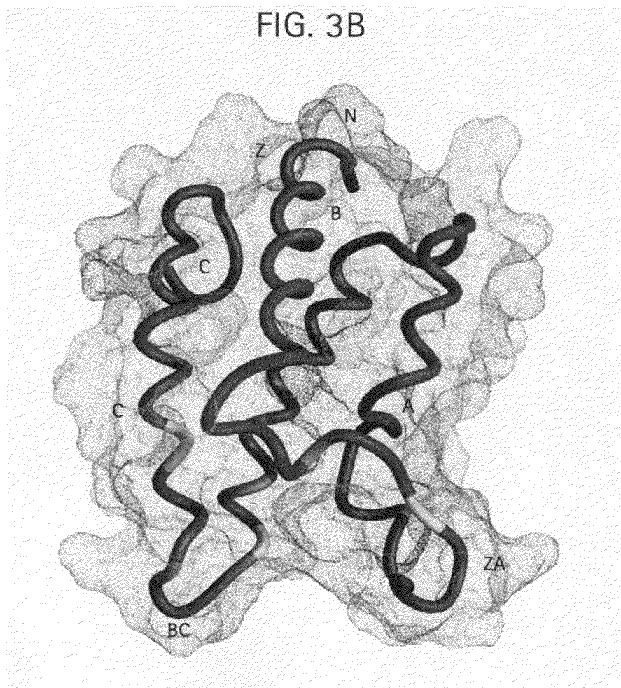

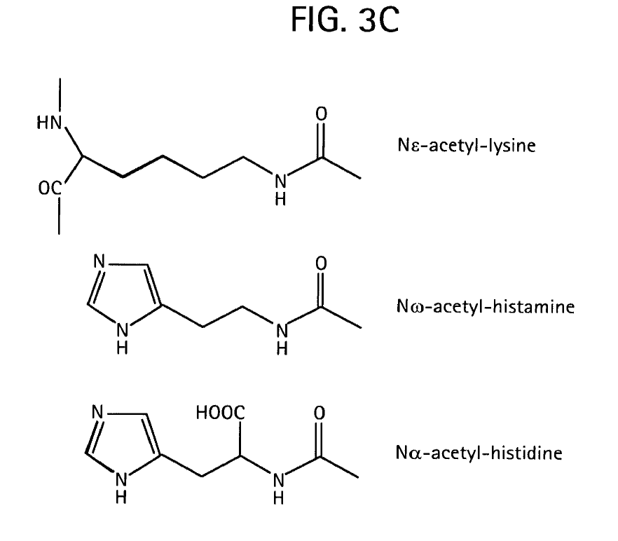

The modular bromodomain structure supports the idea that the bromodomain can act as a functional unit for protein-protein interactions. The observation that bromodomains are found in nearly all known nuclear HATs (A-type) that are known to promote transcription-related acetylation of histones on specific lysine residues, but not present in cytoplasmic HATs (B-type), prompted the determination of whether bromodomains can interact with acetyl-lysine (AcK). The NMR titration of the P/CAF bromodomain were performed with a peptide (SGRGKGG-AcK-GLGK) derived from histone H4, in which Lys8 is acetylated (Lys8 is the major acetylation site in H4 for GCN5, a yeast homologue of P/CAF). Remarkably, the bromodomain could indeed bind the AcK peptide. Moreover, this interaction appeared to be specific, based on the ¹⁵N-HSQC spectra which showed that only a limited number of residues underwent chemical shift changes as a function of peptide concentration (FIG. 3A). Conversely, the NMR titration of the bromodomain with a non-acetylated, but otherwise identical H4 peptide, showed no noticeable chemical shift changes, demonstrating that the interaction between the bromodomain and the lysine-acetylated H4 peptide was dependent upon acetylation of lysine. The dissociation constant (K_{D}) for the AcK peptide was estimated to be 346±54 μM. This binding is likely reinforced through additional interactions between bromodomain-containing proteins and target proteins. Notably, many chromatin-associated proteins contain two or multiple bromodomains (FIG. 1). Indeed, binding with another lysine-acetylated peptide (RKSTGG-AcK-APRKQ) derived from the major acetylation site on histone H3 (residues 9-20) was also observed. Together, these data demonstrate that the P/CAF bromodomain has the ability to bind AcK peptides in an acetylation dependent manner.

Intriguingly, the bromodomain residues that exhibited the most significant ¹H and ¹⁵N chemical shift changes on peptide binding are located near the hydrophobic pocket between the ZA and BC loops (FIG. 3B). Because a similar pattern of amide chemical shift changes was observed with the two different AcK-containing peptides, it was surmised that the hydrophobic cavity is the primary binding site for AcK. This hypothesis was further supported by titration with acetyl-histamine, which mimics the chemical structure of the AcK side-chain (FIG. 3C). Both ¹⁵N- and ¹³C-HSQC spectra showed that interaction with acetyl-histamine was also acetylation-dependent, involving the same set of residues that showed chemical shift perturbations with similar concentration dependence. It should be noted that the bromodomain did not bind to the amino acids acetyl-lysine or acetyl-histidine alone, possibly due to the presence of the charged amino, carboxyl, or caboxylate group adjacent to the acetyl moiety (FIG. 3C). Taken together, these results strongly suggest that the P/CAF bromodomain can interact with acetyl-lysine-containing proteins in a specific manner, and that this interaction is localized to the bromodomain hydrophobic cavity.

To identify the key residues involved in bromodomain-AcK recognition, the NMR structure of the P/CAF bromodomain in complex with acetyl-histamine was elucidated. As anticipated, the acetylated moiety binds in the bromodomain hydrophobic pocket (FIG. 4). The intermolecular interactions are largely hydrophobic in nature, with the methyl group of acetyl-histamine making extensive contacts with the side-chains of Val752, Ala757, and Tyr760, and the methylene groups of acetyl-histamine displaying specific NOEs to Val752, Ala757, Tyr760, Tyr802, and Tyr809. No intermolecular NOEs were observed for the imidazole ring of acetyl-histamine. From the spectral analysis it is clear that the structure of the bromodomain is very similar in both the free and complex forms. It is worth noting that the bromodomain-AcK recognition is reminiscent of the interactions between the histone acetyltransferase Hat1 and acetyl-CoA. Although the binding pockets of these two otherwise structurally unrelated proteins are composed of different secondary structural elements, the nature of acetyl-lysine recognition has striking similarities. In particular, Tyr809, Tyr802, Tyr760, and Val752 in the bromodomain appear to be related to Phe220, Phe261, Val254, and Ile217 of Hat1, respectively, in their interactions with the acetyl moiety. This observation may suggest an evolutionary convergent mechanism of acetyl-lysine recognition between bromodomains and histone acetyltransferases. To determine the relative contributions of residues within the hydrophobic cavity in bromodomain-AcK binding, site-directed mutagenesis was used to alter residues Tyr809, Tyr802, Tyr760, and Val752.

TABLE 1
Structural and Functional Analysis
of the P/CAF Bromodomain Mutants

| Bromodomain Protein | Structural Integrity ^{a} | H4 AcK-Peptide Binding |
|---|---|---|
| Wild-Type | ++++ | 346 ± 54 |
| Tyr809Ala | ++++ | No Binding ^{c} |
| Tyr802Ala | +++ | >10,000 ^{d} |
| Tyr760Ala | +++ | >10,000 |
| Val752Ala | ++ | >10,000 |

^{a} The effects of mutations on the structural integrity of the bromodomain were assessed by using the ¹⁵N-HSQC spectra. The amide ¹H/¹⁵N resonances of the mutant proteins were compared to those of the wild-type bromodomain to determine if the particular mutations lead to global or local structure disruption. Severe line-broadening of the amide resonances would indicate protein conformational exchange due to a decrease of structure stability resulting from point mutations. Structural integrity of the mutant proteins is expressed here relative to that of the wild-type, using the signs of “++++” for as stable as the wild-type, “+++” for mildly destabilized, “++” for moderately destabilized, and “−” for completely unfolded.
^{b} The ligand binding affinity (K_{D}) of the bromodomain proteins was estimated by following chemical shift changes of amide peaks in the ¹⁵N-HSQC spectra as a function of the ligand concentration.
^{c} No detectable ligand binding observed in the NMR titration.
^{d} Ligand binding affinity was significantly reduced and beyond the limit for reliable measurements by NMR titration.

Substitution of Ala for Tyr809 completely abrogated the bromodomain binding to the lysine-acetylated H4 peptide, while the Tyr802Ala, Tyr760Ala, and Val752Ala mutants had significantly reduced ligand binding affinity. To assess whether these mutations disrupted the overall bromodomain fold, the ¹⁵N-HSQC spectra of the mutants was compared to that of the wild-type protein. For the Tyr809Ala mutant, the amide chemical shifts were only affected for a few residues near the mutation site. However, mutations of the other residues in the hydrophobic binding pocket perturbed the local protein conformation to greater extents, particularly the ZA loop (Table 1; Table 7 of U.S. Ser. No. 09/784,553, filed Feb. 16, 2001, now U.S. Pat. No. 7,589,167, herein incorporated by reference). Thus, the NMR structural analysis and the mutagenesis studies show that Tyr809, which is structurally supported by Trp746 and Asn803 (FIG. 4), is essential for the bromodomain interaction with the acetyl group of acetyl-lysine, while residues of Tyr802, Tyr760, and Val752 likely play both structural and functional roles in the recognition. These residues are highly conserved throughout the bromodomain family (FIG. 1), suggesting that recognition of acetyl-lysine may be a feature of bromodomains, in general. Therefore, Val752, Ala757, Tyr760, Tyr802, Asn803, and Tyr809 are key amino acid residues for the P/CAF bromodomain binding to acetyl-lysine.

TABLE 2
Amino Acid Sequences of Bromodomains Identified in FIG. 1

| PROTEIN | SEQ ID | GenBank |
|---|---|---|
| hsp/CAF | 7 | U57317 |
| hsGCN5 | 8 | U57136 |
| ttP55 | 9 | U47321 |
| scGCN5 | 10 | Q03330 |
| hsP300 | 11 | A54277 |
| hsCBP | 12 | S39162 |
| mmCBP | 13 | S39161 |
| ceYNJ1 | 14 | P34545 |
| hsCCG1-1 | 15 | P21675 |
| msCCG1-1 | 16 | D26114 |
| hsCCG1-2 | 17 | |
| msCCG1-2 | 18 | |
| hsRing3-1 | 19 | P25440 |
| hsORFX-1 | 20 | D26362 |
| dmFSH-1 | 21 | P13709 |
| scBDF1-1 | 22 | P35817 |
| hsRing3-2 | 23 | |
| hsORFX-2 | 24 | |
| dmFSH-2 | 25 | |
| scBDF1-2 | 26 | |
| hsBR140 | 27 | JC2069 |
| hsSMAP | 28 | X87613 |
| ggPB1-1 | 29 | X90849 |
| ggPB1-2 | 30 | |
| ggPB1-3 | 31 | |
| ggPB1-4 | 32 | |
| ggPB1-5 | 33 | |
| spBRO-1 | 34 | S54260 |
| spBRO-2 | 35 | |
| hsSNF2a | 36 | S45251 |
| hsBRG1 | 37 | S39039 |
| ggBRM | 38 | X91638 |
| ggBRG1 | 39 | X91637 |
| hsTIF1b | 40 | X97548 |
| mmTIF1b | 41 | X99644 |
| mmTIF1a | 42 | S78219 |

### EXAMPLE 2

### Structural Insights Into HIV-1 TAT Transactivation via P/CAF

Whereas the life cycle of HIV is still being elucidated, it is currently accepted that HIV binds to CD4 protein of a host T cell or macrophage and with the aid of a chemokine receptor (e.g., CCR5 or CXCR4) enters the host cell. Once in the host cell, the retrovirus, HIV-1, is converted to a DNA by reverse transcriptase and the expression of the HIV-1 genome is dependent on a complex series of events that are believed to be under the control of two viral regulatory proteins, Tat and Rev (Romano et al., J. Cell Biochem. 75(3):357-368 (1999)). Rev controls post-translational events, whereas, Tat (the trans-activator protein) functions to stimulate the production of full-length HIV transcripts and viral replication in infected cells. The Tat protein transactivates the transcription of HIV-1 starting at the 5′ long terminal repeat (LTR) (Romano et al., J. Cell Biochem. 75(3):357-368 (1999)) by recruiting one or more carboxyl-terminal domain kinases to the HIV-1 promoter. More specifically, Tat stimulates transcription from the LTR at a hairpin element, the transactivation responsive region (TAR) (Kiernan et al., EMBO J. 18:6106-6118 (1999)) at least in part by interacting with and thereby recruiting the carboxyl-terminal domain kinase, i.e., the positive transcriptional elongation factor (P-TEFb) to the TAR RNA element (Garber et al., Mol. Cell. Biol. 20(18):6958-6969 (2000)). P-TEFb is a muti-subunit kinase that minimally comprises a heterodimer consisting of the regulatory cyclin T1 and its corresponding catalytic subunit, cyclin-dependent kinase 9 (CDK9). P-TEFb acts by phosphorylating the carboxyl-terminal domain of RNA polymerase II (Peng et al., J. Biol. Chem. 274 (49):34527-34530 (1999); Romano et al., J. Cell Biochem. 75(3):357-368 (1999)).

Recently, it has been shown that HIV-1 Tat transcription activity is regulated through lysine acetylation by, and association with the histone acetyltransferases (HATs) p300/CBP and the p300/CBP-associating factor (P/CAF), which specifically acetylate Lysine 50 (K50) and Lysine 28 (K28) of the Tat protein, respectively (Kiernan et al., EMBO J. 18:6106-6118 (1999); Ott et al., Curr. Biol. 9:1489-1492 (1999)). Notably, the acetylation of K50 by the transcriptional co-activator p300/CBP is on the C-terminal arginine-rich motif (ARM) of Tat, which is essential for its binding to the TAR RNA element and for nuclear localization, (Kiernan et al., EMBO J. 18:6106-6118 (1999); Ott et al., Curr. Biol. 9:1489-1492 (1999)). Acetylation of K28 of Tat by P/CAF enhances Tat binding to P-TEFb, whereas acetylation of K50 of Tat by P300/CBP promotes the dissociation of Tat from the TAR RNA element. This dissociation of Tat from the TAR RNA element occurs during early transcription elongation (Kiernan et al., EMBO J. 18:6106-6118 (1999)). However, heretofore, little else was known regarding the relationship of these HATs with Tat after the acetylation has occurred.

Sample preparation: The bromodomain of P/CAF (residues 719-832) was subcloned into the pET14b expression vector (Novagen) and expressed in Escherichia coli BL21(DE3) cells. Uniformly ¹⁵N- and ¹⁵N/¹³C-labeled proteins were prepared by growing bacteria in a minimal medium containing ¹⁵NH₄Cl with or without ¹³C₆-glucose. A uniformly ¹⁵N/¹³C-labeled and fractionally deuterated protein sample was prepared by growing the cells in 75% ²H₂O. The bromodomain was purified by affinity chromatography on a nickel-IDA column (Invitrogen) followed by the removal of poly-His tag by thrombin cleavage. The final purification of the protein was achieved by size-exclusion chromatography. The acetyl-lysine-containing peptides were prepared on a MilliGen 9050 peptide synthesizer (Perkin Elmer) using Fmoc/HBTU chemistry. Acetyl-lysine was incorporated using the reagent Fmoc-Ac-Lys with HBTU/DIPEA activation. NMR samples contained ˜0.5 mM protein in complex with the lysine-acetylated Tat peptide in 100 mM phosphate buffer of pH 6.5 and 5 mM perdeuterated DTT and 0.5 mM EDTA in H₂O/²H₂O (9/1) or ²H₂O. The bromodomain-containing constructs from P/CAF, CBP and TIF-1 were cloned into pGEX4T-3 vector (Pharmacia). These recombinant GST-fusion proteins were expressed in BL21 (DE3) codon plus cell line, and purified by using glutathione sepharose column.

NMR spectroscopy: All NMR spectra were acquired at 30° C. on a Bruker DRX600 or DRX500 spectrometer. The backbone assignments of the ¹H, ¹³C, and ¹⁵N resonances were achieved using deuterium-decoupled triple-resonance experiments of HNCACB and HN(CO)CACB (Yamazaki et al., J. Am. Chem. Soc. 116:11655-11666 (1994)) recorded using the uniformly ¹⁵N/¹³C-labelled and fractionally deuterated protein. The side-chain atoms were assigned from 3D HCCH-TOCSY (Clore, et al., Meth. Enzymol. 239:249-363 (1994)) and (H)C(CO)NH-TOCSY (Logan et al., J. Biolmol. NMR 3:225-231 (1993)) data collected on the uniformly ¹⁵N/¹³C-labeled protein. Stereospecific assignments of methyl groups of the valine and leucine residues were obtained using a fractionally ¹³C-labeled sample (Neri et al., Biochemistry 28:7510-7516 (1989)). The NOE-derived distance restraints were obtained from ¹⁵N- or ¹³C-edited 3D NOESY spectra (Clore, et al., Meth. Enzymol. 239:249-363 (1994)). □-angle restraints were determined based on the ³J_{HN,H} coupling constants measured in a 3D BNHA spectrum (Clore et al., Meth. Enzymol. 239:249-363 (1994)). Slowly exchanging amide protons were identified from a series of 2D ¹⁵N-HSQC spectra recorded after the H₂O buffer was changed to a ²H₂O buffer. The intermolecular NOEs used in defining the structure of the bromodomain/Ac-histamine complex were detected in ¹³C-edited (F₁), ¹³C/¹⁵N-filtered (F₃) 3D NOESY spectrum (Clore et al., Meth. Enzymol. 239:249-363 (1994)). All NMR spectra were processed with the NMRPipe/NMRDraw programs and analyzed using NMRView (Johnson, et al., J. Biomol., NMR 4:603-614 (1994)).

Ligand titration experiments were performed by recording a series of 2D ¹⁵N-HSQC spectra on the uniformly ¹⁵N-labelled bromodomain (˜0.3 mM), respectively, in the presence of different amounts of ligand concentration ranging from 0 to ˜2.0 mM. The protein sample and the stock solutions of the ligands were all prepared in the same aqueous buffer containing 100 mM phosphate and 5 mM perdeuterated DTT at pH 6.5.

Structure calculations. Structures of the bromodomain were calculated with a distance geometry/simulated annealing protocol using the X-PLOR program (Brunger, X-PLOR Version 3.1: A system for X-Ray crystallography and NMR, Yale University Press, New Haven, Conn., (1993)). A total of 1324 manually assigned NOE-derived distance restraints were obtained from the ¹⁵N- and ¹³C-edited NOE spectra. Further analysis of the NOE spectra was carried out by the iterative automated assignment procedure by using ARIA (Nilges, et al., Prog. NMR Spectroscopy 32:107-139 (1998)), which integrates with X-PLOR for structure calculations. The ARIA-assigned distance restraints were in agreement with the structures calculated using only the manually assigned NOE distance restraints, hydrogen-bond distance restraints, and 54 φ-angle restraints. The final structure calculations employed a total of 2903 NMR experimental restraints obtained from the manual and the ARIA-assisted assignments. For the ensemble of the final 30 structures, no distance and torsional angle restraints were violated by more than 0.3 Å and 5 Å, respectively. The Lennard-Jones potential which was not used during any refinement stage, and stereochemistry of the final structures was validated with Ramachandran plot analysis by using Procheck-NMR (Laskowski et al., J. Biolmol. NMR 8:477-486 (1996)).

Site directed mutagenesis. Site directed mutagenesis was performed on selected residues of P/CAF Bromodomain using quick-change kit (Stratagene). The mutants were confirmed by sequencing and proteins were expressed and purified as above.

Peptide binding assay. Equal amount (10 μM) of GST, GST-P/CAF bromodomain and its mutant proteins, as well as various GST-fusion bromodomains from CBP and TIF1 were incubated for at least two hours at room temperature with the N-terminal biotinylated and lysine-acetylated Tat peptide (50 μM) in a 50 mM Tris buffer of pH 7.5, containing 50 mM NaCl, 0.1% BSA and 1 mM DTT. Streptavidin agarose (10 μL) was added to mixture and the beads were washed twice in the Tris buffer with 500 mM NaCl and 0.1% NP-40. Proteins were eluted from the argarose beads in SDS buffer and separated on a 14% SDS-PAGE. The resolved proteins were transferred onto nitrocellulose membrane (Pharmacia), and the membrane was blocked overnight with 5% non-fat milk in washing buffer of 20 mM Tris, pH 7.5, plus 150 mM NaCl and 0.1% Tween-20 at 4° C. Western blotting was performed with anti-GST antibody (Sigma) and goat anti-rabbit IgG conjugated with horseradish-peroxidase (Promega) and developed by chemiluminescence. Peptide competition experiments were performed by incubating various non-biotinylated and mutant Tat peptide with the P/CAF bromodomain and the biotinylated and wild type Tat peptide. The molar ratio of the wild type and mutant Tat peptides in the mixture were kept at 1:2. The binding results were analyses by using the procedure as described above. The full length protein sequence of the Human Immunodeficiency Virus type 1 Tat was obtained from GenBank, Accession No: AAA83395 (SEQ ID NO:45).

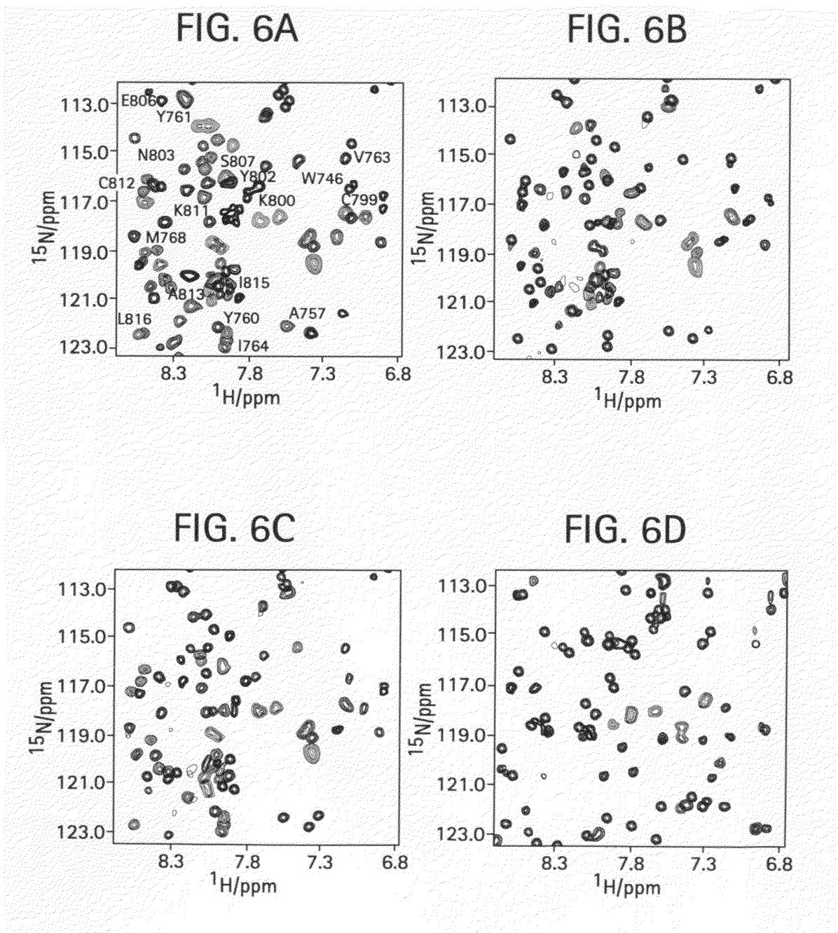

Results. To test whether or not the bromodomains of these HATs can bind to the lysine-acetylated Tat, in vitro binding assays were performed by using recombinant and purified bromodomains and lysine-acetylated peptides derived from the acetylation sites in Tat. While the bromodomains of CBP and TIF1 did not show any binding, the P/CAF bromodomain binds tightly only to the Tat peptide containing AcK50 (where AcK stands for an N-acetyl lysine residue) (FIGS. 5A-5B). NMR binding studies further confirmed the specific interaction of the P/CAF bromodomain and lysine-acetylated Tat peptide. Because NMR resonances of amide protons are highly sensitive to local chemical environment and conformational change in a protein, two-dimensional ¹H-¹⁵N heteronuclear single quantum correlation (HSQC) spectrum can be used to detect even weak but specific interactions between a protein and its binding ligand. As shown in 2D HSQC spectra (FIGS. 6A-6D), the bromodomain of P/CAF binds weakly to the lysine-acetylated peptides derived from known acetylation sites of K28 on Tat and of K16 on histone H4 by only interacting with the acetyl-lysine residue in the peptides (K_{d}<300 μM). This is reflected the relatively small chemical shift perturbation of the amide proton signals of the protein upon addition of ligand. On the other hand, the P/CAF bromodomain interacts strongly with the Tat AcK50 peptide, which involves many protein residues in addition to those for acetyl-lysine binding with an estimated K_{d} of ˜20 μM. Binding of peptide residues flanking the acetyl-lysine may explain the high specificity of the P/CAF bromodomain for the acetylated Tat. Furthermore, the p300/CBP bromodomain did not bind the lysine-acetylated Tat peptide in a specific manner except its weak interaction with the acetyl-lysine residue in the peptide (FIGS. 6A-6D). Together, these results demonstrate the P/CAF bromodomain can specifically recognize the lysine-acetylated Tat involving K50.

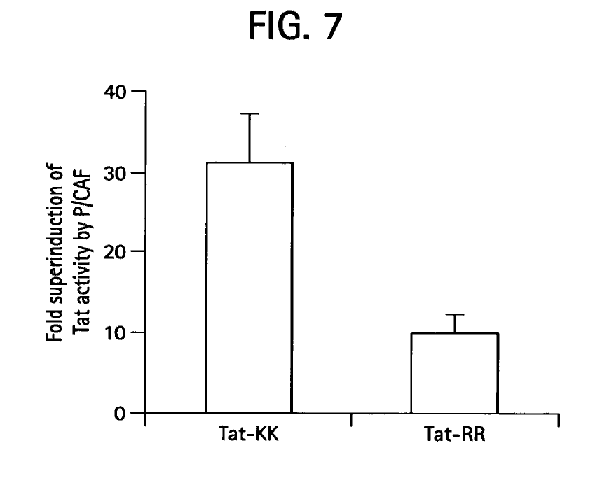

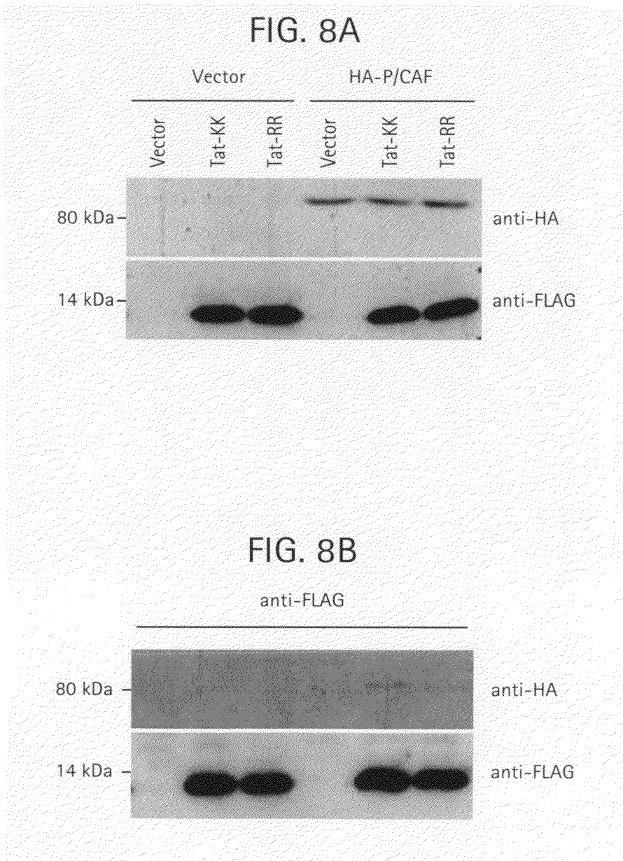

To determine how the P/CAF binding affects Tat function in vivo, transactivation activity of Tat was measured. Superinduction of Tat transactivation activity exhibited as much as a 30-fold increase upon P/CAF stimulation (FIG. 7). This profound P/CAF effect requires acetylation at K50 on Tat, as a double mutant of K50 and K51 substituted with arginines resulted in a nearly two-thirds reduction of the enhancement. Further, specific interaction between P/CAF and wild type Tat in cells was also detected, but not with the Tat double mutant containing K50R/K51R (FIGS. 8A-8B). Taken together, these results confirm that P/CAF can directly interact via its bromodomain with the lysine-acetylated Tat, which possibly regulates Tat transactivation activity.

To further understand the molecular basis of the P/CAF bromodomain recognition of the lysine-acetylated Tat, the three-dimensional structure was determined for the P/CAF bromodomain in complex with an 11-residue Tat peptide containing AcK50. A total of 2,903 NMR-derived distance and dihedral angle restraints were used. The structure of the bromodomain in the peptide-bound form consists of an up-and-down four-helix bundle (helices _{Z, A, B,} and _{C}) with a left-handed twist, and a long intervening loop between helices _{Z} and _{A} (termed the ZA loop) (FIG. 9). The overall structure of the complex is well defined, and similar to the structure of the free bromodomain (Dhalluin et al., Nature 399:491-496 (1999)) except that the ZA and BC loops, which compose the acetyl-lysine binding pocket, undergo local conformational changes in order to accommodate their interactions with the peptide residues.

TABLE 3
NMR Structural Statistics of the P/CAF Bromodomain/Tat Peptide Complex

| | |
|---|---|
| Total Experimental Restraints | 2903 |
| Distance Restraints ^{a} | 2822 |
| Total Ambiguous | 122 |
| Total Unambiguous | 2700 |
| Intra-residue (i = j) | 1118 (41.40%) |
| Sequential (\|i − j\| = 1) | 487 (18.04%) |
| Medium (2.\|i − j\|. 4) | 547 (20.26%) |
| Long range (\|i − j\| > 4) | 478 (17.70%) |
| Intermolecular | 78 (2.39%) |
| Hydrogen Bond Restraints | 28 |
| Dihedral Angle Restraints | 53 |
| Final Energies (kcalmol⁻¹) | |
| E_{Total} | 366.35 ± 31.11 |
| E_{NOE} | 58.05 ± 12.57 |
| E_{Dihedral} | 0.57 ± 0.31 |
| E_{L-J} ^{b} | −569.47 ± 22.42 |

| | Protein/Peptide Complex | Secondary Structure |
|---|---|---|
| Ramachandran Plot (%) | | |
| Most Favorable Region | 72.06 ± 2.29 | 91.95 ± 3.04 |
| Additionally Allowed Region | 22.91 ± 2.41 | 7.42 ± 3.08 |
| Generously Allowed region | 3.64 ± 1.40 | 0.62 ± 0.82 |
| Disallowed Region | 1.33 ± 0.64 | 0.00 ± 0.0 |
| RMSDs of Atomic Coordinates (Å) | | |
| Protein (aa 9-116) | | |
| Backbone | 0.66 ± 0.14 | 0.39 ± 0.05 |
| Heavy atoms | 1.25 ± 0.18 | 0.96 ± 0.07 |
| Peptide (aa 202-206, 208-209) | | |
| Backbone | 0.50 ± 0.16 | |
| Heavy atoms | 1.83 ± 0.50 | |
| Complex (aa 9-116, 202-206, 208-209) | | |
| Backbone | 0.72 ± 0.15 | 0.54 ± 0.09 |
| Heavy atoms | 1.39 ± 0.20 | 1.24 ± 0.16 |

^{a} Of the total 2903 NOE-derived distance restraints, only 341 were obtained by using ARIA program, of which 122 are classified as ambiguous NOEs. The latter resonance signals in the spectra match with more than one proton atom in both the chemical shift assignment and the final NMR structures.
^{b} The Lennard-Jones potential was not used during any refinement stage.
^{c} None of these final structures exhibit NOE-derived distance restraint violations greater than 0.5 Å or dihedral angle restraint violations greater than 5°.

The Tat AcK50 peptide adopts an extended conformation and lies between the ZA and BC loops (FIG. 9). The acetyl-lysine side-chain intercalates deep into a preformed hydrophobic and aromatic cavity located between the ZA and BC loops opposite to the N- and C-termini, and interacts extensively with residues V752, Y760, I764, Y802, and Y809. While the peptide residues S(AcK-4), K(AcK+1), R(AcK+2), R(AcK+5) do not interact directly with the protein, the residues Y(AcK-3), G(AcK-2), R(AcK-1), R(AcK+3), and Q(AcK+4) showed numerous intermolecular NOEs with the protein. Particularly, Y(AcK-3) and Q(AcK+4) form extensive contacts with V763 and E756, respectively, suggesting that these two residues contribute significantly to specificity of the bromodomain/Tat recognition.

To identify the amino acid residues of the P/CAF bromodomain that are important for complex formation, mutant proteins were tested for binding to the biotinylated and lysine-acetylated Tat peptide that is immobilized onto streptavidin agarose (FIG. 10A). As expected, proteins containing alanine point mutation at the residue Y809, Y802, V752, or F748, which interact directly with the acetyl-lysine residue, showed nearly complete loss or significantly reduced binding to the Tat peptide. Moreover, when the residue V763 or E756 was mutated to alanine, a nearly complete loss in binding to the Tat AcK50 peptide was observed, indicating that these two amino acid residues provide essential contributions to the Tat recognition by interacting with the residues flanking the acetyl-lysine. The results from the mutational analysis agree with the observations of intermolecular NOEs in the NMR spectra.

To further determine Tat sequence preference for P/CAF interaction, various mutant peptides were synthesized and their binding to the P/CAF bromodomain tested in a competition assay by using a western blot with the antibody against the GST-fusion bromodomain (FIG. 10B). Because of high sensitivity of this detection method, the binding assay was performed at protein concentration (˜10 □M) much lower than that in the NMR binding studies, which ensured specificity of protein-peptide interactions. In agreement with the binding results described above (e.g., see FIGS. 5A-5B, 6A-6D, 7, and 8A-8B), lysine-acetylated peptides derived from acetylation sites at K50 or K28 in Tat, or from histone H4 at K16 showed almost no competition with the Tat AcK50 peptide in binding to the P/CAF bromodomain, confirming that the latter interaction is tight and specific. Additionally, while substitution of residue R(AcK−1), K(AcK+1), R(AcK+2), or R(AcK+3) to alanine slightly weakened Tat peptide binding to the bromodomain, mutation of Y(AcK−3) or Q(AcK+4) resulted in significant loss in binding to the protein. These data can be explained by the observation of extensive pair-wise interactions between Y(AcK−3) and V763, and between Q(AcK+4) and E756, which agrees perfectly with the site-directed mutatagenesis results obtained with the protein (FIG. 10A). Together, these results demonstrate that the specificity of P/CAF bromodomain and acetylated Tat complex formation is achieved through specific interactions with acetyl-lysine as well as amino acid residues at (AcK−3) and (AcK+4) positions.

The HIV-1 Tat is a versatile protein and elicits many cellular functions. In addition to its lysine-acetylation and interaction with P/CAF as disclosed herein, this portion of arginine-rich motif (named ARM) has also been shown to interact with the TAR RNA element as well as protein nuclear localization, particularly involving arginine52 and arginine53. The findings disclosed herein that are based on the detailed structural and mutational analyses indicate that the lysine-acetylated Tat specifically is associated with P/CAF via a bromodomain interaction in vivo, and that this interaction is important for transactivation activity of Tat in cells. Further, the data disclosed herein reveal that in addition to the acetylated-lysine (K50) the flanking residues, tyrosine (AcK−3) and glutamine at (AcK+4) positions in Tat are also uniquely important for the specificity of the Tat and P/CAF bromodomain recognition, but not with its other functions. This new information is extremely useful in applying mutational analysis in in vivo studies to further elucidate the biological importance of the Tat-P/CAF association in molecular mechanisms by which Tat transactivates gene transcription of HIV-1 via chromatin remodeling.

### EXAMPLE 3

### Synthesis of the Compounds of Formula I

Sample preparation. The PCAF bromodomain (residues 719-832) was expressed in E. coli BL21(DE3) cells using the pET14b vector (Novagen) (Dhalluin, et al., Nature (1999) 399, 491-496). Isotope-labeled proteins were prepared from cells grown on a minimal medium containing ¹⁵NH₄Cl with or without ¹³C₆-glucose in either H₂O or 75% ²H₂O. The protein was purified by affinity chromatography on a nickel-IDA column (Invitrogen), followed by the removal of poly-His tag by thrombin cleavage. GST-fusion PCAF bromodomain was expressed in E. coli BL21 (DE3) codon plus cells using the pGEX4T-3 vector (Pharmacia), and purified with a glutathione sepharose column. The lysine-acetylated peptide was ordered from Biosynthesis, Inc.

Protein structure determination by NMR. NMR samples contained the bromodomain (0.5 mM) in complex with a chemical ligand (˜2 mM) in 100 mM phosphate buffer of pH 6.5, containing 5 mM perdeuterated DTT and 0.5 mM EDTA in H₂O/²H₂O (9/1) or ²H₂O. All NMR spectra were acquired at 30° C. on a Bruker 500 or 600 MHz NMR spectrometer. The backbone ¹H, ¹³C and ¹⁵N resonances were assigned using 3D HNCACB and HN(CO)CACB spectra. The side-chain atoms were assigned from 3D HCCH-TOCSY and (H)C(CO)NH-TOCSY data. The NOE-derived distance restraints were obtained from ¹⁵N- or ¹³C-edited 3D NOESY spectra. The ³J_{HN,H═} Coupling constants measured from 3D HNHA data were used to determine □-angle restraints. Slowly exchanging amide protons were identified from a series of 2D ¹⁵N-HSQC spectra recorded after H₂O/²H2O exchange. The intermolecular NOEs used in defining the structure of the PCAF bromodomain/ligand complex were detected in ¹³C-edited (F₁), ¹³C/¹⁵N-filtered (F₃) 3D NOESY spectra (Clore et al., Meth. Enzymol. (1994) 239, 249-363). Protein structures were calculated with a distance geometry-simulated annealing protocol with X-PLOR (Brunger, X-PLOR Version 3.1: A system for X-Ray crystallography and NMR. version 3.1 ed. 1993, New Haven, Conn.: Yale University Press). Initial structure calculations were performed with manually assigned NOE-derived distance restraints. Hydrogen-bond distance restraints, generated from the H/D exchange data, were added at a later stage of structure calculations for residues with characteristic NOEs. The converged structures were used for iterative automated NOE assignment by ARIA for refinement (Nilges et al., Prog. NMR Spectroscopy (1998) 32, 107-139). Structure quality was assessed by Procheck-NMR (Laskowski et al, J. Biomol. NMR (1996) 8, 477-486). The structure of the protein/ligand complex was determined using intermolecular NOE-derived distance restraints.

Chemical screening by NMR. A chemical library was constructed with small-molecules obtained from Chembridge Corp. (San Diego, Calif.), which were selected on the basis of molecular weight (<250 Da), non-reactivity, drug-like chemical framework (i.e. ring systems, linker atoms, side-chain atoms and framework), functional moieties (for hydrogen-bond or electrostatic interactions, but not reactive) and good solubility in aqueous solution. All the stock solutions of the chemical compounds are prepared in predeuterated DMSO. NMR-based screening was conducted with compounds (˜1 mM) and the PCAF bromodomain (50-200 μM) using methods including 1D NOE-pumping (Chen et al., J. Am. Chem. Soc. (1998) 120, 10258-10259) and saturation transfer difference (Kwak et al., J. Biol. Chem. (1995) 270, 1156-1160; Klein et al., J. Am. Chem. Soc. (1999) 121, 5336-5337), as well as 2D ¹⁵N-HSQC spectra (Hajduk et al., Quarterly Reviews of Biophysics (1999) 32, 211-240; Moore, Curr. Opin. in Biotech. (1999) 10, 54-58). The latter is particularly helpful for selective screening to identify compounds that bind to a specific site of the target protein.

Ligand binding to the PCAF bromodomain. The ELISA assay was carried on a 96-well microplate (Nunc) that was pre-coated with anti-GST antibody (Sigma-Aldrich) overnight at 4° C. in 100 μL carbonate/bicarbonate buffer, and then washed with PBS buffer supplemented with 1% Tween-20. Non-specific binding sites were minimized by treatment with PBS buffer containing 2% BSA and 1% Tween-20 for 2 hours at room temperature. GST-PCAF bromodomain (1 μg per well) was added to the plate and incubated for two hours at room temperature for binding to anti-GST antibody. The plate was washed and blocked with PBS buffer containing 10% BSA and 1% Tween-20. The biotinylated HIV Tat-AcK50 peptide (Biotin-GISYGR-AcK-KKRRQRRRP) (5 μM) and increasing concentrations of a given compound were added and allowed to bind to the PCAF bromodomain overnight at 4° C. Plate was washed with washing buffer, and bromodomain-bound peptide was determined by incubating 100 μL of a neutravidin-conjugated HRP (Pierce) solution (0.1 μg/ml) for 1 hour at room temperature, followed by washes and incubation with 100 μL of tetramethyl benzidine (Pierce) as an HRP substrate. The reaction was stopped by addition of 100 μL of 2.0 M sulfuric acid. The absorbance of the colored product was measured at 450 nm. Absorbance in each well was corrected for the blank obtained in a corresponding well subjected to the complete procedure but containing no PCAF bromodomain.

Chemistry. Melting points were recorded on an XT-4 micro-melting point apparatus and were uncorrected. ¹H and ¹³C NMR spectra were recorded on a 300 MHz Bruker NMR spectrometer using tertramethylsilane as internal standard and the data were reported as the following: chemical shifts in ppm (δ), number of protons, multiplicity (s, singlet; d, doublet; t, triplet; m, multiplet), coupling constants in hertz. IR spectra were measured on Bruker EQUINOX55 spectrometer. Mass spectra were recorded on HRMS (LC-TOF spectrometer (micromass)) (EI/CI). Elemental analyses were recorded on Elementar Vario EL-III spectrometer. All chromatographic purifications were performed with silica gel (100-200 mesh). All purchased materials were used without further purifications. All solvents were reagent grades.

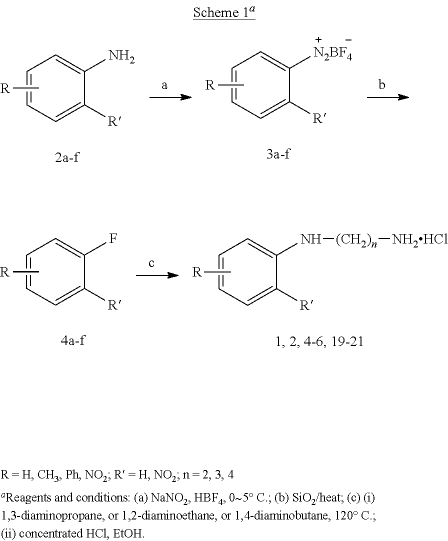

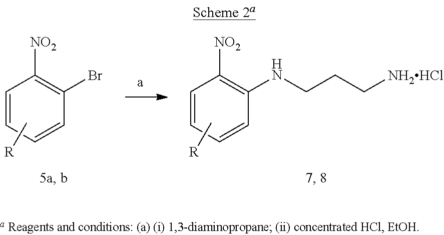

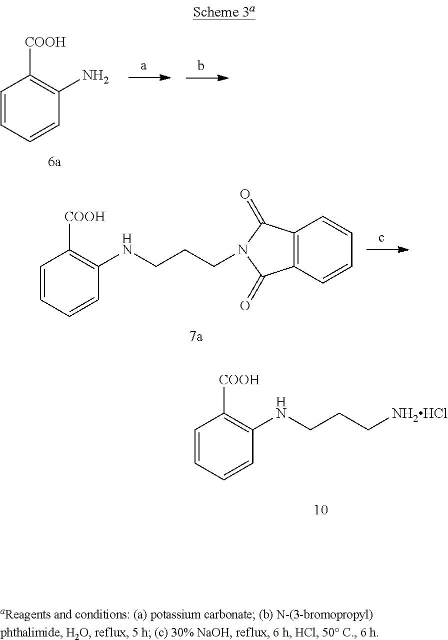

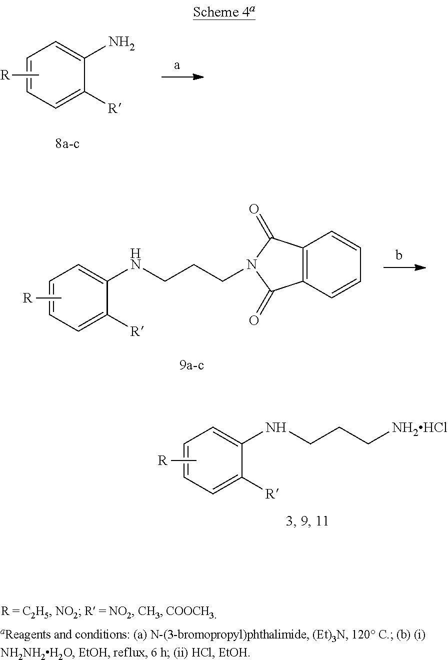

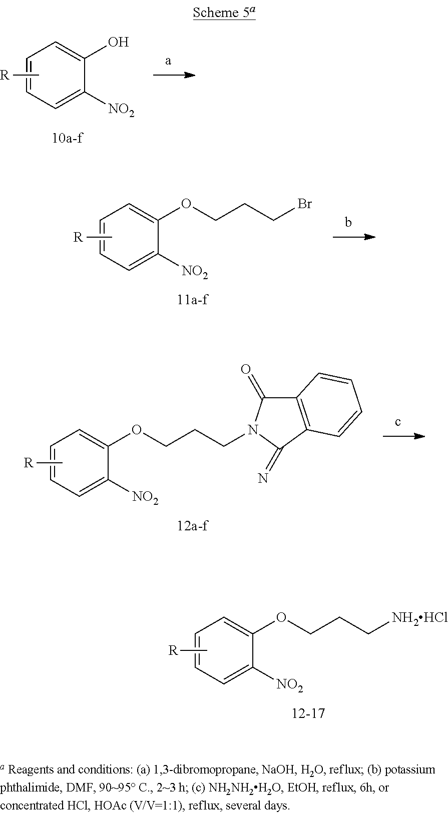

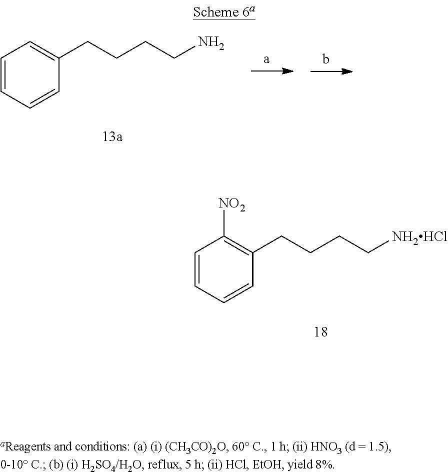

N₁-(2-nitro-phenyl)-propane-1,3-diamine monohydrochloride. Yield: 78%; mp >169° C.; FTIR (KBr) ν̅ cm⁻¹: 2988.48, 1614.95, 1592.93, 1530.24, 1499.85, 1382.35, 1347.25, 1224.06, 1154.59, 1134.19, 1004.51, 918.02, 858.90, 789.80, 747.36; ¹H NMR (D₂O, 4.79) δ: 7.80 (1H, m, Ar—H), 7.35 (1H, m, Ar—H), 6.76 (1H, m, Ar—H), 6.50 (1H, m, Ar—H), 3.31 (2H, t, J=6.90 Hz, 3-CH₂), 3.05 (2H, t, J=7.40 Hz, 1-CH₂), 1.96 (2H, m, 2-CH₂); ¹³C NMR (D₂O+acetone, acetone (CH₃):30.60) δ: 145.28, 137.35, 130.64, 126.44, 116.27, 114.44, 39.87, 37.64, 26.48; HRMS(CI) calculated for C₉K₃N₃O₂: (M+1) 196.1086, found 196.1080.

N₁-(4-methyl-2-nitro-phenyl)-propane-1,3-diamine monohydrochloride. Yield: 60.8%; mp 184-186° C.; FTIR (KBr) ν̅ cm⁻¹: 2964.50, 136.57, 1566.58, 1526.08, 1398.59, 1351.62, 1229.25, 1167.67, 1059.48, 851.53, 814.01, 764.74; ¹H NMR (D₂O, 4.79) δ: 7.79 (1H, s, 3-Ar—H), 7.36 (1H, d, J=8.69 Hz, 5-Ar—H), 6.88 (1H, d, J=8.67 Hz, 6-Ar—H), 3.47 (2H, t, =6.62 Hz, 3-CH₂), 3.15 (2H, t, J=7.43 Hz, 1-CH₂), 2.21 (3H, s, —CH₃), 2.08 (2H, m, 2-CH₂); ¹³C NMR (D₂O+acetone, acetone (CH₃):30.60) δ: 144.06, 139.14, 130.62, 126.21, 125.53, 114.43, 39.81, 37.67, 26.65, 19.38; HRMS(CI) calculated for C₁₀H₁₅N₃O₂: (M+1) 210.1243, found 210.1251.

N₁-(4-ethyl-2-nitro-phenyl)-propane-1,3-diamine monohydrochloride. Yield: 72.7%; mp 183-185° C.; FTIR(KBr) ν̅ cm⁻¹: 2965.79, 1634.14, 1569.30, 1523.56, 1407.80, 1351.13, 1269.12, 1225.26, 1165.22, 1069.81, 817.62, 762.33; ¹H NMR (D₂O, 4.79)δ7.76 (1H, s, 3-Ar—H), 7.37 (1H, dd, J=7.01, 1.83 Hz, 5-Ar—H), 6.87 (1H, d, J=8.87 Hz, 6-Ar—H), 3.44 (2H, t, J=6.82 Hz, NH—CH₂), 3.12 (2H, t, J=7.57 Hz, CH₂—NH₂), 2.48 (2H, q, J=7.53 Hz, Ar—CH₂), 2.05 (2H, m, 2-CH₂), 1.14 (3H, s, —CH₃); ¹³C NMR (D₂O+acetone, acetone (CH₃):30.60) δ144.21, 138.16, 132.47, 130.58, 124.08, 114.52, 39.82, 37.65, 27.04, 26.68, 14.59; HRMS(CI) Calcd for C₁₁H₁₇N₃O₂: (M+1) 224.1399, found 224.1398.

N₁-(3-methyl-2-nitro-phenyl)-propane-1,3-diamine monohydrochloride. Yield: 81.5%; mp 180-182° C.; FTIR (KBr) ν̅ cm⁻¹: 2972.09, 1606.43, 1576.89, 1534.85, 1501.40, 1351.61, 1250.77, 1161.47, 1064.62, 1033.92, 852.11, 796.63, 769.27; ¹H NMR (D₂O, 4.79) δ: 7.35 (1H, t, J=7.88 Hz, 5-Ar—H), 6.86 (1H, d, J=8.50 Hz, 4-Ar—H), 6.70 (1H, d, J=7.36 Hz, 6-Ar—H), 3.38 (2H, t, J=6.85 Hz, 3-CH₂), 3.11 (2H, t, J=7.41 Hz, 1-CH₂), 2.36 (3H, s, —CH₃), 2.03 (2H, m, 2-CH₂); ¹³C NMR (D₂O+acetone, acetone (CH₃):30.60) δ141.92, 136.58, 135.56, 134.11, 121.53, 113.10, 41.22, 37.66, 26.27, 20.34; HRMS(CI) calculated for C₁₀H₁₅N₃O₂: (M+1) 210.1243, found 210.1251.

N₁-(5-methyl-2-nitro-phenyl)-propane-1,3-diamine monohydrochloride. Yield: 67.1%; mp >215° C.; FTIR (KBr) ν̅ cm⁻¹: 2925.75, 1628.10, 1577.27, 1488.41, 1408.98, 1338.69, 1252.10, 1217.79, 1182.52, 1060.46, 752.22; ¹H NMR (D₂O, 4.79) δ: 7.99 (1H, m, 3-Ar—H), 6.83 (1H, s, 6-Ar—H), 6.58 (1H, d, J=6.76 Hz, 4-Ar—H), 3.52 (2H, t, J=6.64 Hz, 3-CH₂), 3.17 (2H, t, J=7.60 Hz, 1-CH₂), 2.36 (3H, s, —CH₃), 2.10 (2H, m, 2-CH₂); ¹³C NMR (D₂O+acetone, acetone (CH₃):30.60) δ: 150.00, 145.91, 129.12, 126.62, 118.14, 113.71, 39.64, 37.65, 26.59, 21.68; HRMS(CI) calculated for C₁₀H₁₅N₃O₂: (M+1) 210.1243, found 210.1234.

N₁-(3-nitro-biphenyl-4-yl)-propane-1,3-diamine monohydrochloride. mp >190° C.; FTIR (KBr) ν̅ cm⁻¹:3435.01, 2971.02, 1635.79, 1561.44, 1404.99, 1359.55, 1241.99, 1222.08, 1169.70, 158.97, 698.40; ¹H NMR (D₂O, 4.79) δ: 8.34 (1H, d, J=2.01 Hz, Ar—H), 7.83 (1H, m, Ar—H), 7.64 (2H, d, J=7.26 Hz, Ar—H), 7.50 (2H, m, Ar—H), 7.42 (1H, m, Ar—H), 7.08 (1H, d, J=9.02 Hz, Ar—H), 3.53 (2H, t, J=6.96 Hz, 3-CH₂), 3.13 (2H, t, J=7.59 Hz, 1-CH₂), 2.08 (2H, m, 2-CH₂); ¹³C NMR (DMSO-d₆) δ: 144.19, 144.06, 138.05, 134.84, 131.49, 129.07, 127.15, 127.11, 125.80, 123.36, 115.45, 36.44, 36.35, 26.16; HRMS(CI) calculated for C₁₅H₁₇N₃O₂: (M+1) 272.1399, found 272.1390.

N₁-(4-cyano-2-nitro-phenyl)-propane-1,3-diamine monohydrochloride. mp >230° C.; FTIR (KBr) ν̅ cm⁻¹: 3364.13, 2929.80, 2222.94, 1625.89, 1561.22, 1524.98, 1410.25, 1364.46, 1261.01, 1176.10, 921.75, 819.69; ¹H NMR (D₂O, 4.79) δ: 8.58 (1H, d, J=1.54 Hz, 3-Ar—H), 7.73 (1H, dd, J=1.70, 9.13 Hz, 5-Ar—H), 7.12 (1H, d, J=9.15 Hz, 6-Ar—H), 3.58 (2H, t, J=6.91 Hz, 3-CH₂), 3.13 (2H, t, J=8.01 Hz, 1-CH₂), 2.08 (2H, m, 2-CH₂); ¹³C NMR (DMSO-d₆) δ: 146.68, 137.58, 131.90, 131.01, 118.22, 115.90, 96.29, 36.06, 29.78, 25.77; HRMS(CI) calculated for C₁₀H₁₂N₄O₂: (M+1) 221.1039, found 221.1040.

N₁-(5-cyano-2-nitro-phenyl)-propane-1,3-diamine monohydrochloride. mp >185° C.; FTIR (KBr) ν̅ cm⁻¹: 3234.94, 2228.70, 1602.69, 1470.61, 1308.98, 1266.12, 1158.97, 1070.74, 874.89, 829.64, 750.00; ¹H NMR (D₂O, 4.79) δ: 8.20 (1H, d, J=8.73 Hz, 3-Ar—H), 7.91 (1H, brs, Ar—NH), 7.66 (1H, s, 6-Ar—H), 7.04 (1H, dd, J=1.38, 8.76 Hz, 4-Ar—H), 3.52 (2H, t, J=6.39 Hz, 3-CH₂), 2.86 (2H, brs, 1-CH₂), 1.86 (2H, m, 2-CH₂); ¹³C NMR (DMSO-d₆) δ: 144.15, 133.43, 127.68, 119.35, 118.11, 117.65, 116.81; 36.43, 30.61, 25.96; HRMS(CI) calculated for C₁₀H₁₂N₄O₂: (M+1) 221.1039, found 221.1045.

N₁-(2-methyl-5-nitro-phenyl)-propane-1,3-diamine monohydrochloride. Yield: 81%; mp >152° C.; FTIR(KBr) ν̅ cm⁻¹: 3246.44, 2984.42, 1627.55, 1534.15, 1510.90, 1349.34, 1167.14, 1130.54, 917.28, 853.18, 737.04; ¹H NMR (D₂O, 4.79)δ7.77 (1H, dd, J=2.15, 8.24 Hz, 4-Ar—H), 7.70 (1H, d, J=1.94 Hz, 6-Ar—H), 7.38 (1H, d, J=8.27 Hz, 3-Ar—H), 3.43 (2H, t, J=7.2 Hz, 3-CH₂), 3.14 (2H, t, J=7.51 Hz, 1-CH₂), 2.32 (3H, s, —CH₃), 2.10 (2H, m, 2-CH₂); ¹³C NMR (D₂O+acetone, acetone (CH₃):30.60) δ146.78, 137.91, 132.96, 121.03, 114.10, 45.80, 37.24, 24.59, 17.36; HRMS(CI) calculated for C₁₀H₁₅N₃O₂: (M+1) 210.1243, found 210.1238.

N₁-(2-carboxyl-phenyl)-propane-1,3-diamine monohydrochloride. Yield: 51%; mp >135° C.; FTIR (KBr) ν̅ ⁻¹: 3328.13, 3045.98, 1687.79, 1605.86, 1579.99, 1379.37, 1198.87, 1158.54, 755.76; ¹H NMR (DMSO-d₆) δ7.69 (1H, d, J=7.73 Hz, 3-Ar—H), 7.41 (1H, t, J=7.35, 8.02 Hz, 5-Ar—H), 7.15 (1H, d, J=8.02 Hz, 6-Ar—H), 7.08 (1H, t, J=7.73, 7.35 Hz, 4-Ar—H), 3.47 (2H, t, J=6.27 Hz, 3-CH₂), 3.31 (2H, t, J=4.89 Hz, 1-CH₂), 1.69 (2H, m, 2-CH₂); ¹³C NMR (DMSO-d₆) δ167.28, 131.92, 128.45, 122.23, 121.14, 116.86, 114.13, 58.59, 36.50, 32.19; HRMS(CI) calculated for C₁₀H₁₄N₂O₂: (M+1) 195.1134, found 195.1119.

N₁-(2-carboxymethyl-phenyl)-propane-1,3-diamine monohydrochloride. mp 141-143° C.; FTIR(KBr) ν̅ cm⁻¹:3344.52, 2952.20, 1697.49, 1605.34, 1574.36, 1508.79, 1438.17, 1258.09, 1222.79, 1169.82, 1088.74, 751.55, 706.79; ¹H NMR (D₂O, 4.79)67.95 (1H, dd, J=1.49, 8.03 Hz, 3-Ar—H), 7.52 (1H, t, J=8.53 Hz, 5-Ar—H), 6.92 (1H, d, J=8.45 Hz, 4-Ar—H), 3.89 (3H, s, —OCH₃), 3.40 (2H, t, J=6.83 Hz, 3-CH₂), 3.14 (2H, t, J=7.64 Hz, 1-CH₂), 2.05 (2H, m, 2-CH₂); ¹³C NMR (D₂O+acetone, acetone (CH₃): 30.60) δ170.31, 150.60, 135.69, 132.17, 116.13, 112.46, 110.80, 52.26, 39.78, 37.83, 26.72; HRMS(CI) calculated for C₁₁H₁₆N₂O₂: (M⁺) 208.1212, found 208.1228.

3-(2-nitro-phenoxy)-1-propylamine monohydrochloride. Yield 79%; mp 160-162° C.; FTIR (KBr) ν̅ cm⁻¹:2957.52, 1612.32, 1581.01, 1525.19, 1398.45, 1339.86, 1271.26, 1254.75, 1167.97, 1059.83, 854.61, 740.78; ¹H NMR (D₂O, 4.79) δ: 8.06 (1H, d, J=8.19 Hz, 3-Ar—H), 7.75 (1H, t, J=8.28 Hz, 5-Ar—H), 7.34 (1H, d, J=8.51 Hz, 4-Ar—H), 7.21 (1H, t, J=7.99 Hz, 6-Ar—H), 4.42 (2H, t, J=5.45 Hz, 3-CH₂), 3.34 (2H, t, J=6.40 Hz, 1-CH₂), 2.30 (2H, m, 2-CH₂); ¹³C NMR (D₂O+acetone, acetone (CH₃): 30.60) δ: 152.26, 138.47, 136.33, 126.42, 121.41, 115.20, 68.13, 38.36, 26.47; HRMS(EI) Calcd for C₉H₁₂N₂O₃: (M+1) 197.0926, found 197.0928.

3-(4-methyl-2-nitro-phenoxy)-1-propylamine monohydrochloride. Yield 83%; mp 158-160° C.; FTIR(KBr) ν̅ cm⁻¹: 2952.95, 1628.51, 1572.03, 1533.89, 1399.08, 1340.97, 1265.53, 1249.95, 1162.89, 1060.92, 910.02809.57, 792.09; ¹H NMR (D₂O, 4.79) δ: 7.85 (1H, t, J=1.5 Hz, 3-Ar—H), 7.55 (1H, tt, J=1.64, 6.33 Hz, 5-Ar—H), 7.20 (1H, d, J=8.66 Hz, 6-Ar—H), 4.36 (2H, t, J=5.53 Hz,3-CH₂), 3.34 (2H, t, J=6.52 Hz, 1-CH₂), 2.36 (3H, s, Ar—CH₃), 2.28 (2H, m, 2-CH₂); ¹³C NMR (D₂O+acetone, acetone (CH₃): 30.60) δ: 150.21, 138.38, 136.71, 131.59, 126.03, 115.31, 68.03, 38.19, 26.65, 19.61; HRMS(EI) calculated for C₁₀H₁₄N₂O₃: (M+1) 211.1083, found 211.1076.

3-(4-methoxy-2-nitro-phenoxy)-1-propylamine monohydrochloride. Yield 73.5%; mp 154-156° C.; FTIR (KBr) ν̅ cm⁻¹: 2950.19, 1576.28, 1342.42, 1286.70, 1265.19, 1222.72, 1157.96, 1059.63, 1040.04, 871.77, 823.40, 792.10; ¹H NMR (D₂O, 4.79) δ: 7.45 (1H, d, J=2.94 Hz, 3-Ar—H), 7.22-7.06 (2H, m, 5,6-Ar—H), 4.22 (2H, t, J=5.58 Hz, 3-CH₂), 3.75 (3H, s, —OCH₃), 3.21 (2H, t, J=6.39 Hz, 1-CH₂), 2.15 (2H, m, 2-CH₂); ¹³C NMR (D₂O+acetone, acetone (CH₃): 30.60) δ: 152.86, 146.75, 138.38, 122.40, 116.89, 110.63, 68.59, 56.38, 38.30, 26.62; HRMS(EI) calculated for C₁₀H₁₄N₂O₄: (M+1) 227.1032, found 227.1027.

3-(4-chloro-2-nitro-phenoxy)-1-propylamine monohydrochloride. Yield 82%; mp 184-186° C.; FTIR (KBr) ν̅ cm⁻¹: 2970.91, 1614.90, 1530.26, 1343.08, 1291.04, 1266.11, 1161.89, 1063.10, 883.30, 818.43; ¹H NMR (D₂O, 4.79) δ: 8.11 (1H, t, J=2.61 Hz, 3-Ar—H), 7.76-7.71 (1H, m, 5-Ar—H),7.34-7.30 (1H, m, 6-Ar—H), 4.40 (2H, t, J=5.49 Hz, 3-CH₂), 3.32 (2H, t, J=6.51 Hz, 1-CH₂), 2.29 (2H, m, 2-CH₂); ¹³C NMR (D₂O+acetone, acetone (CH₃): 30.60) δ: 151.22, 138.67, 135.78, 125.94, 125.54, 116.83, 68.47, 38.20, 26.48; HRMS(EI) calculated for C₉H₁₁ClN₂O₃: (M+1) 231.0536, found 231.0528.

3-(5-methyl-2-nitro-phenoxy)-1-propylamine monohydrochloride. Yield 80.6%; mp 211-213° C.; FTIR (KBr) ν̅cm⁻¹:2954.26, 1611.32, 1589.03, 1513.38, 1341.30, 1272.89, 1182.24, 1059.49, 897.54, 817.55; ¹H NMR (D₂O, 4.79) δ: 7.93 (1H, d, J=8.39 Hz, 2-Ar—H), 7.11 (1H, s, 6-Ar—H), 6.96 (1H, d, J=8.28 Hz, 4-Ar—H), 4.34 (2H, t, J=5.20 Hz, 3-CH₂), 3.30 (2H, t, J=5.69 Hz, 1-CH₂), 2.41 (3H, s, Ar—CH₃), 2.24 (2H, m, 2-CH₂); ¹³C NMR (D₂O+acetone, acetone (CH₃): 30.60) δ: 152.83, 149.43, 135.89, 126.90, 122.39, 115.66, 68.53, 38.83, 26.71, 21.74; HRMS(EI) Calcd for C₁₀H₁₄N₂O₃: (M+1) 211.1083, found 211.1080.

3-(3-methyl-2-nitro-phenoxy)-1-propylamine monohydrochloride. Yield 77%; mp 160-162° C.; FTIR (KBr) ν̅ cm⁻¹: 2892.96, 1628.38, 1581.93, 1534.27, 1476.82, 1369.63, 1279.42, 1091.30, 852.71, 776.96, 747.35; ¹H NMR (D₂O, 4.79) δ: 7.45 (1H, t, J=7.94 Hz, 5-Ar—H), 7.08 (1H, d, J=8.23 Hz, 4-Ar—H), 7.01 (1H, d, J=7.38 Hz, 6-Ar—H), 4.28 (2H, t, J=5.34 Hz, 3-CH₂), 3.22 (2H, t, J=6.84 Hz, 1-CH₂), 2.19 (2H, m, 2-CH₂); ¹³C NMR (D₂O+acetone, acetone (CH₃): 30.60) δ: 148.93, 140.68, 131.36, 130.72, 122.77, 111.06, 66.36, 36.79, 25.95, 15.70; HRMS (EI) Calcd for C₁₀H₁₄N₂O₃: (M+1) 211.1083, found 211.1084.

4-(2-nitro-phenyl)-butylamine monohydrochloride. Yield 8%; oil; FTIR (film) ν̅ cm⁻¹:2927.59, 1526.99, 1455.22, 1346.86, 1276.80, 1123.55, 1076.11, 779.06; ¹H NMR (D₂O, 4.79) δ:7.97 (1H, d, J=6.16 Hz, 3-Ar—H), 7.68 (1H, t, J=5.67 Hz, 5-Ar—H), 7.52-7.45 (2H, m, Ar—H), 3.08 (2H, t, J=5.16 Hz, —CH₂NH₂), 2.93(2H, t, J=5.73 Hz, Ar—CH₂), 1.78 (4H, m, CH₂—CH₂); ¹³C NMR (D₂O+acetone, acetone (CH₃): 30.60) δ:148.80, 136.83, 133.90, 132.19, 127.55, 124.85, 39.42, 31.72, 26.91, 26.71; HRMS (CI) Calcd for C₁₀H₁₄N₂O₂: (M+1) 195.1089, found 195.1085.

N₁-(2-nitro-phenyl)-butane-1,4-diamine monohydrochloride. Yield: 50.4%; mp 173-176° C.; FTIR (KBr) ν̅ cm⁻¹:3380.74, 2938.61, 1625.67, 1573.05, 1516.63, 1417.23, 1358.38, 1256.09, 1229.27, 1158.77, 1036.31, 732.93; ¹H NMR (D₂O, 4.79) δ8.11 (1H, m, Ar—H), 7.52 (1H, m, Ar—H), 7.01 (1H, m, Ar—H), 6.72 (1H, m, Ar—H), 3.43 (2H, m, 4-CH₂), 3.06 (2H, m, 1-CH₂), 1.80 (4H, m, 2,3-CH₂); ¹³C NMR (D₂O+acetone, acetone (CH₃): 30.60) δ146.12, 137.56, 130.82, 126.69, 116.05, 114.65, 42.20, 39.66, 25.66, 24.84; HRMS (CI) Calcd for C₁₀H₁₅N₃O₂: (M+1) 210.1234, found 210.1237.

N₁-(4-nitro-phenyl)-ethane-1,2-diamine monohydrochloride. Yield: 67%; mp 198° C.; FTIR (KBr) ν̅ cm⁻¹:3272.73, 3069.16, 1601.46, 1543.80, 1504.85, 1469.89, 1328.32, 1118.56, 934.92, 835.07, 803.03, 750.56; ¹H NMR (D₂O, 4.79) δ7.99 (2H, dd, J=1.90, 5.37 Hz, 3,5-Ar—H), 6.66 (2H, dd, J=1.98, 5.29 Hz, 2, 6-Ar—H), 3.61 (2H, t, J=5.96 Hz, 2-CH₂), 3.27 (2H, t, J=6.07 Hz, 1-CH₂); ¹³C NMR (D₂O+acetone, acetone (CH₃):30.60) δ154.33, 137.09, 126.97, 111.68, 40.09, 38.57; HRMS (CI) Calcd for C₈H₁₁N₃O₂: (M+1) 182.0930, found 182.0922.

N₁-(4-nitro-phenyl)-butane-1,4-diamine monohydrochloride. Yield: 70.5%; mp 183-185° C.; FTIR (KBr) ν̅ cm⁻¹: 3273.67, 2954.13, 1605.10, 1543.48, 1500.61, 1434.47, 1329.48, 1118.68, 896.13, 833.95, 754.75; ¹H NMR (D₂O, 4.79) δ8.06 (2H, d, J=9.28 Hz, 3,5-Ar—H), 6.68 (2H, d, J=9.38 Hz, 2, 6-Ar—H), 3.29 (2H, t, J=6.44 Hz, 4-CH₂), 3.05 (2H, t, J=7.16 Hz, 1-CH₂), 1.76 (4H, m, 2,3-CH₂); ¹³C NMR (D₂O+acetone, acetone (CH₃): 30.60) δ154.66, 136.54, 127.16, 111.95, 42.67, 39.59, 25.37, 24.77; HRMS (CI) Calcd for C₁₀H₁₅N₃O₂: (M+1) 210.1234, found 210.1242.

Structure coordinates: Coordinates for the three-dimensional NMR structures of the PCAF bromodomain in complex with the lead compounds N₁-(2-nitro-phenyl)-propane-1,3-diamine monohydrochloride or N,-(4-methyl-2-nitro-phenyl)-propane-1,3-diamine monohydrochloride have been deposited in the Brookhaven Protein Data Bank under accession numbers 1WUM and 1WUG, respectively.

TABLE 4
NMR Structural Statistics for the PCAF BRD/Ligand Complexes

| | BRD/1 complex | | BRD/2 complex | |
|---|---|---|---|---|
| Total Experimental Restraints | 3371 | | 3410 | |
| Total NOE Distance Restraints | 3269 | | 3308 | |
| Total Ambiguous | 206 | | 112 | |
| Total Unambiguous | 3063 | | 3196 | |
| Manually assigned | 2895 | | 3072 | |
| ARIA assigned | 168 | | 124 | |
| Intra-residue | 1242 | | 1263 | |
| Inter-residue | 1821 | | 1933 | |
| Sequential (\|i − j\| = 1) | 608 | | 615 | |
| Medium (2 ≦ \|i − j\| ≦ 4) | 555 | | 584 | |
| Long range (\|i − j\| > 4) | 658 | | 734 | |
| Intermolecular | 51 | | 54 | |
| Hydrogen Bond Restraints | 44 | | 44 | |
| Dihedral Angle Restraints | 58 | | 58 | |
| Final Energies (kcal · mol⁻¹) ^{a} | | | | |
| E_{Total} | 270.1 ± 23.1 | | 264.2 ± 16.1 | |
| E_{NOE} | 43.6 ± 12.5 | | 39.5 ± 10.7 | |
| E_{Dihedral} | 0.0 ± 0.0 | | 0.01 ± 0.03 | |
| E_{L-J} ^{b} | −614.4 ± 15.6 | | −600.9 ± 20.0 | |
| | | Secondary | | Secondary |
| Ramachandran Plot (%) | Full Protein | Structure | Full Protein | Structure |
| Most Favorable Region | 83.7 ± 3.0 | 97.1 ± 1.8 | 82.4 ± 2.0 | 95.7 ± 1.6 |
| Additionally Allowed Region | 14.6 ± 2.8 | 2.9 ± 1.8 | 14.7 ± 1.9 | 4.3 ± 1.6 |
| Generously Allowed region | 1.6 ± 0.8 | 0.0 ± 0.0 | 2.5 ± 0.9 | 0.0 ± 0.0 |
| Disallowed Region | 0.2 ± 0.4 | 0.0 ± 0.0 | 0.4 ± 0.7 | 0.0 ± 0.0 |
| Cartesian coordinate RMSDs (Å) ^{c} | | | | |
| Backbone atoms (N, C□, C′) | 0.44 ± 0.09 | 0.32 ± 0.05 | 0.48 ± 0.07 | 0.33 ± 0.06 |
| Heavy atoms | 0.96 ± 0.07 | 0.84 ± 0.05 | 0.97 ± 0.05 | 0.81 ± 0.05 |
| Heavy atoms ^{d} | 1.04 ± 0.07 | | 1.05 ± 0.05 | |

Notes:
^{a} None of these final structures exhibit NOE-derived distance restraint violations greater than 0.3 Å or dihedral angle restraint violations greater than 5°.
^{b} The Lennard-Jones potential was not used during any refinement stage.
^{c} Protein residues 723-830. The residues in the secondary structure of the protein are 727-741, 772-777, 785-802 and 808-826.
^{d} Protein and ligand complex.

### EXAMPLE 4

### Synthesis of the Compounds of Formula III

The following compounds represented in Table 5 were prepared according to synthesis techniques well known to those of skill in the art.

TABLE 5

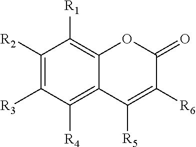

| ID | R₁ | R₂ | R₃ | R₄ | R₅ | R₆ | K_{d} (μM) |
|---|---|---|---|---|---|---|---|
| 5110065 | —OH | —OH | H | H | —CH₃ | H | 149 □ 22 |
| CM1 | —OH | —OH | H | H | —CH₂—CH₂—CH₃ | H | 147 □ 6 |
| 7910894 | —OH | —OH | H | H | —(CH₂)₂—CH₃ | H | 225 □ 20 |
| 6163501 | —OH | —OH | H | H | —Ph | H | 335 □ 35 |
| 6148450 | —OH | —OH | H | H | -cyclopentane | H | 327 □ 12 |
| 5102141 | H | —OH | H | H | —CH₃ | H | 222 □ 17 |
| 5752339 | H | —OH | H | H | —CH₂—CH₂—CH₃ | H | 204 □ 17 |
| 5771728 | H | —OH | H | —OH | —CH₂—CH₂—CH₃ | H | 367 ± 40 |
| 6147652 | H | —CH₃ | H | —OH | —CH₂—CH₂—CH₃ | H | 819 ± 75 |
| 6141880 | H | —CH₃ | H | —OH | —CH₂—CH₃ | H | 2,294 |
| 6140532 | H | —O—CH₃ | H | H | —CH₂—CH₃ | H | 357 ± 33 |
| 5768797 | H | —O—CH₃ | H | H | —CH₂—CH₂—CH₃ | H | 400 ± 74 |
| 6153095 | H | —O—CH₂—CH₃ | H | H | —CH₂—CH₃ | H | 523 ± 34 |
| 5754756 | H | —O—CH₂—CH₃ | H | H | —CH₂—CH₂—CH₃ | H | 337 ± 37 |
| 6147035 | H | —O—CH(CH₃)₂ | H | H | —CH₃ | H | 545 ± 50 |
| 5754213 | H | —O—CO—CH₃ | H | H | —CH₂—CH₂—CH₃ | H | 183 ± 12 |
| 6155412 | H | —O—CH₂—CO—OH | H | H | —CH₂—CH₃ | H | 176 ± 12 |
| 5749750 | H | —O—CH₂—CO—OH | H | H | —(CH₂)₂—CH₃ | H | 119 ± 14 |
| 5757535 | H | —O—CH(CH₃)—CO—OH | H | H | —(CH₂)₂—CH₃ | H | 184 ± 14 |
| 6144139 | H | —O—CH₂—CO—CH₃ | H | H | —CH₂—CH₃ | H | 316 ± 20 |
| 6139643 | H | —O—CH₂—CO—NH₂ | H | H | —CH₂—CH₃ | H | 765 ± 50 |
| 56 | H | —O—CO—CH₃ | H | H | —CH₃ | H | 65 ± 5 |
| 5279012 | H | —O—CO—CH(CH₃)₂ | H | H | —CH₃ | H | 321 ± 10 |
| 6085633 | H | —O—CH₂—CO—CH₃ | H | H | —CH₃ | H | 155 ± 9 |
| 5807139 | H | —O—CH₂—CH₂—OH | H | H | —CH₃ | H | 167 ± 16 |
| 6141510 | H | —O—CH₂—CH₂—CH₃ | H | H | —CH₃ | H | 426 ± 25 |
| 6146310 | H | —O—CH₂—CH₂—CH₃ | H | H | H | H | 415 ± 30 |
| 6094981 | H | —O—(CH₂)₃—CH₃ | H | H | H | H | 678 ± 50 |
| 6155336 | H | —O—CH(CH₃)—CO—OCH₃ | H | H | H | H | 271 ± 22 |
| 6149123 | H | —O—CH₂—CO—N(CH₃)₂ | H | H | H | H | 214 ± 19 |
| 6238869 | H | —O—CH(CH₃)₂ | H | H | -cyclophentane | H | 2,000 |
| 5313498 | H | —O—CH₂—CH₃ | —O—CH₂—CH₃ | H | —CH₃ | H | 190 ± 15 |
| 6153309 | H | —CH₃ | —NH—CO—CH₃ | H | —CH₃ | H | 365 ± 20 |
| 5244700 | H | H | H | H | —NH—(CH₂)₃—N(CH₃)₂ | —NH₂ | 662 ± 45 |
| 5244722 | H | H | H | H | —NH—(CH₂)₂—N(CH₃)₂ | —NH₂ | 205 ± 14 |
| 5354863 | H | H | H | H | —NH—(CH₂)₂—OH | —NH₂ | 238 ± 27 |
| 5162478 | H | H | H | H | —NH—(CH₂)₃—CH₃ | —NH₂ | 4,476 |
| 5162468 | H | H | H | H | —NH—CH₃ | —NH₂ | 156 ± 8 |
| 5162465 | H | H | H | H | —NH2 | —NH₂ | 109 ± 8 |

### EXAMPLE 5

To develop selective small-molecule inhibitors for blocking Tat/PCAF association, we conducted NMR-based chemical screening for the BRD by monitoring ligand-induced protein signal changes in 2D ¹⁵N-HSQC spectra (Hajduk, et al., Q. Rev. Biophys. (1999) 32, 211-40). We placed an emphasis on identifying compounds that bind selectively the BRD near but not just the AcK binding pocket, as the former may be more selective for this BRD. From screening of a few thousands of small-molecules from commercial libraries, we discovered several compounds including 1 that meet this criterion. Compound 1 binds the PCAF BRD with an affinity comparable to that of the Tat-AcK50 peptide (see below)(Mujtaba, et al., Mol Cell. (2004) 13, 251-63). Importantly, these compounds do not bind the structurally similar BRDs from CBP and TIF1β at millimolar concentration.

We next synthesized a series of analogs of compound 1 to probe the structure-activity relationship (SAR) (Table 6). We assessed their binding to the PCAF BRD by measuring an IC₅₀ in an ELISA assay, in which a compound competes against a biotinylated Tat-AcK50 peptide for binding to the GST-fusion BRD immobilized to glutathione-coated 96-well microtiter plate. The SAR study reveals salient features of BRD recognition of 1. First, the BRD prefers a 4-methyl group on the aniline ring, which improves IC₅₀ by 3-fold to 1.6 μM (2 vs. 1). While substitution of a 4-ethyl, 3- or 5-methyl group on the aniline ring slightly weakens the binding (3-5 vs. 1), addition of a 4-phenyl group nearly abolishes the binding (6 vs. 1). Adding a 4- or 5-cyano group weakens the binding by ˜7-12-fold (7 and 8 vs. 1). Second, a 2-nitro group on the aniline ring is vital for the binding. Swapping of 2-nitro and 5-methyl causes a 7-fold reduction in binding (9 vs. 5). Surprisingly, substitution of 2-nitro with 2-caroxylate or 2-caroxyl ester abrogates the binding (10 and 11 vs. 1). Third, the functional importance of the 2-nitro is further supported by the effects of changing the NH to an O linkage in the aniline, which severely compromises the binding to the PCAF BRD (12-17 vs. 1-5). Moreover, changing to a carbon linkage eliminates the binding (18 vs. 1). Fourth, the BRD prefers an amino three-carbon aliphatic chain in 1—a four-carbon chain reduces the binding by 30-fold (19 vs. 1) and a two-carbon chain nearly loses the binding (20 vs. 1). Alteration of 1 by two key elements, i.e. changing to a four-carbon chain and 4-nitro, abolishes the binding (21 vs. 1). Finally, the terminal amine group is also an important functional moiety for the BRD binding (22-24 vs. 1).

TABLE 6
Structure-Activity Relationship Data for Derivatives of 1

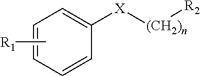

| Cmpd | R₁ | X | n | R₂ | IC₅₀ (μM) |
|---|---|---|---|---|---|
| 1 | 2-NO₂ | NH | 3 | —NH₃⁺ | 5.1 ± 0.1 |
| 2 | 2-NO₂, 4-CH₃ | NH | 3 | —NH₃⁺ | 1.6 ± 0.1 |
| 3 | 2-NO₂, 4-CH₂—CH₃ | NH | 3 | —NH₃⁺ | 7.2 ± 0.1 |
| 4 | 2-NO₂, 3-CH₃ | NH | 3 | —NH₃⁺ | 5.9 ± 0.1 |
| 5 | 2-NO₂, 5-CH₃ | NH | 3 | —NH₃⁺ | 10.8 ± 0.1 |
| 6 | 2-NO₂, 4-Ph | NH | 3 | —NH₃⁺ | >10,000 |
| 7 | 2-NO₂, 4-CN | NH | 3 | —NH₃⁺ | 34.9 ± 0.1 |
| 8 | 2-NO₂, 5-CN | NH | 3 | —NH₃⁺ | 63.4 ± 0.6 |
| 9 | 2-CH₃, 5-NO₂ | NH | 3 | —NH₃⁺ | 77.8 ± 0.4 |
| 10 | 2-COO⁻ | NH | 3 | —NH₃⁺ | >10,000 |
| 11 | 2-COOCH₃ | NH | 3 | —NH₃⁺ | >10,000 |
| 12 | 2-NO₂ | O | 3 | —NH₃⁺ | 125.6 ± 0.6 |
| 13 | 2-NO₂, 4-CH₃ | O | 3 | —NH₃⁺ | 180.0 ± 0.5 |
| 14 | 2-NO₂, 4-CH₃O | O | 3 | —NH₃⁺ | 102.7 ± 0.4 |
| 15 | 2-NO₂, 4-Cl | O | 3 | —NH₃⁺ | 215.1 ± 0.6 |
| 16 | 2-NO₂, 5-CH₃ | O | 3 | —NH₃⁺ | 203.6 ± 0.6 |
| 17 | 2-NO₂, 3-CH₃ | O | 3 | —NH₃⁺ | 164.8 ± 0.8 |
| 18 | 2-NO₂ | CH₂ | 3 | —NH₃⁺ | >10,000 |
| 19 | 2-NO₂ | NH | 4 | —NH₃⁺ | 145.9 ± 0.7 |
| 20 | 4-NO₂ | NH | 2 | —NH₃⁺ | >2,000 |
| 21 | 4-NO₂ | NH | 4 | —NH₃⁺ | >10,000 |
| 22 | 3-NH₂, 4-NO₂ | NH | 3 | —COO⁻ | >2,000 |
| 23 | 2-NO₂, 4-Cl | NH | 2 | —(OH)CH₃ | >10,000 |
| 24 | 2-Cl, 4-NO₂ | NH | 2 | —(OH)CH₃ | >10,000 |

To understand ligand selectivity of the PCAF BRD, we solved the 3D structures of the protein bound to 1 and 2. The two ligands are bound in the protein structure in nearly the same manner. For clarity, only the 2-bound structure is reported here, which is similar to the free structure except for the ZA and BC loops that move closer to each other by clamping onto the ligand (FIG. 1B). 2 is engulfed by residues in the ZA and BC loops outside the AcK binding pocket, blocking BRD binding to AcK of a target protein. The 2-nitro group of 2 possibly forms a hydrogen bond with the phenolic —OH of Y809 and/or Y802, and the terminal —NH₃⁺ interacts eletrostatically with the side-chain carboxylate of E750. The functional importance of the 2-nitro and the aniline NH likely results from a possible six-member ring structure formed between these two groups. However, it is not clear why substitution of 2-nitro with 2-carboxylate abrogates the BRD binding (10 vs. 1). The aromatic ring of 2 is sandwiched between the side-chains of Y802 and A757 on one side, and Y809 and E756 on the other side; and the propane carbon chain is surrounded by the hydrophobic portions of the side-chains of P747, E756 and V752. Finally, the 4-methyl of 2 fills a small hydrophobic cavity formed by side-chains of A757, Y802 and Y809, contributing to a 3-fold increase over 1 in binding to the PCAF BRD. Notably, out of the ligand- interacting residues, only Y802 is among the conserved residues at the AcK binding site in BRDs, thus explaining the selective binding by this class of compounds to the PCAF BRD over the structurally similar CBP and TIF1β BRDs.

The present invention therefore provides a class of novel small molecules that can effectively inhibit the PCAF BRD/Tat-AcK50 association in vitro by selectively binding to the BRD (FIG. 13). The detailed SAR understanding of the lead compounds 1 and 2 will facilitate our efforts to optimize their affinity and selectivity by branching out to interact with the neighboring AcK binding pocket by the tethering techniques.⁹ Such small-molecule inhibitors will help validate the novel anti-HIV/AIDS therapeutic strategy by targeting a cellular protein to block HIV transcription and replication.

Bromodomain. The present invention utilizes detailed structural information regarding a bromodomain and a bromodomain complexed with its acetylated binding partner. The present invention therefore provides the three-dimensional structure of the bromodomain and a bromodomain acetylated binding partner complex. Since the interaction of the bromodomain with a histone for example, can play a significant role in chromatin remodeling/regulation, the structural information provided herein can be employed in methods of identifying drugs that can modulate basic cell processes by modulating the transcription. In a particular embodiment, the three-dimensional structural information is used in the design of a small organic molecule for the treatment of cancer or as disclosed below, HIV-1 infection and/or AIDs. In addition, the present invention provides a critical structural feature for a class of inhibitors (acetyl-lysine analogs) of the interaction between bromodomains and their protein binding partners which contain an acetylated-lysine (e.g., Tat with P/CAF), see FIG. 11, as well as a compilation of compounds that share this critical feature, see FIG. 12.

Indeed, the bromodomain and lysine-acetylated protein interaction can now be implicated to play a causal role in the development of a number of diseases including cancers such as leukemia. For example, chromatin remodeling plays a central role in the etiology of viral infection and cancer (Archer, et al., Curr. Opin. Genet. Biol. 9:171-174 (1999); Jacobson, et al., Curr. Opin. Genet. Biol. 9:175-184 (1999)). Both altered histone acetylation/deacetylation and aberrant forms of chromatin-remodeling complexes are associated with human diseases. Furthermore, chromosomal translocation of various cellular genes with those encoding HATs and subunits of chromatin remodeling complexes have been implicated in leukomogenesis. The MOZ (monocytic leukemia zinc finger) and MLL/ALL-1 genes are frequently fused to the gene encoding the co-activator HAT CBP (Sobulo et al., Proc. Natl. Acad. Sci. USA 94:8732-8737(1997)). The resulting fusion protein MLL-CBP contains the tandem bromodomain-PHD finger-HAT domain of CBP. It also has been shown that both the bromodomain and HAT domain of CBP are required for leukomogenesis, because deletion of either the bromodomain or the HAT domain results in loss of the MLL-CBP fusion protein's ability for cell transform. These results indicate that the CBP bromodomain, and more particularly, the ZA loop of the CBP bromodomain, is an excellent target for developing drugs that interfere with the bromodomain acetyl-lysine interaction that can be used in the treatment of human acute leukemia. In addition, an antibody (e.g., a humanized antibody) raised specifically against a peptide from the ZA loop of the CBP bromodomain could also be effective for treating these conditions.

In addition, it now known that the human immunodeficiency virus type 1 (HIV-1) trans-activator protein, Tat, is tightly regulated by lysine acetylation (Kiernan et al., EMBO Journal 18:6106-6118 (1999)). HIV-1 Tat transcriptional activity is absolutely required for productive HIV viral replication (Jeang, et al., Curr. Top. Microbiol. Immunol., 188:123-144(1994)). Therefore, the interaction of the acetyl-lysine of Tat with one or more bromodomain-containing proteins associated with chromatin remodeling could mediate gene transcription. More particularly, it is disclosed herein that acetylated lysine50 of Tat specifically binds to the bromodomain of P/CAF. Therefore, this particular bromodomain/lysine-acetylated Tat interaction serves as a drug target for blocking HIV replication in cells. As indicated above, an antibody raised specifically against a peptide from the ZA loop of the P/CALF bromodomain could also be effective for treating and/or preventing HIV infections including those that lead to AIDs.

In addition, based on the new structural information disclosed herein, the key amino acid residues for the binding of a given bromodomain and its binding partner can be identified and further elucidated using basic mutagenesis and standard isothermal titration calorimetry, for example. Indeed, both the critical amino acids for the bromodomain and the binding partner (i.e., apart from the acetyl-lysine) can be readily determined and are also part of the present invention.

Compounds may be active to bind to two nearby sites on the bromodomain. In this case, a compound that binds a first site of the bromodomain does not bind a second nearby site. Binding to the second site can be determined by monitoring changes in a different set of amide chemical shifts in either the original screen or a second screen conducted in the presence of a ligand (or potential ligand) for the first site. From an analysis of the chemical shift changes the approximate location of a potential ligand for the second site is identified. Optimization of the second ligand for binding to the site is then carried out by screening structurally related compounds (e.g., analogs as described above). When ligands for the first site and the second site are identified, their location and orientation in the ternary complex can be determined experimentally either by NMR spectroscopy or X-ray crystallography. On the basis of this structural information, a linked compound is synthesized in which the ligand for the first site and the ligand for the second site are linked. In a preferred embodiment of this type the two ligands are covalently linked. This linked compound is tested to determine if it has a higher binding affinity for the bromodomain than either of the two individual ligands. A linked compound is selected as a ligand when it has a higher binding affinity for the bromodomain than either of the two ligands. In a preferred embodiment the affinity of the linked compound with the bromodomain is determined monitoring the ¹⁵N- or ¹H-amide chemical shift changes in two dimensional ¹⁵N-heteronuclear single-quantum correlation (¹⁵N-HSQC) spectra upon the addition of the linked compound to the ¹⁵N-labeled bromodomain as described above. A larger linked compound can be constructed in an analogous manner, e.g., linking three ligands which bind to three nearby sites on the bromodomain to form a multilinked compound that has an even higher affinity for the bromodomain than the linked compound.

### EXAMPLE 6

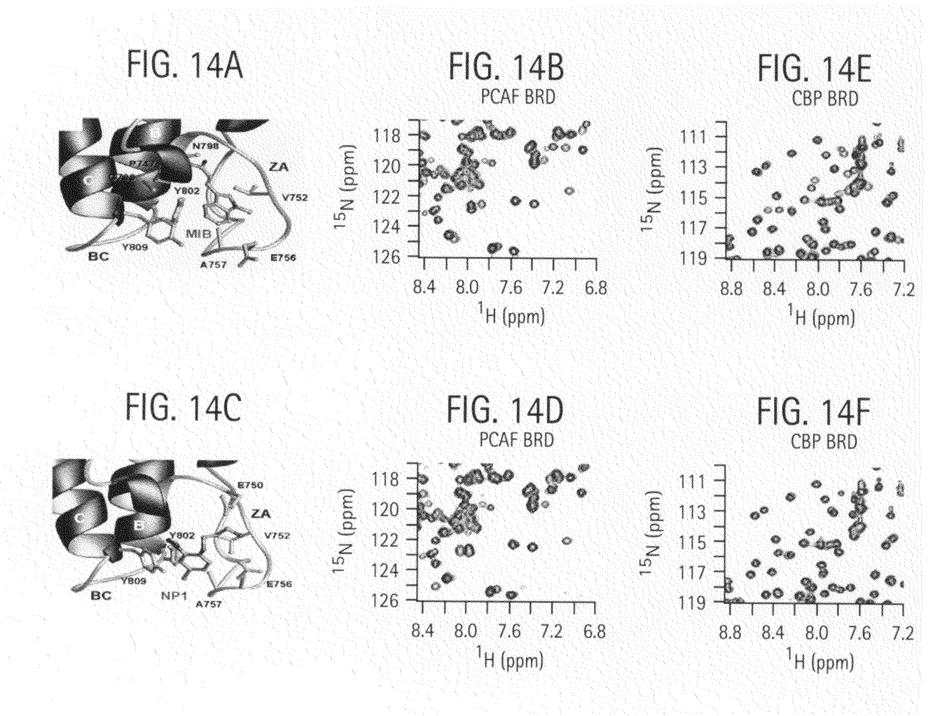

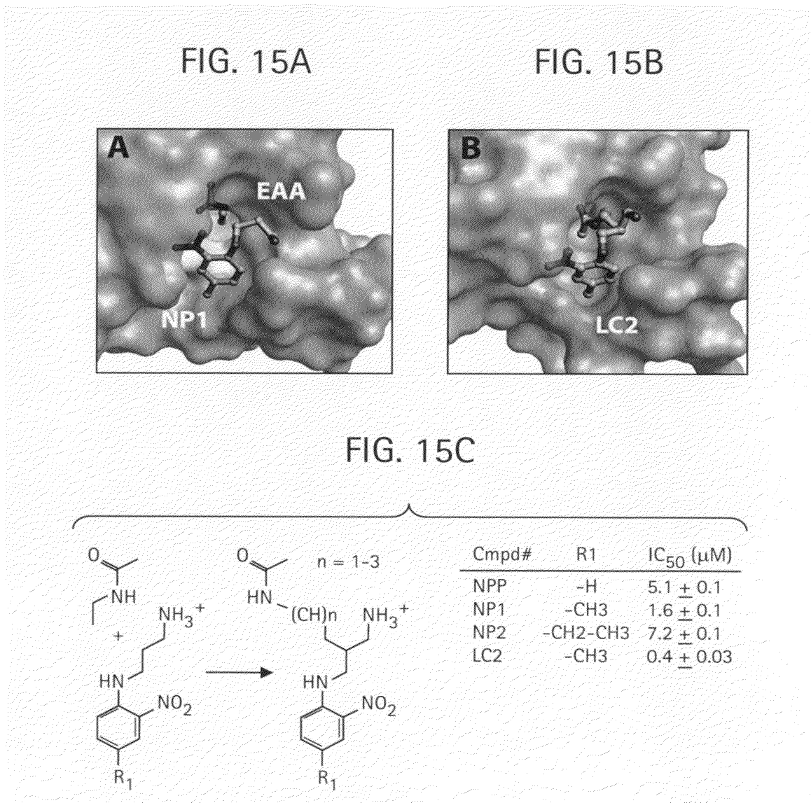

The PCAF bromodomain (BRD) recognizes small molecule compounds MIB and NP1 at two proximal sites in the acetyl-lysine (Kac) binding pocket between the ZA and BC loops (FIGS. 14A, 14C). The protein may concurrently bind NP1 and EAA (an acetyl moiety) as confirmed by a new structure of such a ternary complex (FIG. 15A). The initial NP1/EAA linked ligand, LC2 that binds to the protein (FIG. 15B) in the same mode as that of NP1 and EAA exhibits a 4-fold enhancement in IC50 (0.4 μM vs. 1.6 μM of NP1) in blocking PCAF BRD binding to HIV Tat-K50ac peptide (FIG. 15C). These results demonstrate that a linked fragment strategy may be used to develop additional and improved small-molecule BRD modulators.

The HIV Tat/PCAF association via the PCAF BRD is highly selective as compared to the bromodomains of human CBP or transcriptional intermediate factor TWO (FIG. 16A). The importance of Tat/PCAF interaction for Tat trans-activation of the integrated HIV transcription is confirmed by data that transfected PCAF BRD but not CBP BRD acts as a dominant negative in inhibiting Tat-mediated HIV-LTR activation via blocking Tat/PCAF binding (FIG. 16B).

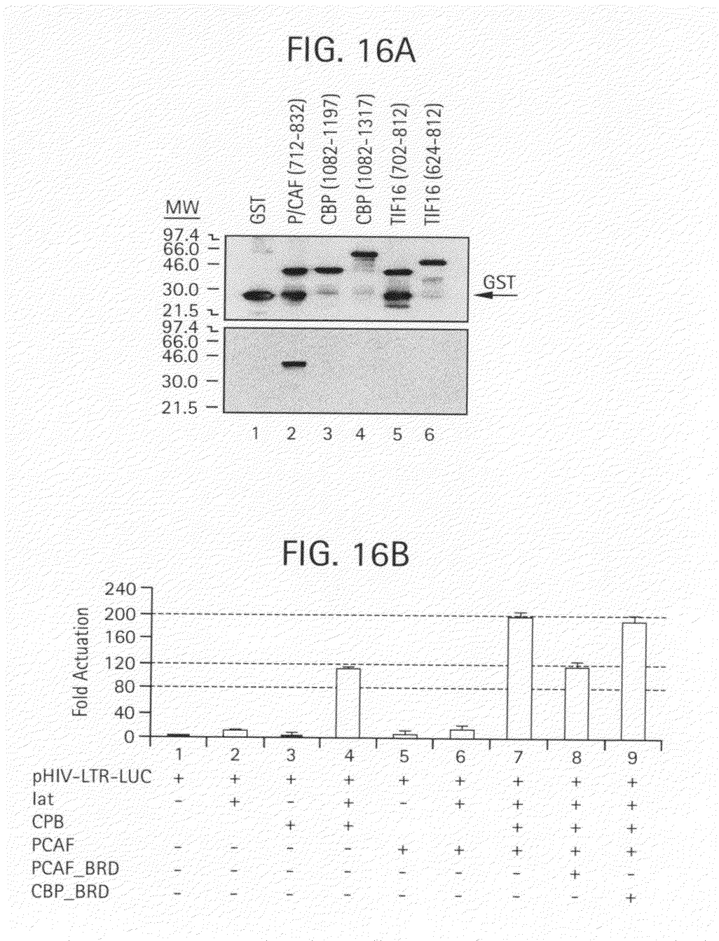

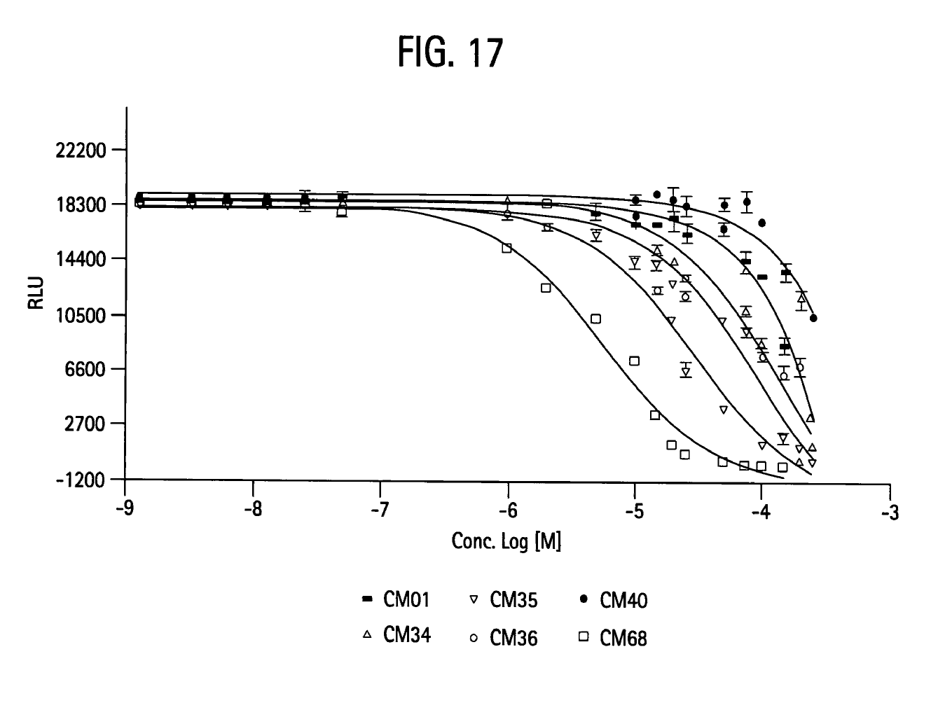

To test functional efficacy of small-molecule BRD ligands in the cell, we developed an efficient HIV-LTR luciferase reporter gene assay similar to that reported previously (Bieniasz et al., EMBO J, 1998. 17: p. 7056-7065; Madore et al., J. Virol., 1993. 67: p. 3703-3711). Specifically, in this assay, we first propagated human 293T cells in Dulbecco's modified Eagle's medium with 10% fetal calf serum and then transfected the cells with DNA plasmids encoding HIV Tat (pcTat) and HIV LTR-luciferase gene construct (pHIV-LTR-Luc) by using the calcium phosphate co-precipitation method. The transfected 293T cells were incubated in the cell culture media in the presence of a specified amount of a small-molecule BRD as listed in Table 1. The cells were incubated with the ligand for 24 hours and then lysed and assayed for luciferase activity of the cell extracts using a luciferase-based assay system (Promega). Luciferase activities derived from HIV-1 LTR were normalized to a co-transfected vector expressing beta-galactosidase. As illustrated in FIG. 17, the results obtained from this Tat-induced HIV transcription luciferase reporter assay demonstrate that PCAF BRD-specific small-molecule chemical ligands can penetrate the cell membrane and enter the cell nucleus, and exert inhibitory activity in HIV transcription by interfering with Tat/PCAF BRD binding in a dose dependent manner.

### EXAMPLE 7

### Efficacy of the Small-Molecule Inhibitors of PCAF Bromodomain (BRD)/HIV-1 Tat-AcK50 Complex on HIV Tat-Mediated Transactivation

Inhibition of Tat-mediated transactivation by small-molecule inhibitors that block the PCAF bromodomain interaction with HIV-1 Tat-AcK50. The effect was assessed by a microinjection study as described previously by Dorr et al. (EMBO J. 21; 2715-2723, 2002). In this microinjection assay, HeLa-Tat cells were grown on Cellocate coverslips and microinjected at room temperature with an automated injection system (Carl Zeiss). Samples were prepared as a 20 μl injection mix containing the LTR-luciferase (100 ng/ml) and CMV-GFP (50 ng/ml) constructs together with 5 mg/ml a chemical compound or pre-immune IgGs. Live cells were examined on a Zeiss Axiovert microscope to determine the number of GFP-positive cells. Four hours after injection, cells were washed in cold phosphate buffer and processed for luciferase assays (Promega). FIG. 15 demonstrates inhibition of Tat-mediated transactivation by the PCAF BRD inhibitor. (A) Schematic diagram of the microinjection assay. (B) Effect of a lead compound 765 on Tat-mediated transactivation as compared to DMSO control.

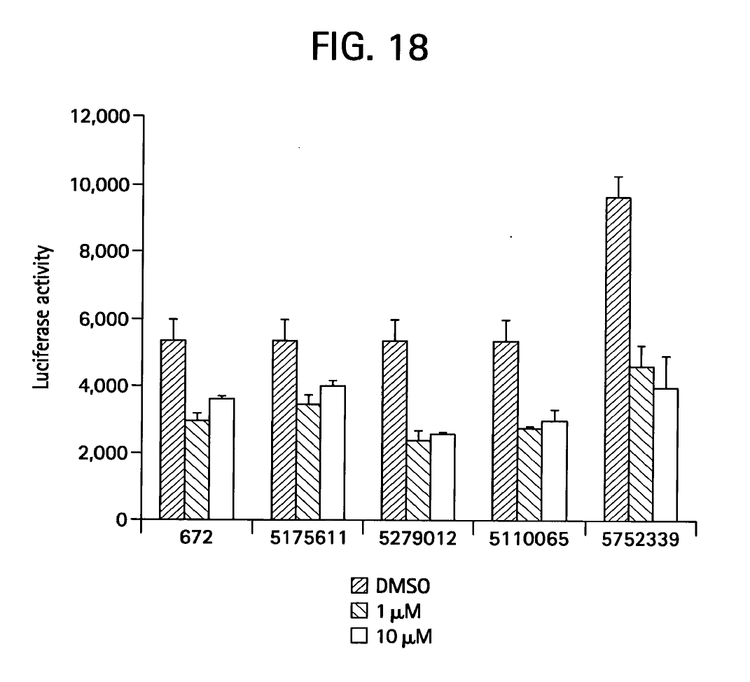

Effect of PCAF bromodomain inhibitors on Tat transactivation. FIG. 18 demonstrates the effect of PCAF BRD inhibitors on Tat transactivation. HeLa cells were transfected with LTR-luciferase and 20 ng Tat-expression vector. PCAF BRD inhibitors were added after transfection, and cells were harvested after 8 hours.

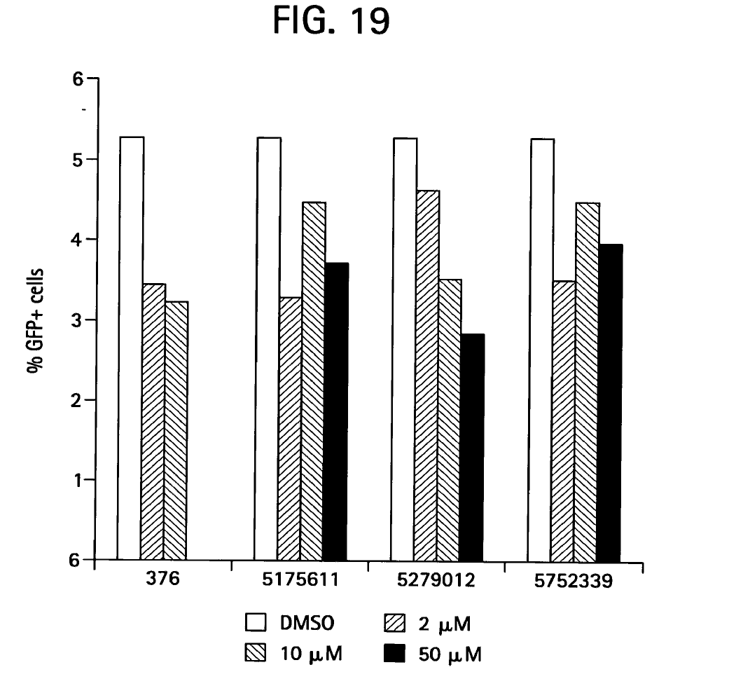

Effect of the PCAF Bromodomain inhibitors on viral infection, using a procedure described by Pagans et al. (PLoS Biol. 3(2): e41, 2005). FIG. 19 demonstrates the effect of PCAF BRD inhibitors on viral infection. Jurkat T cells were infected with an LTR-Tat-IRES-GFP virus. PCAF BRD inhibitors were added after overnight infection. The percentage of infection was monitored 48 hours later by FACS analysis.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are hereby incorporated by reference herein in their entireties.

| Sequence CWU 1 | |
|---|---|
| 63 1 3014 DNA Homo sapiens 1 | |
| ggggccgcgt cgacgcggaa aagaggccgt ggggggcctc ccagcgctgg cagacaccgt | 60 |
| gaggctggca gccgccggca cgcacaccta gtccgcagtc ccgaggaaca tgtccgcagc | 120 |
| cagggcgcgg agcagagtcc cgggcaggag aaccaaggga gggcgtgtgc tgtggcggcg | 180 |
| gcggcagcgg cagcggagcc gctagtcccc tccctcctgg gggagcagct gccgccgctg | 240 |
| ccgccgccgc caccaccatc agcgcgcggg gcccggccag agcgagccgg gcgagcggcg | 300 |
| cgctaggggg agggcggggg cggggagggg ggtgggcgaa gggggcggga gggcgtgggg | 360 |
| ggagggtctc gctctcccga ctaccagagc ccgagggaga ccctggcggc ggcggcggcg | 420 |
| cctgacactc ggcgcctcct gccgtgctcc ggggcggcat gtccgaggct ggcggggccg | 480 |
| ggccgggcgg ctgcggggca ggagccgggg caggggccgg gcccggggcg ctgcccccgc | 540 |
| agcctgcggc gcttccgccc gcgcccccgc agggctcccc ctgcgccgct gccgccgggg | 600 |
| gctcgggcgc ctgcggtccg gcgacggcag tggctgcagc gggcacggcc gaaggaccgg | 660 |
| gaggcggtgg ctcggcccga atcgccgtga agaaagcgca actacgctcc gctccgcggg | 720 |
| ccaagaaact ggagaaactc ggagtgtact ccgcctgcaa ggccgaggag tcttgtaaat | 780 |
| gtaatggctg gaaaaaccct aacccctcac ccactccccc cagagccgac ctgcagcaaa | 840 |
| taattgtcag tctaacagaa tcctgtcgga gttgtagcca tgccctagct gctcatgttt | 900 |
| cccacctgga gaatgtgtca gaggaagaaa tgaacagact cctgggaata gtattggatg | 960 |
| tggaatatct ctttacctgt gtccacaagg aagaagatgc agataccaaa caagtttatt | 1020 |
| tctatctatt taagctcttg agaaagtcta ttttacaaag aggaaaacct gtggttgaag | 1080 |
| gctctttgga aaagaaaccc ccatttgaaa aacctagcat tgaacagggt gtgaataact | 1140 |
| ttgtgcagta caaatttagt cacctgccag caaaagaaag gcaaacaata gttgagttgg | 1200 |
| caaaaatgtt cctaaaccgc atcaactatt ggcatctgga ggcaccatct caacgaagac | 1260 |
| tgcgatctcc caatgatgat atttctggat acaaagagaa ctacacaagg tggctgtgtt | 1320 |
| actgcaacgt gccacagttc tgcgacagtc tacctcggta cgaaaccaca caggtgtttg | 1380 |
| ggagaacatt gcttcgctcg gtcttcactg ttatgaggcg acaactcctg gaacaagcaa | 1440 |
| gacaggaaaa agataaactg cctcttgaaa aacgaactct aatcctcact catttcccaa | 1500 |
| aatttctgtc catgctagaa gaagaagtat atagtcaaaa ctctcccatc tgggatcagg | 1560 |
| attttctctc agcctcttcc agaaccagcc agctaggcat ccaaacagtt atcaatccac | 1620 |
| ctcctgtggc tgggacaatt tcatacaatt caacctcatc ttcccttgag cagccaaacg | 1680 |
| cagggagcag cagtcctgcc tgcaaagcct cttctggact tgaggcaaac ccaggagaaa | 1740 |
| agaggaaaat gactgattct catgttctgg aggaggccaa gaaaccccga gttatggggg | 1800 |
| atattccgat ggaattaatc aacgaggtta tgtctaccat cacggaccct gcagcaatgc | 1860 |
| ttggaccaga gaccaatttt ctgtcagcac actcggccag ggatgaggcg gcaaggttgg | 1920 |
| aagagcgcag gggtgtaatt gaatttcacg tggttggcaa ttccctcaac cagaaaccaa | 1980 |
| acaagaagat cctgatgtgg ctggttggcc tacagaacgt tttctcccac cagctgcccc | 2040 |
| gaatgccaaa agaatacatc acacggctcg tctttgaccc gaaacacaaa acccttgctt | 2100 |
| taattaaaga tggccgtgtt attggtggta tctgtttccg tatgttccca tctcaaggat | 2160 |
| tcacagagat tgtcttctgt gctgtaacct caaatgagca agtcaagggc tatggaacac | 2220 |
| acctgatgaa tcatttgaaa gaatatcaca taaagcatga catcctgaac ttcctcacat | 2280 |
| atgcagatga atatgcaatt ggatacttta agaaacaggg tttctccaaa gaaattaaaa | 2340 |
| tacctaaaac caaatatgtt ggctatatca aggattatga aggagccact ttaatgggat | 2400 |
| gtgagctaaa tccacggatc ccgtacacag aattttctgt catcattaaa aagcagaagg | 2460 |
| agataattaa aaaactgatt gaaagaaaac aggcacaaat tcgaaaagtt taccctggac | 2520 |
| tttcatgttt taaagatgga gttcgacaga ttcctataga aagcattcct ggaattagag | 2580 |
| agacaggctg gaaaccgagt ggaaaagaga aaagtaaaga gcccagagac cctgaccagc | 2640 |
| tttacagcac gctcaagagc atcctccagc aggtgaagag ccatcaaagc gcttggccct | 2700 |
| tcatggaacc tgtgaagaga acagaagctc caggatatta tgaagttata aggttcccca | 2760 |
| tggatctgaa aaccatgagt gaacgcctca agaataggta ctacgtgtct aagaaattat | 2820 |
| tcatggcaga cttacagcga gtctttacca attgcaaaga gtacaacgcc gctgagagtg | 2880 |
| aatactacaa atgtgccaat atcctggaga aattcttctt cagtaaaatt aaggaagctg | 2940 |
| gattaattga caagtgattt tttttccccc tctgcttctt agaaactcac caagcagtgt | 3000 |
| gcctaaagca aggt | 3014 |
| 2 832 PRT Homo sapiens 2 | |
| Met Ser Glu Ala Gly Gly Ala Gly Pro Gly Gly Cys Gly Ala Gly Ala | |
| 1 5 10 15 | |
| Gly Ala Gly Ala Gly Pro Gly Ala Leu Pro Pro Gln Pro Ala Ala Leu | |
| 20 25 30 | |
| Pro Pro Ala Pro Pro Gln Gly Ser Pro Cys Ala Ala Ala Ala Gly Gly | |
| 35 40 45 | |
| Ser Gly Ala Cys Gly Pro Ala Thr Ala Val Ala Ala Ala Gly Thr Ala | |
| 50 55 60 | |
| Glu Gly Pro Gly Gly Gly Gly Ser Ala Arg Ile Ala Val Lys Lys Ala | |
| 65 70 75 80 | |
| Gln Leu Arg Ser Ala Pro Arg Ala Lys Lys Leu Glu Lys Leu Gly Val | |
| 85 90 95 | |
| Tyr Ser Ala Cys Lys Ala Glu Glu Ser Cys Lys Cys Asn Gly Trp Lys | |
| 100 105 110 | |
| Asn Pro Asn Pro Ser Pro Thr Pro Pro Arg Ala Asp Leu Gln Gln Ile | |
| 115 120 125 | |
| Ile Val Ser Leu Thr Glu Ser Cys Arg Ser Cys Ser His Ala Leu Ala | |
| 130 135 140 | |
| Ala His Val Ser His Leu Glu Asn Val Ser Glu Glu Glu Met Asn Arg | |
| 145 150 155 160 | |
| Leu Leu Gly Ile Val Leu Asp Val Glu Tyr Leu Phe Thr Cys Val His | |
| 165 170 175 | |
| Lys Glu Glu Asp Ala Asp Thr Lys Gln Val Tyr Phe Tyr Leu Phe Lys | |
| 180 185 190 | |
| Leu Leu Arg Lys Ser Ile Leu Gln Arg Gly Lys Pro Val Val Glu Gly | |
| 195 200 205 | |
| Ser Leu Glu Lys Lys Pro Pro Phe Glu Lys Pro Ser Ile Glu Gln Gly | |
| 210 215 220 | |
| Val Asn Asn Phe Val Gln Tyr Lys Phe Ser His Leu Pro Ala Lys Glu | |
| 225 230 235 240 | |
| Arg Gin Thr Ile Val Glu Leu Ala Lys Met Phe Leu Asn Arg Ile Asn | |
| 245 250 255 | |
| Tyr Trp His Leu Glu Ala Pro Ser Gln Arg Arg Leu Arg Ser Pro Asn | |
| 260 265 270 | |
| Asp Asp Ile Ser Gly Tyr Lys Glu Asn Tyr Thr Arg Trp Leu Cys Tyr | |
| 275 280 285 | |
| Cys Asn Val Pro Gln Phe Cys Asp Ser Leu Pro Arg Tyr Glu Thr Thr | |
| 290 295 300 | |
| Gln Val Phe Gly Arg Thr Leu Leu Arg Ser Val Phe Thr Val Met Arg | |
| 305 310 315 320 | |
| Arg Gln Leu Leu Glu Gln Ala Arg Gln Glu Lys Asp Lys Leu Pro Leu | |
| 325 330 335 | |
| Glu Lys Arg Thr Leu Ile Leu Thr His Phe Pro Lys Phe Leu Ser Met | |
| 340 345 350 | |
| Leu Glu Glu Glu Val Tyr Ser Gln Asn Ser Pro Ile Trp Asp Gln Asp | |
| 355 360 365 | |
| Phe Leu Ser Ala Ser Ser Arg Thr Ser Gln Leu Gly Ile Gln Thr Val | |
| 370 375 380 | |
| Ile Asn Pro Pro Pro Val Ala Gly Thr Ile Ser Tyr Asn Ser Thr Ser | |
| 385 390 395 400 | |
| Ser Ser Leu Glu Gln Pro Asn Ala Gly Ser Ser Ser Pro Ala Cys Lys | |
| 405 410 415 | |
| Ala Ser Ser Gly Leu Glu Ala Asn Pro Gly Glu Lys Arg Lys Met Thr | |
| 420 425 430 | |
| Asp Ser His Val Leu Glu Glu Ala Lys Lys Pro Arg Val Met Gly Asp | |
| 435 440 445 | |
| Ile Pro Met Glu Leu Ile Asn Glu Val Met Ser Thr Ile Thr Asp Pro | |
| 450 455 460 | |
| Ala Ala Met Leu Gly Pro Glu Thr Asn Phe Leu Ser Ala His Ser Ala | |
| 465 470 475 480 | |
| Arg Asp Glu Ala Ala Arg Leu Glu Glu Arg Arg Gly Val Ile Glu Phe | |
| 485 490 495 | |
| His Val Val Gly Asn Ser Leu Asn Gln Lys Pro Asn Lys Lys Ile Leu | |
| 500 505 510 | |
| Met Trp Leu Val Gly Leu Gln Asn Val Phe Ser His Gln Leu Pro Arg | |
| 515 520 525 | |
| Met Pro Lys Glu Tyr Ile Thr Arg Leu Val Phe Asp Pro Lys His Lys | |
| 530 535 540 | |
| Thr Leu Ala Leu Ile Lys Asp Gly Arg Val Ile Gly Gly Ile Cys Phe | |
| 545 550 555 560 | |
| Arg Met Phe Pro Ser Gln Gly Phe Thr Glu Ile Val Phe Cys Ala Val | |
| 565 570 575 | |
| Thr Ser Asn Glu Gln Val Lys Gly Tyr Gly Thr His Leu Met Asn His | |
| 580 585 590 | |
| Leu Lys Glu Tyr His Ile Lys His Asp Ile Leu Asn Phe Leu Thr Tyr | |
| 595 600 605 | |
| Ala Asp Glu Tyr Ala Ile Gly Tyr Phe Lys Lys Gln Gly Phe Ser Lys | |
| 610 615 620 | |
| Glu Ile Lys Ile Pro Lys Thr Lys Tyr Val Gly Tyr Ile Lys Asp Tyr | |
| 625 630 635 640 | |
| Glu Gly Ala Thr Leu Met Gly Cys Glu Leu Asn Pro Arg Ile Pro Tyr | |
| 645 650 655 | |
| Thr Glu Phe Ser Val Ile Ile Lys Lys Gln Lys Glu Ile Ile Lys Lys | |
| 660 665 670 | |
| Leu Ile Glu Arg Lys Gln Ala Gln Ile Arg Lys Val Tyr Pro Gly Leu | |
| 675 680 685 | |
| Ser Cys Phe Lys Asp Gly Val Arg Gln Ile Pro Ile Glu Ser Ile Pro | |
| 690 695 700 | |
| Gly Ile Arg Glu Thr Gly Trp Lys Pro Ser Gly Lys Glu Lys Ser Lys | |
| 705 710 715 720 | |
| Glu Pro Arg Asp Pro Asp Gln Leu Tyr Ser Thr Leu Lys Ser Ile Leu | |
| 725 730 735 | |
| Gln Gln Val Lys Ser His Gln Ser Ala Trp Pro Phe Met Glu Pro Val | |
| 740 745 750 | |
| Lys Arg Thr Glu Ala Pro Gly Tyr Tyr Glu Val Ile Arg Phe Pro Met | |
| 755 760 765 | |
| Asp Leu Lys Thr Met Ser Glu Arg Leu Lys Asn Arg Tyr Tyr Val Ser | |
| 770 775 780 | |
| Lys Lys Leu Phe Met Ala Asp Leu Gln Arg Val Phe Thr Asn Cys Lys | |
| 785 790 795 800 | |
| Glu Tyr Asn Ala Ala Glu Ser Glu Tyr Tyr Lys Cys Ala Asn Ile Leu | |
| 805 810 815 | |
| Glu Lys Phe Phe Phe Ser Lys Ile Lys Glu Ala Gly Leu Ile Asp Lys | |
| 820 825 830 | |
| 3 25 PRT Artificial Sequence synthetic bromodomain peptide 3 | |
| Phe Xaa Xaa Xaa Pro Xaa Xaa Xaa Xaa Xaa Xaa Xaa Xaa Xaa Xaa Tyr | |
| 1 5 10 15 | |
| Xaa Xaa Xaa Xaa Xaa Xaa Pro Xaa Asp | |
| 20 25 | |
| 4 12 PRT Artificial Sequence synthetic bromodomain peptide 4 | |
| Ile Ser Tyr Gly Arg Xaa Lys Arg Arg Gln Arg Arg | |
| 1 5 10 | |
| 5 14 PRT Artificial Sequence synthetic bromodomain peptide 5 | |
| Ala Arg Lys Ser Thr Gly Gly Xaa Ala Pro Arg Lys Gln Leu | |
| 1 5 10 | |
| 6 14 PRT Artificial Sequence synthetic bromodomain peptide 6 | |
| Gln Ser Thr Ser Arg His Lys Xaa Leu Met Phe Lys Thr Glu | |
| 1 5 10 | |
| 7 110 PRT Homo sapiens bromodomain peptide 7 | |
| Ser Lys Glu Pro Arg Asp Pro Asp Gln Leu Tyr Ser Thr Leu Lys Ser | |
| 1 5 10 15 | |
| Ile Leu Gln Gln Val Lys Ser His Gln Ser Ala Trp Pro Phe Met Glu | |
| 20 25 30 | |
| Pro Val Lys Arg Thr Glu Ala Pro Gly Tyr Tyr Glu Val Ile Arg Ser | |
| 35 40 45 | |
| Pro Met Asp Leu Lys Thr Met Ser Glu Arg Leu Lys Asn Arg Tyr Tyr | |
| 50 55 60 | |
| Val Ser Lys Lys Leu Phe Met Ala Asp Leu Gln Arg Val Phe Thr Asn | |
| 65 70 75 80 | |
| Cys Lys Glu Tyr Asn Ala Pro Glu Ser Glu Tyr Tyr Lys Cys Ala Asn | |
| 85 90 95 | |
| Ile Leu Glu Lys Phe Phe Phe Ser Lys Ile Lys Glu Ala Gly | |
| 100 105 110 | |
| 8 110 PRT Homo sapiens 8 | |
| Gly Lys Glu Leu Lys Asp Pro Asp Gln Leu Tyr Thr Thr Leu Lys Asn | |
| 1 5 10 15 | |
| Leu Leu Ala Gln Ile Lys Ser His Pro Ser Ala Trp Pro Phe Met Glu | |
| 20 25 30 | |
| Pro Val Lys Lys Ser Glu Ala Pro Asp Tyr Tyr Glu Val Ile Arg Phe | |
| 35 40 45 | |
| Pro Ile Asp Leu Lys Thr Met Thr Glu Arg Leu Arg Ser Arg Tyr Tyr | |
| 50 55 60 | |
| Val Thr Arg Lys Leu Phe Val Ala Asp Leu Gln Arg Val Ile Ala Asn | |
| 65 70 75 80 | |
| Cys Arg Glu Tyr Asn Pro Pro Asp Ser Glu Tyr Cys Arg Cys Ala Ser | |
| 85 90 95 | |
| Ala Leu Glu Lys Phe Phe Tyr Phe Lys Leu Lys Glu Gly Gly | |
| 100 105 110 | |
| 9 109 PRT Tetrahymena thermophila 9 | |
| Leu Lys Lys Ser Lys Glu Arg Ser Phe Asn Leu Gln Cys Ala Asn Val | |
| 1 5 10 15 | |
| Ile Glu Asn Met Lys Arg His Lys Gln Ser Trp Pro Phe Leu Asp Pro | |
| 20 25 30 | |
| Val Asn Lys Asp Asp Val Pro Asp Tyr Tyr Asp Val Ile Thr Asp Pro | |
| 35 40 45 | |
| Ile Asp Ile Lys Ala Ile Glu Lys Lys Leu Gln Asn Asn Gln Tyr Val | |
| 50 55 60 | |
| Asp Lys Asp Gln Phe Ile Lys Asp Val Lys Arg Ile Phe Thr Asn Ala | |
| 65 70 75 80 | |
| Lys Ile Tyr Asn Gln Pro Asp Thr Ile Tyr Tyr Lys Ala Ala Lys Glu | |
| 85 90 95 | |
| Leu Glu Asp Phe Val Glu Pro Tyr Leu Thr Lys Leu Lys | |
| 100 105 | |
| 10 109 PRT Saccharomyces cerevisiae 10 | |
| Ala Gln Arg Pro Lys Arg Gly Pro His Asp Ala Ala Ile Gln Asn Ile | |
| 1 5 10 15 | |
| Leu Thr Glu Leu Gln Asn His Ala Ala Ala Trp Pro Phe Leu Gln Pro | |
| 20 25 30 | |
| Val Asn Lys Glu Glu Val Pro Asp Tyr Tyr Asp Phe Ile Lys Glu Pro | |
| 35 40 45 | |
| Met Asp Leu Ser Thr Met Glu Ile Lys Leu Glu Ser Asn Lys Tyr Gln | |
| 50 55 60 | |
| Lys Met Glu Asp Phe Ile Tyr Asp Ala Arg Leu Val Phe Asn Asn Cys | |
| 65 70 75 80 | |
| Arg Met Tyr Asn Gly Glu Asn Thr Ser Tyr Tyr Lys Tyr Ala Asn Arg | |
| 85 90 95 | |
| Leu Glu Lys Phe Phe Asn Asn Lys Val Lys Glu Ile Pro | |
| 100 105 | |
| 11 112 PRT Homo sapiens 11 | |
| Lys Lys Ile Phe Lys Pro Glu Glu Leu Arg Gln Ala Leu Met Pro Thr | |
| 1 5 10 15 | |
| Leu Glu Ala Leu Tyr Arg Gln Asp Pro Glu Ser Leu Pro Phe Arg Gln | |
| 20 25 30 | |
| Pro Val Asp Pro Gln Leu Leu Gly Ile Pro Asp Tyr Phe Asp Ile Val | |
| 35 40 45 | |
| Lys Ser Pro Met Asp Leu Ser Thr Ile Lys Arg Lys Leu Asp Thr Gly | |
| 50 55 60 | |
| Gln Tyr Gln Glu Pro Trp Gln Tyr Val Asp Asp Ile Trp Leu Met Phe | |
| 65 70 75 80 | |
| Asn Asn Ala Trp Leu Tyr Asn Arg Lys Thr Ser Arg Val Tyr Lys Tyr | |
| 85 90 95 | |
| Cys Ser Lys Leu Ser Glu Val Phe Glu Gln Glu Ile Asp Pro Val Met | |
| 100 105 110 | |
| 12 112 PRT Homo sapiens 12 | |
| Lys Lys Ile Phe Lys Pro Glu Glu Leu Arg Gln Ala Leu Met Pro Thr | |
| 1 5 10 15 | |
| Leu Glu Ala Leu Tyr Arg Gln Asp Pro Glu Ser Leu Pro Phe Arg Gln | |
| 20 25 30 | |
| Pro Val Asp Pro Gln Leu Leu Gly Ile Pro Asp Tyr Phe Asp Ile Val | |
| 35 40 45 | |
| Lys Asn Pro Met Asp Leu Ser Thr Ile Lys Arg Lys Leu Asp Thr Gly | |
| 50 55 60 | |
| Gln Tyr Gln Glu Pro Trp Gln Tyr Val Asp Asp Val Trp Leu Met Phe | |
| 65 70 75 80 | |
| Asn Asn Ala Trp Leu Tyr Asn Arg Lys Thr Ser Arg Val Tyr Lys Phe | |
| 85 90 95 | |
| Cys Ser Lys Leu Ala Glu Val Phe Glu Gln Glu Ile Asp Pro Val Met | |
| 100 105 110 | |
| 13 112 PRT Mus musculus 13 | |
| Lys Lys Ile Phe Lys Pro Glu Glu Leu Arg Gln Ala Leu Met Pro Thr | |
| 1 5 10 15 | |
| Leu Glu Ala Leu Tyr Arg Gln Asp Pro Glu Ser Leu Pro Phe Arg Gln | |
| 20 25 30 | |
| Pro Val Asp Pro Gln Leu Leu Gly Ile Pro Asp Tyr Phe Asp Ile Val | |
| 35 40 45 | |
| Lys Asn Pro Met Asp Leu Ser Thr Ile Lys Arg Lys Leu Asp Thr Gly | |
| 50 55 60 | |
| Gln Tyr Gln Glu Pro Trp Gln Tyr Val Asp Asp Val Arg Leu Met Phe | |
| 65 70 75 80 | |
| Asn Asn Ala Trp Leu Tyr Asn Arg Lys Thr Ser Arg Val Tyr Lys Phe | |
| 85 90 95 | |
| Cys Ser Lys Leu Ala Glu Val Phe Glu Gln Glu Ile Asp Pro Val Met | |
| 100 105 110 | |
| 14 111 PRT Caenorhabditis elegans 14 | |
| Asp Thr Val Phe Ser Gln Glu Asp Leu Ile Lys Phe Leu Leu Pro Val | |
| 1 5 10 15 | |
| Trp Glu Lys Leu Asp Lys Ser Glu Asp Ala Ala Pro Phe Arg Val Pro | |
| 20 25 30 | |
| Val Asp Ala Lys Leu Leu Asn Ile Pro Asp Tyr His Glu Ile Ile Lys | |
| 35 40 45 | |
| Arg Pro Met Asp Leu Glu Thr Val His Lys Lys Leu Tyr Ala Gly Gln | |
| 50 55 60 | |
| Tyr Gln Asn Ala Gly Gln Phe Cys Asp Asp Ile Trp Leu Met Leu Asp | |
| 65 70 75 80 | |
| Asn Ala Trp Leu Tyr Asn Arg Lys Asn Ser Lys Val Tyr Lys Tyr Gly | |
| 85 90 95 | |
| Leu Lys Leu Ser Glu Met Phe Val Ser Glu Met Asp Pro Val Met | |
| 100 105 110 | |
| 15 110 PRT Homo sapiens 15 | |
| Arg Arg Arg Thr Asp Pro Met Val Thr Leu Ser Ser Ile Leu Glu Ser | |
| 1 5 10 15 | |
| Ile Ile Asn Asp Met Arg Asp Leu Pro Asn Thr Tyr Pro Phe His Thr | |
| 20 25 30 | |
| Pro Val Asn Ala Lys Val Val Lys Asp Tyr Tyr Lys Ile Ile Thr Arg | |
| 35 40 45 | |
| Pro Met Asp Leu Gln Thr Leu Arg Glu Asn Val Arg Lys Arg Leu Tyr | |
| 50 55 60 | |
| Pro Ser Arg Glu Glu Phe Arg Glu His Leu Glu Leu Ile Val Lys Asn | |
| 65 70 75 80 | |
| Ser Ala Thr Tyr Asn Gly Pro Lys His Ser Leu Thr Gln Ile Ser Gln | |
| 85 90 95 | |
| Ser Met Leu Asp Leu Cys Asp Glu Lys Leu Lys Glu Lys Glu | |
| 100 105 110 | |
| 16 110 PRT Mesocricetus auratus 16 | |
| Arg Arg Arg Thr Asp Pro Met Val Thr Leu Ser Ser Ile Leu Glu Ser | |
| 1 5 10 15 | |
| Ile Ile Asn Asp Met Arg Asp Leu Pro Asn Thr Tyr Pro Phe His Thr | |
| 20 25 30 | |
| Pro Val Asn Ala Lys Val Val Lys Asp Tyr Tyr Lys Ile Ile Thr Arg | |
| 35 40 45 | |
| Pro Met Asp Leu Gln Thr Leu Arg Glu Asn Val Arg Lys Arg Leu Tyr | |
| 50 55 60 | |
| Pro Ser Arg Glu Glu Phe Arg Glu His Leu Glu Leu Ile Val Lys Asn | |
| 65 70 75 80 | |
| Ser Ala Thr Tyr Asn Gly Pro Lys His Ser Leu Thr Gln Ile Ser Gln | |
| 85 90 95 | |
| Ser Met Leu Asp Leu Cys Asp Glu Lys Leu Lys Glu Lys Glu | |
| 100 105 110 | |
| 17 111 PRT Homo sapiens 17 | |
| Leu Leu Asp Asp Asp Asp Gln Val Ala Phe Ser Phe Ile Leu Asp Asn | |
| 1 5 10 15 | |
| Ile Val Thr Gln Lys Met Met Ala Val Pro Asp Ser Trp Pro Phe His | |
| 20 25 30 | |
| His Pro Val Asn Lys Lys Phe Val Pro Asp Tyr Tyr Lys Val Ile Val | |
| 35 40 45 | |
| Asn Pro Met Asp Leu Glu Thr Ile Arg Lys Asn Ile Ser Lys His Lys | |
| 50 55 60 | |
| Tyr Gln Ser Arg Glu Ser Phe Leu Asp Asp Val Asn Leu Ile Leu Ala | |
| 65 70 75 80 | |
| Asn Ser Val Lys Tyr Asn Gly Pro Glu Ser Gln Tyr Thr Lys Thr Ala | |
| 85 90 95 | |
| Gln Glu Ile Val Asn Val Cys Tyr Gln Thr Leu Thr Glu Tyr Asp | |
| 100 105 110 | |
| 18 111 PRT Mesocricetus auratus 18 | |
| Leu Leu Asp Asp Asp Asp Gln Val Ala Phe Ser Phe Ile Leu Asp Asn | |
| 1 5 10 15 | |
| Ile Val Thr Gln Lys Met Met Ala Val Pro Asp Ser Trp Pro Phe His | |
| 20 25 30 | |
| His Pro Val Asn Lys Lys Phe Val Pro Asp Tyr Tyr Lys Val Ile Val | |
| 35 40 45 | |
| Ser Pro Met Asp Leu Glu Thr Ile Arg Lys Asn Ile Ser Lys His Lys | |
| 50 55 60 | |
| Tyr Gln Ser Arg Glu Ser Phe Leu Asp Asp Val Asn Leu Ile Leu Ala | |
| 65 70 75 80 | |
| Asn Ser Val Lys Tyr Asn Gly Ser Glu Ser Gln Tyr Thr Lys Thr Ala | |
| 85 90 95 | |
| Gln Glu Ile Val Asn Val Cys Tyr Gln Thr Leu Thr Glu Tyr Asp | |
| 100 105 110 | |
| 19 111 PRT Homo sapiens 19 | |
| Lys Pro Gly Arg Val Thr Asn Gln Leu Gln Tyr Leu His Lys Val Val | |
| 1 5 10 15 | |
| Met Lys Ala Leu Trp Lys His Gln Phe Ala Trp Pro Phe Arg Gln Pro | |
| 20 25 30 | |
| Val Asp Ala Val Lys Leu Gly Leu Pro Asp Tyr His Lys Ile Ile Lys | |
| 35 40 45 | |
| Gln Pro Met Asp Met Gly Thr Ile Lys Arg Arg Leu Glu Asn Asn Tyr | |
| 50 55 60 | |
| Tyr Trp Ala Ala Ser Glu Cys Met Gln Asp Phe Asn Thr Met Phe Thr | |
| 65 70 75 80 | |
| Asn Cys Tyr Ile Tyr Asn Lys Pro Thr Asp Asp Ile Val Leu Met Ala | |
| 85 90 95 | |
| Gln Thr Leu Glu Lys Ile Phe Leu Gln Lys Val Ala Ser Met Pro | |
| 100 105 110 | |
| 20 111 PRT Homo sapiens 20 | |
| Lys Pro Gly Arg Lys Thr Asn Gln Leu Gln Tyr Met Gln Asn Val Val | |
| 1 5 10 15 | |
| Val Lys Thr Leu Trp Lys His Gln Phe Ala Trp Pro Phe Tyr Gln Pro | |
| 20 25 30 | |
| Val Asp Ala Ile Lys Leu Asn Leu Pro Asp Tyr His Lys Ile Ile Lys | |
| 35 40 45 | |
| Asn Pro Met Asp Met Gly Thr Ile Lys Lys Arg Leu Glu Asn Asn Tyr | |
| 50 55 60 | |
| Tyr Trp Ser Ala Ser Glu Cys Met Gln Asp Phe Asn Thr Met Phe Thr | |
| 65 70 75 80 | |
| Asn Cys Tyr Ile Tyr Asn Lys Pro Thr Asp Asp Ile Val Leu Met Ala | |
| 85 90 95 | |
| Gln Ala Leu Glu Lys Ile Phe Leu Gln Lys Val Ala Gln Met Pro | |
| 100 105 110 | |
| 21 111 PRT Drosophila melanogaster 21 | |
| Arg Pro Gly Arg Asn Thr Asn Gln Leu Gln Tyr Leu Ile Lys Thr Val | |
| 1 5 10 15 | |
| Met Lys Val Ile Trp Lys His His Phe Ser Trp Pro Phe Gln Gln Pro | |
| 20 25 30 | |
| Val Asp Ala Lys Lys Leu Asn Leu Pro Asp Tyr His Lys Ile Ile Lys | |
| 35 40 45 | |
| Gln Pro Met Asp Met Gly Thr Ile Lys Lys Arg Leu Glu Asn Asn Tyr | |
| 50 55 60 | |
| Tyr Trp Ser Ala Lys Glu Thr Ile Gln Asp Phe Asn Thr Met Phe Asn | |
| 65 70 75 80 | |
| Asn Cys Tyr Val Tyr Asn Lys Pro Gly Glu Asp Val Val Val Met Ala | |
| 85 90 95 | |
| Gln Thr Leu Glu Lys Val Phe Leu Gln Lys Ile Glu Ser Met Pro | |
| 100 105 110 | |
| 22 109 PRT Saccharomyces cerevisiae 22 | |
| Asn Pro Ile Pro Lys His Gln Gln Lys His Ala Leu Leu Ala Ile Lys | |
| 1 5 10 15 | |
| Ala Val Lys Arg Leu Lys Asp Ala Arg Pro Phe Leu Gln Pro Val Asp | |
| 20 25 30 | |
| Pro Val Lys Leu Asp Ile Pro Phe Tyr Phe Asn Tyr Ile Lys Arg Pro | |
| 35 40 45 | |
| Met Asp Leu Ser Thr Ile Glu Arg Lys Leu Asn Val Gly Ala Tyr Glu | |
| 50 55 60 | |
| Val Pro Glu Gln Ile Thr Glu Asp Phe Asn Leu Met Val Asn Asn Ser | |
| 65 70 75 80 | |
| Ile Lys Phe Asn Gly Pro Asn Ala Gly Ile Ser Gln Met Ala Arg Asn | |
| 85 90 95 | |
| Ile Gln Ala Ser Phe Glu Lys His Met Leu Asn Met Pro | |
| 100 105 | |
| 23 113 PRT Homo sapiens 23 | |
| Lys Lys Gly Lys Leu Ser Glu Gln Leu Lys His Cys Asn Gly Ile Leu | |
| 1 5 10 15 | |
| Lys Glu Leu Leu Ser Lys Lys His Ala Ala Tyr Ala Trp Pro Phe Tyr | |
| 20 25 30 | |
| Lys Pro Val Asp Ala Ser Ala Leu Gly Leu His Asp Tyr His Asp Ile | |
| 35 40 45 | |
| Ile Lys His Pro Met Asp Leu Ser Thr Val Lys Arg Lys Met Glu Asn | |
| 50 55 60 | |
| Arg Asp Tyr Arg Asp Ala Gln Glu Phe Ala Ala Asp Val Arg Leu Met | |
| 65 70 75 80 | |
| Phe Ser Asn Cys Tyr Lys Tyr Asn Pro Pro Asp His Asp Val Val Ala | |
| 85 90 95 | |
| Met Ala Arg Lys Leu Gln Asp Val Phe Glu Phe Arg Tyr Ala Lys Met | |
| 100 105 110 | |
| Pro 24 113 PRT Homo sapiens 24 | |
| Lys Lys Gly Lys Leu Ser Glu His Leu Arg Tyr Cys Asp Ser Ile Leu | |
| 1 5 10 15 | |
| Arg Glu Met Leu Ser Lys Lys His Ala Ala Tyr Ala Trp Pro Phe Tyr | |
| 20 25 30 | |
| Lys Pro Val Asp Ala Glu Ala Leu Glu Leu His Asp Tyr His Asp Ile | |
| 35 40 45 | |
| Ile Lys His Pro Met Asp Leu Ser Thr Val Lys Arg Lys Met Asp Gly | |
| 50 55 60 | |
| Arg Glu Tyr Pro Asp Ala Gln Gly Phe Ala Ala Asp Val Arg Leu Met | |
| 65 70 75 80 | |
| Phe Ser Asn Cys Tyr Lys Tyr Asn Pro Pro Asp His Glu Val Val Ala | |
| 85 90 95 | |
| Met Ala Arg Lys Leu Gln Asp Val Phe Glu Met Arg Phe Ala Lys Met | |
| 100 105 110 | |
| Pro 25 113 PRT Drosophila melanogaster 25 | |
| Asn Lys Glu Lys Leu Ser Asp Ala Leu Lys Ser Cys Asn Glu Ile Leu | |
| 1 5 10 15 | |
| Lys Glu Leu Phe Ser Lys Lys His Ser Gly Tyr Ala Trp Pro Phe Tyr | |
| 20 25 30 | |
| Lys Pro Val Asp Ala Glu Met Leu Gly Leu His Asp Tyr His Asp Ile | |
| 35 40 45 | |
| Ile Lys Lys Pro Met Asp Leu Gly Thr Val Lys Arg Lys Met Asp Asn | |
| 50 55 60 | |
| Arg Glu Tyr Lys Ser Ala Pro Glu Phe Ala Ala Asp Val Arg Leu Ile | |
| 65 70 75 80 | |
| Phe Thr Asn Cys Tyr Lys Tyr Asn Pro Pro Asp His Asp Val Val Ala | |
| 85 90 95 | |
| Met Gly Arg Lys Leu Gln Asp Val Phe Glu Met Arg Tyr Ala Asn Ile | |
| 100 105 110 | |
| Pro 26 113 PRT Saccharomyces cerevisiae 26 | |
| Lys Ser Lys Arg Leu Gln Gln Ala Met Lys Phe Cys Gln Ser Val Leu | |
| 1 5 10 15 | |
| Lys Glu Leu Met Ala Lys Lys His Ala Ser Tyr Asn Tyr Pro Phe Leu | |
| 20 25 30 | |
| Glu Pro Val Asp Pro Val Ser Met Asn Leu Pro Thr Tyr Phe Asp Tyr | |
| 35 40 45 | |
| Val Lys Glu Pro Met Asp Leu Gly Thr Ile Ala Lys Lys Leu Asn Asp | |
| 50 55 60 | |
| Trp Gln Tyr Gln Thr Met Glu Asp Phe Glu Arg Glu Val Arg Leu Val | |
| 65 70 75 80 | |
| Phe Lys Asn Cys Tyr Thr Phe Asn Pro Asp Gly Thr Ile Val Asn Met | |
| 85 90 95 | |
| Met Gly His Arg Leu Glu Glu Val Phe Asn Ser Lys Trp Ala Asp Arg | |
| 100 105 110 | |
| Pro 27 108 PRT Homo sapiens 27 | |
| Met Glu Met Gln Leu Thr Pro Phe Leu Ile Leu Leu Arg Lys Thr Leu | |
| 1 5 10 15 | |
| Glu Gln Leu Gln Glu Lys Asp Thr Gly Asn Ile Phe Ser Glu Pro Val | |
| 20 25 30 | |
| Pro Leu Ser Glu Val Pro Asp Tyr Leu Asp His Ile Lys Lys Pro Met | |
| 35 40 45 | |
| Asp Phe Phe Thr Met Lys Gln Asn Leu Glu Ala Tyr Arg Tyr Leu Asn | |
| 50 55 60 | |
| Phe Asp Asp Phe Glu Glu Asp Phe Asn Leu Ile Val Ser Asn Cys Leu | |
| 65 70 75 80 | |
| Lys Tyr Asn Ala Lys Asp Thr Ile Phe Tyr Arg Ala Ala Val Arg Leu | |
| 85 90 95 | |
| Arg Glu Gln Gly Gly Ala Val Val Arg Gln Ala Arg | |
| 100 105 | |
| 28 113 PRT Homo sapiens 28 | |
| Ser Glu Asp Gln Glu Ala Ile Gln Ala Gln Lys Ile Trp Lys Lys Ala | |
| 1 5 10 15 | |
| Ile Met Leu Val Trp Arg Ala Ala Ala Asn His Arg Tyr Ala Asn Val | |
| 20 25 30 | |
| Phe Leu Gln Pro Val Thr Asp Asp Ile Ala Pro Gly Tyr His Ser Ile | |
| 35 40 45 | |
| Val Gln Arg Pro Met Asp Leu Ser Thr Ile Lys Lys Asn Ile Glu Asn | |
| 50 55 60 | |
| Gly Leu Ile Arg Ser Thr Ala Glu Phe Gln Arg Asp Ile Met Leu Met | |
| 65 70 75 80 | |
| Phe Gln Asn Ala Val Met Tyr Asn Ser Ser Asp His Asp Val Tyr His | |
| 85 90 95 | |
| Met Ala Val Glu Met Gln Arg Asp Val Leu Glu Gln Ile Gln Gln Phe | |
| 100 105 110 | |
| Leu 29 106 PRT Gallus gallus 29 | |
| Asn Leu Pro Thr Val Asp Pro Ile Ala Val Cys His Glu Leu Tyr Asn | |
| 1 5 10 15 | |
| Thr Ile Arg Asp Tyr Lys Asp Glu Gln Gly Arg Leu Leu Cys Glu Leu | |
| 20 25 30 | |
| Phe Ile Arg Ala Pro Lys Arg Arg Asn Gln Pro Asp Tyr Tyr Glu Val | |
| 35 40 45 | |
| Val Ser Gln Pro Ile Asp Leu Met Lys Ile Gln Gln Lys Leu Lys Met | |
| 50 55 60 | |
| Glu Glu Tyr Asp Asp Val Asn Val Leu Thr Ala Asp Phe Gln Leu Leu | |
| 65 70 75 80 | |
| Phe Asn Asn Ala Lys Ala Tyr Tyr Lys Pro Asp Ser Pro Glu Tyr Lys | |
| 85 90 95 | |
| Ala Ala Cys Lys Leu Trp Glu Leu Tyr Leu | |
| 100 105 | |
| 30 112 PRT Gallus gallus 30 | |
| Ser Ser Pro Gly Tyr Leu Lys Glu Ile Leu Glu Gln Leu Leu Glu Ala | |
| 1 5 10 15 | |
| Val Ala Val Ala Thr Asn Pro Ser Gly Arg Leu Ile Ser Glu Leu Phe | |
| 20 25 30 | |
| Gln Lys Leu Pro Ser Lys Val Gln Tyr Pro Asp Tyr Tyr Ala Ile Ile | |
| 35 40 45 | |
| Lys Glu Pro Ile Asp Leu Lys Thr Ile Ala Gln Arg Ile Gln Asn Gly | |
| 50 55 60 | |
| Thr Tyr Lys Ser Ile His Ala Met Ala Lys Asp Ile Asp Leu Leu Ala | |
| 65 70 75 80 | |
| Lys Asn Ala Lys Thr Tyr Asn Glu Pro Gly Ser Gln Val Phe Lys Asp | |
| 85 90 95 | |
| Ala Asn Ala Ile Lys Lys Ile Phe Asn Met Lys Lys Ala Glu Ile Glu | |
| 100 105 110 | |
| 31 112 PRT Gallus gallus 31 | |
| Thr Ser Phe Met Asp Thr Ser Asn Pro Leu Tyr Gln Leu Tyr Asp Thr | |
| 1 5 10 15 | |
| Val Arg Ser Cys Arg Asn Asn Gln Gly Gln Leu Ile Ser Glu Pro Phe | |
| 20 25 30 | |
| Phe Gln Leu Pro Ser Lys Lys Lys Tyr Pro Asp Tyr Tyr Gln Gln Ile | |
| 35 40 45 | |
| Lys Thr Pro Ile Ser Leu Gln Gln Ile Arg Ala Lys Leu Lys Asn His | |
| 50 55 60 | |
| Glu Tyr Glu Thr Leu Asp Gln Leu Glu Ala Asp Leu Asn Leu Met Phe | |
| 65 70 75 80 | |
| Glu Asn Ala Lys Arg Tyr Asn Val Pro Asn Ser Ala Ile Tyr Lys Arg | |
| 85 90 95 | |
| Val Leu Lys Met Gln Gln Val Met Gln Ala Lys Lys Lys Glu Leu Ala | |
| 100 105 110 | |
| 32 113 PRT Gallus gallus 32 | |
| Ser Lys Lys Asn Met Arg Lys Gln Arg Met Lys Ile Leu Tyr Asn Ala | |
| 1 5 10 15 | |
| Val Leu Glu Ala Arg Glu Ser Gly Thr Gln Arg Arg Leu Cys Asp Leu | |
| 20 25 30 | |
| Phe Met Val Lys Pro Ser Lys Lys Asp Tyr Pro Asp Tyr Tyr Lys Ile | |
| 35 40 45 | |
| Ile Leu Glu Pro Met Asp Leu Lys Met Ile Glu His Asn Ile Arg Asn | |
| 50 55 60 | |
| Asp Lys Tyr Val Gly Glu Glu Ala Met Ile Asp Asp Met Lys Leu Met | |
| 65 70 75 80 | |
| Phe Arg Asn Ala Arg His Tyr Asn Glu Glu Gly Ser Gln Val Tyr Asn | |
| 85 90 95 | |
| Asp Ala His Met Leu Glu Lys Ile Leu Lys Glu Lys Arg Lys Glu Leu | |
| 100 105 110 | |
| Gly 33 115 PRT Gallus gallus 33 | |
| Lys Lys Ser Lys Tyr Met Thr Pro Met Gln Gln Lys Leu Asn Glu Val | |
| 1 5 10 15 | |
| Tyr Glu Ala Val Lys Asn Tyr Thr Asp Lys Arg Gly Arg Arg Leu Ser | |
| 20 25 30 | |
| Ala Ile Phe Leu Arg Leu Pro Ser Arg Ser Glu Leu Pro Asp Tyr Tyr | |
| 35 40 45 | |
| Ile Thr Ile Lys Lys Pro Val Asp Met Glu Lys Ile Arg Ser His Met | |
| 50 55 60 | |
| Met Ala Asn Lys Tyr Gln Asp Ile Asp Ser Met Val Glu Asp Phe Val | |
| 65 70 75 80 | |
| Met Met Phe Asn Asn Ala Cys Thr Tyr Asn Glu Pro Glu Ser Leu Ile | |
| 85 90 95 | |
| Tyr Lys Asp Ala Leu Val Leu His Lys Val Leu Leu Glu Thr Arg Arg | |
| 100 105 110 | |
| Glu Ile Glu | |
| 115 | |
| 34 112 PRT Unknown Organism Description of Unknown Organism see | |
| Jeanmouginet al., Trends in Biochem. Sci. 22151-153 (1997) 34 | |
| His Asn Ala Pro Phe Asp Lys Thr Lys Phe Asp Glu Val Leu Glu Ala | |
| 1 5 10 15 | |
| Leu Val Gly Leu Lys Asp Asn Glu Gly Asn Pro Phe Asp Asp Ile Phe | |
| 20 25 30 | |
| Glu Glu Leu Pro Ser Lys Arg Tyr Phe Pro Asp Tyr Tyr Gln Ile Ile | |
| 35 40 45 | |
| Gln Lys Pro Ile Cys Tyr Lys Met Met Arg Asn Lys Ala Lys Thr Gly | |
| 50 55 60 | |
| Lys Tyr Leu Ser Met Gly Asp Phe Tyr Asp Asp Ile Arg Leu Met Val | |
| 65 70 75 80 | |
| Ser Asn Ala Gln Thr Tyr Asn Met Pro Gly Ser Leu Val Tyr Glu Cys | |
| 85 90 95 | |
| Ser Val Leu Ile Ala Asn Thr Ala Asn Ser Leu Glu Ser Lys Asp Gly | |
| 100 105 110 | |
| 35 113 PRT Unknown Organism Description of Unknown Organism see | |
| Jeanmougin et al., Trends in Biochem. Sci. 22151-153 (1997) 35 | |
| Gly Thr Asn Glu Ile Asp Val Pro Lys Val Ile Gln Asn Ile Leu Asp | |
| 1 5 10 15 | |
| Ala Leu His Glu Glu Lys Asp Glu Gln Gly Arg Phe Leu Ile Asp Ile | |
| 20 25 30 | |
| Phe Ile Asp Leu Pro Ser Lys Arg Leu Tyr Pro Asp Tyr Tyr Glu Ile | |
| 35 40 45 | |
| Ile Lys Ser Pro Met Thr Ile Lys Met Leu Glu Lys Arg Phe Lys Lys | |
| 50 55 60 | |
| Gly Glu Tyr Thr Thr Leu Glu Ser Phe Val Lys Asp Leu Asn Gln Met | |
| 65 70 75 80 | |
| Phe Ile Asn Ala Lys Thr Tyr Asn Ala Pro Gly Ser Phe Val Tyr Glu | |
| 85 90 95 | |
| Asp Ala Glu Lys Leu Ser Gln Leu Ser Ser Ser Leu Ile Ser Ser Phe | |
| 100 105 110 | |
| Ser 36 113 PRT Homo sapiens 36 | |
| Gly Thr Asn Glu Ile Asp Val Pro Lys Val Ile Gln Asn Ile Leu Asp | |
| 1 5 10 15 | |
| Ala Leu His Glu Glu Lys Asp Glu Gln Gly Arg Phe Leu Ile Asp Ile | |
| 20 25 30 | |
| Phe Ile Asp Leu Pro Ser Lys Arg Leu Tyr Pro Asp Tyr Tyr Glu Ile | |
| 35 40 45 | |
| Ile Lys Ser Pro Met Thr Ile Lys Met Leu Glu Lys Arg Phe Lys Lys | |
| 50 55 60 | |
| Gly Glu Tyr Thr Thr Leu Glu Ser Phe Val Lys Asp Leu Asn Gln Met | |
| 65 70 75 80 | |
| Phe Ile Asn Ala Lys Thr Tyr Asn Ala Pro Gly Ser Phe Val Tyr Glu | |
| 85 90 95 | |
| Asp Ala Glu Lys Leu Ser Gln Leu Ser Ser Ser Leu Ile Ser Ser Phe | |
| 100 105 110 | |
| Ser 37 114 PRT Homo sapiens 37 | |
| Ser Pro Asn Pro Pro Asn Leu Thr Lys Lys Met Lys Lys Ile Val Asp | |
| 1 5 10 15 | |
| Ala Val Ile Lys Tyr Lys Asp Ser Ser Ser Gly Arg Gln Leu Ser Glu | |
| 20 25 30 | |
| Val Phe Ile Gln Leu Pro Ser Arg Lys Glu Leu Pro Glu Tyr Tyr Glu | |
| 35 40 45 | |
| Leu Ile Arg Lys Pro Val Asp Phe Lys Lys Ile Lys Glu Arg Ile Arg | |
| 50 55 60 | |
| Asn His Lys Tyr Arg Ser Leu Asn Asp Leu Glu Lys Asp Val Met Leu | |
| 65 70 75 80 | |
| Leu Cys Gln Asn Ala Gln Thr Phe Asn Leu Glu Gly Ser Leu Ile Tyr | |
| 85 90 95 | |
| Glu Asp Ser Ile Val Leu Gln Ser Val Phe Thr Ser Val Arg Gln Lys | |
| 100 105 110 | |
| Ile Glu 38 113 PRT Gallus gallus 38 | |
| Ser Pro Asn Pro Pro Lys Leu Thr Lys Gln Met Asn Ala Ile Ile Asp | |
| 1 5 10 15 | |
| Thr Val Ile Asn Tyr Lys Asp Ser Ser Gly Arg Gln Leu Ser Glu Val | |
| 20 25 30 | |
| Phe Ile Gln Leu Pro Ser Arg Lys Glu Leu Pro Glu Tyr Tyr Glu Leu | |
| 35 40 45 | |
| Ile Arg Lys Pro Val Asp Phe Lys Lys Ile Lys Glu Arg Ile Arg Asn | |
| 50 55 60 | |
| His Lys Tyr Arg Ser Leu Gly Asp Leu Glu Lys Asp Val Met Leu Leu | |
| 65 70 75 80 | |
| Cys His Asn Ala Gln Thr Phe Asn Leu Glu Gly Ser Gln Ile Tyr Glu | |
| 85 90 95 | |
| Asp Ser Ile Val Leu Gln Ser Val Phe Lys Ser Ala Arg Gln Lys Ile | |
| 100 105 110 | |
| Ala 39 114 PRT Gallus gallus 39 | |
| Ser Pro Asn Pro Pro Asn Leu Thr Lys Lys Met Lys Lys Ile Val Asp | |
| 1 5 10 15 | |
| Ala Val Ile Lys Tyr Lys Asp Ser Ser Ser Gly Arg Gln Leu Ser Glu | |
| 20 25 30 | |
| Val Phe Ile Gln Leu Pro Ser Arg Lys Glu Leu Pro Glu Tyr Tyr Glu | |
| 35 40 45 | |
| Leu Ile Arg Lys Pro Val Asp Phe Lys Lys Ile Lys Glu Arg Ile Arg | |
| 50 55 60 | |
| Asn His Lys Tyr Arg Ser Leu Asn Asp Leu Glu Lys Asp Val Met Leu | |
| 65 70 75 80 | |
| Leu Cys Gln Asn Ala Gln Thr Phe Asn Leu Glu Val Ser Leu Ile Tyr | |
| 85 90 95 | |
| Glu Asp Ser Ile Val Leu Gln Ser Val Phe Thr Ser Val Arg Gln Lys | |
| 100 105 110 | |
| Ile Glu 40 105 PRT Homo sapiens 40 | |
| Ala Lys Leu Ser Pro Ala Asn Gln Arg Lys Cys Glu Arg Val Leu Leu | |
| 1 5 10 15 | |
| Ala Leu Phe Cys His Glu Pro Cys Arg Pro Leu His Gln Leu Ala Thr | |
| 20 25 30 | |
| Asp Ser Thr Phe Ser Leu Asp Gln Pro Gly Gly Thr Leu Asp Leu Thr | |
| 35 40 45 | |
| Leu Ile Arg Ala Arg Leu Gln Glu Lys Leu Ser Pro Pro Tyr Ser Ser | |
| 50 55 60 | |
| Pro Gln Glu Phe Ala Gln Asp Val Gly Arg Met Phe Lys Gln Phe Asn | |
| 65 70 75 80 | |
| Lys Leu Thr Glu Asp Lys Ala Asp Val Gln Ser Ile Ile Gly Leu Gln | |
| 85 90 95 | |
| Arg Phe Phe Glu Thr Arg Met Asn Glu | |
| 100 105 | |
| 41 105 PRT Mus musculus 41 | |
| Ala Lys Leu Ser Pro Ala Asn Gln Arg Lys Cys Glu Arg Val Leu Leu | |
| 1 5 10 15 | |
| Ala Leu Phe Cys His Glu Pro Cys Arg Pro Leu His Gln Leu Ala Thr | |
| 20 25 30 | |
| Asp Ser Thr Phe Ser Met Glu Gln Pro Gly Gly Thr Leu Asp Leu Thr | |
| 35 40 45 | |
| Leu Ile Arg Ala Arg Leu Gln Glu Lys Leu Ser Pro Pro Tyr Ser Ser | |
| 50 55 60 | |
| Pro Gln Glu Phe Ala Gln Asp Val Gly Arg Met Phe Lys Gln Phe Asn | |
| 65 70 75 80 | |
| Lys Leu Thr Glu Asp Lys Ala Asp Val Gln Ser Ile Ile Gly Leu Gln | |
| 85 90 95 | |
| Arg Phe Phe Glu Thr Arg Met Asn Asp | |
| 100 105 | |
| 42 108 PRT Mus sp. 42 | |
| Thr Lys Leu Thr Pro Ile Asp Lys Arg Lys Cys Glu Arg Leu Leu Leu | |
| 1 5 10 15 | |
| Phe Leu Tyr Cys His Glu Met Ser Leu Ala Phe Gln Asp Pro Val Pro | |
| 20 25 30 | |
| Leu Thr Val Pro Asp Tyr Tyr Lys Ile Ile Lys Asn Pro Met Asp Leu | |
| 35 40 45 | |
| Ser Thr Ile Lys Lys Arg Leu Gln Glu Asp Tyr Cys Met Tyr Thr Lys | |
| 50 55 60 | |
| Pro Glu Asp Phe Val Ala Asp Phe Arg Leu Ile Phe Gln Asn Cys Ala | |
| 65 70 75 80 | |
| Glu Phe Asn Glu Pro Asp Ser Glu Val Ala Asn Ala Gly Ile Lys Leu | |
| 85 90 95 | |
| Glu Ser Tyr Phe Glu Glu Leu Leu Lys Asn Leu Tyr | |
| 100 105 | |
| 43 27 PRT Artificial Sequence synthetic bromodomain peptide 43 | |
| Xaa Xaa Phe Xaa Xaa Xaa Pro Xaa Xaa Xaa Xaa Xaa Xaa Xaa Xaa Xaa | |
| 1 5 10 15 | |
| Xaa Tyr Xaa Xaa Xaa Xaa Xaa Xaa Pro Xaa Asp | |
| 20 25 | |
| 44 20 PRT Artificial Sequence synthetic bromodomain peptide 44 | |
| Trp Pro Phe Met Glu Pro Val Lys Arg Thr Glu Ala Pro Gly Tyr Tyr | |
| 1 5 10 15 | |
| Glu Val Ile Arg | |
| 20 | |
| 45 101 PRT Human immunodeficiency virus type 1 Tat protein 45 | |
| Met Glu Pro Val Asp Pro Arg Leu Glu Pro Trp Lys His Pro Gly Ser | |
| 1 5 10 15 | |
| Gln Pro Lys Thr Ala Ser Asn Asn Cys Tyr Cys Lys Arg Cys Cys Leu | |
| 20 25 30 | |
| His Cys Gln Val Cys Phe Thr Lys Lys Gly Leu Gly Ile Ser Tyr Gly | |
| 35 40 45 | |
| Arg Lys Lys Arg Arg Gln Arg Arg Arg Ala Pro Gln Asp Ser Lys Thr | |
| 50 55 60 | |
| His Gln Val Ser Leu Ser Lys Gln Pro Ala Ser Gln Pro Arg Gly Asp | |
| 65 70 75 80 | |
| Pro Thr Gly Pro Lys Glu Ser Lys Lys Lys Val Glu Arg Glu Thr Glu | |
| 85 90 95 | |
| Thr Asp Pro Glu Asp | |
| 100 | |
| 46 9 PRT Artificial Sequence Synthetic HIV-1 Tat peptide 46 | |
| Tyr Gly Arg Lys Xaa Xaa Xaa Arg Gln | |
| 1 5 | |
| 47 10 PRT Artificial Sequence synthetic HIV-1 Tat peptide 47 | |
| Ser Tyr Gly Arg Lys Lys Arg Arg Gln Arg | |
| 1 5 10 | |
| 48 21 PRT Artificial Sequence Synthetic HIV-1 Tat peptide 48 | |
| Phe Xaa Xaa Xaa Xaa Val Xaa Xaa Xaa Xaa Glu Xaa Xaa Xaa Xaa Tyr | |
| 1 5 10 15 | |
| Xaa Xaa Xaa Val Xaa | |
| 20 | |
| 49 62 PRT Artificial Sequence synthetic bromodomain peptide 49 | |
| Phe Met Glu Pro Val Lys Arg Thr Glu Ala Pro Gly Tyr Tyr Glu Val | |
| 1 5 10 15 | |
| Ile Arg Phe Pro Met Asp Leu Lys Thr Met Ser Glu Arg Leu Lys Asn | |
| 20 25 30 | |
| Arg Tyr Tyr Val Ser Lys Lys Leu Phe Met Ala Asp Leu Gln Arg Val | |
| 35 40 45 | |
| Phe Thr Asn Cys Lys Glu Tyr Asn Ala Ala Glu Ser Glu Tyr | |
| 50 55 60 | |
| 50 11 PRT Artificial Sequence synthetic HIV-1 Tat peptide 50 | |
| Ser Tyr Gly Arg Xaa Lys Arg Arg Gln Arg Cys | |
| 1 5 10 | |
| 51 11 PRT Artificial Sequence synthetic HIV-1 Tat peptide 51 | |
| Ser Ala Gly Arg Xaa Lys Arg Arg Gln Arg Cys | |
| 1 5 10 | |
| 52 11 PRT Artificial Sequence synthetic HIV-1 Tat peptide 52 | |
| Ser Tyr Gly Ala Xaa Lys Arg Arg Gln Arg Cys | |
| 1 5 10 | |
| 53 11 PRT Artificial Sequence synthetic HIV-1 Tat peptide 53 | |
| Ser Tyr Gly Arg Xaa Ala Arg Arg Gln Arg Cys | |
| 1 5 10 | |
| 54 11 PRT Artificial Sequence synthetic HIV-1 Tat peptide 54 | |
| Ser Tyr Gly Arg Xaa Lys Ala Arg Gln Arg Cys | |
| 1 5 10 | |
| 55 11 PRT Artificial Sequence synthetic HIV-1 Tat peptide 55 | |
| Ser Tyr Gly Arg Xaa Lys Arg Ala Gln Arg Cys | |
| 1 5 10 | |
| 56 11 PRT Artificial Sequence synthetic HIV-1 Tat peptide 56 | |
| Ser Tyr Gly Arg Xaa Lys Arg Arg Ala Arg Cys | |
| 1 5 10 | |
| 57 11 PRT Artificial Sequence synthetic HIV-1 Tat peptide 57 | |
| Ser Tyr Gly Arg Lys Xaa Arg Arg Gln Arg Cys | |
| 1 5 10 | |
| 58 11 PRT Artificial Sequence synthetic HIV-1 Tat peptide 58 | |
| Thr Asn Cys Tyr Cys Lys Xaa Cys Cys Phe His | |
| 1 5 10 | |
| 59 20 PRT Artificial Sequence synthetic histone H4 AcK16 | |
| peptide 59 | |
| Ser Gly Arg Gly Lys Gly Gly Lys Gly Leu Gly Lys Gly Gly Ala Xaa | |
| 1 5 10 15 | |
| Arg His Arg Lys | |
| 20 | |
| 60 11 PRT Artificial Sequence synthetic HIV-1 Tat peptide 60 | |
| Ser Tyr Gly Arg Lys Lys Arg Arg Gln Arg Cys | |
| 1 5 10 | |
| 61 6 PRT Artificial Sequence hexahistidine tag 61 | |
| His His His His His His | |
| 1 5 | |
| 62 12 PRT Artificial Sequence synthetic peptide 62 | |
| Ser Gly Arg Gly Lys Gly Gly Xaa Gly Leu Gly Lys | |
| 1 5 10 | |
| 63 12 PRT Artificial Sequence synthetic peptide 63 | |
| Arg Lys Ser Thr Gly Gly Xaa Ala Pro Arg Lys Gln | |
| 1 5 10 | |

What is claimed is:

1. A method of inhibiting Tat-P/CAF interaction comprising administering an agent that inhibits the binding of p300/CRP-associated factor (P/CAF) and transcriptional activation protein (Tat) and/or destabilizes the Tat-P/CAF complex.

2. The method of claim 1, wherein the agent is a peptidic acetyl lysine analog or a small molecule.

3. A method of inhibiting Tat-P/CAF interaction according to claim 1 further comprising comprising administering an agent that modulates the stability of a binding complex formed between a p300/CBP-associating factor (P/CAF) and trans-activator protein (Tat) identified by rational drug design with a set of atomic coordinates obtained from one or more of Tables 1-5 and 10-14 of U.S. Pat. No. 7,589,167 in conjunction with computer modeling by:
(a) contacting a bromodomain of P/CAF or a fragment thereof with a binding partner in the presence of the acetyl lysine analog, wherein the bromodomain of P/CAF and the binding partner bind in the absence of the compound; and wherein the binding partner is selected from the group consisting of Tat that is acetylated at the lysine residue at position 50 of SEQ ID NO:45, a fragment of Tat comprising an acetyl-lysine at position 50, and an analog of the fragment of Tat comprising an acetyl-lysine at position 50; and
(b) measuring the stability of the binding complex between the bromodomain of P/CAF or a fragment thereof and the binding partner; wherein a compound is identified as a compound that modulates the stability of the Tat-P/CAF complex when there is a change in the stability of the binding complex between the bromodomain of P/CAF or a fragment thereof and the binding partner in the presence of the compound.

4. A method of inhibiting Tat-P/CAF interaction according to claim 1 wherein the agent modulates the affinity of P/CAF for Tat.

5. The method of claim 1, wherein the agent is selected from the group consisting of SEQ ID NO: 4, SEQ ID NO: 5 and SEQ ID NO: 6.

6. The method of claim 1, wherein the agent is selected from the group consisting of SEQ ID NOs: 46 and 47.

7. A method for inhibiting the binding of bromodomains to acetyl-lysine residues of proteins comprising the step of administering a therapeutically effective amount of a compound of the following general formula (I) 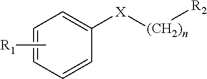
wherein R¹ is selected from the group consisting of hydrogen, lower alkyl, aryl, aralkyl;
substituted aralkyl, heteroaryl; substituted heteroaryl, phenyl, SO₂, NH₂, NO₂, SO₂, CH₃, CH₂CH₃, OCH₃, OCOCH₃, CH₂COCH₃, OH, CN and halogen;
R² is selected from the group consisting of hydrogen; lower alkyl, aryl, phenyl, aralkyl; substituted aralkyl, heteroaryl; substituted heteroaryl, SO₂, NH₂, NH₃⁺NO₂, SO₂, CH₃, CH₂CH₃, OCH₃, OCOCH₃, CH₂COCH₃, OH, halogen, carboxy, and alkoxy;
X is selected from the group consisting of lower alkyl, SO₂, NH, NO₂, O, carboxy, and alkoxy; and
n is an integer from 0 to 10;
and their pharmaceutically acceptable salts of acids or bases.

8. A method of treating viral infection in an individual comprising administering to the individual a compound of Formula I: 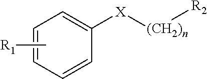
wherein R¹ is selected from the group consisting of hydrogen, lower alkyl, aryl, aralkyl;
substituted aralkyl, heteroaryl; substituted heteroaryl, phenyl, SO₂, NH₂, NO₂, SO₂, CH₃, CH₂CH₃, OCH₃, OCOCH₃, CH₂COCH₃, OH, CN and halogen;
R² is selected from the group consisting of hydrogen; lower alkyl, aryl, phenyl, aralkyl; substituted aralkyl, heteroaryl; substituted heteroaryl, SO₂, NH₂, NH₃⁺NO₂, SO₂, CH₃, CH₂CH₃, OCH₃, OCOCH₃, CH₂COCH₃, OH, halogen, carboxy, and alkoxy;
X is selected from the group consisting of lower alkyl, SO₂, NH, NO₂, O, carboxy, and alkoxy; and
n is an integer from 0 to 10;
and their pharmaceutically acceptable salts of acids or bases.

9. The method of claim 8 wherein the viral infection is HIV infection.

10. A method for inhibiting the binding of bromodomains to acetyl-lysine residues of proteins comprising the step of administering a therapeutically effective amount of a pharmaceutical composition comprising a compound of the following general formula (II) wherein: 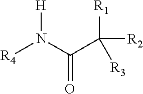
R¹ R² and R³ are independently selected from the group consisting of hydrogen; lower alkyl, aryl, phenyl, aralkyl; substituted aralkyl, heteroaryl; substituted heteroaryl, SO₂, NH₂, NH₃⁺NO₂, SO₂, CH₃, CH₂CH₃, OCH₃, OCOCH₃, CH₂COCH₃, OH, SH, halogen, carboxy, and alkoxy;
R⁴ is selected from the group consisting of lower alkyl, aryl, SO₂, NH, NO₂, CH₃, CH₂CH₃, OCH₃, OCOCH₃, CH₂COCH₃, OH, carboxy, and alkoxy;
and their pharmaceutically acceptable salts of acids or bases.

11. A method according to claim 10 wherein R¹ R² and R³ are independently selected from the group consisting of hydrogen, lower alkyl, NH₃⁺, OH, SH, and halogen.

12. A method according to claim 10 wherein R⁴ isselected from the group consisting of lower alkyl and aryl.

13. A method for treating viral infection comprising the step of administering a therapeutically effective amount of a pharmaceutical composition comprising a compound of the following general formula (H) wherein: 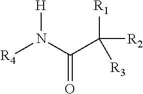
R¹ R² and R³ are independently selected from the group consisting of hydrogen; lower alkyl, aryl, phenyl, aralkyl; substituted aralkyl, heteroaryl; substituted heteroaryl, SO₂, NH₂, NH₃⁺NO₂, SO₂, CH₃, CH₂CH₃, OCH₃, OCOCH₃, CH₂COCH₃, OH, SH, halogen, carboxy, and alkoxy;
R⁴ is selected from the group consisting of lower alkyl, aryl, SO₂, NH, NO₂, CH₃, CH₂CH₃, OCH₃, OCOCH₃, CH₂COCH₃, OH, carboxy, and alkoxy;
and their pharmaceutically acceptable salts of acids or bases.

14. A method according to claim 13 wherein R¹ R² and R³ are independently selected from the group consisting of hydrogen, lower alkyl, NH₃⁺, OH, SH, and halogen.

15. A method according to claim 13 wherein R⁴ is selected from the group consisting of lower alkyl and aryl.

16. A method according to claim 13 wherein the viral infection is HIV infection.

17. A method for inhibiting the binding of bromodomains to acetyl-lysine residues of proteins comprising the step of administering a therapeutically effective amount of a pharmaceutical composition comprising a compound of the following general formula (III) wherein 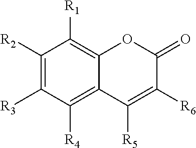
R¹ R², R³, R⁴ R⁵, and R⁶ are independently selected from the group consisting of hydrogen; lower alkyl, aryl, phenyl, aralkyl; substituted aralkyl, heteroaryl, substituted heteroaryl, SO₂, NH₂, NH₃⁺, NO₂, SO₂, CH₃, CH₂CH₃, OCH₃, OCOCH₃, CH₂COCH₃, OCH₂CH₃, OCH(CH₃)₂, OCH₂COOH, OCHCH₃COOH, OCH₂COCH₃, OCH₂CONH₂, OCOCH(CH₃)₂, OCH₂CH₂OH, OCH₂CH₂CH₃, O(CH₂)₃CH₃, OCHCH₃COOCH₃, OCH₂CON(CH₃)₂, NH(CH₂)₃N(CH₃)₂, NH(CH₂)₂N(CH₃)₂, NH(CH₂)₂0H, NH(CH₂)₃CH₃, NHCH₃, SH, halogen, carboxy, and alkoxy.

18. A method according to claim 17 wherein R¹ and R⁴ are selected from the group consisting of hydrogen and OH; R² is selected from the group consisting of hydrogen, OH, and CH₃, CH₂CH₃, OCH₃, OCOCH₃, CH₂COCH₃, OCH₂CH₃, OCH(CH₃)₂, OCH₂COOH, OCHCH₃COOH, OCH₂COCH₃, OCH₂CONH₂, OCOCH(CH₃)₂, OCH₂CH₂OH, OCH₂CH₂CH₃, O(CH₂)₃CH₃, OCHCH₃COOCH₃, OCH₂CON(CH₃)₂; R³ is selected from the group consisting of hydrogen, OCH₂CH₃, and NHCOCH₃; R⁵ is selected from the group consisting of hydrogen, lower alkyl, aryl, phenyl, aralkyl, NH(CH₂)₃N(CH₃)₂, NH(CH₂)₂N(CH₃)₂, NH(CH₂)₂OH, NH(CH₂)₃CH₃, and NHCH₃; and R⁶ is selected from the group consisting of hydrogen, and NH₂.

19. A method for treating a viral infection comprising the step of administering a therapeutically effective amount of a pharmaceutical composition comprising a compound of the following general formula (III) wherein 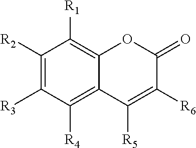
R¹ R², R³, R⁴ R⁵, and R⁶ are independently selected from the group consisting of hydrogen; lower alkyl, aryl, phenyl, aralkyl; substituted aralkyl, heteroaryl, substituted heteroaryl, SO₂, NH_{2,} NH₃⁺, NO₂, SO₂, CH₃, CH₂CH₃, OCH₃, OCOCH₃, CH₂COCH₃, OCH₂CH₃, OCH(CH₃)₂, OCH₂COOH, OCHCH₃COOH, OCH₂COCH₃, OCH₂CONH₂, OCOCH(CH₃)₂, OCH₂CH₂OH, OCH₂CH₂CH₃, O(CH₂)₃CH₃, OCHCH₃COOCH₃, OCH₂CON(CH₃)₂, NH(CH₂)₃N(CH₃)₂, NH(CH₂)₂N(CH₃)₂, NH(CH₂)₂OH, NH(CH₂)₃CH₃, NHCH₃, SH, halogen, carboxy, and alkoxy.

20. A method according to claim 19 wherein R¹ and R⁴ are selected from the group consisting of hydrogen and OH; R² is selected from the group consisting of hydrogen, OH, and CH₃, CH₂CH₃, OCH₃, OCOCH₃, CH₂COCH₃, OCH₂CH₃, OCH(CH₃)₂, OCH₂COOH, OCHCH₃COOH, OCH₂COCH₃, OCH₂CONH₂, OCOCH(CH₃)₂, OCH₂CH₂OH, OCH₂CH₂CH₃, O(CH₂)₃CH₃, OCHCH₃COOCH₃, OCH₂CON(CH₃)₂; R³ is selected from the group consisting of hydrogen, OCH₂CH₃, and NHCOCH₃; R⁵ is selected from the group consisting of hydrogen, lower alkyl, aryl, phenyl, aralkyl, NH(CH₂)₃N(CH₃)₂,NH(CH₂)₂N(CH₃)₂, NH(CH₂)₂OH, NH(CH₂)₃CH₃, and NHCH₃; and R⁶ is selected from the group consisting of hydrogen, and NH₂.

21. A method according to claim 19 wherein the viral infection is HIV infection.

22. A method for treating a viral infection comprising the step of administering a therapeutically effective amount of a pharmaceutical composition comprising a compound selected from the group consisting of

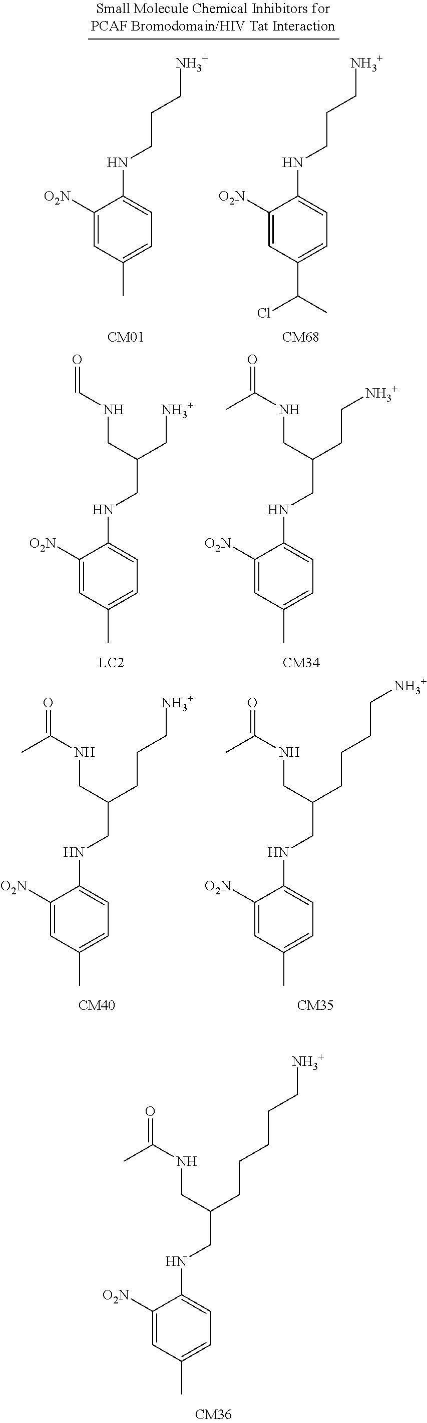

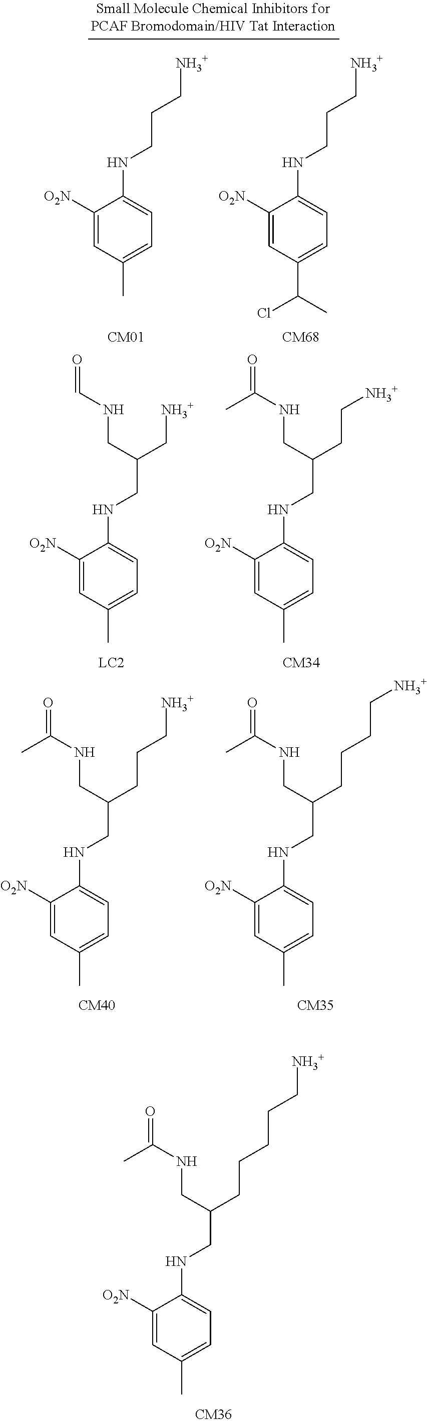

23. A method according to claim 22 wherein the viral infection is HIV infection.

24. A method for inhibiting the binding of bromodomains to acetyl-lysine residues of proteins comprising the step of administering a therapeutically effective amount of a pharmaceutical composition comprising a compound selected from the group consisting of [chemical structure]
